(19) United States
(12) Patent Application Publication
(10) Pub. No.: US 2006/0128729 A1
(43) Pub. Date: Jun. 15, 2006

(54) Novel bicyclic heterocyclic compounds, process for their preparation and compositions containing them

(76) Inventors: Manojit Pal, Hyderabad (IN); Yeleswarapu Koteswar Rao, Hyderabad (IN); Ish Khanna, Alpharetta, GA (US); Nalivela Kumara Swamy, Hyderabad (IN); Venkataraman Subramanian, Chicago, IL (US); Venkateswara Rao Batchu, Hyderabad (IN); Javed Iqbal, Hyderabad (IN); Sivaram Pillarisetti, Norcross, GA (US)

Correspondence Address: Att: David E. Wigley, Ph.D.;Womble Carlyle Sandridge & Rice, PLLC, P.O. Box 7037, Atlanta, GA, 30357-0037, US

(21) Appl. No.: 11/285,982

(22) Filed: Nov. 23, 2005

Related U.S. Application Data
(60) Provisional application No. 60/630,604, filed on Nov. 23, 2004.

(51) Int. Cl. A61K 31/519; C07D 487/02

(52) U.S. Cl. 514/262.1; 544/262

(57) ABSTRACT

The present invention provides, among other things, new bicyclo heterocyclic compounds, compositions comprising these heterocyclic compounds, methods of making the heterocyclic compounds, and methods of using these heterocyclic compounds for treating a variety of conditions and disease states associated with, for example, cellular proliferation, inflammation, glycosidase expression, or the low expression of Perlecan.

### RELATED APPLICATION DATA

This application claims the benefit of U.S. Provisional Patent Application Ser. No. 60/630,604, filed Nov. 23, 2004, which is incorporated herein by reference in its entirety.

### FIELD OF THE INVENTION

The present invention relates to bicyclo heterocyclic compounds, methods and compositions for making and using the heterocyclic compounds, and methods for treating conditions or diseases associated with cellular proliferation, inflammation, or glycosidase expression.

### BACKGROUND OF THE INVENTION

Novel compounds for new therapeutic interventions are needed for many areas of medicine and disease treatment. For example, chronic and acute inflammatory conditions form the basis for diseases affecting all organ systems including, but not limited to, asthma, acute inflammatory diseases, vascular inflammatory disease, chronic inflammation, atherosclerosis, angiopathy, myocarditis, nephritis, Crohn's disease, arthritis, type I and II diabetes and associated vascular pathologies. The incidence of these inflammatory conditions is on the rise in the population as a whole, with diabetes alone affecting 16 million people. Therefore, synthesis of novel compounds leads to new possibilities for discovery of novel therapeutic interventions.

While inflammation in and of itself is a normal immune response, chronic inflammation leads to complications and ongoing system damage due to the interactions of unknown cellular factors. In particular, chronic inflammation can cause endothelial damage resulting in vascular complications. Coronary artery, cerbrovascular and peripheral vascular disease resulting from atherosclerotic and thromboembolic macroangiopathy are the primary causes of mortality in chronic inflammatory diseases.

Many humans and animals have limited lifespans and lifestyles because of conditions relating to lifestyle choices, such as diet and exercise, or because of genetic predispositions to develop a disease. For example, vascular smooth muscle cell (SMC) proliferation is a common consequence of endothelial injury and is believed to be an early pathogenetic event in the formation of atherosclerotic plaques or complications related to vascular injury or as a result surgical interventions. Abnormal vascular SMC proliferation is thought to contribute to the pathogenesis of vascular occlusive lesions, including arteriosclerosis, atherosclerosis, restenosis, and graft atherosclerosis after organ transplantation.

One disease that rapidly growing in the industrialized countries is the occurrence of diabetes and all of its attendant sequellae. One of the factors important in the damage associated with diabetes is the presence of glycated proteins. Glycated proteins and advanced glycation end products (AGE) contribute to cellular damage, particularly, diabetic tissue injury. One potential mechanism by which hyperglycemia can be linked to microangiopathies is through the process of non-enzymatic glycation of critical proteins. These are a highly reactive group of molecules whose interaction with specific receptors on the cell-surface which are thought to lead to pathogenic outcomes.

Another major area of unwanted cellular growth, that is unchecked by the body's regulatory systems, is cancer or oncological conditions. Many therapies have been used and are being used in an effort to restore health or at least stop the unwanted cell growth. Many times, therapeutic agents can have an effect individually, but often, therapeutic regimes require combinations of different pharmacological agents with treatments such as surgery or radiation.

There is a present need for treatments of chronic or acute diseases, such as atherosclerosis, unwanted cellular growth or cellular proliferation, diabetes, inflammatory conditions and vascular occlusive pathologic conditions. Because of occurrence is frequent, the currently available treatments are costly and the conditions are refractory to many pharmacological therapies. The mechanisms involved in the control or prevention of such diseases are not clear and there exists a need for preventive and therapeutic treatments of these and other diseases. Thus, what is presently needed are novel compounds that find utility in methods and compositions for treatment and prevention of chronic and acute diseases.

### SUMMARY OF THE INVENTION

The present invention is directed to novel bicyclo heterocyclic compounds, novel compositions comprising these heterocyclic compounds, and novel methods employing such bicyclo heterocycles and their compositions. Disclosed herein are methods for making bicyclo heterocyclic compounds, compositions comprising these heterocycles, and methods and compositions for using these bicyclic heterocycles. The heterocyclic compounds and compositions comprising these compounds have utility in treatment of a variety of diseases.

In one aspect, compounds in accordance with the present invention, and compositions comprising these compounds, comprise substituted bicyclo heterocyclic compounds of formula: 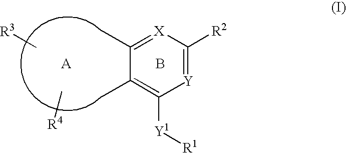
or a salt, including a pharmaceutically acceptable or a non-pharmaceutically acceptable salt, a prodrug, a diastereomeric mixture, an enantiomer, a tautomer, a racemic mixture thereof, or any combination thereof,

wherein:

ring A is a substituted or an unsubstituted pyrazole ring,

R³ and R⁴ are selected independently from a substituted or an unsubstituted alkyl or a substituted or an unsubstituted aryl, any of which having up to 12 carbon atoms, or hydrogen;

X and Y are selected independently from CH or N, with a proviso that at least one of X or Y represents N;

Y¹ is >NR⁵, —C≡C—, >O, or a direct a bond between ring B and R¹;

R¹ is a substituted or an unsubstituted aryl, heterocyclyl, or heteroaryl, any of which having up to 12 carbon atoms; wherein any heteroaryl or heterocyclyl comprises at least one heteroatom or heterogroup selected from >O, >N—, >S, >NR⁶, >SO₂, or >CO;

R² is a substituted or an unsubstituted alkyl, haloalkyl, aryl, heterocyclyl, or heteroaryl, any of which having up to 12 carbon atoms; wherein any heteroaryl or heterocyclyl comprises at least one heteroatom or heterogroup selected from >O, >N—, >S, >NR⁶, >SO₂, or >CO;

R⁵ is a substituted or an unsubstituted alkyl having up to 12 carbon atoms, or hydrogen; and

any of R¹, R², R³, R⁴, and R⁵ is optionally substituted with at least one group selected independently from: 1) an alkyl, an alkoxy, an alkylthio, a haloalkyl, a haloalkoxy, a cycloalkyl, NR⁶R⁷, —CO₂R⁶, —COR⁸, —CONR⁶R⁷, —SO₂R⁸ and —SO₂NR⁶R⁷, NHSO₂R⁸, or NHCOR⁸, any of which having up to 10 carbon atoms; or 2) halogen, hydroxyl, or cyano;

R⁶ and R⁷ are selected independently from an alkyl or an aryl having up to 10 carbon atoms, or hydrogen; and

R⁸ is an alkyl or aryl having up to 10 carbon atoms.

In the compound of formula I, any optional substituents on any group R¹, R², R³, R⁴, and R⁵ are selected independently of any other substituents, therefore, substituents can occur none, one, two, three, or more times, as each group R¹, R², R³, R⁴, and R⁵ allows, and can be the same or can be different.

The present invention also is directed to a method for treating a condition or disease in a mammalian subject, including a human. In some aspects, the method comprises administering to the subject a composition comprising a therapeutically-effective amount of at least one compound disclosed herein, or their pharmaceutically-acceptable salts. In some aspects, the at least one compound is, for example, a compound of formula I, Ia, IIb, IIc, IId, IIe, IIf, IIg, IIh, IIi, IIa-1, or any combination thereof.

Besides being useful for treating a human subject, the methods and compositions of the present invention are useful for treating a variety of mammals such as, for example, companion animals (e.g., cat, dog), primates, ruminant animals, and rodents.

The present invention also is directed to a method for treating a condition or disease associated with a cellular proliferation in a mammalian subject, the method comprising administering to the subject a composition comprising a therapeutically-effective amount of at least one compound disclosed herein, or their pharmaceutically-acceptable salts thereof. In some aspects, the at least one compound is, for example, a compound of formula I, Ia, IIb, IIc, IId, IIe, IIf, IIg, IIh, IIi, IIa-1, or any combination thereof. In some aspects, the condition or disease is a neoplasia. In another aspect, the condition or disease is SMC hyperplasia.

The present invention also is directed to a method for treating a condition or disease related to glycosidase expression. In one aspect, the present invention provides a method for treating a condition or disease associated with glycosidase expression in a mammalian subject, the method comprising administering to the subject a composition comprising a therapeutically-effective amount of at least one compound disclosed herein, or their pharmaceutically-acceptable salts thereof. In some aspects, the at least one compound is, for example, a compound of formula I, IIa, IIb, IIc, IId, IIe, IIf, IIg, IIh, IIi, IIa-1, or any combination thereof.

The present invention also is directed to a method for treating a condition or disease associated with an inflammation in a mammalian subject, the method comprising administering to the subject a composition comprising a therapeutically-effective amount of at least one compound disclosed herein, or their pharmaceutically-acceptable salts thereof. In some aspects, the at least one compound is, for example, a compound of formula I, Ia, IIb, IIc, IId, IIe, IIf, IIg, IIh, IIi, IIa-1, or any combination thereof. In one aspect, the therapeutically effective amount is sufficient to attenuate or inhibit inflammation. In some aspects, the inflammation is associated with accumulation or presence of glycated proteins or AGE.

### DETAILED DESCRIPTION OF THE INVENTION

In accordance with the present invention, novel bicycle heterocyclic compounds and novel compositions comprising these heterocyclic compounds are described herein. In one aspect, compounds in accordance with the present invention can comprise bicyclo heterocyclic compounds, having the following formula: 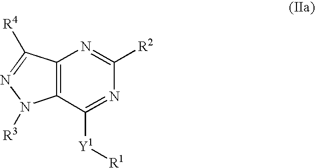
or a salt, including a pharmaceutically acceptable or a non-pharmaceutically acceptable salt, a prodrug, a diastereomeric mixture, an enantiomer, a tautomer, a racemic mixture thereof, or any combination thereof, wherein:

Y¹ is >NR⁵, —C≡C—, >O, or a direct a bond between the 6-membered ring and R¹;

wherein when Y¹ is >NR⁵, NR⁵R¹ can constitute a 5-, 6-, or 7-membered heterocyclic ring, which can optionally comprise one or two additional hetero atoms selected from >O, >S or >N—, in which NR⁵R¹ is optionally substituted with one, two, or three substituents selected indepdently from an alkyl, an alkoxy, or a haloalkyl, any of which having up to 10 carbon atoms, or hydroxyl, halogen, or cyano;

R¹ is a substituted or an unsubstituted aryl, heterocyclyl, or heteroaryl, any of which having up to 12 carbon atoms; wherein any heteroaryl or heterocyclyl comprises at least one heteroatom or heterogroup selected from >O, >N—, >S, >NR⁶, >SO₂, or >CO;

R² is a substituted or an unsubstituted alkyl, haloalkyl, aryl, or heteroaryl, any of which having up to 12 carbon atoms; wherein any heteroaryl comprises at least one heteroatom or heterogroup selected from >O, >N—, >S, or >NR₆;

R³ and R⁴ are selected independently from a substituted or an unsubstituted alkyl or a substituted or an unsubstituted aryl, any of which having up to 12 carbon atoms;

R⁵ is a substituted or an unsubstituted alkyl having up to 12 carbon atoms, or hydrogen;

any of R¹, R², R³, R⁴, and R⁵ can be optionally substituted with at least one group selected independently from: 1) an alkyl, an alkoxy, an alkylthio, a haloalkyl, a haloalkoxy, a cycloalkyl, NR⁶R⁷, —CO₂R⁶, —COR⁸, —CONR⁶R⁷, —SO₂R⁸ and —SO₂NR⁶R⁷, NHSO₂R⁸, or NHCOR⁸, any of which having up to 10 carbon atoms; or 2) halogen, hydroxyl, or cyano;

R⁶ and R⁷ are selected independently from an alkyl or an aryl having up to 10 carbon atoms, or hydrogen; and

R⁸ is an alkyl or aryl having up to 10 carbon atoms.

In the compound of formula IIa, any optional substituents on any group R¹, R², R³, R⁴, and R⁵ are selected independently of any other substituents, therefore, substituents can occur none, one, two, three, or more times, as each group R¹, R², R³, R⁴, and R⁵ allows, and the substituents can be the same or can be different.

In yet another aspect, the present invention provides compounds and compositions comprising these compounds, wherein the compounds have the following formula: 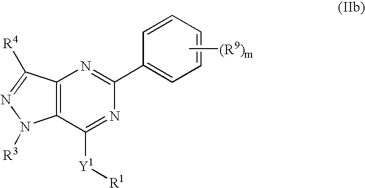
or a salt, including a pharmaceutically acceptable or a non-pharmaceutically acceptable salt, a prodrug, a diastereomeric mixture, an enantiomer, a tautomer, a racemic mixture thereof, or any combination thereof, wherein:

Y¹is >NR⁵, —C≡C—, >O, or a direct a bond between the 6-membered ring and R¹;

R¹ is a substituted or an unsubstituted aryl, heterocyclyl, or heteroaryl, any of which having up to 12 carbon atoms; wherein any heteroaryl or heterocyclyl comprises at least one heteroatom or heterogroup selected from >O, >N—, >S, >NR⁶, >SO₂, or >CO;

R³ and R⁴ are selected independently from a substituted or an unsubstituted alkyl or a substituted or an unsubstituted aryl, any of which having up to 12 carbon atoms;

R⁵ is a substituted or an unsubstituted alkyl having up to 12 carbon atoms, or hydrogen;

R⁹, in each occurrence, is selected independently from: 1) an alkyl, an alkoxy, a haloalkyl, a haloalkoxy, SO₂R⁸, SO₂NR⁶R⁷, CO₂R⁶, COR⁸, or CONR⁶R⁷, any of which having up to 10 carbon atoms; or 2) halogen;

m is an integer from 0 to 3, inclusive;

any of R¹, R³, and R⁴ can be optionally substituted with at least one group selected independently from: 1) an alkyl, an alkoxy, an alkylthio, a haloalkyl, a haloalkoxy, a cycloalkyl, NR⁶R⁷, CO₂R⁶, CO⁸R, CONR⁶R⁷, SO₂R⁸, SO₂NR⁶R⁷, NHSO₂R⁸, or NHCOR⁸, any of which having up to 10 carbon atoms; or 2) halogen, hydroxyl, or cyano;

R⁶ and R⁷ are selected independently from an alkyl or an aryl having up to 10 carbon atoms, or hydrogen; and

R⁸is an alkyl or aryl having up to 10 carbon atoms.

Another aspect of this invention provides compounds, and compositions comprising the compounds, wherein the compounds have the following formula: 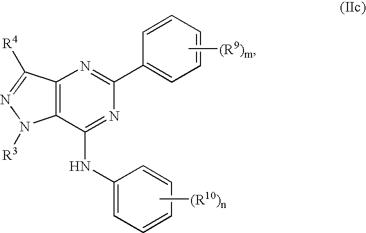
or a salt, including a pharmaceutically acceptable or a non-pharmaceutically acceptable salt, a prodrug, a diastereomeric mixture, an enantiomer, a tautomer, a racemic mixture thereof, or any combination thereof, wherein:

R⁹ and R¹⁰, in each occurrence, are selected independently from: 1) an alkyl, an alkoxy, a haloalkyl, a haloalkoxy, NR⁶R⁷, CO₂R⁶, COR⁸, CONR⁶R⁷, SO₂R , SO₂NR⁶R7, NHSO₂R⁸, or NHCOR⁸, any of which having up to 10 carbon atoms; or 2) halogen or cyano;

m and n are selected independently from an integer from 0 to 3, inclusive;

R⁶ and R⁷ are selected independently from an alkyl or an aryl having up to 10 carbon atoms, or hydrogen;

R is an alkyl or aryl having up to 10 carbon atoms;

R³ and R⁴ are selected independently from a substituted or an unsubstituted alkyl or a substituted or an unsubstituted aryl, any of which having up to 12 carbon atoms; and

any of R³ and R⁴ can be optionally substituted with at least one group selected independently from: 1) an alkyl, an alkoxy, a haloalkyl, a haloalkoxy, or a cycloalkyl, any of which having up to 10 carbon atoms; or 2) halogen or hydroxyl.

Further to this aspect, this disclosure provides heterocyclic compounds, wherein the compound is selected from any of the following compounds, including any combination thereof:

(3-Chloro-4-methoxy-phenyl)-[5-(3,4-dimethoxy-phenyl)-1-methyl-3-propyl-1H-
pyrazolo[4,3-d]pyrimidin-7-yl]-amine hydrochloride;
(3-Chloro-4-methoxy-phenyl)-[5-(4-fluoro-phenyl)-1-methyl-3-propyl-1H-
pyrazolo[4,3-d]pyrimidin-7-yl]-amine hydrochloride;
(3-Fluoro-4-methoxy-phenyl)-[5-(4-fluoro-phenyl)-1-methyl-3-propyl-1H-pyrazolo
[4,3-d]pyrimidin-7-yl]-amine hydrochloride;
(3-Fluoro-4-methoxy-phenyl)-[5-(4-fluoro-phenyl)-1-methyl-3-propyl-1H-pyrazolo
[4,3-d]pyrimidin-7-yl]-amine;
(4-Fluoro-phenyl)-[5-(4-fluoro-phenyl)-1-methyl-3-propyl-1H-pyrazolo[4,3-
d]pyrimidin-7-yl]-amine hydrochloride;
(3,4-Dimethoxy-phenyl)-[5-(4-fluoro-phenyl)-1-methyl-3-propyl-1H-pyrazolo[4,3-
d]pyrimidin-7-yl]-amine hydrochloride;
2-Chloro-4-(1-methyl-5-phenyl-3-propyl-1H-pyrazolo[4,3-d]pyrimidin-7-ylamino)-phenol
hydrochloride;
(3-Chloro-4-methoxy-phenyl)-(1-methyl-5-phenyl-3-propyl-1H-pyrazolo[4,3-
d]pyrimidin-7-yl)-amine hydrochloride;
(3-Chloro-4-methoxy-phenyl)-(1-methyl-5-phenyl-3-propyl-1H-pyrazolo[4,3-
d]pyrimidin-7-yl)-amine;
(3-Fluoro-4-methoxy-phenyl)-(1-methyl-5-phenyl-3-propyl-1H-pyrazolo[4,3-
d]pyrimidin-7-yl)-amine hydrochloride;
2-Chloro-4-[5-(4-fluoro-phenyl)-1-methyl-3-propyl-1H-pyrazolo[4,3-d]pyrimidin-
7-ylamino]-phenol hydrochloride;
3-[7-(3-Chloro-4-methoxy-phenylamino)-1-methyl-3-propyl-1H-pyrazolo[4,3-
d]pyrimidin-5-yl]-4-ethoxy-benzenesulfonamide hydrochloride;
4-Ethoxy-3-[7-(3-fluoro-4-methoxy-phenylamino)-1-methyl-3-propyl-1H-
pyrazolo[4,3-d]pyrimidin-5-yl]-benzenesulfonamide hydrochloride;
(3-Chloro-4-methoxy-phenyl)-[5-(2-ethoxy-phenyl)-1-methyl-3-propyl-1H-
pyrazolo[4,3-d]pyrimidin-7-yl]-amine hydrochloride;
[5-(2-Ethoxy-phenyl)-1-methyl-3-propyl-1H-pyrazolo[4,3-d]pyrimidin-7-yl]-
(3-fluoro-4-methoxy-phenyl)-amine hydrochloride;
(3-Fluoro-4-methoxy-phenyl)-[5-(4-fluoro-phenyl)-1,3-dimethyl-1H-pyrazolo[4,3-
d]pyrimidin-7-yl]-amine hydrochloride;
(3-Chloro-4-methoxy-phenyl)-[5-(4-fluoro-phenyl)-1,3-dimethyl-1H-pyrazolo[4,3-
d]pyrimidin-7-yl]-amine hydrochloride;
2-Chloro-4-[5-(4-fluoro-phenyl)-1,3-dimethyl-1H-pyrazolo[4,3-d]pyrimidin-7-
ylamino]-phenol hydrochloride;
(4-Chloro-3-methoxy-phenyl)-(1-methyl-5-phenyl-3-propyl-1H-pyrazolo[4,3-
d]pyrimidin-7-yl)-amine hydrochloride;
(4-Chloro-3-methoxy-phenyl)-[5-(4-fluoro-phenyl)-1-methyl-3-propyl-1H-
pyrazolo[4,3-d]pyrimidin-7-yl]-amine hydrochloride;
(3-Chloro-4-methoxy-phenyl)-(1,3-dimethyl-5-phenyl-1H-pyrazolo[4,3-
d]pyrimidin-7-yl)-amine hydrochloride;
(1,3-Dimethyl-5-phenyl-1H-pyrazolo[4,3-d]pyrimidin-7-yl)-(3-fluoro-4-methoxy-
phenyl)-amine hydrochloride;
(4-Chloro-3-methoxy-phenyl)-(1,3-dimethyl-5-phenyl-1H-pyrazolo[4,3-
d]pyrimidin-7-yl)-amine hydrochloride;
2-Fluoro-4-[5-(4-fluoro-phenyl)-1-methyl-3-propyl-1H-pyrazolo[4,3-d]pyrimidin-
7-ylamino]-phenol hydrochloride;
Benzo[1,3]dioxol-5-yl-[5-(4-fluoro-phenyl)-1-methyl-3-propyl-1H-pyrazolo[4,3-d]
pyrimidin-7-yl]-amine hydrochloride;
(1,3-Dimethyl-5-phenyl-1H-pyrazolo[4,3-d]pyrimidin-7-yl)-(3-fluoro-phenyl)-
amine hydrochloride;
[5-(4-Fluoro-phenyl)-1-methyl-3-propyl-1H-pyrazolo[4,3-d]pyrimidin-7-yl]-(3-
trifluoromethyl-phenyl)-amine hydrochloride;
[5-(4-Fluoro-phenyl)-1-methyl-3-propyl-1H-pyrazolo[4,3-d]pyrimidin-7-yl]-(4-
trifluoromethoxy-phenyl)-amine hydrochloride;
(1,3-Dimethyl-5-phenyl-1H-pyrazolo[4,3-d]pyrimidin-7-yl)-(4-trifluoromethoxy-
phenyl)-amine hydrochloride;
[5-(4-Fluoro-phenyl)-1,3-dimethyl-1H-pyrazolo[4,3-d]pyrimidin-7-yl]-(4-
rifluoromethyl-phenyl)-amine hydrochloride;
(6-Chloro-pyridin-3-yl)-[5-(4-fluoro-phenyl)-1,3-dimethyl-1H-pyrazolo[4,3-
d]pyrimidin-7-yl]-amine hydrochloride;
N-{5-[5-(4-Fluoro-phenyl)-1,3-dimethyl-1H-pyrazolo[4,3-d]pyrimidin-7-ylamino]-
2-hydroxy-phenyl}-acetamide hydrochloride;
[5-(4-Fluoro-phenyl)-1,3-dimethyl-1H-pyrazolo[4,3-d]pyrimidin-7-yl]-(4-
methanesulfonyl-phenyl)-amine;
(1,3-Dimethyl-5-phenyl-1H-pyrazolo[4,3-d]pyrimidin-7-yl)-(2-methyl-
benzooxazol-5-yl)-amine hydrochloride;
N-[4-(1,3-Dimethyl-5-phenyl-1H-pyrazolo[4,3-d]pyrimidin-7-ylamino)-phenyl]-
methanesulfonamide hydrochloride;
4-[5-(4-Fluoro-phenyl)-1,3-dimethyl-1H-pyrazolo[4,3-d]pyrimidin-7-ylamino]-
N,N-dimethyl-benzenesulfonamide hydrochloride;
4-(1,3-Dimethyl-5-phenyl-1H-pyrazolo[4,3-d]pyrimidin-7-ylamino)-
benzenesulfonamide hydrochloride;
3-[5-(4-Fluoro-phenyl)-1,3-dimethyl-1H-pyrazolo[4,3-d]pyrimidin-7-ylamino]-
benzamide hydrochloride;
3-[5-(4-Fluoro-phenyl)-1,3-dimethyl-1H-pyrazolo[4,3-d]pyrimidin-7-ylamino]-
N-methyl-benzamide hydrochloride;
4-[5-(4-Fluoro-phenyl)-1,3-dimethyl-1H-pyrazolo[4,3-d]pyrimidin-7-ylamino]-
benzenesulfonamide hydrochloride;
4-[5-(4-Fluoro-phenyl)-1,3-dimethyl-1H-pyrazolo[4,3-d]pyrimidin-7-ylamino]-N-
methyl-benzenesulfonamide hydrochloride;
4-[5-(4-Fluoro-phenyl)-1,3-dimethyl-1H-pyrazolo[4,3-d]pyrimidin-7-ylamino]-
benzamide hydrochloride;
4-[5-(4-Fluoro-phenyl)-1,3-dimethyl-1H-pyrazolo[4,3-d]pyrimidin-7-ylamino]-
N-methyl-benzamide hydrochloride; or
any combination thereof.

One more aspect of the present invention provides heterocyclic compounds, and compositions comprising the heterocyclic compounds, wherein the compounds have the following formula: 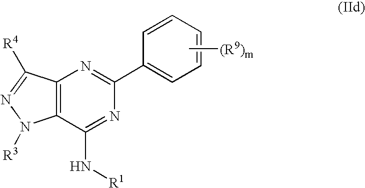
or a salt, including a pharmaceutically acceptable or a non-pharmaceutically acceptable salt, a prodrug, a diastereomeric mixture, an enantiomer, a tautomer, a racemic mixture thereof, or any combination thereof, wherein:

R¹ is a substituted or an unsubstituted aryl, or a substituted or an unsubstituted heteroaryl, any of which having up to 12 carbon atoms; wherein any heteroaryl comprises at least one heteroatom or heterogroup selected from >O, >N—, >S, or >NR⁶;

R⁹, in each occurrence, is selected independently from: 1) an alkyl, an alkoxy, a haloalkyl, a haloalkoxy, SO₂R⁸, SO₂NR⁶R⁷, NR⁶R⁷, CO₂R⁶, COR⁸, or CONR⁶R⁷, any of which having up to 10 carbon atoms; or 2) halogen;

m is an integer from 0 to 3, inclusive;

R³ and R⁴ are selected independently from a substituted or an unsubstituted alkyl or a substituted or an unsubstituted aryl, any of which having up to 12 carbon atoms; and

any of R¹, R³, and R⁴ can be optionally substituted with at least one group selected independently from: 1) an alkyl, an alkoxy, a haloalkyl, or a haloalkoxy, any of which having up to 10 carbon atoms; or 2) halogen or hydroxyl;

Further to this aspect and the formula (IId) presented immediately above, the following substituents of the formula can be selected as follows, while unspecified substitutents are selected as above: R¹ can be an indole, a benzimidazole, a benzoxazole, a benzo[1,3]dioxole, or a pyridine.

Further to this aspect and this formula, this disclosure provides heterocyclic compounds, wherein the compound is selected from any of the following compounds, including any combination thereof:

(1H-Benzoimidazol-5-yl)-(1,3-dimethyl-5-phenyl-1H-pyrazolo[4,3-
d]pyrimidin-7-yl)-amine hydrochloride;
(1,3-Dimethyl-5-phenyl-1H-pyrazolo[4,3-d]pyrimidin-7-yl)-
(2-methyl-1H-benzoimidazol-5-yl)-amine hydrochloride;
Benzo[1,3]dioxol-5-yl-[5-(4-fluoro-phenyl)-1,3-dimethyl-1H-
pyrazolo[4,3-d]pyrimidin-7-yl]-amine hydrochloride.

In still a further aspect, the present disclosure provides compounds and compositions comprising these compounds, wherein the compounds have the following formula: 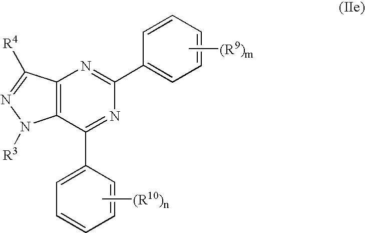
or a salt, including a pharmaceutically acceptable or a non-pharmaceutically acceptable salt, a prodrug, a diastereomeric mixture, an enantiomer, a tautomer, a racemic mixture thereof, or any combination thereof, wherein:

R⁹ and R¹⁰, in each occurrence, are selected independently from: 1) an alkyl, an alkoxy, a haloalkyl, a haloalkoxy, NR⁶R⁷, CO₂R⁶, COR⁸, CONR⁶R⁷, SO₂R⁸, SO₂NR⁶R⁷, NHSO₂R⁸, or NHCOR⁸, any of which having up to 10 carbon atoms; or 2) halogen or cyano;

m and n are selected independently from an integer from 0 to 3, inclusive;

R⁶ and R⁷ are selected independently from an alkyl or an aryl having up to 10 carbon atoms, or hydrogen;

R⁸ is an alkyl or aryl having up to 10 carbon atoms;

R³ and R⁴ are selected independently from a substituted or an unsubstituted alkyl or a substituted or an unsubstituted aryl, any of which having up to 12 carbon atoms; and

any of R³ and R⁴ can be optionally substituted with at least one group selected independently from: 1) an alkyl, an alkoxy, a haloalkyl, a haloalkoxy, or a cycloalkyl, any of which having up to 10 carbon atoms; or 2) halogen or hydroxyl.

Further to this aspect, this disclosure provides heterocyclic compounds, wherein the compound is selected from any of the following compounds, including any combination thereof:

4-[5-(3,4-Dimethoxy-phenyl)-1-methyl-3-propyl-1H-pyrazolo[4,3-d]pyrimidin-7-
yl]-2-methyl-phenol;
2-Methyl-4-(1-methyl-5-phenyl-3-propyl-1H-pyrazolo[4,3-d]pyrimidin-7-yl)-phenol
4-[5-(3-hydroxy,4-methoxy-phenyl)-1-methyl-3-propyl-1H-pyrazolo[4,3-
d]pyrimidin-7-yl]-2-methyl-phenol;
2-Chloro-4-[5-(4-fluoro-phenyl)-1-methyl-3-propyl-1H-pyrazolo[4,3-d]pyrimidin-7-yl]-phenol;
7-(4-Methoxy-3-methyl-phenyl)-1-methyl-5-phenyl-3-propyl-1H-pyrazolo[4,3-
d]pyrimidine;
5-(4-fluoro-phenyl)-1,3-dimethyl-7-phenyl-1H-pyrazolo[4,3-d]pyrimidine;
5-(4-Fluoro-phenyl)-1-methyl-3-propyl-7-p-tolyl-1H-pyrazolo[4,3-d]pyrimidine;
7-(3-Fluoro-4-methoxy-phenyl)-1-methyl-5-phenyl-3-propyl-1H-pyrazolo[4,3-
d]pyrimidine;
5-(4-Fluoro-phenyl)-1-methyl-7-phenyl-3-propyl-1H-pyrazolo[4,3-d]pyrimidine;
5-(4-Fluoro-phenyl)-1-methyl-7-(4-methylsulfanyl-phenyl)-3-propyl-1H-
pyrazolo[4,3-d]pyrimidine;
7-(3-Fluoro-4-methoxy-phenyl)-5-(4-fluoro-phenyl)-1-methyl-3-propyl-1H-
pyrazolo[4,3-d]pyrimidine;
5-(4-Fluoro-phenyl)-7-(4-methoxy-3-methyl-phenyl)-1-methyl-3-propyl-1H-
pyrazolo[4,3-d]pyrimidine;
5-(4-Fluoro-phenyl)-7-(4-methanesulfonyl-phenyl)-1-methyl-3-propyl-1H-
pyrazolo[4,3-d]pyrimidine;
7-(3-Methanesulfonyl-phenyl)-1,3-dimethyl-5-phenyl-1H-pyrazolo[4,3-
d]pyrimidine
5-(4-Fluoro-phenyl)-7-(3-methanesulfonyl-phenyl)-1,3-dimethyl-1H-pyrazolo[4,3-
d]Pyrimidine;
5-(4-Fluoro-phenyl)-1,3-dimethyl-7-(4-trifluoromethoxy-phenyl)-1H-pyrazolo[4,3-
d]pyrimidine.

Still another aspect of this disclosure provides for compounds and compositions comprising these compounds, wherein the compounds have the following formula: 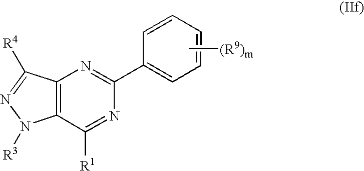
or a salt, including a pharmaceutically acceptable or a non-pharmaceutically acceptable salt, a prodrug, a diastereomeric mixture, an enantiomer, a tautomer, a racemic mixture thereof, or any combination thereof, wherein:

R¹ is a substituted or an unsubstituted heteroaryl, or a substituted or an unsubstituted heterocyclyl, comprising at least one heteroatom or heterogroup selected from —O—, >N—, —S—, >NR⁶, >CO, or >SO₂, any of which having up to 10 carbon atoms;

R⁹, in each occurrence, is selected independently from: 1) an alkyl, an alkoxy, a haloalkyl, a haloalkoxy, SO₂⁸, SO₂NR⁶R⁷, NR⁶R⁷, CO₂R⁶, COR⁸, or CONR⁶R⁷, any of which having up to 10 carbon atoms; or 2) halogen;

m is an integer from 0 to 3, inclusive;

R³ and R⁴ are selected independently from a substituted or an unsubstituted alkyl or a substituted or an unsubstituted aryl, any of which having up to 12 carbon atoms;

any of R¹, R³, and R⁴ can be optionally substituted with at least one group selected independently from: 1) an alkyl, an alkoxy, an alkylthio, a haloalkyl, a haloalkoxy, a cycloalkyl, NR⁶R⁷, —CO₂R⁶, —COR⁸, —CONR⁶R⁷, —SO₂R⁸ and —SO₂NR⁶R⁷, NHSO₂R⁸, or NHCOR⁸, any of which having up to 10 carbon atoms; or 2) halogen or hydroxyl;

R⁶ and R⁷ are selected independently from an alkyl or an aryl having up to 10 carbon atoms, or hydrogen; and

R⁸ is an alkyl or aryl having up to 10 carbon atoms.

Further to this aspect and the formula (IIf) presented immediately above, the following substituents of the formula can be selected as follows, while unspecified substitutents are selected as above: R¹ can be an indole, a benzo[1,3]dioxole, or a piperidine.

Still further to this aspect, this disclosure provides heterocyclic compounds, wherein the compound is selected from any of the following compounds, including any combination thereof:

1-[5-(3,4-Dimethoxy-phenyl)-1-methyl-3-propyl-1H-pyrazolo[4,3-
d]pyrimidin-7-yl]-piperidin-4-ol;
5-(4-Fluoro-phenyl)-7-indol-1-yl-1-methyl-3-propyl-1H-
pyrazolo[4,3-d]pyrimidine;
7-(5-Chloro-indol-1-yl)-5-(4-fluoro-phenyl)-1-methyl-3-propyl-
1H-pyrazolo[4,3-d]pyrimidine;
7-Indol-1-yl-1,3-dimethyl-5-phenyl-1H-pyrazolo[4,3-d]pyrimidine;
7-Benzo[1,3]dioxol-5-yl-1,3-dimethyl-5-phenyl-1H-
pyrazolo[4,3-d]pyrimidine.

In yet a further aspect, the present disclosure provides compounds and compositions comprising these compounds, wherein the compounds have the following formula: 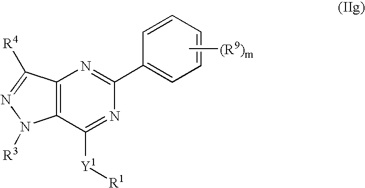
or a salt, including a pharmaceutically acceptable or a non-pharmaceutically acceptable salt, a prodrug, a diastereomeric mixture, an enantiomer, a tautomer, a racemic mixture thereof, or any combination thereof, wherein:

Y¹is —C≡C—, >O, or a direct a bond between the 6-membered ring and R¹;

R¹ is a substituted or an unsubstituted aryl or heteroaryl, any of which having up to 12 carbon atoms; wherein any heteroaryl comprises at least one heteroatom or heterogroup selected from >O, >N—, >S, >NR⁶;

R⁹, in each occurrence, is selected independently from: 1) an alkyl, an alkoxy, a haloalkyl, a haloalkoxy, SO₂R, SO₂NR⁶R⁷, NR⁶R⁷, CO₂R⁶, COR⁸, or CONR⁶R⁷, any of which having up to 10 carbon atoms; or 2) halogen;

m is an integer from 0 to 3, inclusive;

R³ and R⁴ are selected independently from a substituted or an unsubstituted alkyl or a substituted or an unsubstituted aryl, any of which having up to 12 carbon atoms;

any of R¹, R³, and R⁴ can be optionally substituted with at least one group selected independently from: 1) an alkyl, an alkoxy, an alkylthio, a haloalkyl, a haloalkoxy, a cycloalkyl, NR⁶R⁷, CO₂R⁶, COR⁸, CONR⁶R⁷, SO₂R⁸, SO₂NR⁶R⁷, NHSO₂R⁸, or NHCOR⁸, any of which having up to 10 carbon atoms; or 2) halogen or hydroxyl;

R⁶ and R⁷ are selected independently from an alkyl or an aryl having up to 10 carbon atoms, or hydrogen; and

R⁸ is an alkyl or aryl having up to 10 carbon atoms.

In addition to this aspect, this disclosure provides heterocyclic compounds, wherein the compound is selected from any of the following compounds, including any combination thereof:

7-(4-Fluoro-phenoxy)-1-methyl-5-phenyl-3-propyl-
1H-pyrazolo[4,3-d]pyrimidine;
5-(4-Fluoro-phenyl)-1-methyl-7-phenylethynyl-3-propyl-1H-pyrazolo[4,3-
d]pyrimidine.

Still another aspect of the present invention provides for compounds and compositions comprising these compounds, wherein the compounds have the following formula: 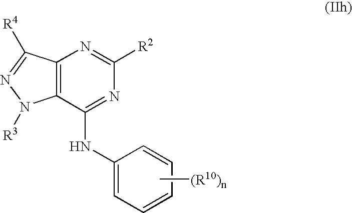
or a salt, including a pharmaceutically acceptable or a non-pharmaceutically acceptable salt, a prodrug, a diastereomeric mixture, an enantiomer, a tautomer, a racemic mixture thereof, or any combination thereof, wherein:

R² is a substituted or an unsubstituted haloalkyl or heteroaryl, any of which having up to 12 carbon atoms; wherein any heteroaryl comprises at least one heteroatom or heterogroup selected from >O, >N—, >S, or >NR⁶;

R¹⁰, in each occurrence, is selected independently from: 1) an alkyl, an alkoxy, a haloalkyl, a haloalkoxy, NR⁶R⁷, OCH₂O, CO₂R , COR⁶, CONR⁶R⁷, SO₂R⁶, SO₂NR⁶R⁷, NHSO₂R⁶, or NHCOR⁶, any of which having up to 10 carbon atoms; or 2) halogen or cyano;

n is an integer from 0 to 3, inclusive;

R³ and R⁴ are selected independently from a substituted or an unsubstituted alkyl or a substituted or an unsubstituted aryl, any of which having up to 12 carbon atoms; and

any of R², R³, and R⁴ can be optionally substituted with at least one group selected independently from: 1) an alkyl, an alkoxy, an alkylthio, a haloalkyl, a haloalkoxy, or a cycloalkyl, any of which having up to 10 carbon atoms; or 2) halogen or hydroxyl.

Further to this aspect and the formula (IIh) presented immediately above, the following substituents of the formula can be selected as follows, while unspecified substitutents are selected as above: R² can be a a thiophene group or CF₃.

Still further, in this aspect, this disclosure provides heterocyclic compounds, wherein the compound is selected from any of the following compounds, including any combination thereof:

(3-Chloro-4-methoxy-phenyl)-(1-methyl-3-propyl-5-thiophen-2-yl-1H-
pyrazolo[4,3-d]pyrimidin-7-yl)-amine hydrochloride;
(3-Fluoro-4-methoxy-phenyl)-(1-methyl-3-propyl-5-thiophen-2-yl-1H-
pyrazolo[4,3-d]pyrimidin-7-yl)-amine hydrochloride;
(1,3-Dimethyl-5-thiophen-2-yl-1H-pyrazolo[4,3-d]pyrimidin-7-yl)-(3-
fluoro-4-methoxy-phenyl)-amine hydrochloride;
(4-Chloro-3-methoxy-phenyl)-(1,3-dimethyl-5-thiophen-2-yl-1H-
pyrazolo[4,3-d]pyrimidin-7-yl)-amine hydrochloride;
(3-Chloro-4-methoxy-phenyl)-(1,3-dimethyl-5-thiophen-2-yl-1H-
pyrazolo[4,3-d]pyrimidin-7-yl)-amine hydrochloride;
2-Chloro-4-(1,3-dimethyl-5-thiophen-2-yl-1H-pyrazolo[4,3-d]pyrimidin-7-
ylamino)-phenol hydrochloride;
(3-Fluoro-4-methoxy-phenyl)-(1-methyl-3-propyl-5-trifluoromethyl-1H-
pyrazolo[4,3-d]pyrimidin-7-yl)-amine hydrochloride;
(4-Chloro-3-methoxy-phenyl)-(1-methyl-3-propyl-5-thiophen-2-yl-1H-
pyrazolo[4,3-d]pyrimidin-7-yl)-amine hydrochloride.

Still another aspect of this invention provides compounds and compositions comprising these compounds, wherein the compounds have the following formula: 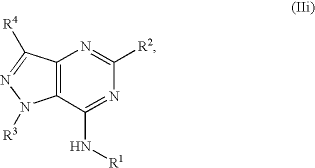
or a salt, including a pharmaceutically acceptable or a non-pharmaceutically acceptable salt, a prodrug, a diastereomeric mixture, an enantiomer, a tautomer, a racemic mixture thereof, or any combination thereof, wherein:

R¹ and R² are independently a substituted or an unsubstituted heteroaryl, any of which having up to 12 carbon atoms; wherein any heteroaryl comprises at least one heteroatom or heterogroup selected from >O, >N—, >S, or >NR⁶;

R³ and R⁴ are selected independently from a substituted or an unsubstituted alkyl or a substituted or an unsubstituted aryl, any of which having up to 12 carbon atoms; and

any of R¹, R², R³, and R⁴ can be optionally substituted with at least one group selected independently from: 1) an alkyl, an alkoxy, an alkylthio, a haloalkyl, a haloalkoxy, a cycloalkyl, NR⁶R⁷, CO₂R⁶, COR⁸, CONR⁶R⁷, SO₂R⁸, SO₂NR⁶R⁷, NHSO₂R⁸, or NHCOR⁸, any of which having up to 10 carbon atoms; or 2) halogen or hydroxyl;

R⁶ and R⁷, in each occurrence, are selected independently from an alkyl or an aryl having up to 10 carbon atoms, or hydrogen; and

R⁸ is an alkyl or aryl having up to 10 carbon atoms.

Substituents on this structure can occur none, one, two, three, or more times, as each R¹, R², R³, and R⁴ group allows, and substituents can be the same or can be different.

Further to this aspect, this disclosure provides heterocyclic compounds, wherein the compound is selected from any of the following compounds, including any combination thereof:

Benzo[1,3]dioxol-5-yl-(1-methyl-3-propyl-5-thiophen-2-yl-
1H-pyrazolo[4,3-d]pyrimidin-7-yl)-amine hydrochloride;
Benzo[1,3]dioxol-5-yl-(1,3-dimethyl-5-thiophen-2-yl-1H-
pyrazolo[4,3-d]pyrimidin-7-yl)-amine hydrochloride;
Benzo[1,3]dioxol-5-yl-(1-methyl-3-propyl-5-thiophen-2-yl-
1H-pyrazolo[4,3-d]pyrimidin-7-yl)-amine hydrochloride.

In yet an additional or a further aspect, the present invention provides compounds and compositions comprising these compounds, wherein the compounds have the following formula: 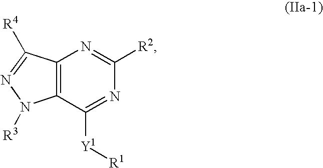
or a salt, including a pharmaceutically acceptable or a non-pharmaceutically acceptable salt, a prodrug, a diastereomeric mixture, an enantiomer, a tautomer, a racemic mixture thereof, or any combination thereof, wherein:

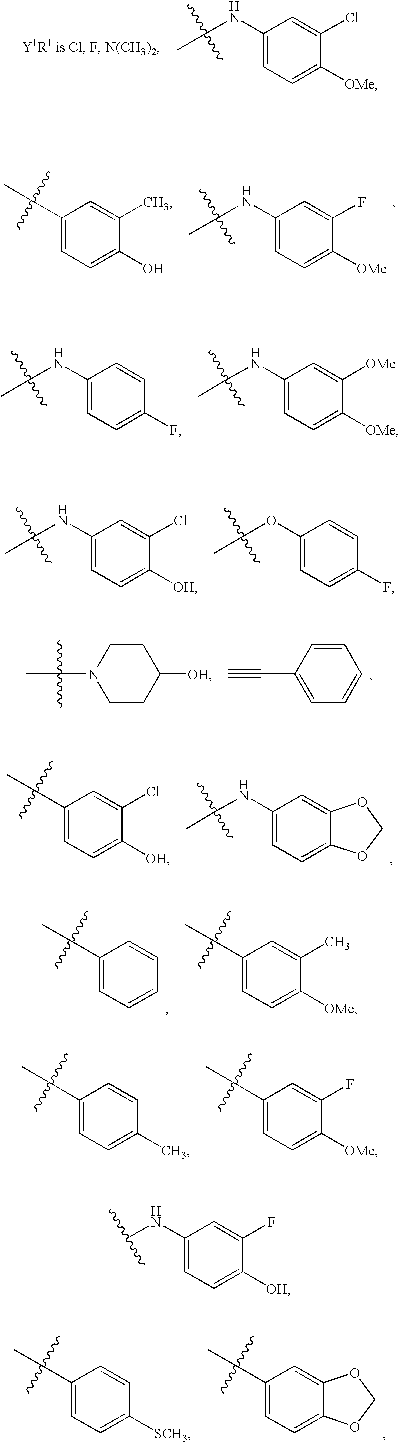

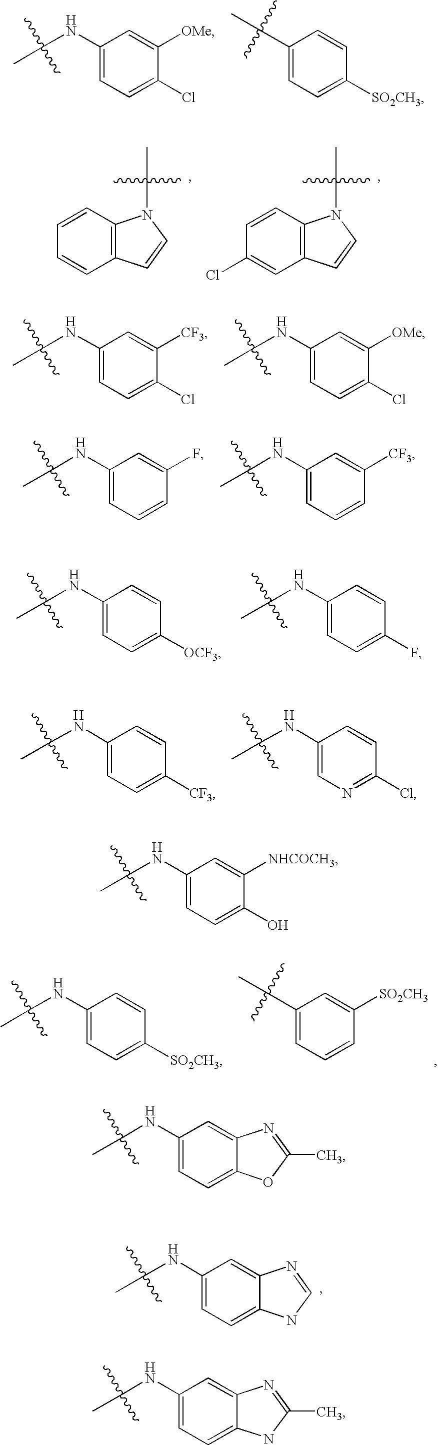

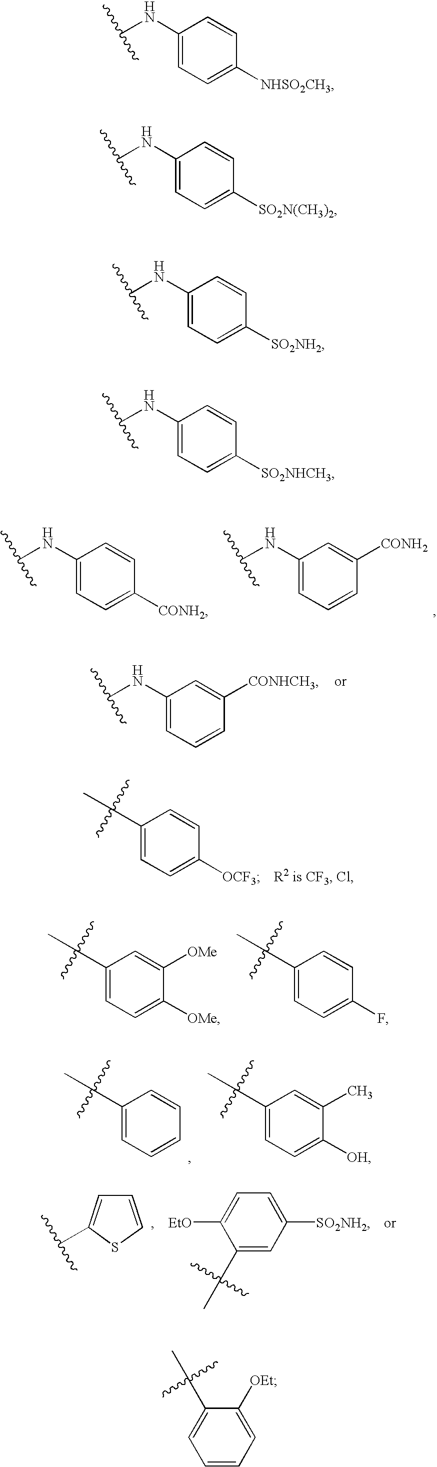

R³ is CH₃; and

R⁴is CH₂CH₂CH₃, CH₂CH₃, or CH₃.

In still another aspect, the present invention provides compounds and compositions comprising these compounds, wherein the compounds have the following formula: 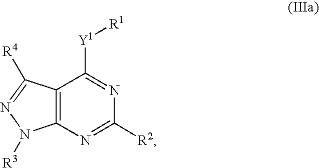
or a salt, including a pharmaceutically acceptable or a non-pharmaceutically acceptable salt, a prodrug, a diastereomeric mixture, an enantiomer, a tautomer, a racemic mixture thereof, or any combination thereof, wherein:

Y¹ is >NR⁵, —C≡C—, >O, or a direct a bond between the 6-membered ring and R¹;

R¹ is a substituted or an unsubstituted aryl, heterocyclyl, or heteroaryl, any of which having up to 12 carbon atoms; wherein any heteroaryl or heterocyclyl comprises at least one heteroatom or heterogroup selected from >O, >N—, >S, >NR⁶, >SO₂, or >CO;

R² is a substituted or an unsubstituted alkyl, haloalkyl, aryl, or heteroaryl, any of which having up to 12 carbon atoms; wherein any heteroaryl comprises at least one heteroatom or heterogroup selected from >O, >N—, >S, or >NR⁶;

R³is a substituted or an unsubstituted alkyl or a substituted or an unsubstituted aryl, any of which having up to 12 carbon atoms;

R⁴ is a substituted or an unsubstituted alkyl or a substituted or an unsubstituted aryl, any of which having up to 12 carbon atoms, or hydrogen;

R⁵is a substituted or an unsubstituted alkyl having up to 12 carbon atoms, or hydrogen;

any of R¹, R², R³, and R⁴ can be optionally substituted with at least one group selected independently from: 1) an alkyl, an alkoxy, an alkylthio, a haloalkyl, a haloalkoxy, a cycloalkyl, NR⁶R⁷, CO₂R⁶, COR⁸, CONR⁶R⁷, SO₂R⁸, SO₂NR⁶R⁷, NHSO₂R⁸, or NHCOR⁸, any of which having up to 10 carbon atoms; or 2) halogen, hydroxyl, or cyano;

R⁶ and R⁷ are selected independently from an alkyl or an aryl having up to 10 carbon atoms, or hydrogen; and

R⁸is an alkyl or aryl having up to 10 carbon atoms.

Further to this aspect and the formula (IIIa) presented immediately above, the following substituents of the formula can be selected as follows, while unspecified substitutents are selected as above:

R² can be a substituted or an unsubstituted haloalkyl, aryl, or thiophenyl, any of which having up to 12 carbon atoms;

R³ can be an alkyl having up to 6 carbon atoms or a phenyl;

R⁴ can be an alkyl having up to 6 carbon atoms, phenyl, or hydrogen; and

any of R¹ or R² can be optionally substituted with at least one group selected independently from an alkyl, an alkoxy, an alkylthio, a haloalkyl, a haloalkoxy, CONR⁶R⁷, SO₂R⁸, SO₂NR⁶R⁷, NHSO₂R⁸, or NHCOR⁸, any of which having up to 10 carbon atoms;

R⁶ and R⁷ are selected independently from an alkyl or an aryl having up to 10 carbon atoms, or hydrogen; and

R⁸ is an alkyl or aryl having up to 10 carbon atoms.

Another aspect of this invention provides compounds, and compositions comprising the compounds, wherein the compounds have the following formula: 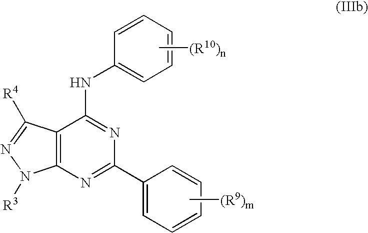
or a salt, including a pharmaceutically acceptable or a non-pharmaceutically acceptable salt, a prodrug, a diastereomeric mixture, an enantiomer, a tautomer, a racemic mixture thereof, or any combination thereof, wherein:

R³ and R⁴ are selected independently from hydrogen, methyl, ethyl, propyl, or phenyl;

R⁹ and R¹⁰, in each occurrence, are selected independently from: 1) an alkyl, an alkoxy, a haloalkyl, a haloalkoxy, NR⁶R⁷, CO₂R⁶, COR⁸, CONR⁶R⁷, SO₂R⁸, or SO₂NR⁶R⁷, any of which having up to 10 carbon atoms; or 2) halogen or cyano;

m and n are selected independently from an integer from 0 to 3, inclusive;

R⁶ and R⁷ are selected independently from H or methyl; and

R⁸ is methyl.

Still further to this aspect of the present invention, this disclosure provides heterocyclic compounds, wherein the compound is selected from any of the following compounds, including any combination thereof:

(3-Chloro-4-methoxy-phenyl)-(1,6-diphenyl-1H-pyrazolo[3,4-
d]pyrimidin-4-yl)-amine hydrochloride;
(3-Fluoro-4-methoxy phenyl)-[6-(4-fluoro-phenyl)-1-phenyl-1H-
pyrazolo [3,4-d]-pyrimidin-4yl]amine hydrochloride;
(4-Chloro-3-trifluoromethyl-phenyl)-[6-(4-fluoro-phenyl)-1-phenyl-1H-
pyrazolo[3,4-d]pyrimidin-4-yl]-amine hydrochloride;
(3-Fluoro-phenyl)-[6-(4-fluoro-phenyl)-1-phenyl-1H-pyrazolo[3,4-
d]pyrimidin-4-yl]-amine hydrochloride;
[6-(4-Fluoro-phenyl)-1-phenyl-1H-pyrazolo[3,4-d]pyrimidin-
4-yl]-(4-trifluoromethoxy-phenyl)-amine hydrochloride.

Still another aspect of this disclosure provides for compounds and compositions comprising these compounds, wherein the compounds have the following formula: 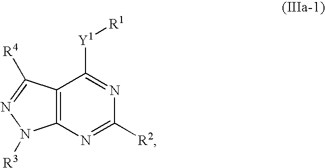
or a salt, including a pharmaceutically acceptable or a non-pharmaceutically acceptable salt, a prodrug, a diastereomeric mixture, an enantiomer, a tautomer, a racemic mixture thereof, or any combination thereof, wherein:

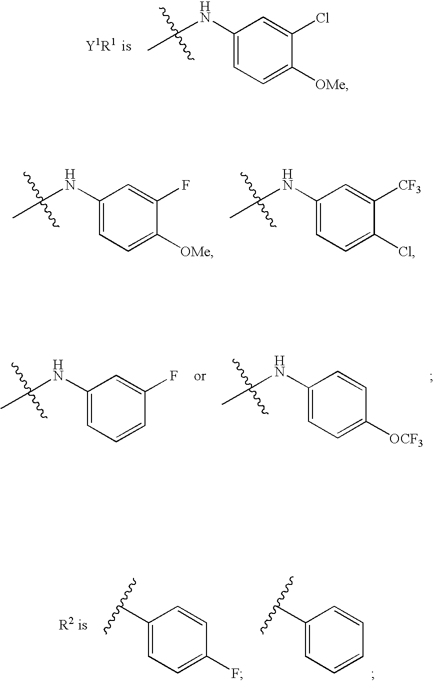

R³is and

R⁴ is H.

In yet another aspect, the present invention provides compounds and compositions comprising these compounds, wherein the compounds have the following formula: 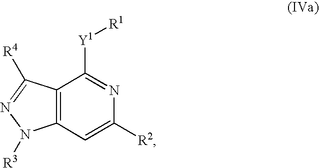
or a salt, including a pharmaceutically acceptable or a non-pharmaceutically acceptable salt, a prodrug, a diastereomeric mixture, an enantiomer, a tautomer, a racemic mixture thereof, or any combination thereof, wherein:

Y¹ is >NR¹, —C≡C—, >O, or a direct a bond between the 6-membered ring and R¹;

R¹ is a substituted or an unsubstituted aryl, heterocyclyl, or heteroaryl, any of which having up to 12 carbon atoms; wherein any heteroaryl or heterocyclyl comprises at least one heteroatom or heterogroup selected from >O, >N—, >S, >NR⁶, >SO₂, or >CO;

R² is a substituted or an unsubstituted alkyl, haloalkyl, aryl, or heteroaryl, any of which having up to 12 carbon atoms; wherein any heteroaryl comprises at least one heteroatom or heterogroup selected from >O, >N—, >S, or >NR⁶;

R³ and R⁴ are selected independently from a substituted or an unsubstituted alkyl or a substituted or an unsubstituted aryl, any of which having up to 12 carbon atoms;

R⁵ is an alkyl having up to 12 carbon atoms or hydrogen;

any of R¹, R², R³, and R⁴ is optionally substituted with at least one group selected independently from: 1) an alkyl, an alkoxy, an alkylthio, a haloalkyl, a haloalkoxy, a cycloalkyl, NR⁶R⁷, CO₂R⁶, COR⁸, CONR⁶R⁷, SO₂R⁸, SO₂NR⁶R⁷, NHSO₂R⁸, or NHCOR⁸, any of which having up to 10 carbon atoms; or 2) halogen, hydroxyl, or cyano;

R⁶ and R⁷ are selected independently from an alkyl or an aryl having up to 10 carbon atoms, or hydrogen; and

R⁸ is an alkyl or aryl having up to 10 carbon atoms.

Further to this aspect, this disclosure provides heterocyclic compounds, wherein the compound is selected from any of the following compounds, including any combination thereof:

4-Benzo[1,3]dioxol-5-yl-6-(4-fluoro-phenyl)-1,3-dimethyl-
1H-pyrazolo[4,3-c]pyridine;
(6-Chloro-pyridin-3-yl)-[6-(4-fluoro-phenyl)-1,3-dimethyl-1H-
pyrazolo[4,3-c]pyridin-4-yl]-amine hydrochloride;
6-(4-Fluoro-phenyl)-4-(3-methanesulfonyl-phenyl)-1,3-dimethyl-
1H-pyrazolo[4,3-c]pyridine;
6-(4-Fluoro-phenyl)-4-(4-methanesulfonyl-phenyl)-1,3-dimethyl-
1H-pyrazolo[4,3-c]pyridine;
6-(4-Fluoro-phenyl)-1,3-dimethyl-4-(4-trifluoromethoxy-phenyl)-
1H-pyrazolo[4,3-c]pyridine.

Another aspect of this invention provides compounds, and compositions comprising the compounds, wherein the compounds have the following formula: 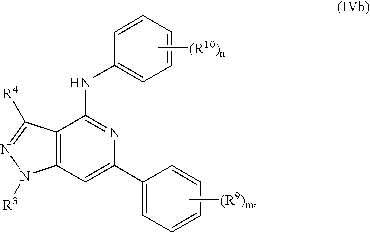
or a salt, including a pharmaceutically acceptable or a non-pharmaceutically acceptable salt, a prodrug, a diastereomeric mixture, an enantiomer, a tautomer, a racemic mixture thereof, or any combination thereof, wherein:

R³ and R⁴ are selected independently from methyl, ethyl, propyl, or phenyl;

R⁹ and R¹⁰, in each occurrence, are selected independently from: 1) an alkyl, an alkoxy, a haloalkyl, a haloalkoxy, NR⁶R⁷, CO₂R⁶, COR⁸, CONR⁶R⁷, SO₂R⁸, or SO₂NR⁶R⁷, any of which having up to 10 carbon atoms; or 2) halogen or cyano;

m and n are selected independently from an integer from 0 to 3, inclusive;

R⁶ and R⁷ are selected independently from H or methyl; and

R⁸ is methyl.

Further, in this aspect, the present invention provides for heterocyclic compounds, wherein the compound is selected from any of the following compounds, including any combination thereof:

(3-Chloro-4-methoxy-phenyl)-[6-(4-fluoro-phenyl)-1,3-dimethyl-1H-
pyrazolo[4,3-c]pyridin-4-yl]-amine hydrochloride;
(3-Fluoro-4-methoxy-phenyl)-[6-(4-fluoro-phenyl)-1,3-dimethyl-
1H-pyrazolo[4,3-c]pyridin-4-yl]-amine hydrochloride;
(4-Chloro-3-trifluoromethyl-phenyl)-[6-(4-fluoro-phenyl)-1,3-dimethyl-
1H-pyrazolo[4,3-c]pyridin-4-yl]-amine hydrochloride;
[6-(4-Fluoro-phenyl)-1,3-dimethyl-1H-pyrazolo[4,3-c]pyridin-4-yl]-(3-
trifluoromethyl-phenyl)-amine hydrochloride;
[6-(4-Fluoro-phenyl)-1,3-dimethyl-1H-pyrazolo[4,3-c]pyridin-4-yl]-(4-
methanesulfonyl-phenyl)-amine;
(1,3-dimethyl-6-(4-fluoro phenyl)-1H-pyrazolo[4,3-c]pyridin-4-yl)-(4-
trifluoromethoxy-phenyl)-amine hydrochloride;
4-[6-(4-Fluoro-phenyl)-1,3-dimethyl-1H-pyrazolo[4,3-c]pyridin-4-
ylamino]-N-methyl-benzenesulfonamide hydrochloride;
N-{4-[6-(4-Fluoro-phenyl)-1,3-dimethyl-1H-pyrazolo[4,3-c]pyridin-4-
ylamino]-phenyl}-methanesulfonamide hydrochloride.

Yet another aspect of this invention provides compounds, and compositions comprising the compounds, wherein the compounds have the following formula: 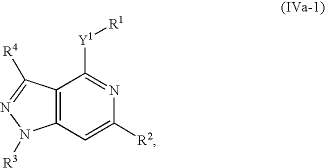
or a salt, including a pharmaceutically acceptable or a non-pharmaceutically acceptable salt, a prodrug, a diastereomeric mixture, an enantiomer, a tautomer, a racemic mixture thereof, or any combination thereof, wherein:

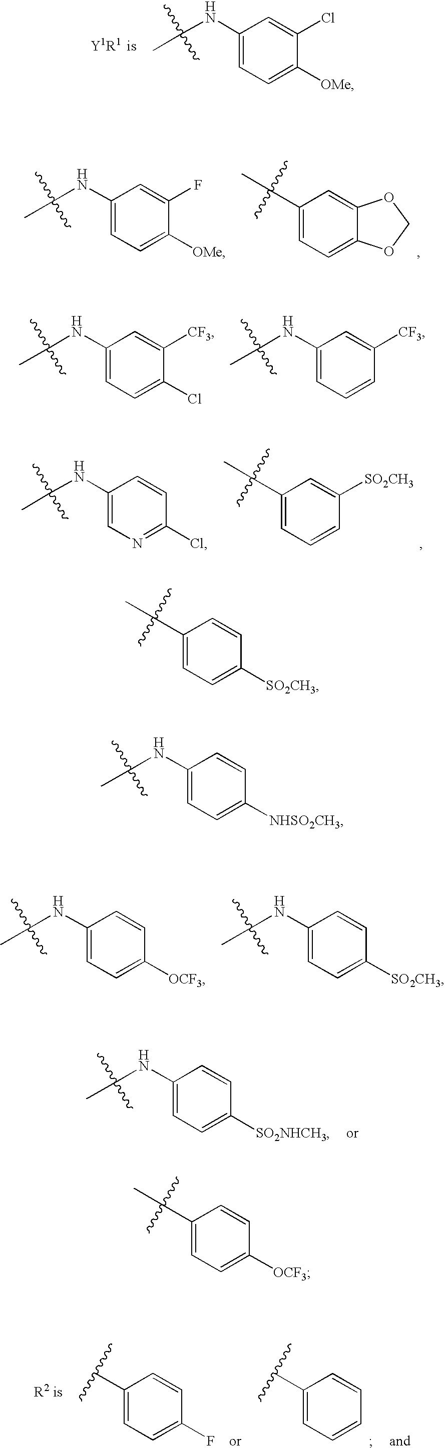

R₃ and R4 are CH₂CH₂CH₃, CH₂CH₃, or CH₃.

A further aspect of this invention provides compounds, and compositions comprising the compounds, wherein the compounds have the following formula: 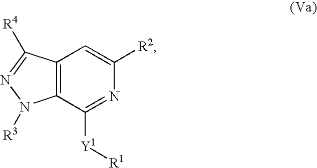
or a salt, including a pharmaceutically acceptable or a non-pharmaceutically acceptable salt, a prodrug, a diastereomeric mixture, an enantiomer, a tautomer, a racemic mixture thereof, or any combination thereof, wherein:

Y¹ is >NR⁵, —C≡C—, >O, or a direct a bond between the 6-membered ring and R¹;

R¹ is a substituted or an unsubstituted aryl, heterocyclyl, or heteroaryl, any of which having up to 12 carbon atoms; wherein any heteroaryl or heterocyclyl comprises at least one heteroatom or heterogroup selected from >O, >N—, >S, >NR⁶, >SO₂, or >CO;

R² is a substituted or an unsubstituted alkyl, haloalkyl, aryl, or heteroaryl, any of which having up to 12 carbon atoms; wherein any heteroaryl comprises at least one heteroatom or heterogroup selected from >O, >N—, >S, or >NR⁶;

R³ and R⁴ are selected independently from a substituted or an unsubstituted alkyl or a substituted or an unsubstituted aryl, any of which having up to 12 carbon atoms, or hydrogen;

R⁵ is a substituted or an unsubstituted alkyl having up to 12 carbon atoms, or hydrogen;

any of R¹, R², R³, and R⁴ can be optionally substituted with at least one group selected independently from: 1) an alkyl, an alkoxy, an alkylthio, a haloalkyl, a haloalkoxy, a cycloalkyl, NR⁶R⁷, —CO₂R⁶, —COR⁸, —CONR⁶R⁷, —SO₂R⁸, —SO₂NR⁶ R⁷, NHSO₂R⁸, or NHCOR⁸, any of which having up to 10 carbon atoms; or 2) halogen, hydroxyl, or cyano;

R⁶ and R⁷ are selected independently from an alkyl or an aryl having up to 10 carbon atoms, or hydrogen; and

R⁸ is an alkyl or aryl having up to 10 carbon atoms.

Further to this aspect of the present invention and the formula (Va) presented immediately above, the following substituents of the formula can be selected as follows, while unspecified substitutents are selected as above:

R² can be a substituted or an unsubstituted haloalkyl, aryl, or thiophenyl, any of which having up to 12 carbon atoms;

R³ and R⁴ can be selected indepdently from a substituted or unsubstituted alkyl having up to 6 carbon atoms or a substituted or unsubstituted phenyl; and

any of R¹, R², R³, and R⁴ can be optionally substituted with at least one group selected independently from: 1) an alkyl, an alkoxy, an alkylthio, a haloalkyl, a haloalkoxy, CONR⁶R⁷, SO₂R⁸, SO₂NR⁶R⁷, NHSO₂R⁸, or NHCOR⁸, any of which having up to 10 carbon atoms; or 2) halogen or hydroxyl.

Still further to this aspect of the present invention and the formula (Va) presented above, the following substituents of the formula can be selected as follows, while unspecified substitutents are selected as above:

Y¹ can be >NH or a direct a bond between the 6-membered ring and R¹;

R¹ can be a substituted or an unsubstituted phenyl, indolyl, benzo[1,3]dioxolyl, benzooxazolyl, or benzimidazolyl;

R² can be a substituted or an unsubstituted phenyl, a substituted or an unsubstituted thiophenyl, or trifluromethyl;

R³ and R⁴ can be selected independently from methyl, ethyl, propyl, or phenyl;

R⁵ is hydrogen;

R¹ can be optionally substituted with at least one group selected independently from: 1) an alkyl, an alkoxy, an alkylthio, a haloalkyl, a haloalkoxy, CONR⁶R⁷, SO₂R⁸, SO₂NR⁶R⁷, NHSO₂R⁸, or NHCOR⁸, any of which having up to 10 carbon atoms; or 2) halogen or hydroxyl.

R² can be optionally substituted with at least one group selected independently from: 1) an alkoxy or SO₂NR⁶R⁷, any of which having up to 10 carbon atoms; or 2) halogen or hydroxyl.

R⁶ and R⁷ are selected independently from an alkyl or an aryl having up to 10 carbon atoms, or hydrogen; and

R⁸is an alkyl or aryl having up to 10 carbon atoms.

Still another aspect of this invention provides compounds, and compositions comprising the compounds, wherein the compound has the following formula: 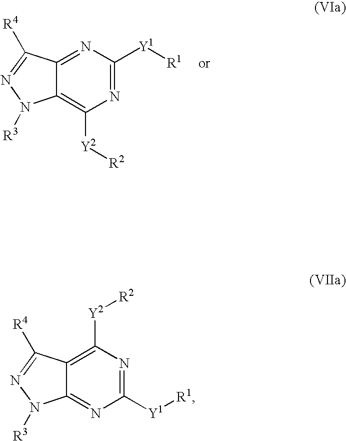
or a salt, including a pharmaceutically acceptable or a non-pharmaceutically acceptable salt, a prodrug, a diastereomeric mixture, an enantiomer, a tautomer, a racemic mixture thereof, or any combination thereof, wherein:

Y¹ and Y² are selected independently from —(CH₂)n- wherein n is 0 or 1, >NH, or —O—;

R¹ and R² are selected independently from CF₃, NMe₂,

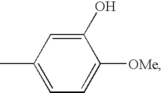

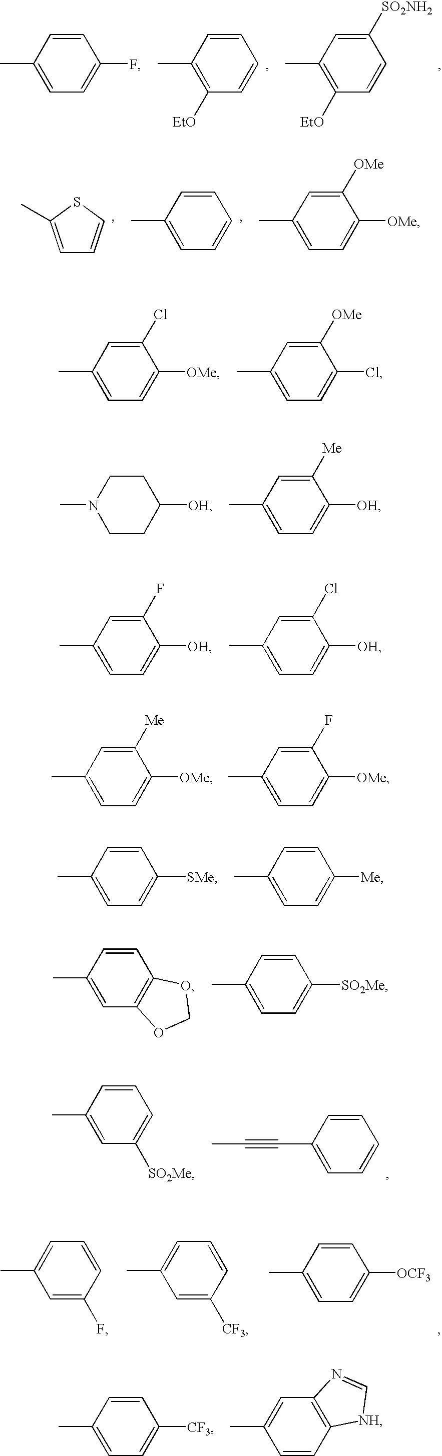

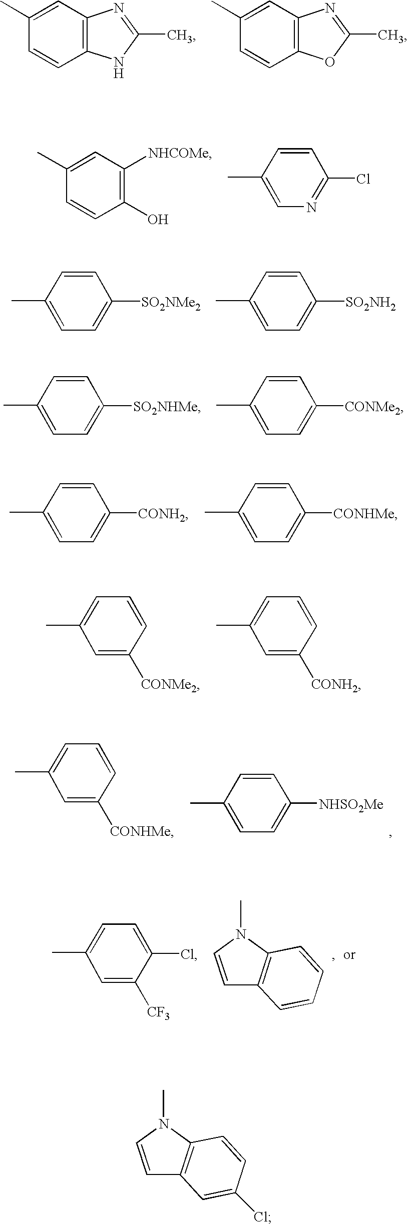

and

R³ and R⁴ are selected independently from H, Me, Et, n-Pr, or

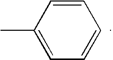

In still another aspect, the present invention provides compounds and compositions comprising compounds, in which the compounds have the following formula: 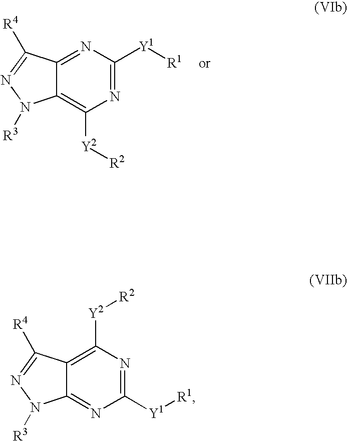
or a salt, including a pharmaceutically acceptable or a non-pharmaceutically acceptable salt, a prodrug, a diastereomeric mixture, an enantiomer, a tautomer, a racemic mixture thereof, or any combination thereof, wherein:

Y¹R¹ and Y²R² are selected independently from F, Cl, CF₃, NMe₂, NEt₂,

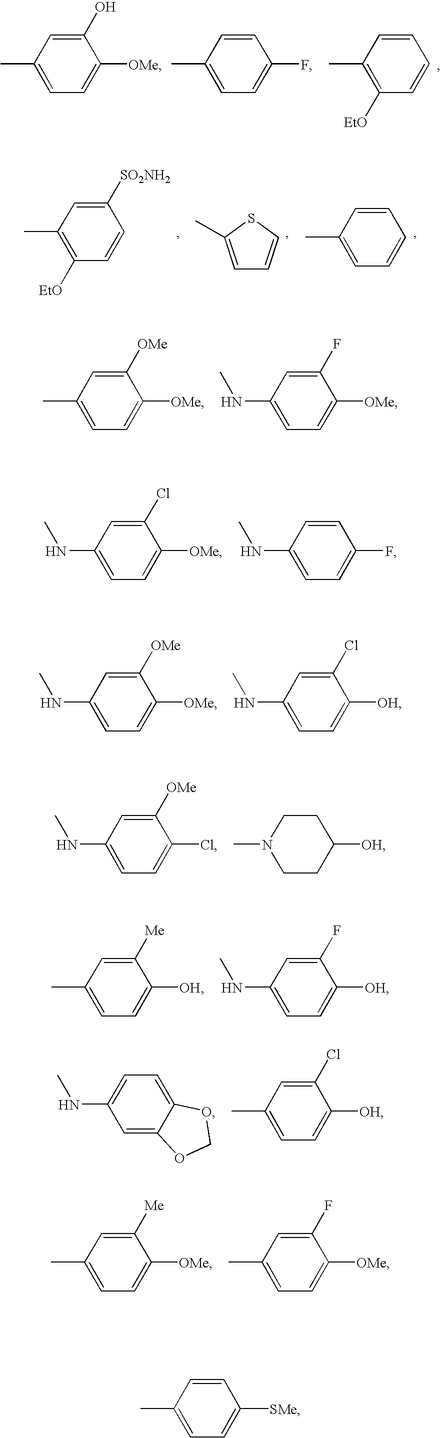

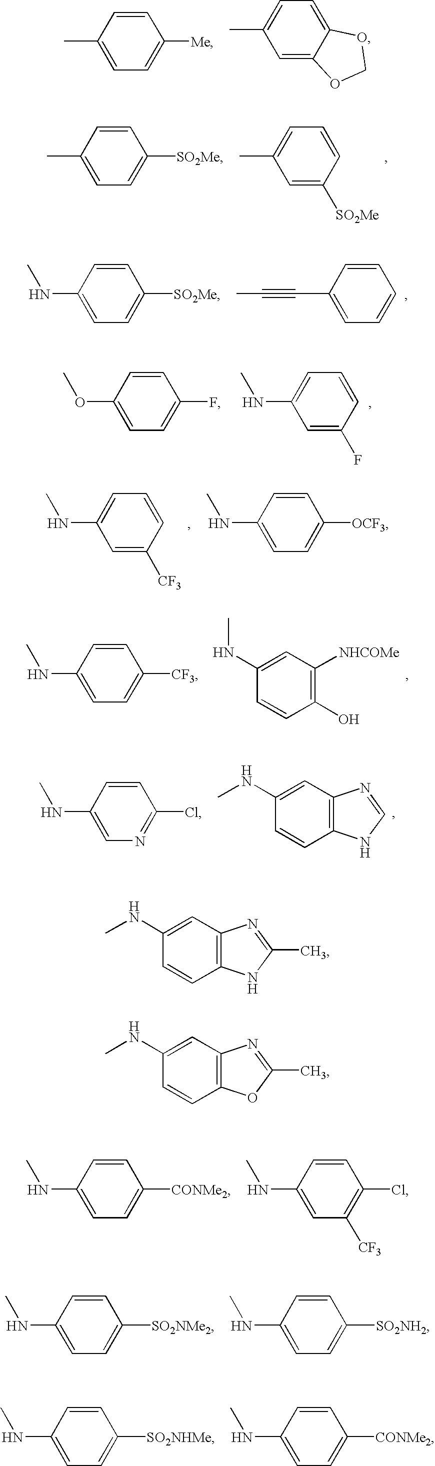

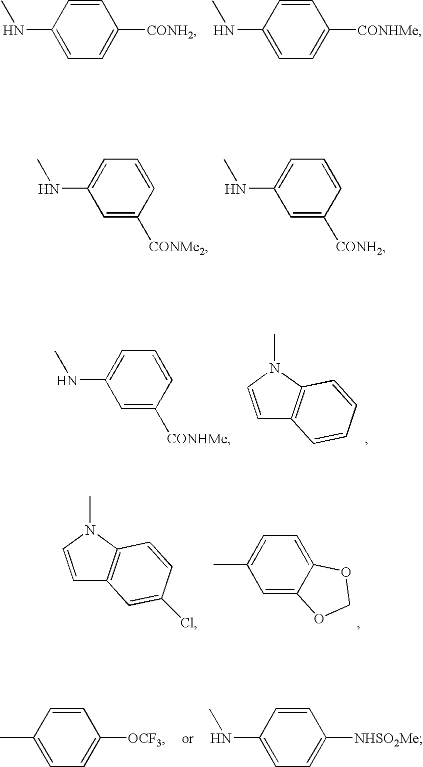

and

R³ and R⁴ are selected independently from H, Me, Et, n-Pr, or

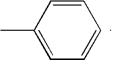

In yet another aspect, the present invention provides compounds and composition comprising compounds, wherein the compounds have the following formula: 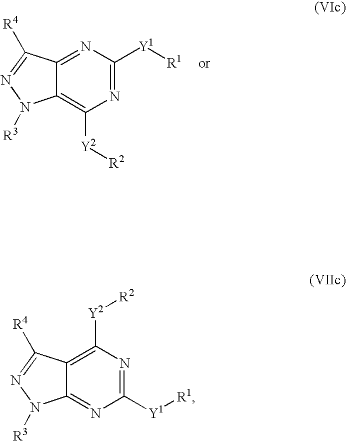
or a salt, including a pharmaceutically acceptable or a non-pharmaceutically acceptable salt, a prodrug, a diastereomeric mixture, an enantiomer, a tautomer, a racemic mixture thereof, or any combination thereof, wherein:

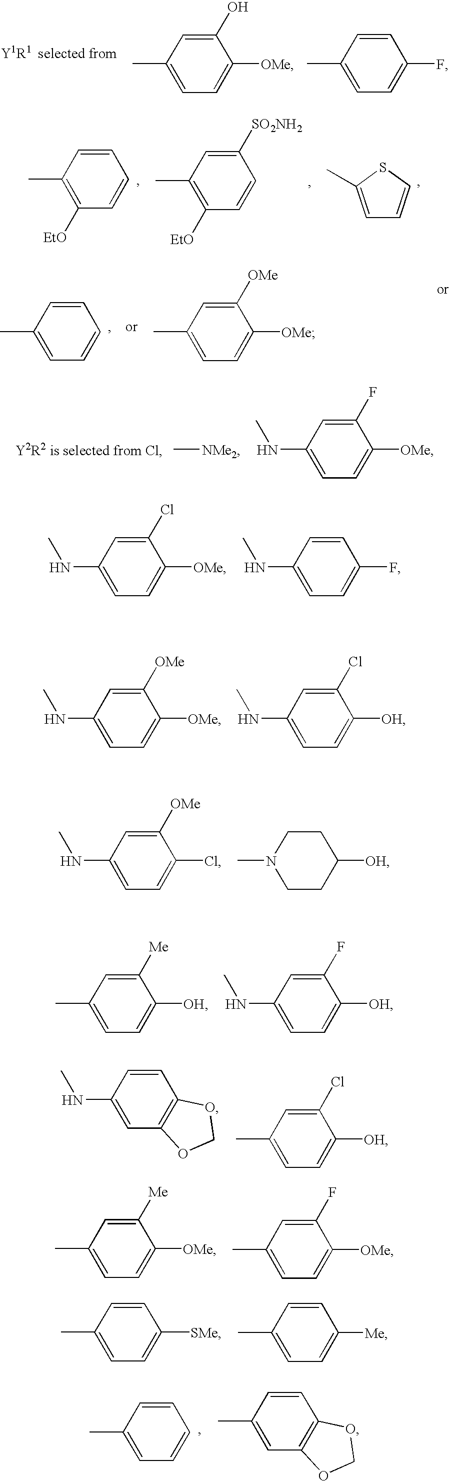

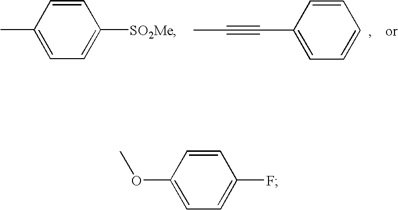

R³ is selected from Me or 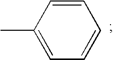
and

R⁴ is selected from H, Me, or n-Pr.

In another aspect, the present invention provides compounds and composition comprising compounds having the following formula: 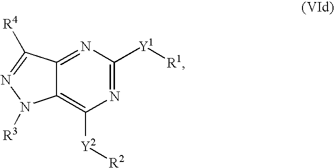
or a salt, including a pharmaceutically acceptable or a non-pharmaceutically acceptable salt, a prodrug, a diastereomeric mixture, an enantiomer, a tautomer, a racemic mixture thereof, or any combination thereof, wherein:

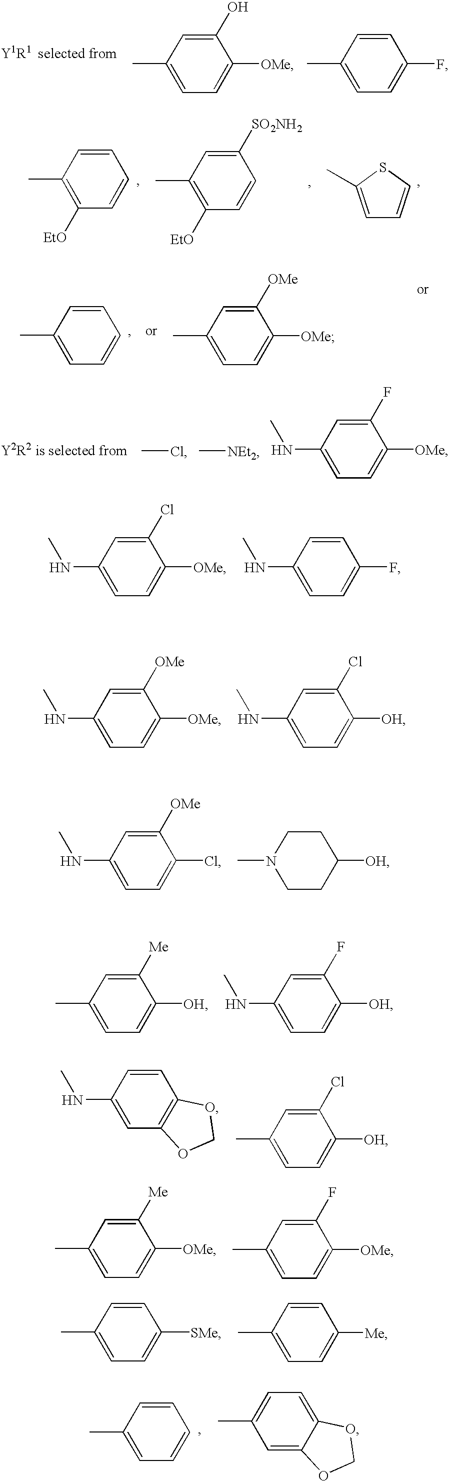

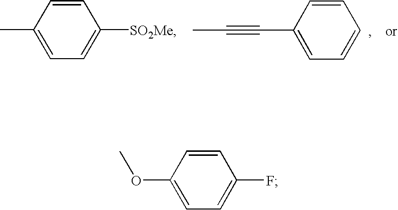

R³ is Me; and

R⁴ is selected from Me or n-Pr.

In still another aspect, the present invention provides compounds and composition comprising compounds, wherein the compounds can have the following formula: 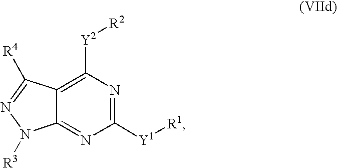
or a salt, including a pharmaceutically acceptable or a non-pharmaceutically acceptable salt, a prodrug, a diastereomeric mixture, an enantiomer, a tautomer, a racemic mixture thereof, or any combination thereof, wherein:

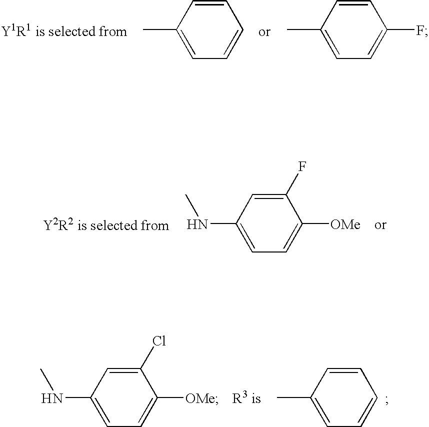

and

R⁴ is H.

Also in another aspect, the present invention provides compounds and composition comprising compounds having the following formula: 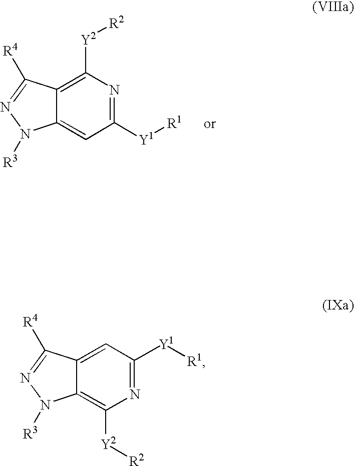
or a salt, including a pharmaceutically acceptable or a non-pharmaceutically acceptable salt, a prodrug, a diastereomeric mixture, an enantiomer, a tautomer, a racemic mixture thereof, or any combination thereof, wherein:

Y¹ and Y² are selected independently from —(CH₂)n- wherein n is 0 or 1, >NH, or —O—;

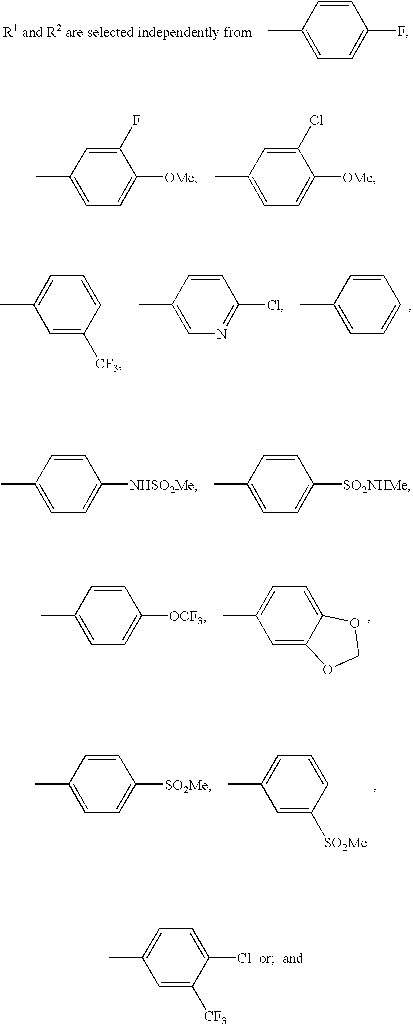

R³ and R⁴ are selected independently from H, Me, Et, n-Pr, or Ph.

Another aspect of this invention is the preparation and use of compounds and composition comprising compounds having the following formula: 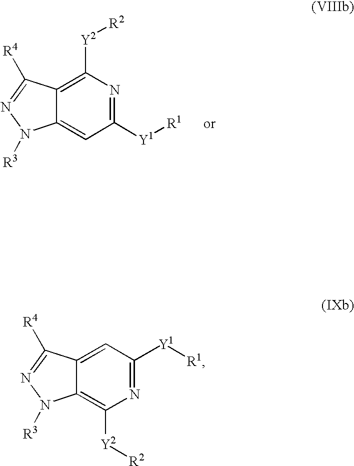
or a salt, including a pharmaceutically acceptable or a non-pharmaceutically acceptable salt, a prodrug, a diastereomeric mixture, an enantiomer, a tautomer, a racemic mixture thereof, or any combination thereof, wherein:

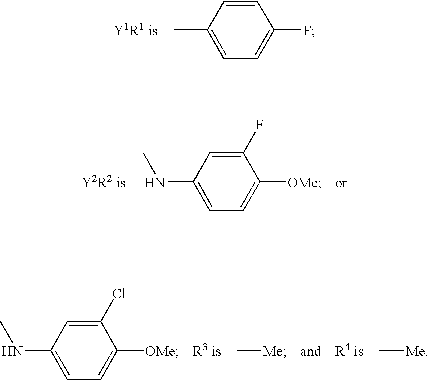

Another aspect of the present invention is the preparation and use of compounds and composition comprising compounds that can have the following formula: 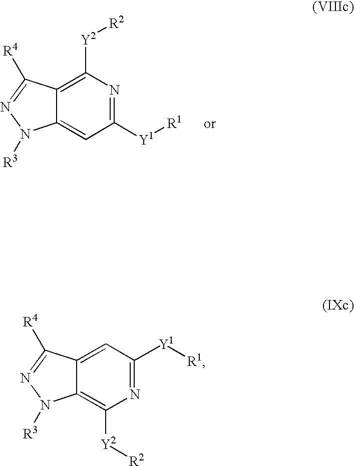
or a salt, including a pharmaceutically acceptable or a non-pharmaceutically acceptable salt, a prodrug, a diastereomeric mixture, an enantiomer, a tautomer, a racemic mixture thereof, or any combination thereof, wherein:

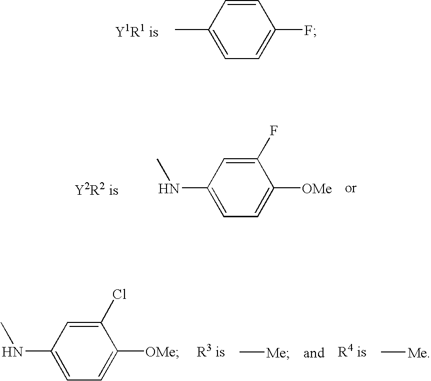

In another aspect, the present invention provides compounds and composition comprising compounds, wherein the compounds have the following formula: 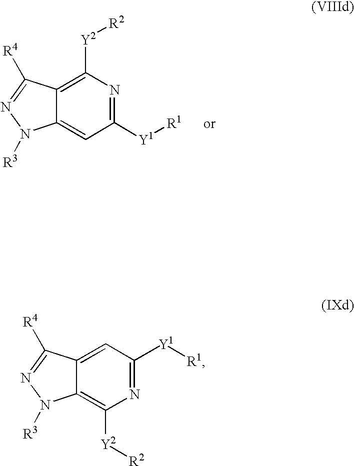
or a salt, including a pharmaceutically acceptable or a non-pharmaceutically acceptable salt, a prodrug, a diastereomeric mixture, an enantiomer, a tautomer, a racemic mixture thereof, or any combination thereof, wherein:

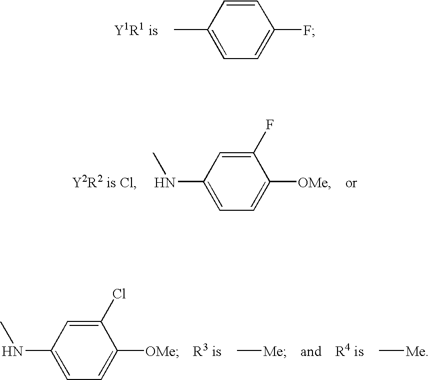

In yet a further aspect, the present invention provides compounds and compositions comprising these compounds, wherein the compounds have the following formula: 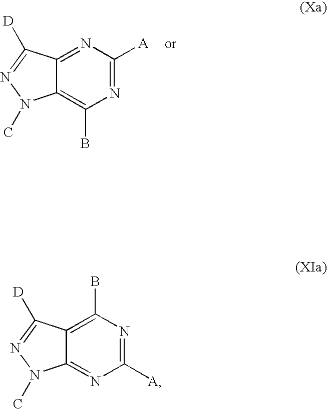
or a salt, including a pharmaceutically acceptable or a non-pharmaceutically acceptable salt, a prodrug, a diastereomeric mixture, an enantiomer, a tautomer, a racemic mixture thereof, or any combination thereof, wherein: A and B are selected independently from A1, A2, or A3, wherein: 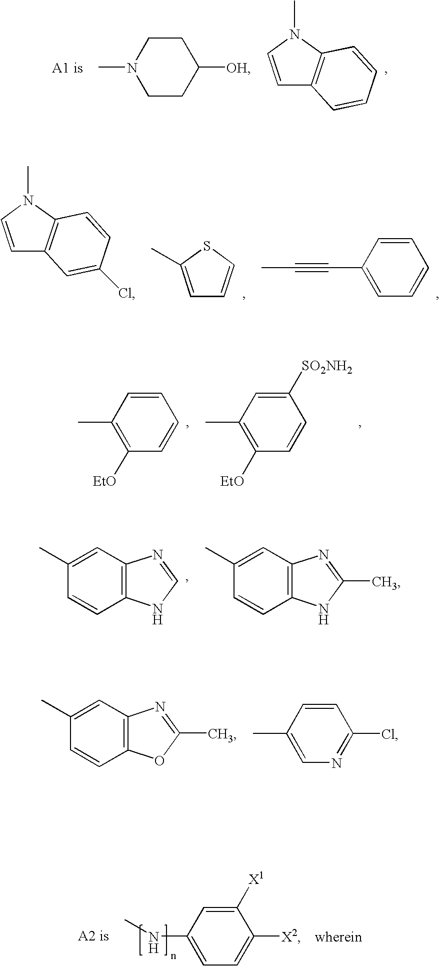
n is 0 or 1;
X¹ is H, F, Cl, OH, OMe, Me, SO₂Me, SO₂NH₂, SO₂NHMe, SO₂NMe₂, CF₃, NHCOMe, C(O)Me, C(O)NH₂, C(O)NHMe, or C(O)NMe₂; and
X² is H, F, Cl, OH, OMe, OCH₂CH₃, SMe, CH₃, CF₃, OCF₃, SO₂Me, SO₂NH₂, SO₂NHMe, SO₂NMe₂, C(O)Me, C(O)NH₂, C(O)NHMe, C(O)NMe₂, NHSO₂Me, or X¹ and X² form a fused 1,3-dioxolane ring; and

A3 is H, F, Cl, CF₃, NMe₂, or NEt₂; and
C and D are selected independently from H, Me, Et, n-Pr, or

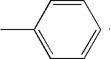

Further to this aspect of the invention and to the formulas (Xa) or (XIa) presented immediately above, the following substituents of the formulas (Xa) or (XIa) can be selected as indicated, while unspecified substitutents are selected as above:

1) A can be selected from A1, A2, or A3, and B can be selected from A1;

2) A can be selected from A1, A2, or A3, and B can be selected from A2;

3) A can be selected from A1, A2, or A3, and B can be selected from A3;

4) A can be selected from A1 or A2, and B can be selected from A1;

5) A can be selected from A1 or A2, and B can be selected from A2;

6) A can be selected from A1 or A2, and B can be selected from A3;

7) A can be selected from A1 and B can be selected from A1;

8) A can be selected from A1 and B can be selected from A2;

9) A can be selected from A1 and B can be selected from A3;

10) A can be selected from A2 and B can be selected from A1;

11) A can be selected from A2 and B can be selected from A2;

12) A can be selected from A2 and B can be selected from A3;

13) A can be selected from A3 and B can be selected from A1;

14) A can be selected from A3 and B can be selected from A2; or

15) A can be selected from A3 and B can be selected from A3.

Additionally, and further to this aspect of the invention and to the formulas (Xa) or (XIa) presented above, the following substituents of the formulas (Xa) or (XIa) can be selected as indicated, while unspecified substitutents are selected as above:

1) B can be selected from A1, A2, or A3, and A can be selected from A1;

2) B can be selected from A1, A2, or A3, and A can be selected from A2;

3) B can be selected from A1, A2, or A3, and A can be selected from A3;

4) B can be selected from A1 or A2, and A can be selected from A1;

5) B can be selected from A1 or A2, and A can be selected from A2;

6) B can be selected from A1 or A2, and A can be selected from A3;

7) B can be selected from A1 and A can be selected from A1;

8) B can be selected from A1 and A can be selected from A2;

9) B can be selected from A1 and A can be selected from A3;

10) B can be selected from A2 and A can be selected from A1;

11) B can be selected from A2 and A can be selected from A2;

12) B can be selected from A2 and A can be selected from A3;

13) B can be selected from A3 and A can be selected from A1;

14) B can be selected from A3 and A can be selected from A2; or

15) B can be selected from A3 and A can be selected from A3.

In a further aspect, the present invention provides compounds and compositions comprising these compounds, wherein the compounds have the following formula: 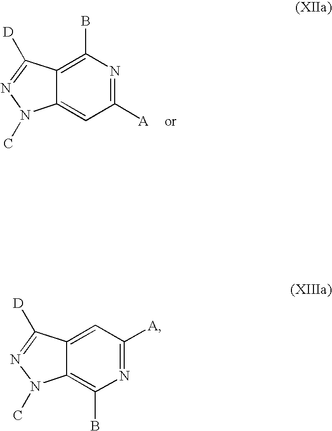
or a salt, including a pharmaceutically acceptable or a non-pharmaceutically acceptable salt, a prodrug, a diastereomeric mixture, an enantiomer, a tautomer, a racemic mixture thereof, or any combination thereof, wherein: A and B are selected independently from A1, A2, or A3, wherein: 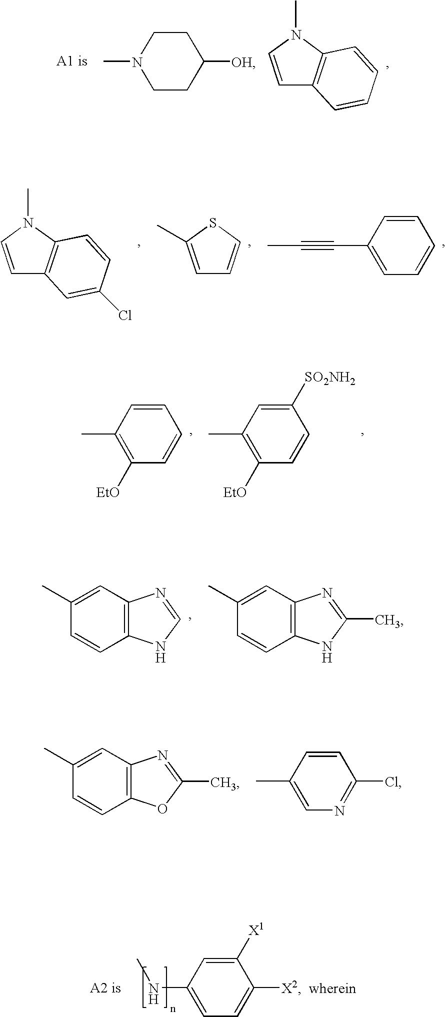
n is 1 or 1;
X¹ is H, F, Cl, OH, OMe, Me, SO₂Me, SO₂NH₂, SO₂NHMe, SO₂NMe₂, CF₃, NHCOMe, C(O)Me, C(O)NH₂, C(O)NHMe, or C(O)NMe₂; and
X² is H, F, Cl, OH, OMe, OCH₂CH₃, SMe, CH₃, CF₃, OCF₃, SO₂Me, SO₂NH₂, SO₂NHMe, SO₂NMe₂, C(O)Me, C(O)NH₂, C(O)NHMe, C(O)NMe₂, NHSO₂Me, or X¹ and X² form a fused 1,3-dioxolane ring; and

A3 is H, F, Cl, CF₃, NMe₂, or NEt₂; and
C and D are selected independently from H, Me, Et, n-Pr, or

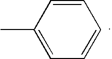

Further to this aspect of the invention and to the formulas (XIIa) or (XIIIa) presented immediately above, the following substituents of the formulas (XIa) or (XIIa) can be selected as indicated, while unspecified substitutents are selected as above:

1) A can be selected from A1, A2, or A3, and B can be selected from A1;

2) A can be selected from A1, A2, or A3, and B can be selected from A2;

3) A can be selected from A1, A2, or A3, and B can be selected from A3;

4) A can be selected from A1 or A2, and B can be selected from A1;

5) A can be selected from A1 or A2, and B can be selected from A2;

6) A can be selected from A1 or A2, and B can be selected from A3;

7) A can be selected from A1 and B can be selected from A1;

8) A can be selected from A1 and B can be selected from A2;

9) A can be selected from A1 and B can be selected from A3;

10) A can be selected from A2 and B can be selected from A1;

11) A can be selected from A2 and B can be selected from A2;

12) A can be selected from A2 and B can be selected from A3;

13) A can be selected from A3 and B can be selected from A1;

14) A can be selected from A3 and B can be selected from A2; or

15) A can be selected from A3 and B can be selected from A3.

Additionally, and further to this aspect of the invention and to the formulas (XIIa) or (XIIIa) presented above, the following substituents of the formulas (XIIa) or (XIIa) can be selected as indicated, while unspecified substitutents are selected as above:

1) B can be selected from A1, A2, or A3, and A can be selected from A1;

2) B can be selected from A1, A2, or A3, and A can be selected from A2;

3) B can be selected from A1, A2, or A3, and A can be selected from A3;

4) B can be selected from A1 or A2, and A can be selected from A1;

5) B can be selected from A1 or A2, and A can be selected from A2;

6) B can be selected from A1 or A2, and A can be selected from A3;

7) B can be selected from A1 and A can be selected from A1;

8) B can be selected from A1 and A can be selected from A2;

9) B can be selected from A1 and A can be selected from A3;

10) B can be selected from A2 and A can be selected from A1;

11) B can be selected from A2 and A can be selected from A2;

12) B can be selected from A2 and A can be selected from A3.

13) B can be selected from A3 and A can be selected from A1;

14) B can be selected from A3 and A can be selected from A2; or

15) B can be selected from A3 and A can be selected from A3.

According to another aspect of this invention, and consistent with the definitions provided herein, the present invention also provides for compounds of the following general structures: 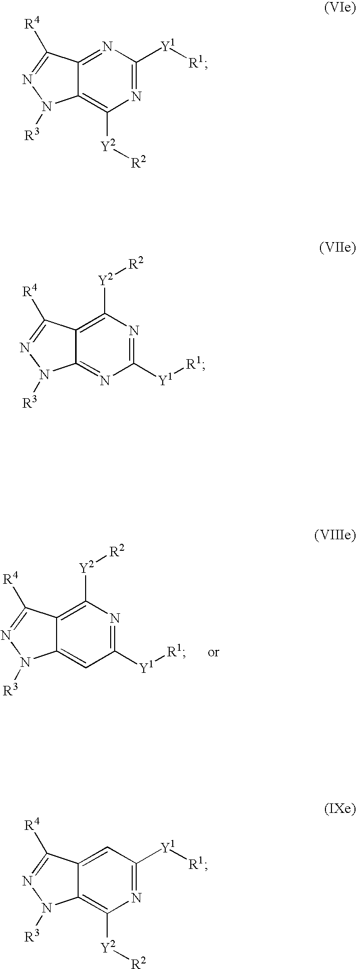
(IXe); or a salt, including a pharmaceutically acceptable or a non-pharmaceutically acceptable salt, a prodrug, a diastereomeric mixture, an enantiomer, a tautomer, a racemic mixture thereof, or any combination thereof, wherein within each structure, the substituents Y¹, R¹, Y², R², R³ and R⁴ can be selected according to the following listings, wherein each substituent is defined in the following table.

The substituent Y¹ and Y² can be selected independently from Y^{A}, Y^{B}, Y^{C}, Y^{D}, Y^{E}, Y^{F}, Y^{G}, Y^{H}, Y^{I}, or Y^{J}.

The substituent R¹ can be selected independently from R^{1A}, R^{1B}, R^{1C}, R^{1D}, R^{1E}, R^{1F}, R^{1G1}, R^{1G2}, R^{1G3}, R^{1G4}, R^{1G5}, R^{1G6}, R^{1H1}, R^{1H2}, R^{1H3}, R^{1I}, R^{1J}, R^{1K}, R^{1L}, R^{1M}, R^{1N}, R^{1O}, R^{1P}, or R^{1Q}.

The substituent R² can be selected independently from R^{2A}, R^{2B}, R^{2C}, R^{2D}, R^{2E}, R^{2F}, R^{2G1}, R^{2G2}, R^{2G3}, R^{2G4}, R^{2G5}, R^{2G6}, R^{2H1}, R^{2H2}, R^{2H3}, R^{2I}, R^{2J}, R^{2K}, R^{2L}, R^{2M}, R^{2N}, R^{2O}, R^{2P}, or R^{2Q}.

Alternatively, the moieties Y¹R¹ and Y²R²can be selected independently from YR^{A}, YR^{B}, YR^{C}, YR^{D}, YR^{E}, YR^{F}, YR^{G}, YR^{H}, YR^{I}, YR^{J}, YR^{K}, or YR^{L}, as defined herein.

The substituent R³ can be selected independently from R^{3A}, R^{3B}, R^{3C}, R^{3D}, R^{3E}, R^{3F}, R^{3G}, R^{3H}, R^{3I}, R^{3J}, R^{3K}, R^{3L}, R^{3M}, R^{3N}, R^{3O}, R^{3P1}, R^{3P2}, R^{3P3}, R^{3P4}, R^{3P5}, R^{3P6}, R^{3Q1}, R^{3Q2}, R^{3Q3}, R^{3R}, R^{3S}, R^{3T}, R^{3U}, or R^{3V}.

The substituent R⁴can be selected independently from R^{4A}, R^{4A}, R^{4C}, R^{4D}, R^{4E}, R^{4F}, R^{4G}, R^{4H}, R^{4I}, R^{4J}, R^{4K}, R^{4L}, R^{4M}, R^{4N}, R^{4O}, R^{4P1}, R^{4P2}, R^{4P3}, R^{4P4}, R^{4P5}, R^{4P6}, R^{4Q1}, R^{4Q2}, R^{4Q3}, R^{4R}, R^{4S}, R^{4T}, R^{4U} , or R^{4V}.

The substituents recited above are defined as follows, consistent with the definitions provided herein.

TABLE 1
Substituent abbreviations.

| | |
|---|---|
| Y^{A} | >NR⁵, wherein R⁵ is defined below |
| Y^{B} | —(CH₂)n—, n is 0 to 3 |
| Y^{C} | —(CH₂)p(CH═CH)(CH₂)q—, p and q are independently 0 to 3 |
| Y^{D} | >CR⁵R⁶, wherein R⁵ and R⁶ are defined below |
| Y^{E} | —(CH₂)p(C≡C)(CH₂)q—, p and q are independently 0 to 3 |
| Y^{F} | >O |
| Y^{G} | >CO |
| Y^{H} | >S |
| Y^{I} | >SO |
| Y^{J} | >SO₂ |
| YR^{A} | saturated or unsaturated carbocyclic or N-heterocyclic ring having up to 12 |
| | carbon atoms |
| YR^{B} | saturated or unsaturated carbocyclic or N-heterocyclic ring having |
| | up to 12 carbon atoms, further comprising >O in the ring |
| YR^{C} | saturated or unsaturated carbocyclic or N-heterocyclic ring having |
| | up to 12 carbon atoms, further comprising >N— in the ring |
| YR^{D} | saturated or unsaturated carbocyclic or N-heterocyclic ring having |
| | up to 12 carbon atoms, further comprising >S in the ring |
| YR^{E} | saturated or unsaturated carbocyclic or N-heterocyclic ring having |
| | up to 12 carbon atoms, further comprising >NR⁶ in the ring, wherein |
| | R⁶ is defined below |
| YR^{F} | saturated or unsaturated carbocyclic or N-heterocyclic ring having |
| | up to 12 carbon atoms, further comprising >SO₂ in the ring |
| YR^{G} | saturated or unsaturated carbocyclic or N-heterocyclic ring having |
| | up to 12 carbon atoms, further comprising >CO in the ring |
| YR^{H} | substituted or an unsubstituted morpholinyl |
| YR^{I} | substituted or an unsubstituted piperazinyl |
| YR^{J} | substituted or an unsubstituted thiomorpholinyl |
| YR^{K} | substituted or an unsubstituted pyrrolidinyl |
| YR^{L} | substituted or an unsubstituted piperidinyl |
| R^{1A}, R^{2A} | Alkyl having up to 12 carbon atoms |
| R^{1B}, R^{2B} | Aryl having up to 12 carbon atoms |
| R^{1C}, R^{2C} | Alkoxyalkyl having up to 12 carbon atoms |
| R^{1D}, R^{2D} | Cycloalky having up to 12 carbon atoms |
| R^{1E}, R^{2E} | —COR⁵ having up to 12 carbon atoms, wherein R⁵ is defined below |
| R^{1F}, R^{2F} | Aralkyl having up to 12 carbon atoms |
| R^{1G1}, R^{2G1} | Heterocyclyl having up to 12 carbon atoms, comprising >O |
| R^{1G2}, R^{2G2} | Heterocyclyl having up to 12 carbon atoms, comprising >N— |
| R^{1G3}, R^{2G3} | Heterocyclyl having up to 12 carbon atoms, comprising >S |
| R^{1G4}, R^{2G4} | Heterocyclyl having up to 12 carbon atoms, comprising >NR⁶, |
| | wherein R⁶ is defined below |
| R^{1G5}, R^{2G5} | Heterocyclyl having up to 12 carbon atoms, comprising >SO₂ |
| R^{1G6}, R^{2G6} | Heterocyclyl having up to 12 carbon atoms, comprising >CO |
| R^{1H1}, R^{2H1} | Heteroaryl having up to 12 carbon atoms, comprising >O |
| R^{1H2}, R^{2H2} | Heteroaryl having up to 12 carbon atoms, comprising >N— or >NR⁶, |
| | wherein R⁶ is defined below |
| R^{1H3}, R^{2H3} | Heteroaryl having up to 12 carbon atoms, comprising >S |
| R^{1I}, R^{2I} | Hydrogen |
| R^{1J}, R^{2J} | Halogen |
| R^{1K}, R^{2K} | Cyano |
| R^{1L}, R^{2L} | Hydroxyl |
| R^{1M}, R^{2M} | Alkoxy having up to 12 carbon atoms |
| R^{1N}, R^{2N} | Alkenyl having up to 12 carbon atoms |
| R^{1O}, R^{2O} | Alkynyl having up to 12 carbon atoms |
| R^{1P}, R^{2P} | —CO₂R⁵ having up to 12 carbon atoms, wherein R⁵ is defined below |
| R^{1Q}, R^{2Q} | —COR⁵ having up to 12 carbon atoms, wherein R⁵ is defined below |
| R^{3A}, R^{4A} | Alkyl having up to 12 carbon atoms |
| R^{3B}, R^{4B} | Alkenyl having up to 12 carbon atoms |
| R^{3C}, R^{4C} | Alkynyl having up to 12 carbon atoms |
| R^{3D}, R^{4D} | Alkoxy having up to 12 carbon atoms |
| R^{3E}, R^{4E} | Cycloalkyl having up to 12 carbon atoms |
| R^{3F}, R^{4F} | Haloalkyl having up to 12 carbon atoms |
| R^{3G}, R^{4G} | Haloalkoxy having up to 12 carbon atoms |
| R^{3H}, R^{4H} | Alkylthio having up to 12 carbon atoms |
| R^{3I}, R^{4I} | Alkylsulfonyl having up to 12 carbon atoms |
| R^{3J}, R^{4J} | Aryl having up to 12 carbon atoms |
| R^{3K}, R^{4K} | —CO₂R⁵ having up to 12 carbon atoms, wherein R⁵ is defined below |
| R^{3L}, R^{4L} | —COR⁵ having up to 12 carbon atoms, wherein R⁵ is defined below |
| R^{3M}, R^{4M} | —NR⁵R⁶ having up to 12 carbon atoms, wherein R⁵ and R⁶ are |
| | defined below |
| R^{3N}, R^{4N} | —SO₂NR⁵R⁶ having up to 12 carbon atoms, wherein R⁵ and R⁶ are |
| | defined below |
| R^{3O}, R^{4O} | —SO₃R⁵ having up to 12 carbon atoms, wherein R⁵ is defined below |
| R^{3P1}, R^{4P1} | Heterocyclyl having up to 12 carbon atoms, comprising >O |
| R^{3P2}, R^{4P2} | Heterocyclyl having up to 12 carbon atoms, comprising >N— |
| R^{3P3}, R^{4P3} | Heterocyclyl having up to 12 carbon atoms, comprising >S |
| R^{3P4}, R^{4P4} | Heterocyclyl having up to 12 carbon atoms, comprising >NR⁶, |
| | wherein R⁶ is defined below |
| R^{3P5}, R^{4P5} | Heterocyclyl having up to 12 carbon atoms, comprising >SO₂ |
| R^{3P6}, R^{4P6} | Heterocyclyl having up to 12 carbon atoms, comprising >CO |
| R^{3Q1}, R^{4Q1} | Heteroaryl having up to 12 carbon atoms, comprising >O |
| R^{3Q2}, R^{4Q2} | Heteroaryl having up to 12 carbon atoms, comprising >N—or >NR⁶, |
| | wherein R⁶ is defined below |
| R^{3Q3}, R^{4Q3} | Heteroaryl having up to 12 carbon atoms, comprising >S |
| R^{3R}, R^{4R} | Hydrogen |
| R^{3S}, R^{4S} | Halogen |
| R^{3T}, R^{4T} | Hydroxyl |
| R^{3U}, R^{4U} | Cyano |
| R^{3V}, R^{4V} | Y¹R¹, independent of the selection of Y¹R¹ |
| R⁵ | R^{5A} R^{5B}, R^{5C}, R^{5D1}, R^{5D2}, R^{5D3}, R^{5E}, R^{5F1}, R^{5F2}, R^{5F3}, R^{5F4}, R^{5F5}, |
| | R^{5F6}, or R^{5G} |
| R⁶ | R^{6A} R^{6B}, R^{6C}, R^{6D1}, R^{6D2}, R^{6D3}, R^{6E}, R^{6F1}, R^{6F2}, R^{6F3}, R^{6F4}, R^{6F5}, |
| | R^{6F6}, or R^{6G} |
| R^{5A}, R^{6A} | Alkyl having up to 12 carbon atoms |
| R^{5B}, R^{6B} | Aryl having up to 12 carbon atoms |
| R^{5C}, R^{6C} | Alkoxyalkyl having up to 12 carbon atoms |
| R^{5D1}, R^{6D1} | Heteroaryl having up to 12 carbon atoms, comprising >O |
| R^{5D2}, R^{6D2} | Heteroaryl having up to 12 carbon atoms, comprising >N— or >NR⁶, |
| | wherein R⁶ is defined below |
| R^{5D3}, R^{6D3} | Heteroaryl having up to 12 carbon atoms, comprising >S |
| R^{5E}, R^{6E} | Cycloalkyl having up to 12 carbon atoms |
| R^{5F1}, R^{6F1} | Heterocyclyl having up to 12 carbon atoms, comprising >O |
| R^{5F2}, R^{6F2} | Heterocyclyl having up to 12 carbon atoms, comprising >N— |
| R^{5F3}, R^{6F3} | Heterocyclyl having up to 12 carbon atoms, comprising >S |
| R^{5F4}, R^{6F4} | Heterocyclyl having up to 12 carbon atoms, comprising >NR⁶, |
| | wherein R⁶ and R⁶ is defined above |
| R^{5F5}, R^{6F5} | Heterocyclyl having up to 12 carbon atoms, comprising >SO₂ |
| R^{5F6}, R^{6F6} | Heterocyclyl having up to 12 carbon atoms, comprising >CO |
| R^{5G}, R^{6G} | Hydrogen |

In these selections, unless otherwise indicated, the number of carbon atoms on the substituents refers to the carbon atoms on the base chemical moiety, and does not include the carbon atoms in any optional substituent. Again, unless otherwise indicated, any substituents are limited in size by the carbon atoms listed in the definitions of the substitutents.

In these selections, the following features are applicable. Any carbocyclic ring, N-heterocyclic ring, morpholinyl, piperazinyl, thiomorpholinyl, pyrrolidinyl, or piperidinyl can be optionally substituted with at least one hydroxyl, halogen, alkyl, alkoxy, haloalkyl, cycloalkyl, aryl, or heteroaryl any of which having up to 6 carbon atoms. Further any when a piperazinyl moiety is present in the substituted heterocyclic compound, the piperazine nitrogen is optionally substituted by an alkyl, a cycloalkyl, an acyl, a haloalkyl, an alkoxyalkyl, SO₂R⁷, SO₂NR⁷₂, or CO₂R⁷, wherein R⁷ is independently selected from: a) an alkyl or an aryl having up to 8 carbon atoms; or b) hydrogen.

Any of the R¹, R², R⁵, or R⁶ moieties that do not constitute hydrogen, halogen, cyano, or hydroxyl (for example, R^{1A} through R^{1H}, R^{1M} through R^{1Q}, R^{2A} through R^{2H}, R^{2M} through R^{2Q}, R^{3A} through R^{3Q} and R^{3V}, R^{4A} through R^{4Q} and R^{4V}, R^{5A} through R^{5F}, and R^{6A} through R^{6F}) can be optionally substituted with at least one group independently selected from: 1) alkyl; alkoxy; alkylthio; haloalkyl; cycloalkyls; aryl; heterocyclyl or heteroaryl comprising at least one heteroatom or heterogroup selected from >O, >N—, >S, >NR⁶, >SO₂, or >CO; haloalkoxy; —OCH₂O—; —OCOR⁹; N(R⁸)₂; —COR⁹; —CON(R⁸)₂; —(CH₂)_{b}CO₂R⁸ wherein b is an integer from 0 to 3; —OCO(CH₂)_{b}—CO₂R¹⁰ wherein b is an integer from 0 to 3; —SO₂R⁹; —NHSO₂R⁹; or —SO₂N(R⁸)₂; any of which having up to 12 carbon atoms; or 2) hydrogen, halogen, hydroxyl, or cyano. In these groups, R⁸, in each occurrence, is independently: 1) an alkyl; a haloalkyl; a heterocyclyl or heteroaryl comprising at least one heteroatom or heterogroup selected from >O, >N—, >S, >NR⁶, >SO₂, or >CO; or an aryl having up to 6 carbon atoms; or 2) hydrogen. Further, in these moieties, R⁹, in each occurrence, is independently an alkyl; a haloalkyl; an aryl; or a heterocyclyl or heteroaryl comprising at least one heteroatom or heterogroup selected from >O, >N—, >S, >NR⁶, >SO₂, or >CO; having up to 8 carbon atoms; wherein R⁹ is optionally substituted with: 1) an alkyl, an alkoxy, a carboxylic acid, or a carboxylic acid ester, any of which having up to 8 carbon atoms; 2) halogen; or 3) hydroxyl.

Any of the R³ or R⁴ moieties that do not constitute hydrogen, halogen, cyano, or hydroxyl can be optionally substituted with at least one group independently selected from: 1) alkyl, alkoxy, haloalkyl, haloalkoxy, cycloalkyl, aryl, heteroaryl, heterocyclyl, alkenyl, alkynyl, —COR¹⁰, —CO₂R¹⁰, —CON(R¹⁰)₂, —SO₂R¹⁰, —SO₂N(R¹⁰)₂, or —N(R¹⁰)₂, any of which having up to 12 carbon atoms; 2) halogen; or 3) hydroxyl; wherein R¹⁰, in each occurrence, is independently: 1) an alkyl or an aryl having up to 6 carbon atoms; or 2) hydrogen.

Representative compounds in accordance with the present invention are presented in the following table. This table is not intended to be an exhaustive listing or exclusive of the compounds of the present invention, but rather exemplary of the heterocyclic compounds that are encompassed by this invention. Further, any listing of a compound as a salt is also intended to be inclusive of the neutral analog of that compound as well, and listing of a neutral compound is also intended to be inclusive of any salt thereof.

TABLE 2
Representative compounds in accordance with the present invention

| Entry | Structure | Name |
|---|---|---|
| 1 | 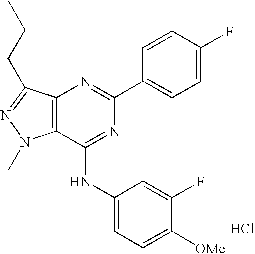 | (3-Fluoro-4-methoxy-phenyl)-[5-(4- fluoro-phenyl)-1-methyl-3-propyl-1H- pyrazolo[4,3-d]pyrimidin-7-yl]-amine hydrochloride |
| 2 | 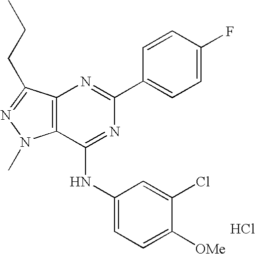 | (3-Chloro-4-methoxy-phenyl)-[5-(4- fluoro-phenyl)-1-methyl-3-propyi-1H- pyrazolo[4,3-d]pyrimidin-7-yl]-amine hydrochloride |
| 3 | 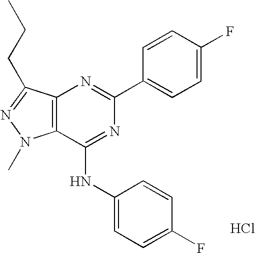 | (4-Fluoro-phenyl)-[5-(4-fluoro-phenyl)- 1-methyl-3-propyl-1H-pyrazolo[4,3- d]pyrimidin-7-yl]-amine hydrochloride |
| 4 | 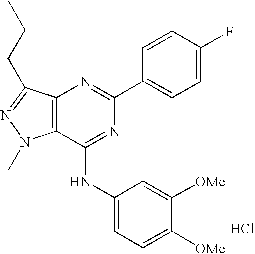 | (3,4-Dimethoxy-phenyl)-[5-(4-fluoro- phenyl)-1-methyl-3-propyl-1H- pyrazolo[4,3-d]pyrimidin-7-yl]-amine hydrochloride |
| 5 | 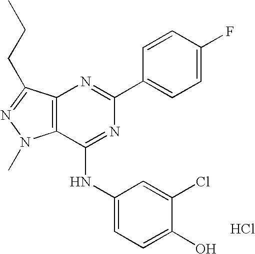 | 2-Chloro-4-[5-(4-fluoro-phenyl)-1- methyl-3-propyl-1H-pyrazolo[4,3- d]pyrimidin-7-ylamino]-phenol hydrochloride |
| 6 | 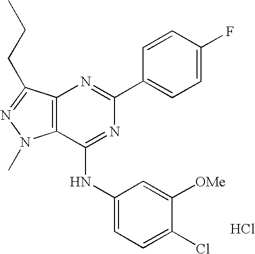 | (4-Chloro-3-methoxy-phenyl)-[5-(4- fluoro-phenyl)-1-methyl-3-propyl-1H- pyrazolo[4,3-d]pyrimidin-7-yl]-amine hydrochloride |
| 7 | 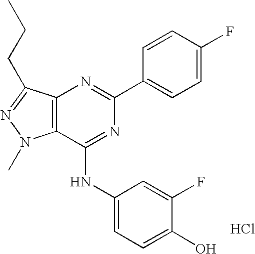 | 2-Fluoro-4-[5-(4-fluoro-phenyl)-1- methyl-3-propyl-1H-pyrazolo[4,3- d]pyrimidin-7-ylamino]-phenol hydrochloride |
| 8 | 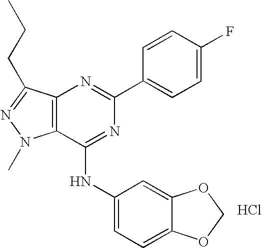 | Benzo[1,3]dioxol-5-yl-[5-(4-fluoro- phenyl)-1-methyl-3-propyl-1H- pyrazolo[4,3-d]pyrimidin-7-yl]-amine hydrochloride |
| 9 | 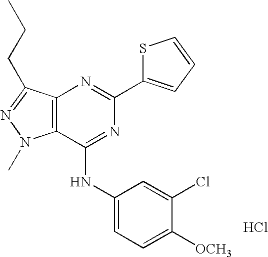 | (3-Chloro-4-methoxy-phenyl)-(1- methyl-3-propyl-5-thiophen-2-yl-1H- pyrazolo[4,3-d]pyrimidin-7-yl)-amine hydrochloride |
| 10 | 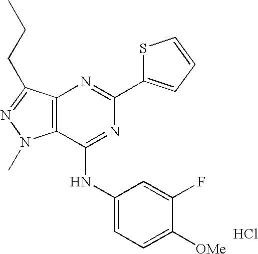 | (3-Fluoro-4-methoxy-phenyl)-(1- methyl-3-propyl-5-thiophen-2-yl-1H- pyrazolo[4,3-d]pyrimidin-7-yl)-amine hydrochloride |
| 11 | 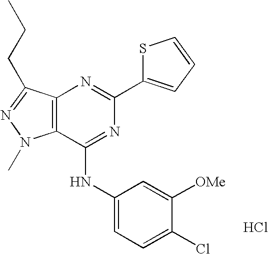 | (4-Chloro-3-methoxy-phenyl)-(1- methyl-3-propyl-5-thiophen-2-yl-1H- pyrazolo[4,3-d]pyrimidin-7-yl)-amine hydrochloride |
| 12 | 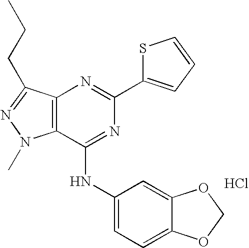 | Benzo[1,3]dioxol-5-yl-(1-methyl-3- propyl-5-thiophen-2-yl-1H- pyrazolo[4,3 -d]pyrimidin-7-yl)-amine hydrochloride |
| 13 | 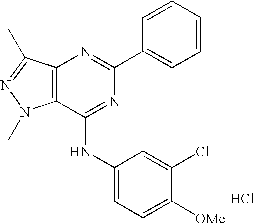 | (3-Chloro-4-methoxy-phenyl)-(1,3- dimethyl-5-phenyl-1H-pyrazolo[4,3- d]pyrimidin-7-yl)-amine hydrochloride |
| 14 | 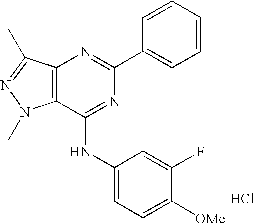 | (1,3-Dimethyl-5-phenyl-1H- pyrazolo[4,3-d]pyrimidin-7-yl)-(3- fluoro-4-methoxy-phenyl)-amine hydrochloride |
| 15 | 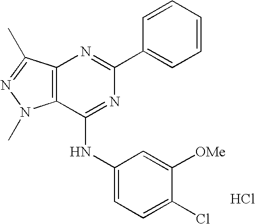 | (4-Chloro-3-methoxy-phenyl)-(1,3- dimethyl-5-phenyl-1H-pyrazolo[4,3- d]pyrimidin-7-yl)-amine hydrochloride |
| 16 | 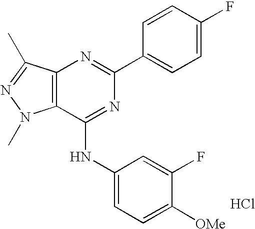 | (3-Fluoro-4-methoxy-phenyl)-[5-(4- fluoro-phenyl)-1,3-dimethyl-1H- pyrazolo[4,3-d]pyrimidin-7-yl]-amine hydrochloride |
| 17 | 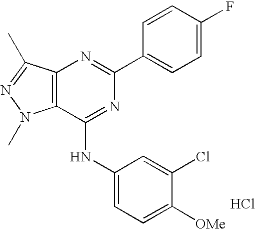 | (3-Chloro-4-methoxy-phenyl)-[5-(4- fluoro-phenyl)-1,3-dimethyl-1H- pyrazolo[4,3-d]pyrimidin-7-yl]-amine hydrochloride |
| 18 | 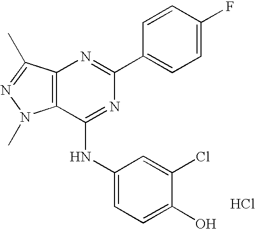 | 2-Chloro-4-[5-(4-fluoro-phenyl)-1,3- dimethyl-1H-pyrazolo[4,3-d]pyrimidin- 7-ylamino]-phenol hydrochloride |
| 19 | 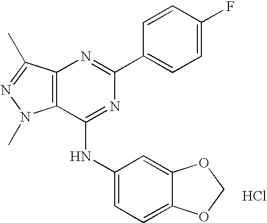 | Benzo[1,3]dioxol-5-yl-[5-(4-fluoro- phenyl)-1,3-dimethyl-1H-pyrazolo[4,3- d]pyrimidin-7-yl]-amine hydrochloride |
| 20 | 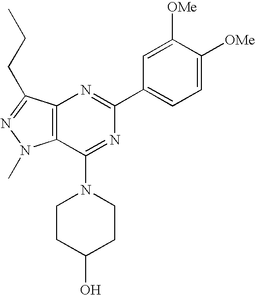 | 1-[5-(3,4-Dimethoxy-phenyl)-1-methyl-3-propyl-1H-pyrazolo[4,3-d]pyrimidin- 7-yl]-piperidin-4-ol |
| 21 | 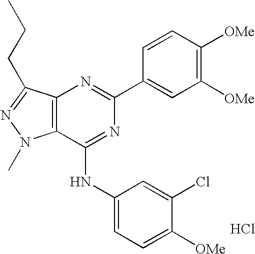 | (3-Chloro-4-methoxy-phenyl)-[5-(3,4- dimethoxy-phenyl)-1-methyl-3-propyl- 1H-pyrazolo[4,3-d]pyrimidin-7-yl]- amine hydrochloride |
| 22 | 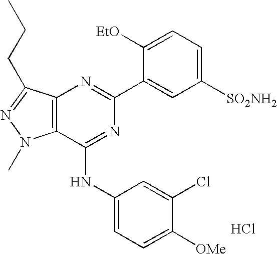 | 3-[7-(3-Chloro-4-methoxy- phenylamino)-1-methyl-3-propyl-1H- pyrazolo[4,3-d]pyrimidin-5-yl]-4- ethoxy-benzenesulfonamide hydrochloride |
| 23 | 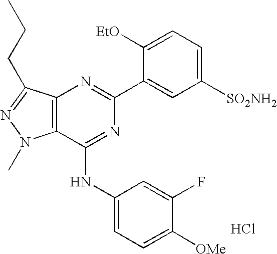 | 4-Ethoxy-3-[7-(3-fluoro-4-methoxy- phenylamino)-1-methyl-3-propyl-1H- pyrazolo[4,3-d]pyrimidin-5-yl]- benzenesulfonamide hydrochloride |
| 24 | 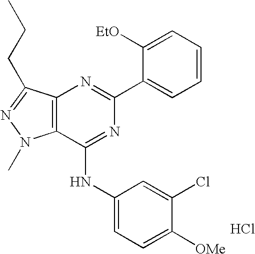 | (3-Chloro-4-methoxy-phenyl)-[5-(2-ethoxy-phenyl)-1-methyl-3-propyl-1H- pyrazolo[4,3-d]pyrimidin-7-yl]-amine hydrochloride |
| 25 | 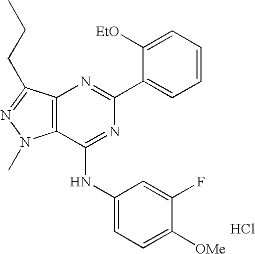 | [5-(2-Ethoxy-phenyl)-1-methyl-3- propyl-1H-pyrazolo[4,3-d]pyrimidin-7- yl]-(3-fluoro-4-methoxy-phenyl)- amine hydrochloride |
| 26 | 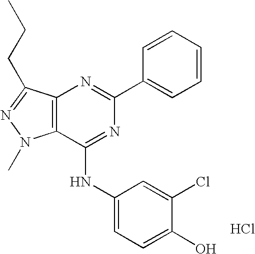 | 2-Chloro-4-(1-methyl-5-phenyl-3- propyl-1H-pyrazolo[4,3-d]pyrimidin-7- ylamino)-phenol hydrochloride |
| 27 | 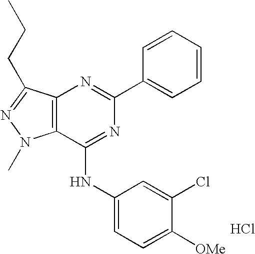 | (3-Chloro-4-methoxy-phenyl)-(1- methyl-5-phenyl-3-propyl-1H- pyrazolo[4,3-d]pyrimidin-7-yl)-amine hydrochloride |
| 28 | 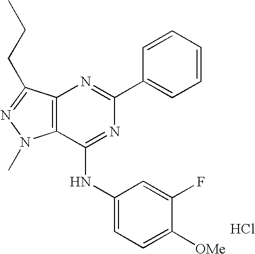 | (3-Fluoro-4-methoxy-phenyl)-(1- methyl-5-phenyl-3-propyl-1H- pyrazolo[4,3-d]pyrimidin-7-yl)-amine hydrochloride |
| 29 | 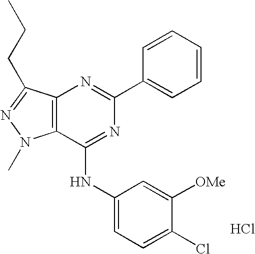 | (4-Chloro-3-methoxy-phenyl)-(1- methyl-5-phenyl-3-propyl-1H- pyrazolo[4,3-d]pyrimidin-7-yl)-amine hydrochloride |
| 30 | 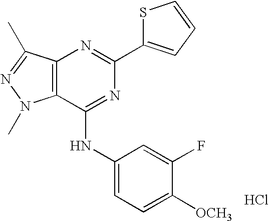 | (1,3-Dimethyl-5-thiophen-2-yl-1H- pyrazolo[4,3-d]pyrimidin-7-yl)-(3- fluoro-4-methoxy-phenyl)-amine hydrochloride |
| 31 | 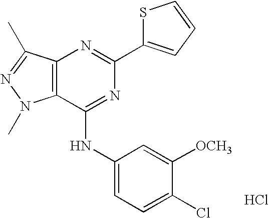 | (4-Chloro-3-methoxy-phenyl)-(1,3- dimethyl-5-thiophen-2-yl-1H- pyrazolo[4,3-d]pyrimidin-7-yl)-amine hydrochloride |
| 32 | 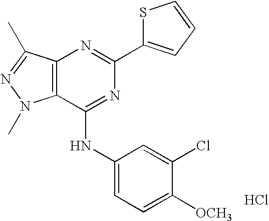 | (3-Chloro-4-methoxy-phenyl)-(1,3- dimethyl-5-thiophen-2-yl-1H- pyrazolo[4,3-d]pyrimidin-7-yl)-amine hydrochloride |
| 33 | 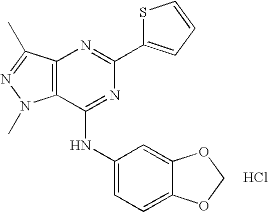 | Benzo[1,3]dioxol-5-yl-(1,3-dimethyl-5- thiophen-2-yl-1H-pyrazolo[4,3- d]pyrimidin-7-yl)-amine hydrochloride |
| 34 | 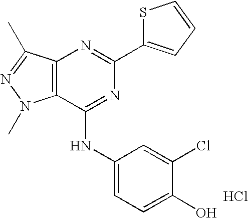 | 2-Chloro-4-(1,3-dimethyl-5-thiophen-2- yl-1H-pyrazolo[4,3-d]pyrimidin-7- ylamino)-phenol hydrochloride |
| 35 | 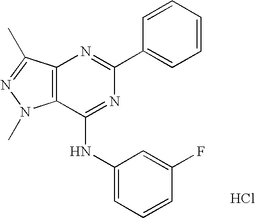 | (1,3-Dimethyl-5-phenyl-1H- pyrazolo[4,3-d]pyrimidin-7-yl)-(3- fluoro-phenyl)-amine hydrochloride |
| 36 | 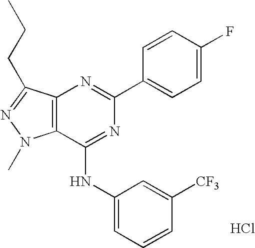 | [5-(4-Fluoro-phenyl)-1-methyl-3- propyl-1H-pyrazolo[4,3-d]pyrimidin-7- yl]-(3-trifluoromethyl-phenyl)-amine hydrochloride |
| 37 | 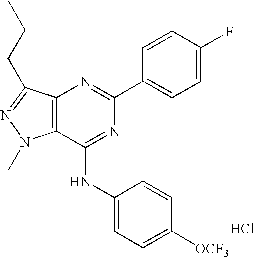 | [5-(4-Fluoro-phenyl)-1-methyl-3- propyl-1H-pyrazolo[4,3-d]pyrimidin-7- yl]-(4-trifluoromethoxy-phenyl)-amine hydrochloride |
| 38 | 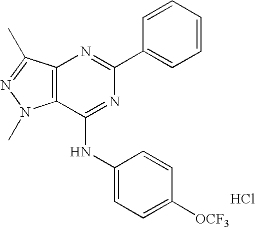 | (1,3-Dimethyl-5-phenyl-1H- pyrazolo[4,3-d]pyrimidin-7-yl)-(4- trifluoromethoxy-phenyl)-amine hydrochloride |
| 39 | 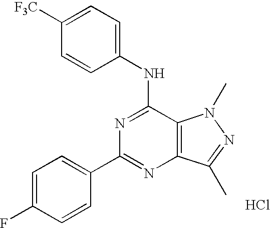 | [5-(4-Fluoro-phenyl)-1,3-dimethyl-1H- pyrazolo[4,3-d]pyrimidin-7-yl]-(4- rifluoromethyl-phenyl)-amine hydrochloride |
| 40 | 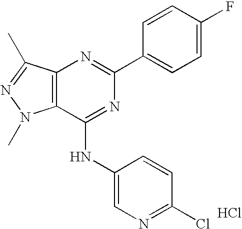 | (6-Chloro-pyridin-3-yl)-[5-(4-fluoro- phenyl)-1,3-dimethyl-1H-pyrazolo[4,3- d]pyrimidin-7-yl]-amine hydrochloride |
| 41 | 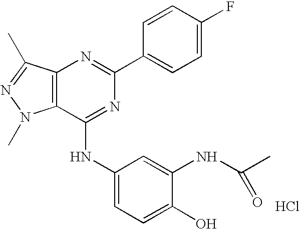 | N-{5-[5-(4-Fluoro-phenyl)-1,3- dimethyl-1H-pyrazolo[4,3-d]pyrimidin- 7-ylamino]-2-hydroxy-phenyl}-acetamide hydrochloride |
| 42 | 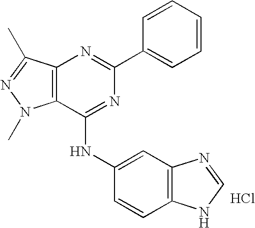 | (1H-Benzoimidazol-5-yl)-(1,3- dimethyl-5-phenyl-1H-pyrazolo[4,3-d]pyrimidin-7-yl)-amine hydrochloride |
| 43 | 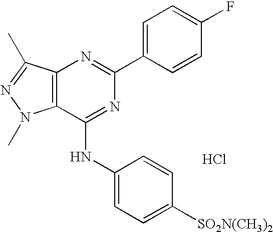 | 4-[5-(4-Fluoro-phenyl)-1,3-dimethyl- 1H-pyrazolo[4,3-d]pyrimidin-7-ylamino]-N,N-dimethyl- benzenesulfonamide hydrochloride |
| 44 | 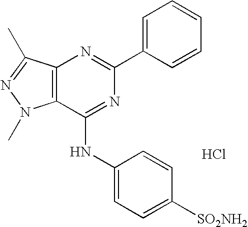 | 4-(1,3-Dimethyl-5-phenyl-1H- pyrazolo[4,3-d]pyrimidin-7-ylamino)- benzenesulfonamide hydrochloride |
| 45 | 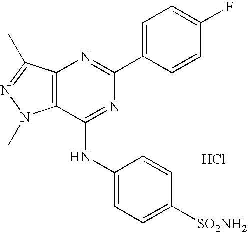 | 4-[5-(4-Fluoro-phenyl)-1,3-dimethyl- 1H-pyrazolo[4,3-d]pyrimidin-7- ylamino]benzenesulfonamide hydrochloride |
| 46 | 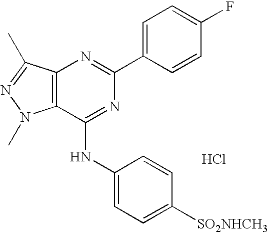 | 4-[5-(4-Fluoro-phenyl)-1,3-dimethyl- 1H-pyrazolo[4,3-d]pyrimidin-7- ylamino]-N-methyl- benzenesulfonamide hydrochloride |
| 47 | 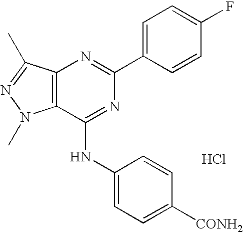 | 4-[5-(4-Fluoro-phenyl)-1,3-dimethyl- 1H-pyrazolo[4,3-d]pyrimidin-7- ylamino]-benzamide hydrochloride |
| 48 | 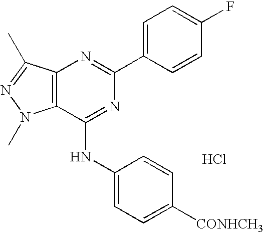 | 4-[5-(4-Fluoro-phenyl)-1,3-dimethyl- 1H-pyrazolo[4,3-d]pyrimidin-7- ylamino]-N-methyl-benzamide hydrochloride |
| 49 | 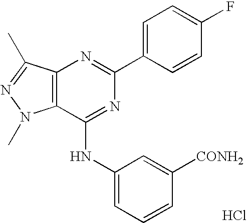 | 3-[5-(4-Fluoro-phenyl)-1,3-dimethyl- 1H-pyrazolo[4,3-d]pyrimidin-7- ylamino]-benzamide hydrochloride |
| 50 | 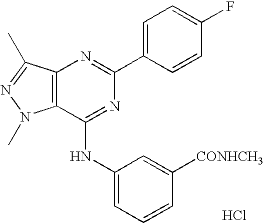 | 3-[5-(4-Fluoro-phenyl)-1,3-dimethyl- 1H-pyrazolo[4,3-d]pyrimidin-7- ylamino]-N-methyl-benzamide hydrochloride |
| 51 | 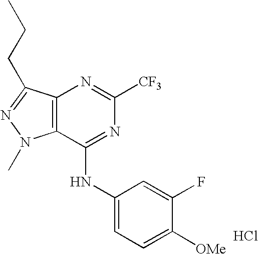 | (3-Fluoro-4-methoxy-phenyl)-(1- methyl-3-propyl-5-trifluoromethyl-1H- pyrazolo[4,3-d]pyrimidin-7-yl)-amine hydrochloride |
| 52 | 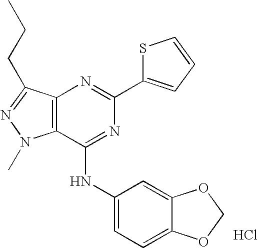 | Benzo[1,3]dioxol-5-yl-(1-methyl-3- propyl-5-thiophen-2-yl-1H- pyrazolo[4,3-d]pyrimidin-7-yl)-amine hydrochloride |
| 53 | 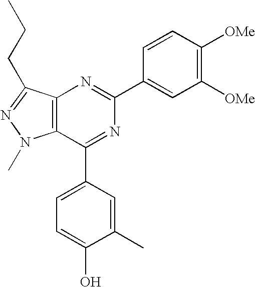 | 4-[5-(3,4-Dimethoxy-phenyl)-1-methyl- 3-propyl-1H-pyrazolo[4,3 -d]pyrimidin- 7-yl]-2-methyl-phenol |
| 54 | 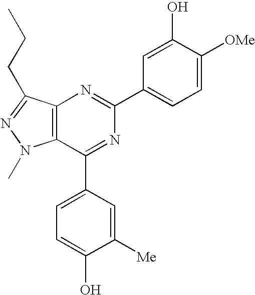 | 4-[5-(3-hydroxy,4-methoxy-phenyl)-1- methyl-3-propyl-1H-pyrazolo[4,3- d]pyrimidin-7-yl]-2-methyl-phenol |
| 55 | 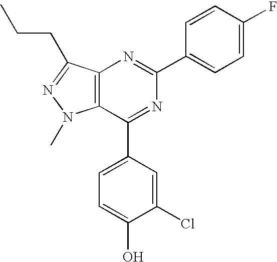 | 2-Chloro-4-[5-(4-fluoro-phenyl)- 1-methyl-3-propyl-1H- pyrazolo[4,3-d]pyrimidin-7-yl]-phenol |
| 56 | 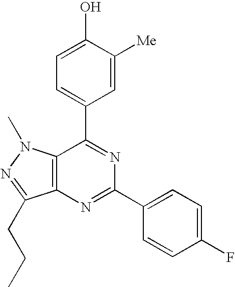 | 5-(4-Fluoro-phenyl)-7-(4-hydroxy-3- methyl-phenyl)-1-methyl-3-propyl-1H- pyrazolo[4,3-d]pyrimidine |
| 57 | 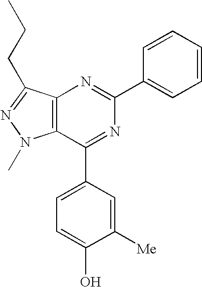 | 2-Methyl-4-(1-methyl-5-phenyl-3- propyl-1H-pyrazolo[4,3-d]pyrimidin-7- yl)-phenol |
| 58 | 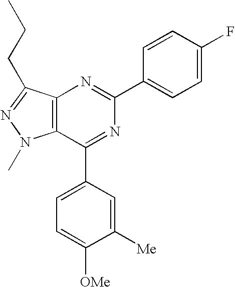 | 5-(4-Fluoro-phenyl)-7-(4-methoxy-3- methyl-phenyl)-1-methyl-3-propyl-1H- pyrazolo[4,3-d]pyrimidine |
| 59 | 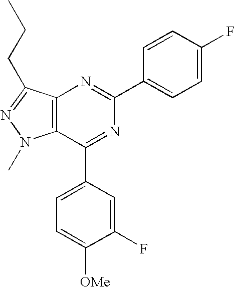 | 7-(3-Fluoro-4-methoxy-phenyl)- 5-(4-fluoro-phenyl)-1-methyl-3-propyl 1H-pyrazolo[4,3-d]pyrimidine |
| 60 | 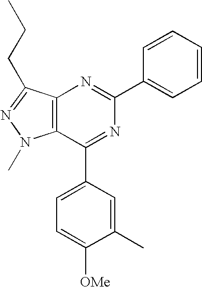 | 7-(4-Methoxy-3-methyl-phenyl)-1- methyl-5-phenyl-3-propyl-1H- pyrazolo[4,3-d]pyrimidine |
| 61 | 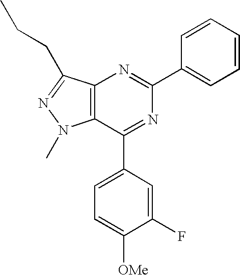 | 7-(3-Fluoro-4-methoxy-phenyl)-1- methyl-5-phenyl-3-propyl-1H- pyrazolo[4,3-d]pyrimidine |
| 62 | 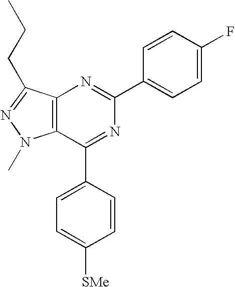 | 5-(4-Fluoro-phenyl)-1-methyl-7-(4- methylsulfanyl-phenyl)-3-propyl-1H- pyrazolo[4,3-d]pyrimidine |
| 63 | 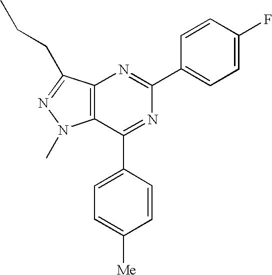 | 5-(4-Fluoro-phenyl)-1-methyl-3-propyl- 7-p-tolyl-1H-pyrazolo[4,3-d]pyrimidine |
| 64 | 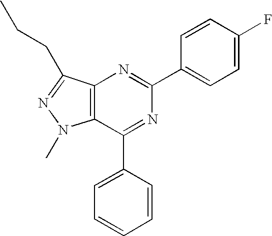 | 5-(4-Fluoro-phenyl)-1-methyl-7-phenyl- 3-propyl-1H-pyrazolo[4,3-d]pyrimidine |
| 65 | 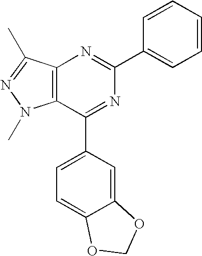 | 7-Benzo[1,3]dioxol-5-yl-1,3-dimethyl- 5-phenyl-1H-pyrazolo[4,3-d]pyrimidine |
| 66 | 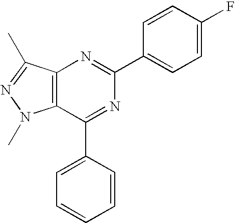 | 5-(4-fluoro-phenyl)-1,3-dimethyl-7- phenyl-1H-pyrazolo[4,3-d]pyrimidine |
| 67 | 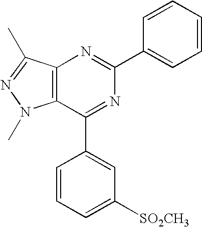 | 7-(3-Methanesulfonyl-phenyl)-1,3- dimethyl-5-phenyl-1H-pyrazolo[4,3- d]pyrimidine |
| 68 | 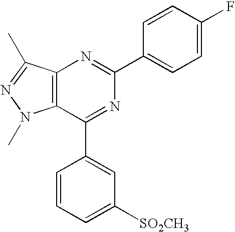 | 5-(4-Fluoro-phenyl)-7-(3- methanesulfonyl-phenyl)-1,3-dimethyl- 1H-pyrazolo[4,3-d]pyrimidine |
| 69 | 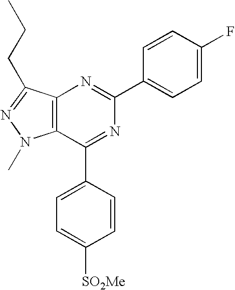 | 5-(4-Fluoro-phenyl)-7-(4- methanesulfonyl-phenyl)-1-methyl-3- propyl-1H-pyrazolo[4,3-d]pyrimidine |
| 70 | 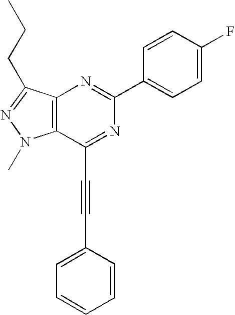 | 5-(4-Fluoro-phenyl)-1-methyl-7- phenylethynyl-3-propyl-1H- pyrazolo[4,3-d]pyrimidine |
| 71 | 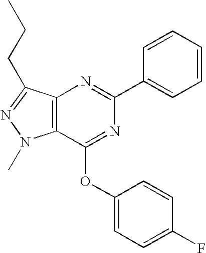 | 7-(4-Fluoro-phenoxy)-1-methyl-5- phenyl-3-propyl-1H-pyrazolo[4,3- d]pyrimidine |
| 72 | 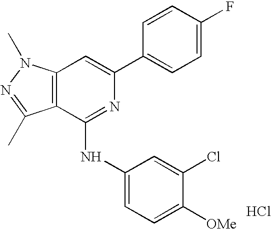 | (3-Chloro-4-methoxy-phenyl)-[6-(4- fluoro-phenyl)-1,3-dimethyl-1H- pyrazolo[4,3-c]pyridin-4-yl]-amine hydrochloride |
| 73 | 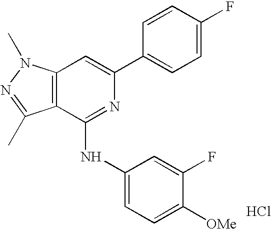 | (3-Fluoro-4-methoxy-phenyl)-[6-(4- fluoro-phenyl)-1,3-dimethyl-1H- pyrazolo[4,3-c]pyridin-4-yl]-amine hydrochloride |
| 74 | 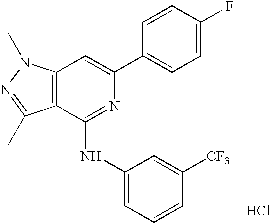 | [6-(4-Fluoro-phenyl)-1,3-dimethyl-1H-pyrazolo[4,3-c]pyridin-4-yl]-(3- trifluoromethyl-phenyl)-amine hydrochloride |
| 75 | 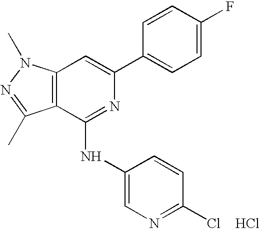 | (6-Chloro-pyridin-3-yl)-[6-(4-fluoro- phenyl)-1,3-dimethyl-1H-pyrazolo[4,3- c]pyridin-4-yl]-amine hydrochloride |
| 76 | 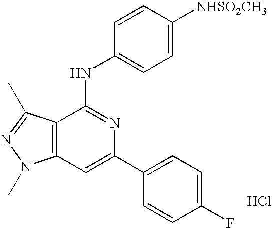 | N-{4-[6-(4-Fluoro-phenyl)-1,3- dimethyl-1H-pyrazolo[4,3-c]pyridin-4- ylamino]-phenyl}-methanesulfonamide hydrochloride |
| 77 | 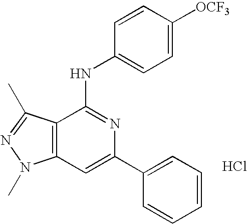 | 6-(4-fluorophenyl)-(1,3-dimethyl-6- phenyl-1H-pyrazolo[4,3-c]pyridin-4-yl)- (4-trifluoromethoxy-phenyl)-amine hydrochloride |
| 78 | 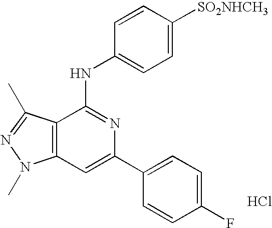 | 4-[6-(4-Fluoro-phenyl)-1,3-dimethyl- 1H-pyrazolo[4,3-c]pyridin-4-ylamino]- N-methyl-benzenesulfonamide hydrochloride |
| 79 | 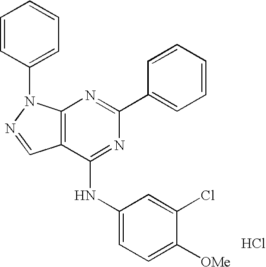 | 3-Chloro-4-methoxy-phenyl)-(1,6- diphenyl-1H-pyrazolo[3,4-d]pyrimidin- 4-yl)-amine hydrochloride |
| 80 | 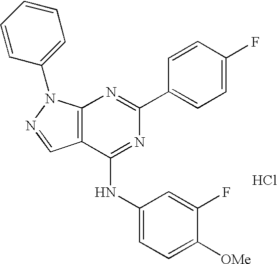 | (3-Fluoro-4-methoxyphenyl)-[6-(4- fluoro-phenyl)-1-phenyl-1H- pyrazolo[3,4-d]-pyrimidin-4yl]amine hydrochloride |
| 81 | 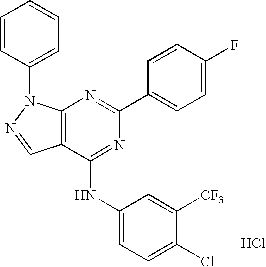 | (4-Chloro-3-trifluoromethyl-phenyl)-[6- (4-fluoro-phenyl)-1-phenyl-1H- pyrazolo[3,4-d]pyrimidin-4-yl[-amine hydrochloride |
| 82 | 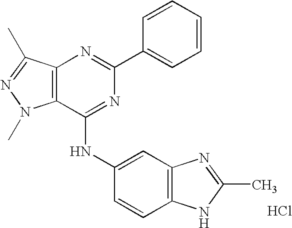 | (1,3-Dimethyl-5-phenyl-1H- pyrazolo[4,3-d]pyrimidin-7-yl)-(2- methyl-1H-benzoimidazol-5-yl)-amine hydrochloride |
| 83 | 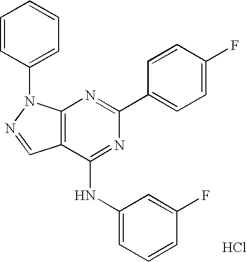 | (3-Fluoro-phenyl)-[6-(4-fluoro-phenyl)- 1-phenyl-1H-pyrazolo[3,4-d]pyrimidin- 4-yl]-amine hydrochloride |
| 84 | 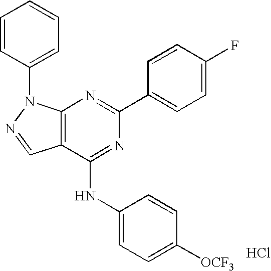 | [6-(4-Fluoro-phenyl)-1-phenyl-1H- pyrazolo[3,4-d]pyrimidin-4-yl]-(4- trifluoromethoxy-phenyl)-amine hydrochloride |
| 85 | 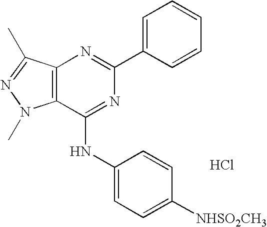 | N-[4-(1,3-Dimethyl-5-phenyl-1H- pyrazolo[4,3-d]pyrimidin-7-ylamino)- phenyl]-methanesulfonamide hydrochloride |
| 86 | 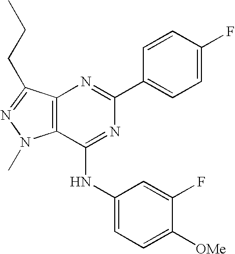 | (3-Fluoro-4-methoxy-phenyl)-[5-(4- fluoro-phenyl)-1-methyl-3-propyl-1H- pyrazolo[4,3-d]pyrimidin-7-yl]-amine |
| 87 | 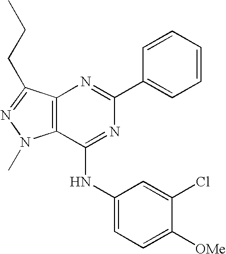 | (3-Chloro-4-methoxy-phenyl)-(1- methyl-5-phenyl-3-propyl-1H- pyrazolo[4,3 -d]pyrimidin-7-yl)-amine |
| 88 | 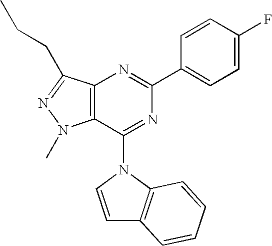 | 5-(4-Fluoro-phenyl)-7-indol-1-yl-1- methyl-3-propyl-1H-pyrazolo[4,3- d]pyrimidine |
| 89 | 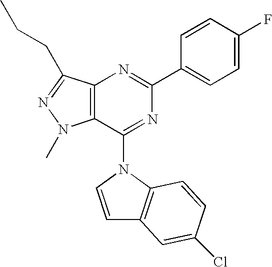 | 7-(5-Chloro-indol-1-yl)-5-(4-fluoro- phenyl)-1-methyl-3-propyl-1H- pyrazolo[4,3-d]pyrimidine |
| 90 | 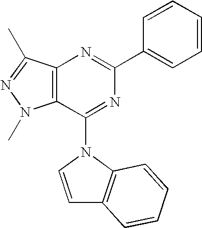 | 7-Indol-1-yl-1,3-dimethyl-5-phenyl-1H- pyrazolo[4,3-d]pyrimidine |
| 91 | 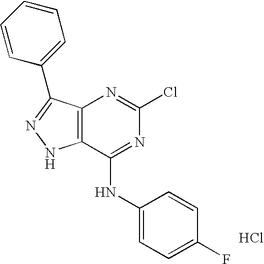 | 5-Chloro-3-phenyl-1H-pyrazolo[4,3- d]pyrimidin-7-yl)-(4-fluoro-phenyl)- amine hydrochloride |
| 92 | 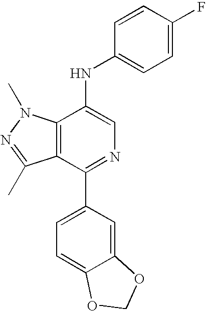 | 4-Benzo[1,3]dioxol-5-yl-6-(4-fluoro- phenyl)-1,3-dimethyl-1H-pyrazolo[4,3-c]pyridine |
| 93 | 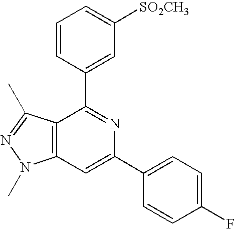 | 6-(4-Fluoro-phenyl)-4-(3- methanesulfonyl-phenyl)-1,3-dimethyl-1H- pyrazolo[4,3-c]pyridine |
| 94 | 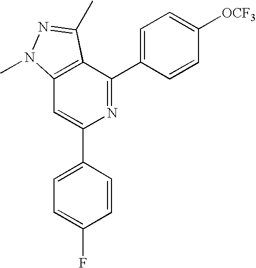 | 6-(4-Fluoro-phenyl)-1,3-dimethyl-4-(4- trifluoromethoxy-phenyl)-1H- pyrazolo[4,3-c]pyridine |
| 95 | 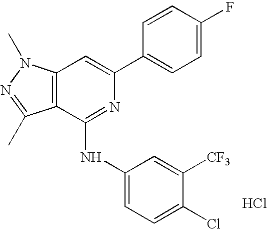 | (4-Chloro-3-trifluoromethyl-phenyl)-[6- (4-fluoro-phenyl)-1,3-dimethyl-1H- pyrazolo[4,3-c]pyridin-4-yl]-amine hydrochloride |
| 96 | 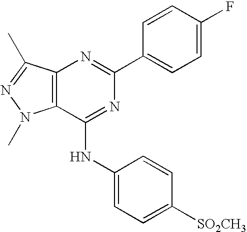 | [5-(4-Fluoro-phenyl)-1,3-dimethyl-1H- pyrazolo[4,3-d]pyrimidin-7-yl]-(4- methanesulfonyl-phenyl)-amine |
| 97 | 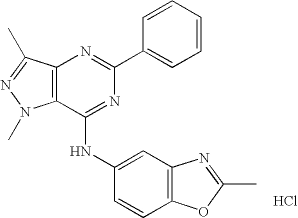 | (1,3-Dimethyl-5-phenyl-1H- pyrazolo[4,3-d]pyrimidin-7-yl)-(2- methyl-benzooxazol-5-yl)- amine hydrochloride |
| 98 | 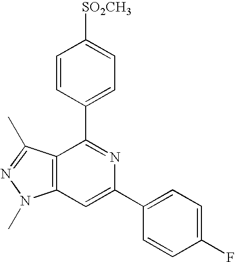 | 6-(4-Fluoro-phenyl)-4-(4- methanesulfonyl-phenyl)-1,3-dimethyl- 1H-pyrazolo[4,3-c]pyridine |
| 99 | 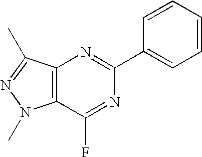 | 7-Fluoro-1,3-dimethyl-5-phenyl-1H- pyrazolo[4,3-d]pyrimidine |
| 100 | 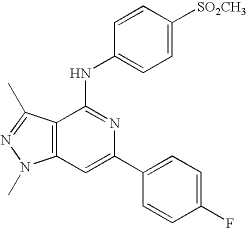 | [6-(4-Fluoro-phenyl)-1,3-dimethyl-1H- pyrazolo[4,3-c]pyridin-4-yl]-(4- methanesulfonyl-phenyl)-amine |
| 101 | 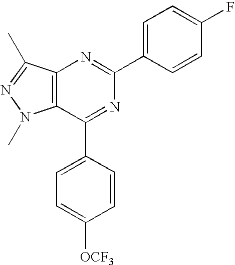 | 5-(4-Fluoro-phenyl)-1,3-dimethyl-7-(4- trifluoromethoxy-phenyl)-1H- pyrazolo[4,3-d]pyrimidine |
| 102 | 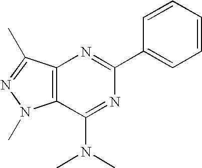 | (1,3-Dimethyl-5-phenyl-1H-pyrazolo[4,3-d]pyrimidin- 7-yl)-dimethyl-amine |

Additional representative compounds in accordance with the present invention are presented in the following table, which include some of the intermediate species in the preparation of the compounds of this invention, as well as other compounds as well. This table is also not intended to be an exhaustive listing, but rather exemplary of the heterocyclic compounds that are encompassed by this invention. Further, any listing of a compound as a salt is also intended to be inclusive of the neutral analog of that compound as well, and listing of a neutral compound is also intended to be inclusive of any salt thereof.

TABLE 3
Representative compounds in accordance with the present invention

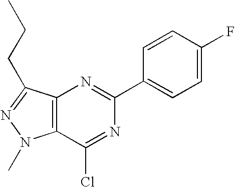

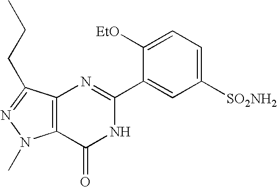

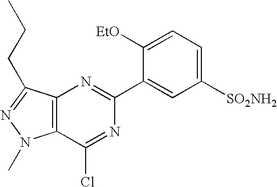

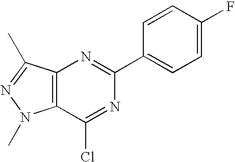

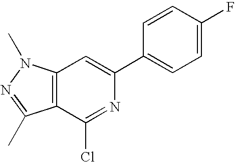

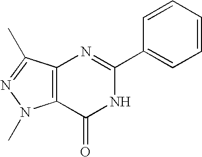

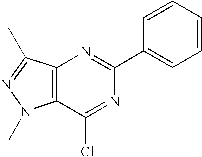

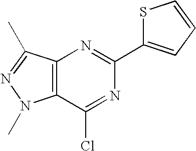

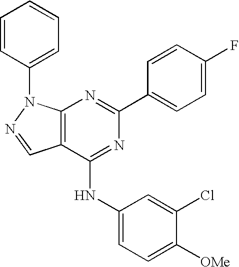

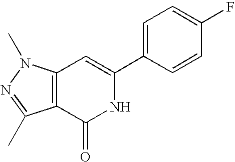

In this aspect of the present invention, compounds provided herein can be chiral or achiral, or they may exist as racemic mixtures, diastereomers, pure enantiomers, a prodrug, a tautomer or any mixture thereof. For chiral compounds, separate enantiomers, separate diastereomers, and any mixture of enantiomers, diastereomers, or both are encompassed herein. Further, the present invention also encompasses any combination of compounds provided herein, including any salts, including pharmaceutically acceptable and non-pharmaceutically acceptable salts, or any mixture thereof.

As used herein, the terms “pharmaceutically acceptable” salt or “pharmacologically acceptable” salt refers generally to a salt or complex of the compound or compounds in which the compound can be either anionic or cationic, and have associated with it a counter cation or anion, respectively, that is generally considered suitable for human or animal consumption. For example, a pharmaceutically acceptable salt can refer to a salt of a compound disclosed herein that forms upon reaction or complexation with an acid whose anion is generally considered suitable for human or animal consumption. In this aspect, pharmacologically acceptable salts include salts with organic acids or inorganic acids. Examples of pharmacologically acceptable salts include, but are not limited to, hydrochloride, hydrobromide, hydroiodide, sulfate, phosphate, propionate, lactate, maleate, malate, succinate, tartarate, and the like.

Salts may also be formed by deprotonating an acid moiety of the compound, such as a carboxylic acid moiety, OH, or NH, and the like, using a base such as an organic base, an inorganic base, an organometallic base, a Lewis base, a Bronsted base, or any combination thereof. In cases where compounds carry an acidic moiety, suitable pharmaceutically acceptable salts can include alkali metal salts, alkaline earth metal salts, or salts with organic basis, and the like. In this aspect, examples of alkali metal salts include, but are not limited to, sodium and potassium salts, and examples of salts with organic basis include, but are not limited to, meglumine salts, and the like. The pharmacologically acceptable salts can be prepared by conventional means. Additional examples of pharmaceutically acceptable salts, and methods of preparing such salts, are found, for example, in Berg et.al., J. Pharma. Sci, 66, 1-19 (1977).

In a further aspect, this invention also provides a composition comprising at least one compound as disclosed herein, including a composition comprising a pharmaceutically acceptable carrier and at least one compound as disclosed herein. In this aspect, the at least one compound can be present as a neutral compound, as a salt, or as any combination thereof. This invention also encompasses a composition comprising at least one compound as disclosed herein, and optionally comprising a pharmaceutically acceptable additive selected from a carrier, an auxiliary, a diluent, an excipient, a preservative, a solvate, or any combination thereof.

Further, this invention encompasses a pharmaceutical composition, comprising at least one compound as disclosed herein, and optionally comprising a pharmaceutically acceptable additive selected from a carrier, an auxiliary, a diluent, an excipient, a preservative, a solvate, or any combination thereof, wherein the pharmaceutical composition is in the form of a tablet, a capsule, a syrup, a cachet, a powder, a granule, a solution, a suspension, an emulsion, a bolus, a lozenge, a suppository, a cream, a gel, a paste, a foam, a spray, an aerosol, a microcapsule, a liposome, or a transdermal patch.

In another aspect, this invention encompasses a pharmaceutical composition, comprising at least one compound as disclosed herein, and optionally comprising a pharmaceutically acceptable additive selected from a carrier, an auxiliary, a diluent, an excipient, a preservative, a solvate, or any combination thereof; and further comprising an agent selected from a chemotherapeutic agent, an immunosuppressive agent, a cytokine, a cytotoxic agent, an anti-inflammatory agent, an antirheumatic agent, an antidyspilidemic agent, a cardiovascular agent, or any combination thereof.

Another aspect of this invention is directed to using the compounds and compositions disclosed herein in a method of treating a condition or disease state mediated by the low expression of Perlecan, comprising administering an amount of at least one compound as disclosed herein, effective to induce Perlecan expression.

A further aspect of this invention is directed to using the compounds and compositions disclosed herein in a method of treating atherosclerosis, arthritis, restenosis, diabetic nephropathy, or dyslipidemia, comprising administering an effective amount of at least one compound as disclosed herein.

Synthetic Methods

The present invention, in another aspect, also provides a general process for the preparation of the bicyclo heterocyclic compounds disclosed herein. In one aspect, simple derivatization of a heterocycle, as illustrated by the reaction scheme given below, provides a synthetic entry to many of the substituted compounds of this invention.

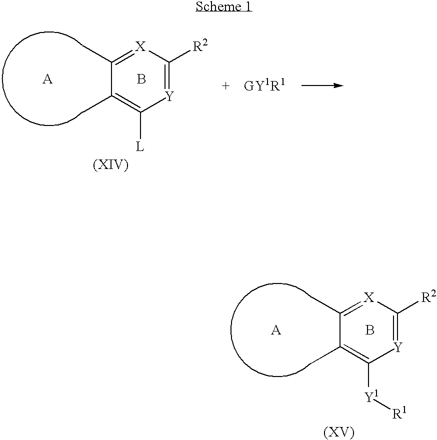

In this scheme, the bicyclic, heterocyclic precursor compound (XIV) comprises a leaving group, L. In one aspect, for example, L can be a halogen, an aryloxy, an alkylsulfinyl, an alkylsulfonyl such as trifluoromethanesulfonyloxy, an arylsulfinyl, an arylsulfonyl, a silyloxy, a cyano, a pyrazolo, a triazolo, and the like, or similar leaving groups. Other substituents on heterocyclic precursor compound (XIV) and heterocyclic product (XV) are as defined herein for structure (I). Thus, compound (XIV) can be converted to heterocyclic product (XV) by its reaction with a compound of formula GY¹R¹, wherein G can be selected from, for example, hydrogen, NH₂, NHR⁵ wherein R⁵ is defined as it is for structure (I), OH, SH, B(OH)₂, Li, MgZ wherein Z is typically a halogen, and the like. In one aspect, when G is NHR⁵, R¹ and R⁵ together can form an optionally substituted cyclic ring along with an adjacent N atom, which can optionally comprise one or more hetero atoms selected from oxygen, nitrogen or sulfur.

In another aspect, the reaction presented in the scheme above can be performed in presence of a base such as sodium hydroxide, potassium hydroxide, potassium carbonate, and the like. Similarly, the reaction presented in the scheme above also can be performed in the presence of a Lewis acid such as aluminum chloride (AlCl₃), or a transition metal catalyst such as a palladium catalyst. For example, a suitable palladium catalyst can be selected from tetrakis(triphenylphosphine)palladium(0) [(PPh₃)₄Pd], bis(triphenylphosphine)-palladium(II)chloride [(PPh₃)₂PdCl₂], and the like, including a combination thereof. In one aspect, the reaction shown in the scheme above can be carried out in a solvent such as acetone, dimethylformamide (DMF), dimethylacetamide (DMA), benzene, toluene, and the like. In another aspect, for example, the temperature of the reaction can be from about 25° C. to about 150° C., though temperatures lower and higher are possible, and the duration of the reaction can be, for example, from about 2 hours to about 24 hours or more.

The following references relate generally to pyrazolopyrimidine class of compounds: Pyrazolo pyrimidines (WO 05049617), 5,7-Diamino pyrazolo 4,3 dipyrimidines with PDE-5 inhibiting activity (WO 05049616), 5,7-Diamino pyrazolo 4,3 dipyrimidines useful in treatment of hypertension (WO 04094810), Synthesis and potential antipsychotic activity of 1H-imidazole[1,2-c]pyrazole[3,e]pyrimidines (Journal of Medicinal Chemistry 1998, 31(2), 454-61), Pyrazolo[4,3-d]pyrimidines, process for their preparation and methods for therapy (EP 1348707).

The following general reaction schemes detail the synthetic approaches to the bicyclic heterocyclic compounds disclosed herein. Compounds disclosed herein could be prepared as shown in Schemes 2-6 and as illustrated in the Examples by using standard synthetic methods and the starting materials, which are either commercially available or can be synthesized from commercially available precursors using synthetic methods known in the art, or variations thereof as appreciated by those skilled in the art. Each variable in the following schemes refer to any group consistent with the description of the compounds provided herein.

The following general procedures could be used in the reactions schemes and in the Examples provided herein.

Halogenation could be carried out by using reagents such as phosphorus oxychloride (POCl₃), thionyl chloride (SOCl₂), and the like, for example, at a temperature from about 80° C. to about 120° C., for about 4 to about 8 hours, followed by pH adjustment of resultant mixture to a pH from about 6 to about 7.

Amination could be carried out by using amines in presence of a solvent chosen from acetone, acetonitrile, dimethylformamide, dimethylacetamide and the like, with or with out a base. Suitable bases include triethylamine, N,N-diisopropyl ethyl amine, potassium carbonate, sodium carbonate, sodium hydride, and the like. The reaction temperature was typically from about 20° C. to about 120° C. The duration of the reaction was typically in the range of from about 4 hours to about 20 hours.

Arylation was carried out by aryl boronic acids, for example in the presence of a palladium catalyst and a base such as sodium carbonate, potassium carbonate, sodium or potassium tert-butoxide, potassium phosphate and the like, at ambient temperature or elevated temperatures using various inert solvents. Examples of suitable solvents include, but are not limited to toluene, dioxane, DMF, n-methyl pyrolidine, ethylene glycol, dimethyl ether, diglyne, and acetonitrile. Commonly employed palladium catalysts include [tetrakis-(triphenylphosphine)palladium (0)] [(PPh₃)₄Pd], tris(dibenzeledine acetone)dipalladium (0) or palladium (II) acetate[Pd(OAc)₂], [bis(triphenylphosphine)palladium(II)chloride] [(PPh₃)₂PdCl₂] (Suzuki reaction, Miyaura and Suzuki (1995, Chemical Reviews 95:2457). Thus one further aspect of the invention relates to the processes of preparing compounds of formulas provided herein. Any compound of any formula disclosed herein can be obtained using procedures provided in the reaction Schemes, as well as procedures provided in the Examples, by selecting suitable starting materials and following analogous procedures. Thus, any compound of any formula disclosed or exemplified herein, can be obtained by using the appropriate starting materials and appropriate reagents, with the desired substitutions, and following procedures analogous to those described herein.

Therefore, it will be readily understood by one of ordinary skill, that the reaction schemes disclosed herein can be adapted to prepare any compound of this disclosure, therefore any discussion of a particular step in a reaction scheme is intended to reflect one method or one set of considitions that can be used to carry out that step. This discussion of a particular step is not intended to be limiting, but rather exemplary, of one particular method and set of conditions by which that step can be effected. For example, when a reaction scheme illustrates a synthetic method to prepare a compound of formula (IIa), it is intended that the substituents R¹, R², R³, R⁴, and Y¹ illustrated on the bicyclic heterocyclic core include at least those substituents identified in the description of compound (IIa) herein, but also include other substituents that could be employed in any step in the reaction scheme or in any precursor, to prepare any compound of any formula disclosed or exemplified herein.

In one aspect of this invention, compounds of this invention can be prepared as follows, as illustrated for compounds of formula (IIa).

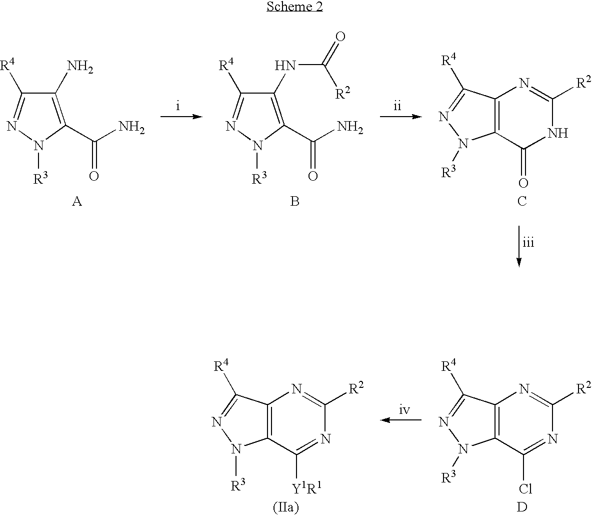

The Scheme 2 starting materials are the pyrazolocarboxilic acids of formula A. Some compounds of formula A are either commercially available and others are well known in the chemical literature and readily prepared. Representative steps of Scheme 2 include the following.
Step i: The carboxylic acids of formula A could be converted to an amide of formula B, either directly or via an acid chloride. This conversion can be achieved by treating acid chloride in the presense of a base such as triethyl amine (TEA) in a suitable solvent such as dichloromethane (DCM). The reaction can be performed at temperatures from about 0° C. to about 40° C.
Step ii: The compound of formula B could be treated with a base such as metal alkoxides, for example potassium t-butoxide, in a polar solvent such as t-butanol, typically at a temperature from about 20° C. to about 100° C.
Step iii: The compound of formula C could treated with a large excess of suitable chlorinating reagent such as POCl₃ or phenyl phosphonyl dichloride in the presense of a tertiary amine such as TEA, at elevated temperatures, for a period of from about 8 to about 48 hours, to provide the corresponding chloro compound of formula D.
Step iv: A solution of the chloride of formula D and an amine such as R¹Y¹H in a suitable solvent such as isopropanol was stirred at elevated temperatures for a time from about 1 hour to about 24 hours, to provide the corresponding compounds of formula Ia.

In another aspect of this invention, compounds of this invention, can be prepared as follows, as illustrated for compounds of formula (IIa).

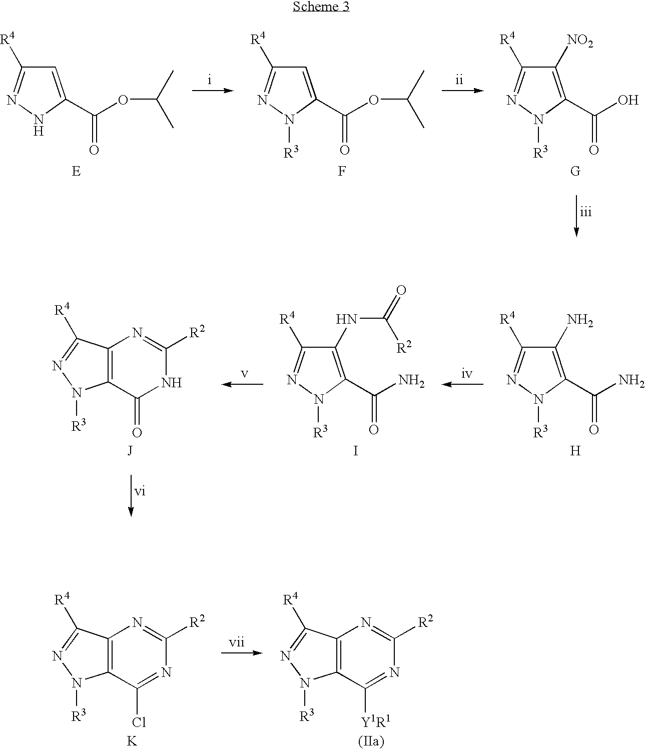

Representative steps of Scheme 3 include the following.
Step i: Pyrazolocarboxilic acid ester of formula E can be be alkylated with dialkyl sulphate, to prepare a compound of formula F.
Step ii: Hydrolysis of the ester followed by nitration of the compounds 15 of formula F, provides the compounds of formula G. The conversion can be accomplished by treating the compounds of formula F, with an alkaline metal hydroxide such as sodium hydroxide, in a suitable solvent, for example at a temperature of from about 10° C. to reflux temperature of the solvent used. Suitable solvents include, but are not limited to, water, methanol, ethanol and mixtures thereof. Nitration of the compounds of formula F can be achieved by using nitrating agent such as HNO₃, or a mixture of HNO₃ and H₂SO₄.
Step iii: Reduction of the compounds of formula G to provide the anmines of the compounds of formula H can be achieved, for example, by the catalytic hydrogenation in the presense of transition metal catalysts such as palladium, optionally at elevated temperatures and pressures, and typically in an alcoholic solvent such as ethanol.
Steps iv-vii: The compounds of formula Ia were obtained following the methods described in Scheme 1, Steps i, ii, iii and iv.

In yet another aspect of this invention, compounds of this invention can be prepared as follows, as illustrated for compounds of formula (IVa).

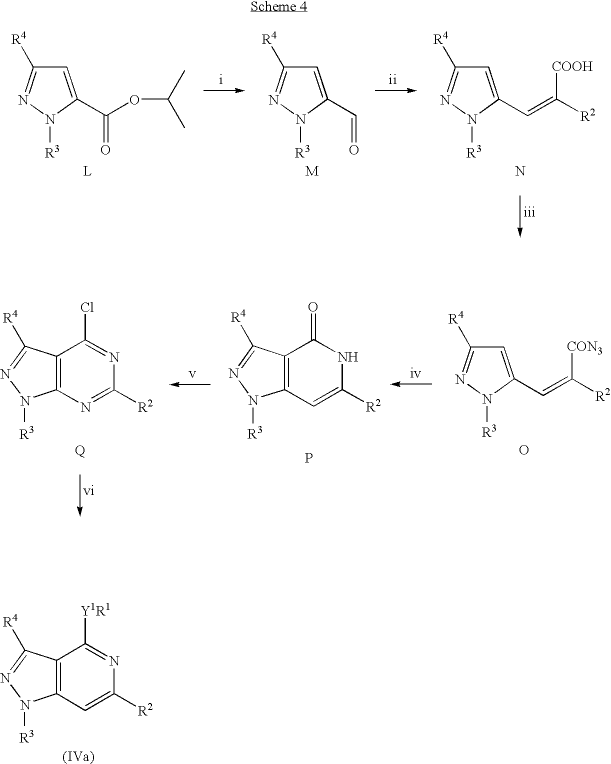

Representative steps of Scheme 4 include the following.

Step i: The ester compounds of formula L can be reduced using, for example, metal hydrides such as LiAlH₄, in solvents such as THF at 0° C., followed by oxidation with pyridinium dichromate, to generate the aldehyde compounds of formula M.

Steps ii and iii: Acid azides of the compounds of formula O can be obtained by reacting the compounds of formula M with acids having an active methylene in acetic anhydride and base, typically at elevated temperatures, followed by treatment with sodium azide.

Step iv: Reacting compounds of formula O with ethyl chloroformate, followed by cyclization in a solvent such as diphenyl ether, affords compounds of formula P.

Step v and vi: The compounds of formula (IVa) can be obtained by following the methods described in Scheme 1, steps iii and iv.

In another aspect of this invention, compounds of this invention, can be prepared as follows, as illustrated for compounds of formula (IIIb′).

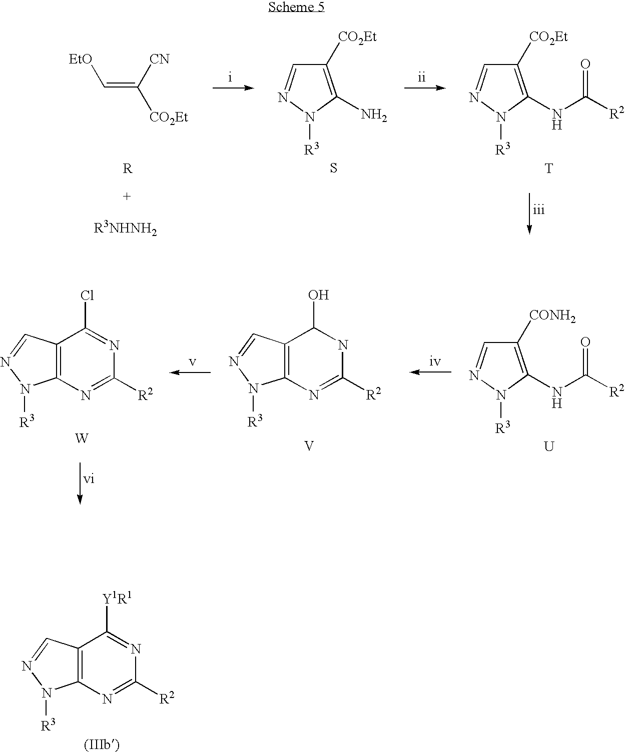

Step i: The cyanoester of formula R can be reacted with hydrazine for the pyrazole synthesis, illustrated by compounds of formula S.

Step ii: Amide compounds of formula T can be prepared by treating a solution of the appropriate acid with an amine in the presense of a coupling agent, such as dicyclohexyl carbodiimide and dimethylamino pyridine, in a suitable solvent, for example, DCM.

Step iii: Compounds of formula T can be converted to compounds of formula U by treating with thionyl chloride and excess ammonia in dioxane solvent.

Step iv: Cyclization of compounds of formula U, in the presence of a base such as potassium t-butoxide affords compounds of formula V.

Steps v and vi: The compounds of formula (IIIb′) can be obtained by following the methods described in Scheme 1, Steps iii and iv.

In another aspect of this invention, compounds of this invention, can be prepared as follows, as illustrated for compounds of formula FF.

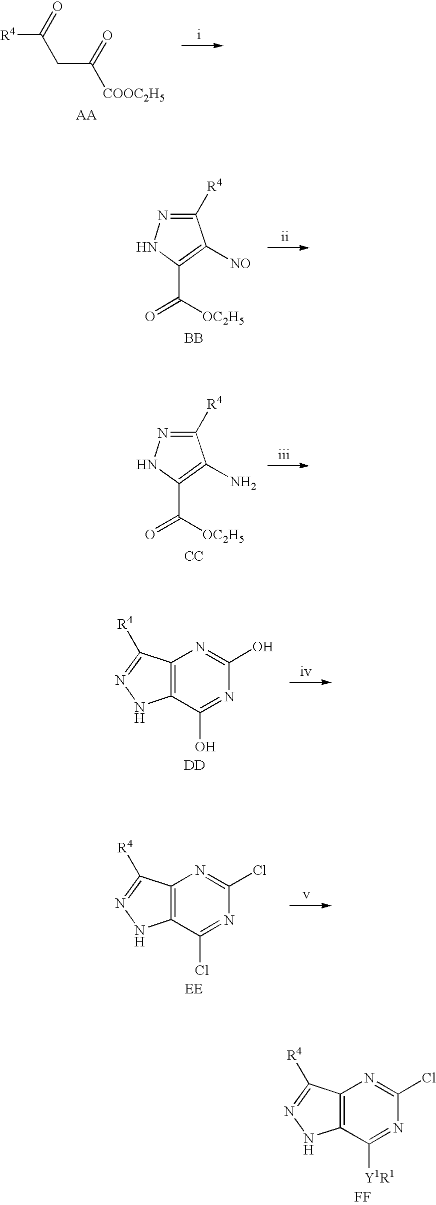

Step i: The 1,3 diketones of formula AA can be reacted with hydrazine, followed by nitration with sodium nitrate, to afford compounds of formula BB.

Step ii: The compounds of formula CC can be obtained following the method described in Step iii of Scheme 3.

Step iii: A solution of pyrazolocarboxamide and phosgene or an equivalent thereof in a suitable solvent, can be stirred at temperature between ambient temperature and the boiling point of the solvent, optionally at elevated pressures, to provide the corresponding pyrazolo pyrimidinediol of formula DD.

Step iv: The diol of formula DD is treated with excess chlorinating agent such as phosphorus oxychloride, in the presense of triethyl amine (TEA) at elevated temperatures, to provide the corresponding dichloropyrazolo pyrimidine of formula EE.

Step v: The compounds of formula FF, can be obtained by following the methods described in Scheme 1, Step iv.

Step vi (not shown): A solution of the monochloride FF and a suitable amine in a dipolar, aprotic solvent, can be stirred at elevated temperatures for between about I hour to about 24 hours, to provide the corresponding compounds of formula (IIa).

Prodrugs

In another aspect of this invention, alternatively, the compounds can be formulated and administered in a prodrug form. In general, prodrugs comprise functional derivatives of the claimed compounds which are capable of being enzymatically activated or converted into the more active parent form. Thus, in the treatment methods of the present invention, the term “administering” encompasses the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in Wihnan, 14 Biochem. Soc. Trans. 375-82 (1986); Stella et al., Prodrugs: A Chemical Approach to Targeted Drug Delivery in Directed Drug Delivery 247-67 (1985).

The prodrugs of present invention include, but are not limited to derivatives of carboxylic acid, sulfonamide, amine, hydroxyl, and the like, including other functional groups and including any combination thereof.

In another aspect, this invention provides a pharmaceutical composition, comprising one or more compounds of any formula in any combination described above and optionally comprising a pharmaceutically acceptable additive selected from a carrier, an auxiliary, a diluent, an excipient, a preservative, a solvate, or any combination thereof. In a related aspect, this invention affords a method of treating a condition or disease state mediated by the low expression of Perlecan, comprising administering at least one compound as disclosed herein, in an amount effective to induce Perlecan expression. In a related aspect, this invention also provides a method of treating atherosclerosis, arthritis, restenosis, diabetic nephropathy, or dyslipidemia, comprising administering an effective amount of at least one compound as disclosed herein.

Cellular Proliferation

Without being held to a particular theory, it is believed that many vascular conditions or diseases, such as cardiovascular diseases, organ transplant sequellae, vascular occlusive conditions including, but not limited to, neointimal hyperplasia, restenosis, transplant vasculopathy, cardiac allograft vasculopathy, atherosclerosis, and arteriosclerosis, are caused by or have collateral damage due to unwanted cellular proliferation, such as SMC hyperplasia.

In one aspect, a compound of the present invention or a composition comprising the compound attenuates or inhibits proliferation of a cell. In one aspect, the cell is a SMC. In other aspects, the present invention provides a method for treating a condition or disease associated with proliferation of a cell in a mammalian subject, the method comprising administering to the subject a composition comprising a therapeutically-effective amount of at least one compound as disclosed herein, or their pharmaceutically-acceptable salts thereof. In one aspect, the condition or disease is a neoplasia. In another aspect, the condition or disease is SMC hyperplasia. In other aspects, the condition or disease is a cardiovascular disease, an organ transplant sequellae, or a vascular occlusive condition. In one aspect, the vascular occlusive condition comprises neointimal hyperplasia, restenosis, transplant vasculopathy, cardiac allograft vasculopathy, atherosclerosis, or arteriosclerosis.

Compounds that are effective in inhibiting SMC proliferation can be administered to a mammalian subject suspected of having or who has, for example, vasculopathy or who has undergone angioplasty or other procedures damaging to the endothelium.

Effective amounts are administered to the subject in dosages and formulations that are safe and effective, including, but not limited to, the ranges taught herein.

As disclosed herein, compositions comprising at least one compound as disclosed herein, or their pharmaceutically-acceptable salts thereof, can be used in conjunction with other therapeutic agents or in methods optionally comprising steps such as altered patient activities, including, but not limited to, changes in exercise or diet.

Examples of compounds of the present invention that can at least affect cellular proliferation are shown in the following table, as measured by the assays taught herein.

TABLE 4
Examples of compounds that at least affect cellular proliferation.

| Entry | Compound |
|---|---|
| 1. | 4-[5-(3,4-Dimethoxy-phenyl)-1-methyl-3-propyl-1H-pyrazolo |
| | [4,3-d]pyrimidin-7-yl]-2-methyl-phenol |
| 2. | (3-Chloro-4-methoxy-phenyl)-[5-(3,4-dimethoxy-phenyl)-1-methyl-3- |
| | propyl-1H-pyrazolo[4,3-d]pyrimidin-7-yl]-amine hydrochloride |
| 3. | (3-Chloro-4-methoxy-phenyl)-[5-(4-fluoro-phenyl)-1-methyl-3-propyl- |
| | 1H-pyrazolo[4,3-d]pyrimidin-7-yl]-amine hydrochloride |
| 4. | (3-Fluoro-4-methoxy-phenyl)-[5-(4-fluoro-phenyl)-1- |
| | methyl-3-propyl-1H-pyrazolo[4,3-d]pyrimidin-7-yl]-amine hydrochloride |
| 5. | (3-Fluoro-4-methoxy-phenyl)-[5-(4-fluoro-phenyl)-1-methyl-3-propyl- |
| | 1H-pyrazolo[4,3-d]pyrimidin-7-yl]-amine |
| 6. | (4-Fluoro-phenyl)-[5-(4-fluoro-phenyl)-1-methyl-3-propyl-1H- |
| | pyrazolo[4,3-d]pyrimidin-7-yl]-amine hydrochloride |
| 7. | (3,4-Dimethoxy-phenyl)-[5-(4-fluoro-phenyl)-1-methyl-3- |
| | propyl-1H-pyrazolo[4,3-d]pyrimidin-7-yl]-amine hydrochloride |
| 8. | 2-Chloro-4-(1-methyl-5-phenyl-3-propyl-1H-pyrazolo[4,3-d]pyrimidin- |
| | 7-ylamino)-phenol hydrochloride |
| 9. | (3-Chloro-4-methoxy-phenyl)-(1-methyl-5-phenyl-3-propyl- |
| | 1H-pyrazolo[4,3-d]pyrimidin-7-yl)-amine hydrochloride |
| 10. | (3-Chloro-4-methoxy-phenyl)-(1-methyl-5-phenyl-3-propyl- |
| | 1H-pyrazolo[4,3-d]pyrimidin-7-yl)-amine |
| 11. | 7-(4-Fluoro-phenoxy)-1-methyl-5-phenyl-3-propyl-1H-pyrazolo[4,3-d]pyrimidine |
| 12. | 2-Methyl-4-(1-methyl-5-phenyl-3-propyl-1H-pyrazolo[4,3-d]pyrimidin- |
| | 7-yl)-phenol |
| 13. | 1-[5-(3,4-Dimethoxy-phenyl)-1-methyl-3-propyl-1H-pyrazolo |
| | [4,3-d]pyrimidin-7-yl]-piperidin-4-ol |
| 14. | 4-[5-(3-hydroxy,4-methoxy-phenyl)-1-methyl-3-propyl-1H-pyrazolo[4,3- |
| | d]pyrimidin-7-yl]-2-methyl-phenol |
| 15. | (3-Fluoro-4-methoxy-phenyl)-(1-methyl-5-phenyl-3-propyl- |
| | 1H-pyrazolo[4,3-d]pyrimidin-7-yl)-amine hydrochloride |
| 16. | (3-Chloro-4-methoxy-phenyl)-(1-methyl-3-propyl-5-thiophen-2-yl-1H- |
| | pyrazolo[4,3-d]pyrimidin-7-yl)-amine hydrochloride |
| 17. | 2-Chloro-4-[5-(4-fluoro-phenyl)-1-methyl-3-propyl-1H- |
| | pyrazolo[4,3-d]pyrimidin-7-ylamino]-phenol hydrochloride |
| 18. | 5-(4-Fluoro-phenyl)-1-methyl-7-phenylethynyl-3-propyl-1H- |
| | pyrazolo[4,3-d]pyrimidine |
| 19. | 3-[7-(3-Chloro-4-methoxy-phenylamino)-1-methyl-3-propyl-1H- |
| | pyrazolo[4,3-d]pyrimidin-5-yl]-4-ethoxy-benzenesulfonamide |
| | hydrochloride |
| 20. | 4-Ethoxy-3-[7-(3-fluoro-4-methoxy-phenylamino)-1-methyl-3-propyl- |
| | 1H-pyrazolo[4,3-d]pyrimidin-5-yl]-benzenesulfonamide hydrochloride |
| 21. | (3-Fluoro-4-methoxy-phenyl)-(1-methyl-3-propyl-5-thiophen-2-yl-1H- |
| | pyrazolo[4,3-d]pyrimidin-7-yl)-amine hydrochloride |
| 22. | (3-Chloro-4-methoxy-phenyl)-[5-(2-ethoxy-phenyl)-1-methyl-3-propyl- |
| | 1H-pyrazolo[4,3-d]pyrimidin-7-yl]-amine hydrochloride |
| 23. | [5-(2-Ethoxy-phenyl)-1-methyl-3-propyl-1H-pyrazolo[4,3-d]pyrimidin- |
| | 7-yl]-(3-fluoro-4-methoxy-phenyl)-amine hydrochloride |
| 24. | (3-Fluoro-4-methoxy-phenyl)-[5-(4-fluoro-phenyl)-1,3- |
| | dimethyl-1H-pyrazolo[4,3-d]pyrimidin-7-yl]-amine hydrochloride |
| 25. | (3-Chloro-4-methoxy-phenyl)-[5-(4-fluoro-phenyl)-1,3-dimethyl- |
| | 1H-pyrazolo[4,3-d]pyrimidin-7-yl]-amine hydrochloride |
| 26. | 2-Chloro-4-[5-(4-fluoro-phenyl)-1,3-dimethyl-1H-pyrazolo[4,3- |
| | d]pyrimidin-7-ylamino]-phenol hydrochloride |
| 27. | (4-Chloro-3-methoxy-phenyl)-(1-methyl-5-phenyl-3-propyl- |
| | 1H-pyrazolo[4,3-d]pyrimidin-7-yl)-amine hydrochloride |
| 28. | (4-Chloro-3-methoxy-phenyl)-[5-(4-fluoro-phenyl)-1-methyl- |
| | 3-propyl-1H-pyrazolo[4,3-d]pyrimidin-7-yl]-amine hydrochloride |
| 29. | (3-Chloro-4-methoxy-phenyl)-[6-(4-fluoro-phenyl)-1,3- |
| | dimethyl-1H-pyrazolo[4,3-c]pyridin-4-yl]-amine hydrochloride |
| 30. | (3-Fluoro-4-methoxy-phenyl)-[6-(4-fluoro-phenyl)-1,3-dimethyl-1H- |
| | pyrazolo[4,3-c]pyridin-4-yl]-amine hydrochloride |
| 31. | (3-Chloro-4-methoxy-phenyl)-(1,3-dimethyl-5-phenyl-1H-pyrazolo[4,3- |
| | d]pyrimidin-7-yl)-amine hydrochloride |
| 32. | (1,3-Dimethyl-5-phenyl-1H-pyrazolo[4,3-d]pyrimidin-7-yl)-(3-fluoro-4- |
| | methoxy-phenyl)-amine hydrochloride |
| 33. | (1,3-Dimethyl-5-thiophen-2-yl-1H-pyrazolo[4,3-d]pyrimidin- |
| | 7-yl)-(3-fluoro-4-methoxy-phenyl)-amine hydrochloride |
| 34. | 2-Chloro-4-[5-(4-fluoro-phenyl)-1-methyl-3-propyl-1H- |
| | pyrazolo[4,3-d]pyrimidin-7-yl]-phenol |
| 35. | (4-Chloro-3-methoxy-phenyl)-(1,3-dimethyl-5-thiophen-2-yl- |
| | 1H-pyrazolo[4,3-d]pyrimidin-7-yl)-amine hydrochloride |
| 36. | (4-Chloro-3-methoxy-phenyl)-(1,3-dimethyl-5-phenyl-1H-pyrazolo[4,3- |
| | d]pyrimidin-7-yl)-amine hydrochloride |
| 37. | (3-Chloro-4-methoxy-phenyl)-(1,3-dimethyl-5-thiophen-2-yl- |
| | 1H-pyrazolo[4,3-d]pyrimidin-7-yl)-amine hydrochloride |
| 38. | Benzo[1,3]dioxol-5-yl-(1,3-dimethyl-5-thiophen-2-yl-1H-pyrazolo[4,3- |
| | d]pyrimidin-7-yl)-amine hydrochloride |
| 39. | Benzo[1,3]dioxol-5-yl-[5-(4-fluoro-phenyl)-1,3-dimethyl-1H- |
| | pyrazolo[4,3-d]pyrimidin-7-yl]-amine hydrochloride |
| 40. | 2-Chloro-4-(1,3-dimethyl-5-thiophen-2-yl-1H-pyrazolo[4,3-d]pyrimidin- |
| | 7-ylamino)-phenol hydrochloride |
| 41. | 7-(4-Methoxy-3-methyl-phenyl)-1-methyl-5-phenyl-3-propyl- |
| | 1H-pyrazolo[4,3-d]pyrimidine |
| 42. | 5-(4-Fluoro-phenyl)-1,3-dimethyl-7-phenyl-1H-pyrazolo[4,3- |
| | d]pyrimidine |
| 43. | 5-(4-Fluoro-phenyl)-1-methyl-3-propyl-7-p-tolyl-1H-pyrazolo[4,3- |
| | d]pyrimidine |
| 44. | 7-(3-Fluoro-4-methoxy-phenyl)-1-methyl-5-phenyl-3-propyl- |
| | 1H-pyrazolo[4,3-d]pyrimidine |
| 45. | 5-(4-Fluoro-phenyl)-1-methyl-7-phenyl-3-propyl-1H-pyrazolo[4,3- |
| | d]pyrimidine |
| 46. | (3-Chloro-4-methoxy-phenyl)-(1,6-diphenyl-1H-pyrazolo |
| | [3,4-d]pyrimidin-4-yl)-amine hydrochloride |
| 47. | (3-Fluoro-4-methoxy phenyl)-[6-(4-fluoro-phenyl)-1-phenyl-1H- |
| | pyrazolo[3,4-d]-pyrimidin-4yl]amine hydrochloride |
| 48. | (1,3-Dimethyl-5-phenyl-1H-pyrazolo[4,3-d]pyrimidin-7-yl)-dimethyl- |
| | amine |
| 49. | 2-Fluoro-4-[5-(4-fluoro-phenyl)-1-methyl-3-propyl-1H-pyrazolo[4,3- |
| | d]pyrimidin-7-ylamino]-phenol hydrochloride |
| 50. | Benzo[1,3]dioxol-5-yl-[5-(4-fluoro-phenyl)-1-methyl- |
| | 3-propyl-1H-pyrazolo[4,3-d]pyrimidin-7-yl]-amine hydrochloride |
| 51. | 5-(4-Fluoro-phenyl)-1-methyl-7-(4-methylsulfanyl-phenyl)-3-propyl-1H- |
| | pyrazolo[4,3-d]pyrimidine |
| 52. | (3-Fluoro-4-methoxy-phenyl)-(1-methyl-3-propyl-5-trifluoromethyl-1H- |
| | pyrazolo[4,3-d]pyrimidin-7-yl)-amine hydrochloride |
| 53. | 7-(3-Fluoro-4-methoxy-phenyl)-5-(4-fluoro-phenyl)-1-methyl-3-propyl- |
| | 1H-pyrazolo[4,3-d]pyrimidine |
| 54. | 5-(4-Fluoro-phenyl)-7-(4-methoxy-3-methyl-phenyl)-1-methyl-3-propyl- |
| | 1H-pyrazolo[4,3-d]pyrimidine |
| 55. | 5-(4-Fluoro-phenyl)-7-(4-hydroxy-3-methyl-phenyl)-1-methyl-3-propyl- |
| | 1H-pyrazolo[4,3-d]pyrimidine |
| 56. | 7-Benzo[1,3]dioxol-5-yl-1,3-dimethyl-5-phenyl-1H-pyrazolo[4,3- |
| | d]pyrimidine |
| 57. | (4-Chloro-3-methoxy-phenyl)-(1-methyl-3-propyl-5-thiophen- |
| | 2-yl-1H-pyrazolo[4,3-d]pyrimidin-7-yl)-amine hydrochloride |
| 58. | Benzo[1,3]dioxol-5-yl-(1-methyl-3-propyl-5-thiophen-2-yl-1H- |
| | pyrazolo[4,3-d]pyrimidin-7-yl)-amine hydrochloride |
| 59. | 5-(4-Fluoro-phenyl)-7-(4-methanesulfonyl-phenyl)-1-methyl-3-propyl- |
| | 1H-pyrazolo[4,3-d]pyrimidine |
| 60. | (4-Chloro-3-trifluoromethyl-phenyl)-[6-(4-fluoro-phenyl)-1-phenyl-1H- |
| | pyrazolo[3,4-d]pyrimidin-4-yl]-amine hydrochloride |
| 61. | 7-Indol-1-yl-1,3-dimethyl-5-phenyl-1H-pyrazolo[4,3-d] |
| | pyrimidine |
| 62. | (1,3-Dimethyl-5-phenyl-1H-pyrazolo[4,3-d]pyrimidin-7-yl)- |
| | (3-fluoro-phenyl)-amine hydrochloride |
| 63. | [5-(4-Fluoro-phenyl)-1-methyl-3-propyl-1H-pyrazolo[4,3-d]pyrimidin-7- |
| | yl]-(3-trifluoromethyl-phenyl)-amine hydrochloride |
| 64. | [5-(4-Fluoro-phenyl)-1-methyl-3-propyl-1H-pyrazolo[4,3-d]pyrimidin-7- |
| | yl]-(4-trifluoromethoxy-phenyl)-amine hydrochloride |
| 65. | (5-Chloro-3-phenyl-1H-pyrazolo[4,3-d]pyrimidin- |
| | 7-yl)-(4-fluoro-phenyl)-amine hydrochloride |
| 66. | (1,3-Dimethyl-5-phenyl-1H-pyrazolo[4,3-d]pyrimidin-7-yl)- |
| | (4-trifluoromethoxy-phenyl)-amine hydrochloride |
| 67. | 4-Benzo[1,3]dioxol-5-yl-6-(4-fluoro-phenyl)-1,3-dimethyl- |
| | 1H-pyrazolo[4,3-c]pyridine |
| 68. | [5-(4-Fluoro-phenyl)-1,3-dimethyl-1H-pyrazolo[4,3-d]pyrimidin-7-yl]- |
| | (4-rifluoromethyl-phenyl)-amine hydrochloride |
| 69. | (6-Chloro-pyridin-3-yl)-[5-(4-fluoro-phenyl)-1,3-dimethyl-1H- |
| | pyrazolo[4,3-d]pyrimidin-7-yl]-amine hydrochloride |
| 70. | (4-Chloro-3-trifluoromethyl-phenyl)-[6-(4-fluoro-phenyl)- |
| | 1,3-dimethyl-1H-pyrazolo[4,3-c]pyridin-4-yl]-amine hydrochloride |
| 71. | [6-(4-Fluoro-phenyl)-1,3-dimethyl-1H-pyrazolo[4,3-c]pyridin-4-yl]-(3- |
| | trifluoromethyl-phenyl)-amine hydrochloride |
| 72. | (6-Chloro-pyridin-3-yl)-[6-(4-fluoro-phenyl)-1,3-dimethyl- |
| | 1H-pyrazolo[4,3-c]pyridin-4-yl]-amine hydrochloride |
| 73. | N-{5-[5-(4-Fluoro-phenyl)-1,3-dimethyl-1H-pyrazolo |
| | [4,3-d]pyrimidin-7-ylamino]-2-hydroxy-phenyl}-acetamide |
| | hydrochloride |
| 74. | [5-(4-Fluoro-phenyl)-1,3-dimethyl-1H-pyrazolo[4,3-d] |
| | pyrimidin-7-yl]-(4-methanesulfonyl-phenyl)-amine |
| 75. | 7-(3-Methanesulfonyl-phenyl)-1,3-dimethyl-5-phenyl- |
| | 1H-pyrazolo[4,3-d]pyrimidine |
| 76. | (1,3-Dimethyl-5-phenyl-1H-pyrazolo[4,3-d]pyrimidin-7- |
| | yl)-(2-methyl-benzooxazol-5-yl)-amine hydrochloride |
| 77. | 5-(4-Fluoro-phenyl)-7-(3-methanesulfonyl-phenyl)-1,3-dimethyl-1H- |
| | pyrazolo[4,3-d]pyrimidine |
| 78. | 6-(4-Fluoro-phenyl)-4-(3-methanesulfonyl-phenyl)-1,3-dimethyl-1H- |
| | pyrazolo[4,3-c]pyridine |
| 79. | (1H-Benzoimidazol-5-yl)-(1,3-dimethyl-5-phenyl-1H-pyrazolo[4,3- |
| | d]pyrimidin-7-yl)-amine hydrochloride |
| 80. | 6-(4-Fluoro-phenyl)-4-(4-methanesulfonyl-phenyl)-1,3-dimethyl-1H- |
| | pyrazolo[4,3-c]pyridine |
| 81. | 7-Fluoro-1,3-dimethyl-5-phenyl-1H-pyrazolo[4,3-d]pyrimidine |
| 82. | (1,3-Dimethyl-5-phenyl-1H-pyrazolo[4,3-d]pyrimidin-7-yl)-(2-methyl- |
| | 1H-benzoimidazol-5-yl)-amine hydrochloride |
| 83. | N-{4-[6-(4-Fluoro-phenyl)-1,3-dimethyl-1H-pyrazolo[4,3-c]pyridin-4-ylamino]- |
| | phenyl}-methanesulfonamide hydrochloride |
| 84. | N-[4-(1,3-Dimethyl-5-phenyl-1H-pyrazolo[4,3-d]pyrimidin-7-ylamino)- |
| | phenyl]-methanesulfonamide hydrochloride |
| 85. | 6-(4-fluoro phenyl)-(1,3-dimethyl-6-phenyl-1H-pyrazolo[4,3-c]pyridin- |
| | 4-yl)-(4-trifluoromethoxy-phenyl)-amine hydrochloride |
| 86. | [6-(4-Fluoro-phenyl)-1,3-dimethyl-1H-pyrazolo[4,3-c]pyridin-4-yl]-(4- |
| | methanesulfonyl-phenyl)-amine |
| 87. | 4-[5-(4-Fluoro-phenyl)-1,3-dimethyl-1H-pyrazolo[4,3-d]pyrimidin-7- |
| | ylamino]-N,N-dimethyl-benzenesulfonamide hydrochloride |
| 88. | 4-(1,3-Dimethyl-5-phenyl-1H-pyrazolo[4,3-d]pyrimidin- |
| | 7-ylamino)-benzenesulfonamide hydrochloride |
| 89. | 4-[5-(4-Fluoro-phenyl)-1,3-dimethyl-1H-pyrazolo[4,3-d]pyrimidin-7- |
| | ylamino]-benzenesulfonamide hydrochloride |
| 90. | 4-[5-(4-Fluoro-phenyl)-1,3-dimethyl-1H-pyrazolo[4,3-d]pyrimidin-7- |
| | ylamino]-N-methyl-benzenesulfonamide hydrochloride |
| 91. | 4-[6-(4-Fluoro-phenyl)-1,3-dimethyl-1H-pyrazolo[4,3-c]pyridin-4- |
| | ylamino]-N-methyl-benzenesulfonamide hydrochloride |
| 92. | 4-[5-(4-Fluoro-phenyl)-1,3-dimethyl-1H-pyrazolo[4,3-d]pyrimidin-7- |
| | ylamino]-benzamide hydrochloride |
| 93. | 4-[5-(4-Fluoro-phenyl)-1,3-dimethyl-1H-pyrazolo[4,3-d]pyrimidin-7-ylamino]- |
| | N-methyl-benzamide hydrochloride |
| 94. | 6-(4-Fluoro-phenyl)-1,3-dimethyl-4-(4-trifluoromethoxy-phenyl)-1H- |
| | pyrazolo[4,3-c]pyridine |

Proteoglycan (PG) expression can affect cellular proliferation. For example, increased expression of a PG such as, for example, a heparin sulfate proteoglycan (HSPG) can attenuate or inhibit cellular proliferation. A compound as described herein or a composition comprising the compound is for example useful as an antiproliferative agent.

As used herein, the term “proteoglycan” also can refer to an active fragment of a proteoglycan.

As used herein, the term “expression” refers to production and/or activity of a substance such as, for example, a protein or a second messenger. In the case of a substance comprising a protein, production can include, for example, transcription of the DNA sequence, translation of the corresponding mRNA sequence, posttranslational modification (e.g., glycosylation, disulfide bond formation, etc.), nuclear transport, secretion/exocytosis, and/or assembly. Non-limiting examples of “activity” of a substance include binding of the substance to a ligand or to a receptor, catalytic activity, signaling activity, the ability to stimulate gene expression, antigenic activity, activity in modulating or maintaining cell/cell interactions (e.g., adhesion), and/or activity in maintaining a structure of a cell (e.g., cell membranes, cytoskeleton). One skilled in the art knows that activity modulation can arise via a variety of mechanisms such as, for example, phosporylation and/or dephosphorylation.

As used herein, the term “affect” refers to direct and/or indirect affects. For example, a compound affecting “expression” of a HSPG via an increase in the rate of transcription of the corresponding gene may itself directly interact with the transcriptional machinery and/or may modulate other proteins or factors that cause an increase in the rate of transcription (e.g., activating a transcription factor).

In one aspect, a compound of the present invention or a composition comprising the compound increases expression of a HSPG. Non-limiting examples of a HSPG include a syndecan, a glypican, and a perlecan. Perlecan is a major extracellular HSPG and can be found, for example, in the blood vessel matrix. Perlecan can interact with extracellular matrix proteins, growth factors, and receptors. Besides blood vessels, perlecan also is present in other basement membranes and extracellular matrix structures.

In one aspect, the present invention provides a method for treating a condition or disease mediated by low expression of a perlecan in a mammalian subject, the method comprising administering to the subject a composition comprising a therapeutically-effective amount of at least one compound as disclosed herein, or their pharmaceutically-acceptable salts thereof, wherein the effective amount is sufficient to increase perlecan expression. In another aspect, the present invention provides a method for treating a condition or disease in a mammalian subject, the method comprising administering to the subject a composition comprising a therapeutically-effective amount of at least one compound as disclosed herein, or their pharmaceutically-acceptable salts thereof, wherein the condition or disease is atherosclerosis, arthritis, restenosis, diabetic nephropathy, or dyslipidemia.

Examples of a condition or disease mediated by low expression of a HSPG such as, for example, perlecan are shown in the following table.

TABLE 5
Examples of conditions or disease states mediated by the low expression of
perlecan in a human or an animal.

| Condition or | | |
|---|---|---|
| Disease State | Reference | |
| Atherosclerosis, | 1. | Endogenous heparin activity deficiency: the ‘missing link’ in |
| cardiovascular | | atherogenesis? Atherosclerosis. 2001 Dec; 159(2): 253-60. |
| | 2. | Holliman J et al, Relationship of sulfated glycosaminoglycans |
| | | and cholesterol content in normal and atherosclerotic human aorta |
| | 3. | Duan W, Paka L, Pillarisetti S. Distinct effects of glucose and |
| | | glucosamine on vascular endothelial and smooth muscle cells: |
| | | evidence for a protective role for glucosamine in atherosclerosis. |
| | | Cardiovasc Diabetol. 2005 Oct 5; 4: 16. |
| | 4. | Pillarisetti, S. Lipoprotein modulation of subendothelial heparan |
| | | sulfate proteoglycans (perlecan) and atherogenicity. Trends |
| | | Cardiovasc Med. 2000 Feb; 10(2): 60-5. |
| Restenosis | 5. | Paka L, Goldberg IJ, Obunike JC, Choi SY, Saxena U, Goldberg |
| | | ID, Pillarisetti S. Perlecan mediates the antiproliferative effect of |
| | | apolipoprotein E on smooth muscle cells. An underlying mechanism |
| | | for the modulation of smooth muscle cell growth? J Biol Chem. 1999 |
| | | Dec 17; 274(51): 36403-8. |
| | 6. | Nugent MA, Nugent HM, Iozzo RV, Sanchack K, Edelman ER. |
| | | Perlecan is required to inhibit thrombosis after deep vascular injury |
| | | and contributes to endothelial cell-mediated inhibition of intimal |
| | | hyperplasia. Proc Natl Acad Sci USA. 2000 Jun 6; 97(12): 6722-7. |
| Thrombosis | 7. | Nugent MA, Nugent HM, Iozzo RV, Sanchack K, Edelman ER. |
| | | Perlecan is required to inhibit thrombosis after deep vascular injury |
| | | and contributes to endothelial cell-mediated inhibition of intimal |
| | | hyperplasia. Proc Natl Acad Sci USA. 2000 Jun 6; 97(12): 6722-7. |
| Diabetic kidney | 8. | Menne J, Park JK, Boehne M, Elger M, Lindschau C, Kirsch T, |
| disease | | Meier M, Gueler F, Fiebeler A, Bahlmann FH, Leitges M, Haller H. |
| | | Diminished loss of proteoglycans and lack of albuminuria in protein |
| | | kinase C-alpha-deficient diabetic mice. Diabetes. 2004 |
| | | Aug; 53(8): 2101-9 |
| | 9. | Jensen T. Pathogenesis of diabetic vascular disease: evidence |
| | | for the role of reduced heparan sulfate proteoglycan. Diabetes. 1997 |
| | | Sep; 46 Suppl 2: S98-100 |
| Inflammation | 10. | Pillarisetti, S, Obunike JC, Goldberg IJ. Lysolecithin-induced |
| | | alteration of subendothelial heparan sulfate proteoglycans increases |
| | | monocyte binding to matrix. J Biol Chem. 1995 Dec |
| | | 15; 270(50): 29760-5 |
| | 11. | Rops AL, van der Vlag J, Lensen JF, Wijnhoven TJ, van den |
| | | Heuvel LP, van Kuppevelt TH, Berden JH. Heparan sulfate |
| | | proteoglycans in glomerular inflammation. Kidney Int. 2004 |
| | | Mar; 65(3): 768-85. |

Screening methods for identifying and determining the effects of a compound that increases proteoglycan expression, such as HSPG expression, are disclosed in U.S. patent application Ser. No. 10/091,357. Assays for determining the effects of the compound in vivo are also known to those skilled in the art. In general, the method comprises adding the compound to an assay and determining its affect on HSPG expression, including, but not limited to, syndecan expression, glypican expression and perlecan expression, for example, syndecans 1, 2 and 4; and glypican-1. In another aspect, perlecan expression is increased/induced or decreased/blocked in cells by certain inducers or inhibitors and the response is measured. Compounds of the present invention are then added to a replicate assay and the effect on perlecan induction is determined. Using such methods, compounds are determined that can either increase or decrease perlecan expression, or that have no effect at all. Those compounds that are effective as therapeutic agents can then be used in animals, humans or patients having a condition or disease associated with cellular proliferation as described herein.

In yet another aspect, a method for determining a compound that affects cellular proliferation comprises adding the compound or a composition comprising the compound suspected of affecting SMC proliferation to SMCs in growth medium or serum-free medium. The change in cell proliferation can be measured by methods known to those skilled in the art, such as incorporation of labeled nucleotides into dividing cells' DNA, and compared to the proliferation of cells which are not treated with the compound. Other measurements include directly determining levels of HSPG expression by measuring the amount or change in amount of HSPG such as with ELISA for HSPGs, and compared to the amount of HSPG synthesis in untreated cells. Other indirect or direct measurements are contemplated by the present invention and are known to those skilled in the art. For example, such methods include, but are not limited to, measurement of RNA levels, RT-PCR, Northern blotting, Western blotting promoter-based assays to identify compounds that affect one or more proteoglycans and assays for proteoglycan biological activity shown by recombinant proteins, partially purified proteins, or lysates from cells expressing proteoglycans in the presence or absence of compounds of interest.

An assay for identifying and determining an effect of a compound of the present invention comprises identifying compounds that interact with the promoter or enhancer regions of a gene (i.e., gene regulatory regions), or interact and affect proteins or factors that interact with the promoter or enhancer region, and are important in the transcriptional regulation of the protein's expression. For example, if perlecan were the protein, in general, the method comprises a vector comprising regulatory sequences of the perlecan gene and an indicator region controlled by the regulatory sequences, such as an enzyme, in a promoter-reporter construct. The protein product of the indicator region is referred to herein as a reporter enzyme or reporter protein. The regulatory region of the sequence of perlecan comprises a range of nucleotides from approximately −4000 to +2000 wherein the transcription initiation site is +1, more preferably, from −2500 to +1200, most preferably, from −1500 to +800 relative to the transcription initiation site. One skilled in the art knows that a gene may have one or more regulatory regions which may exist at a relatively near or relatively far distance from the transcription start site of the gene. One or more compounds according to the present invention can affect one or more known or unknown regulatory regions of a particular gene.

Cells are transfected with a vector comprising the promoter-reporter construct and then treated with one or more compositions comprising at least one compound of the present invention. For example, the transfected cells are treated with a composition comprising a compound suspected of affecting the transcription of perlecan and the level of activity of the perlecan regulatory sequences are compared to the level of activity in cells that were not treated with the compound. The levels of activity of the perlecan regulatory sequences are determined by measuring the amount of the reporter protein or determining the activity of the reporter enzyme controlled by the regulatory sequences. An increase in the amount of the reporter protein or the reporter enzyme activity shows a stimulatory effect on perlecan, by positively effecting the promoter, whereas a decrease in the amount or the reporter protein or the reporter enzyme activity shows a negative effect on the promoter and thus, on perlecan.

Additionally, the present invention comprises methods and compositions that can be used with gene therapy methods and composition, such as those gene therapy methods comprising administering compositions comprising nucleic acids that affect the synthesis or expression of HSPGs, particularly perlecan. Such methods and compositions are disclosed in U.S. patent application Ser. No. 10/091,357.

Glycosidase Modulation

The present invention also provides methods and compositions for modulating glycosidase expression such as, for example, heparanase expression. Without being held to a particular theory, it is believed thatglycosidases and their substrates, such as proteoglycans or glycated proteins, are aspects of a variety of conditions or diseases such as, for example, vascular conditions, including those conditions discussed supra, proteoglycan-associated diseases, associated diseases with vascular components, including but not limited to, kidney disease, ischemic heart disease, cardiovascular disease, generalized vascular disease, proliferative retinopathy, macroangeopathy, inflammatory diseases and metastatic diseases such as cancer, cellular proliferative conditions, and solid and blood borne tumors or other oncological conditions. In some aspects, a compound according to the present invention is for example useful for treating vascular, inflammatory, metastatic, and systemic conditions or diseases by affecting one or more substrates of one or more glycosidases.

Examples of compounds of the present invention that at least affect glycosidase expression are shown in the following table, as measured by the assays taught herein.

TABLE 6
Compounds having at least the activity of modulating
glycosidase enzyme activity.

| Entry | Compound |
|---|---|
| 1. | 4-[5-(3,4-Dimethoxy-phenyl)-1-methyl-3-propyl-1H-pyrazolo |
| | [4,3-d]pyrimidin-7-yl]-2-methyl-phenol |
| 2. | (3-Chloro-4-methoxy-phenyl)-[5-(3,4-dimethoxy-phenyl)-1- |
| | methyl-3-propyl-1H-pyrazolo[4,3-d]pyrimidin-7-yl]-amine |
| | hydrochloride |
| 3. | (3-Chloro-4-methoxy-phenyl)-[5-(4-fluoro-phenyl)-1-methyl- |
| | 3-propyl-1H-pyrazolo[4,3-d]pyrimidin-7-yl]-amine hydrochloride |
| 4. | (3-Fluoro-4-methoxy-phenyl)-[5-(4-fluoro-phenyl)-1- |
| | methyl-3-propyl-1H-pyrazolo[4,3-d]pyrimidin-7-yl]-amine |
| | hydrochloride |
| 5. | 2-Methyl-4-(1-methyl-5-phenyl-3-propyl-1H-pyrazolo[4,3- |
| | d]pyrimidin-7-yl)-phenol |
| 6. | (3-Fluoro-4-methoxy-phenyl)-[5-(4-fluoro-phenyl)-1,3- |
| | dimethyl-1H-pyrazolo[4,3-d]pyrimidin-7-yl]-amine hydrochloride |
| 7. | [6-(4-Fluoro-phenyl)-1,3-dimethyl-1H-pyrazolo[4,3-c]pyridin-4- |
| | yl]-(4-methanesulfonyl-phenyl)-amine |
| 8. | 4-[5-(4-Fluoro-phenyl)-1,3-dimethyl-1H-pyrazolo[4,3-d]pyrimidin- |
| | 7-ylamino]-N,N-dimethyl-benzenesulfonamide hydrochloride |
| 9. | 4-[5-(4-Fluoro-phenyl)-1,3-dimethyl-1H-pyrazolo[4,3-d]pyrimidin- |
| | 7-ylamino]-N-methyl-benzenesulfonamide hydrochloride |

In some aspects, the present invention provides a method for treating or preventing a condition or disease in a mammalian subject, the method comprising administering to the subject a composition comprising a therapeutically-effective amount of at least one compound as disclosed herein, or their pharmaceutically-acceptable salts thereof. In other aspects, the method comprises administering to the subject a composition comprising a therapeutically-effective amount of at least one compound as disclosed herein, or their pharmaceutically-acceptable salts thereof, wherein the therapeutically-effective amount is sufficient to attenuate or inhibit expression of a glycosidase. In one aspect, the glycosidase is heparanase. In some aspects, the condition or disease comprises cancer including, but not limited to, malignant and non-malignant cell growth, and the like. In another aspect, the condition or disease is an inflammatory condition or an autoimmune disease. In one aspect, the condition or disease is diabetic vasculopathy.

In one aspect, the present invention provides a method for treating or preventing an autoimmune condition or disease in a mammalian subject, the method comprising administering to the subject a composition comprising a therapeutically-effective amount of at least one compound as disclosed herein, or their pharmaceutically-acceptable salts thereof. In another aspect, the autoimmune condition or disease is rheumatoid arthritis, juvenile rheumatoid arthritis, systemic onset juvenile rheumatoid arthritis, psoriatic arthritis, ankylosing spondilitis, gastric ulcer, seronegative arthropathies, osteoarthritis, inflammatory bowel disease, ulcerative colitis, systemic lupus erythematosis, antiphospholipid syndrome, iridocyclitis/uveitis/optic neuritis, idiopathic pulmonary fibrosis, systemic vasculitis/wegener's granulomatosis, sarcoidosis, orchitis/vasectomy reversal procedures, allergic/atopic diseases, asthma, allergic rhinitis, eczema, allergic contact dermatitis, allergic conjunctivitis, hypersensitivity pneumonitis, transplants, organ transplant rejection, graft-versus-host disease, systemic inflammatory response syndrome, sepsis syndrome, gram positive sepsis, gram negative sepsis, culture negative sepsis, fungal sepsis, neutropenic fever, urosepsis, meningococcemia, trauma/hemorrhage, burns, ionizing radiation exposure, acute pancreatitis, adult respiratory distress syndrome, rheumatoid arthritis, alcohol-induced hepatitis, chronic inflammatory pathologies, Crohn's pathology, sickle cell anemia, diabetes, nephrosis, atopic diseases, hypersensitity reactions, allergic rhinitis, hay fever, perennial rhinitis, conjunctivitis, endometriosis, asthma, urticaria, systemic anaphalaxis, dermatitis, pernicious anemia, hemolytic disesease, thrombocytopenia, graft rejection of any organ or tissue, kidney translplant rejection, heart transplant rejection, liver transplant rejection, pancreas transplant rejection, lung transplant rejection, bone marrow transplant (BMT) rejection, skin allograft rejection, cartilage transplant rejection, bone graft rejection, small bowel transplant rejection, fetal thymus implant rejection, parathyroid transplant rejection, xenograft rejection of any organ or tissue, allograft rejection, anti-receptor hypersensitivity reactions, Graves disease, Raynoud's disease, type B insulin-resistant diabetes, asthma, myasthenia gravis, type III hypersensitivity reactions, POEMS syndrome (polyneuropathy, organomegaly, endocrinopathy, monoclonal gammopathy, and skin changes syndrome), polyneuropathy, organomegaly, endocrinopathy, monoclonal gammopathy, skin changes syndrome, anti-phospholipid syndrome, pemphigus, scleroderma, mixed connective tissue disease, idiopathic Addison's disease, autoimmune hemolytic anemia, autoimmune hepatitis, idiopathic pulmonary fibrosis, scleroderma, diabetes mellitus, chronic active hepatitis, vitiligo, vasculitis, post-MI cardiotomy syndrome, type IV hypersensitivity, contact dermatitis, hypersensitivity pneumonitis, allograft rejection, granulomas due to intracellular organisms, drug sensitivity, metabolic/idiopathic, Wilson's disease, hemachromatosis, alpha-1-antitrypsin deficiency, diabetic retinopathy, Hashimoto's thyroiditis, osteoporosis, hypothalamic-pituitary-adrenal axis evaluation, primary biliary cirrhosis, thyroiditis, encephalomyelitis, cachexia, cystic fibrosis, neonatal chronic lung disease, chronic obstructive pulmonary disease (COPD), familial hematophagocytic lymphohistiocytosis, dermatologic conditions, psoriasis, alopecia, nephrotic syndrome, nephritis, glomerular nephritis, acute renal failure, hemodialysis, uremia, toxicity, preeclampsia, ankylosing spondylitis, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatricial pemphigoid, CREST syndrome, cold agglutinin disease, discoid lupus, essential mixed cryoglobulinemia, fibromyalgia-fibromyositis, Graves' disease, Guillain-Barré, Hashimoto's thyroiditis, idiopathic thrombocytopenia purpura (ITP), IgA nephropathy, insulin dependent diabetes, juvenile arthritis, lichen planus, ménière's disease, multiple sclerosis, pemphigus vulgaris, polyarteritis nodosa, Cogan's syndrome, polychondritis, polyglandular syndromes, polymyalgia rheumatica, polymyositis and dermatomyositis, primary agammaglobulinemia, Raynaud's phenomenon, Reiter's syndrome, rheumatic fever, Sjögren's syndrome, stiff-man syndrome, Takayasu arteritis, temporal arteritis/giant cell arteritis, Wegener's granulomatosis; okt3 therapy, anti-cd3 therapy, cytokine therapy, chemotherapy, radiation therapy (e.g., including but not limited toasthenia, anemia, cachexia, and the like), chronic salicylate intoxication, and the like.

Illustrative assays or methods suitable for identifying compounds that affect heparanase expression are disclosed in the references cited individually below.
U.S. Pat. No. 4,859,581.
U.S. patent application Ser. No. 09/952,648
Goshen et al., 2 MOL. HUM. REPROD. 679-84 (1996).
Nakajima et al., 31 CANCER LETT. 277-83 (1986).
Vlodasky et al., 12 INVASION METASTASIS 112-27 (1992).
Freeman and Parish, 325 BIOCHEM. J. 229-37 (1997).
Kahn and Newman, 196 ANAL. BIOCHEM. 373-76 (1991).
   Inflammation Modulation

In various other aspects, the present invention provides a method for treating or preventing an inflammatory condition or disease. Without being held to a particular theory, pharmacological inhibition of AGE-induced cell activation provides the basis for therapeutic intervention in many diseases, notably in diabetic complications and Alzheimer's disease. Therapeutic approaches for inhibition of AGE-induced inflammation include, but are not limited to, blocking the glycation of proteins, blocking AGE interactions with receptors, and blocking AGE-induced signaling or signaling-associated inflammatory responses. Compounds described herein are for example useful for modulating inflammation including, but not limited to, inhibiting inflammation and/or its associated cell activation by glycated proteins or AGE, blocking the glycation of proteins, blocking AGE interactions with receptors, blocking AGE-induced signaling or signaling-associated inflammatory responses, affecting cytokine expression, AGE formation, AGE cross-linking, or affecting expression of other inflammation-related molecules including, but not limited to IL-6, VCAM-1, or AGE-induced MCP-1 (monocyte chemoattractant protein 1).

The term “inflammatory condition or disease” herein refers to any condition or disease directly or indirectly associated with inflammation including, for example, cell activation by glycated proteins or AGE. An inflammatory condition or disease can be acute or chronic. Illustratively, inflammatory conditions or diseases include, without limitation, inflammation associated with accumulation or presence of glycated proteins or AGE, vascular complications of type I or type II diabetes, atherosclerosis, rheumatoid arthritis, osteoarthritis, intraoccular inflammation, psoriasis, and asthma.

Examples of compounds of the present invention that modulate inflammation are shown in the following table, as measured by the assays taught herein.

TABLE 7
Examples of compounds of the present invention that affect inflammation.

| Entry | Compound |
|---|---|
| 1. | 4-[5-(3,4-Dimethoxy-phenyl)-1-methyl-3-propyl-1H-pyrazolo |
| | [4,3-d]pyrimidim-7-yl]-2-methyl-phenol |
| 2. | (3-Chloro-4-methoxy-phenyl)-[5-(3,4-dimethoxy-phenyl)-1- |
| | methyl-3-propyl-1H-pyrazolo[4,3-d]pyrimidin-7-yl]-amine |
| | hydrochloride |
| 3. | (3-Chloro-4-methoxy-phenyl)-[5-(4-fluoro-phenyl)-1-methyl-3- |
| | propyl-1H-pyrazolo[4,3-d]pyrimidin-7-yl]-amine hydrochloride |
| 4. | (3-Fluoro-4-methoxy-phenyl)-[5-(4-fluoro-phenyl)-1- |
| | methyl-3-propyl-1H-pyrazolo |
| | [4,3-d]pyrimidin-7-yl]-amine hydrochloride |
| 5. | (3-Fluoro-4-methoxy-phenyl)-[5-(4-fluoro-phenyl)-1- |
| | methyl-3-propyl-1H-pyrazolo[4,3-d]pyrimidin-7-yl]-amine |
| 6. | (4-Fluoro-phenyl)-[5-(4-fluoro-phenyl)-1-methyl-3-propyl-1H- |
| | pyrazolo[4,3-d]pyrimidin-7-yl]-amine hydrochloride |
| 7. | (3,4-Dimethoxy-phenyl)-[5-(4-fluoro-phenyl)-1-methyl-3- |
| | propyl-1H-pyrazolo[4,3-d]pyrimidin-7-yl]-amine hydrochloride |
| 8. | 2-Chloro-4-(1-methyl-5-phenyl-3-propyl-1H-pyrazolo[4,3- |
| | d]pyrimidin-7-ylamino)-phenol hydrochloride |
| 9. | (3-Chloro-4-methoxy-phenyl)-(1-methyl-5-phenyl-3-propyl- |
| | 1H-pyrazolo[4,3-d]pyrimidin-7-yl)-amine hydrochloride |
| 10. | (3-Chloro-4-methoxy-phenyl)-(1-methyl-5-phenyl-3-propyl- |
| | 1H-pyrazolo[4,3-d]pyrimidin-7-yl)-amine |
| 11. | 7-(4-Fluoro-phenoxy)-1-methyl-5-phenyl-3-propyl-1H- |
| | pyrazolo[4,3-d]pyrimidine |
| 12. | 2-Methyl-4-(1-methyl-5-phenyl-3-propyl-1H-pyrazolo[4,3- |
| | d]pyrimidin-7-yl)-phenol |
| 13. | 1-[5-(3,4-Dimethoxy-phenyl)-1-methyl-3-propyl-1H-pyrazolo |
| | [4,3-d]pyrimidin-7-yl]-piperidin-4-ol |
| 14. | 4-[5-(3-hydroxy,4-methoxy-phenyl)-1-methyl-3-propyl-1H- |
| | pyrazolo[4,3-d]pyrimidin-7-yl]-2-methyl-phenol |
| 15. | (3-Fluoro-4-methoxy-phenyl)-(1-methyl-5-phenyl-3-propyl- |
| | 1H-pyrazolo[4,3-d]pyrimidin-7-yl)-amine hydrochloride |
| 16. | (3-Chloro-4-methoxy-phenyl)-(1-methyl-3-propyl-5-thiophen- |
| | 2-yl-1H-pyrazolo[4,3-d]pyrimidin-7-yl)-amine hydrochloride |
| 17. | 2-Chloro-4-[5-(4-fluoro-phenyl)-1-methyl-3-propyl-1H- |
| | pyrazolo[4,3-d]pyrimidin-7-ylamino]-phenol hydrochloride |
| 18. | 3-[7-(3-Chloro-4-methoxy-phenylamino)-1-methyl-3-propyl-1H- |
| | pyrazolo[4,3-d]pyrimidin-5-yl]-4-ethoxy-benzenesulfonamide |
| | hydrochloride |
| 19. | 4-Ethoxy-3-[7-(3-fluoro-4-methoxy-phenylamino)-1-methyl-3- |
| | propyl-1H-pyrazolo[4,3-d]pyrimidin-5-yl]- |
| | benzenesulfonamide hydrochloride |
| 20. | (3-Fluoro-4-methoxy-phenyl)-(1-methyl-3-propyl-5-thiophen-2-yl- |
| | 1H-pyrazolo[4,3-d]pyrimidin-7-yl)-amine hydrochloride |
| 21. | (3-Chloro-4-methoxy-phenyl)-[5-(2-ethoxy-phenyl)-1- |
| | methyl-3-propyl-1H-pyrazolo[4,3-d]pyrimidin-7-yl]-amine |
| | hydrochloride |
| 22. | [5-(2-Ethoxy-phenyl)-1-methyl-3-propyl-1H-pyrazolo |
| | [4,3-d]pyrimidin-7-yl]-(3-fluoro-4-methoxy-phenyl)- |
| | amine hydrochloride |
| 23. | (3-Fluoro-4-methoxy-phenyl)-[5-(4-fluoro-phenyl)-1,3- |
| | dimethyl-1H-pyrazolo[4,3-d]pyrimidin-7-yl]-amine hydrochloride |
| 24. | (3-Chloro-4-methoxy-phenyl)-[5-(4-fluoro-phenyl)-1,3-dimethyl- |
| | 1H-pyrazolo[4,3-d]pyrimidin-7-yl]-amine hydrochloride |
| 25. | 2-Chloro-4-[5-(4-fluoro-phenyl)-1,3-dimethyl-1H-pyrazolo |
| | [4,3-d]pyrimidin-7-ylamino]-phenol hydrochloride |
| 26. | (4-Chloro-3-methoxy-phenyl)-(1-methyl-5-phenyl-3-propyl- |
| | 1H-pyrazolo[4,3-d]pyrimidin-7-yl)-amine hydrochloride |
| 27. | (4-Chloro-3-methoxy-phenyl)-[5-(4-fluoro-phenyl)-1-methyl- |
| | 3-propyl-1H-pyrazolo[4,3-d]pyrimidin-7-yl]-amine hydrochloride |
| 28. | (3-Chloro-4-methoxy-phenyl)-[6-(4-fluoro-phenyl)-1,3- |
| | dimethyl-1H-pyrazolo[4,3-c]pyridin-4-yl]-amine hydrochloride |
| 29. | (3-Fluoro-4-methoxy-phenyl)-[6-(4-fluoro-phenyl)- |
| | 1,3-dimethyl-1H-pyrazolo[4,3-c]pyridin-4-yl]-amine hydrochloride |
| 30. | (3-Chloro-4-methoxy-phenyl)-(1,3-dimethyl-5-phenyl- |
| | 1H-pyrazolo[4,3-d]pyrimidin-7-yl)-amine hydrochloride |
| 31. | (1,3-Dimethyl-5-phenyl-1H-pyrazolo[4,3-d]pyrimidin-7-yl)-(3- |
| | fluoro-4-methoxy-phenyl)-amine hydrochloride |
| 32. | (1,3-Dimethyl-5-thiophen-2-yl-1H-pyrazolo[4,3-d]pyrimidin- |
| | 7-yl)-(3-fluoro-4-methoxy-phenyl)-amine hydrochloride |
| 33. | 2-Chloro-4-[5-(4-fluoro-phenyl)-1-methyl-3-propyl-1H- |
| | pyrazolo[4,3-d]pyrimidin-7-yl]-phenol |
| 34. | (4-Chloro-3-methoxy-phenyl)-(1,3-dimethyl-5-thiophen-2-yl- |
| | 1H-pyrazolo[4,3-d]pyrimidin-7-yl)-amine hydrochloride |
| 35. | 2-Chloro-4-(1,3-dimethyl-5-thiophen-2-yl-1H-pyrazolo |
| | [4,3-d]pyrimidin-7-ylamino)-phenol hydrochloride |
| 36. | 7-(4-Methoxy-3-methyl-phenyl)-1-methyl-5-phenyl-3-propyl- |
| | 1H-pyrazolo[4,3-d]pyrimidine |
| 37. | 5-(4-Fluoro-phenyl)-1,3-dimethyl-7-phenyl-1H-pyrazolo[4,3- |
| | d]pyrimidine |
| 38. | 5-(4-Fluoro-phenyl)-1-methyl-3-propyl-7-p-tolyl-1H- |
| | pyrazolo[4,3-d]pyrimidine |
| 39. | 7-(3-Fluoro-4-methoxy-phenyl)-1-methyl-5-phenyl-3-propyl- |
| | 1H-pyrazolo[4,3-d]pyrimidine |
| 40. | (3-Chloro-4-methoxy-phenyl)-(1,6-diphenyl-1H-pyrazolo |
| | [3,4-d]pyrimidin-4-yl)-amine hydrochloride |
| 41. | (3-Fluoro-4-methoxy phenyl)-[6-(4-fluoro-phenyl)-1-phenyl-1H- |
| | pyrazolo[3,4-d]-pyrimidin-4yl]amine hydrochloride |
| 42. | 2-Fluoro-4-[5-(4-fluoro-phenyl)-1-methyl-3-propyl-1H- |
| | pyrazolo[4,3-d]pyrimidin-7-ylamino]-phenol hydrochloride |
| 43. | Benzo[1,3]dioxol-5-yl-[5-(4-fluoro-phenyl)-1-methyl- |
| | 3-propyl-1H-pyrazolo[4,3-d]pyrimidin-7-yl]-amine hydrochloride |
| 44. | (3-Fluoro-4-methoxy-phenyl)-(1-methyl-3-propyl-5- |
| | trifluoromethyl-1H-pyrazolo[4,3- |
| | d]pyrimidin-7-yl)-amine hydrochloride |
| 45. | 5-(4-Fluoro-phenyl)-7-(4-hydroxy-3-methyl-phenyl)-1-methyl-3- |
| | propyl-1H-pyrazolo[4,3-d]pyrimidine |
| 46. | 5-(4-Fluoro-phenyl)-7-(4-methoxy-3-methyl-phenyl)-1-methyl-3- |
| | propyl-1H-pyrazolo[4,3-d]pyrimidine |
| 47. | (4-Chloro-3-methoxy-phenyl)-(1-methyl-3-propyl-5-thiophen- |
| | 2-yl-1H-pyrazolo[4,3-d]pyrimidin-7-yl)-amine hydrochloride |
| 48. | Benzo[1,3]dioxol-5-yl-(1-methyl-3-propyl-5-thiophen-2-yl-1H- |
| | pyrazolo[4,3-d]pyrimidin-7-yl)-amine hydrochloride |
| 49. | 5-(4-Fluoro-phenyl)-7-indol-1-yl-1-methyl-3-propyl-1H- |
| | pyrazolo[4,3-d]pyrimidine |
| 50. | (4-Chloro-3-trifluoromethyl-phenyl)-[6-(4-fluoro-phenyl)-1-phenyl- |
| | 1H-pyrazolo[3,4-d]pyrimidin-4-yl]-amine hydrochloride |
| 51. | (1,3-Dimethyl-5-phenyl-1H-pyrazolo[4,3-d]pyrimidin-7-yl)- |
| | (3-fluoro-phenyl)-amine hydrochloride |
| 52. | [5-(4-Fluoro-phenyl)-1-methyl-3-propyl-1H-pyrazolo[4,3- |
| | d]pyrimidin-7-yl]-(3-trifluoromethyl-phenyl)-amine hydrochloride |
| 53. | [5-(4-Fluoro-phenyl)-1-methyl-3-propyl-1H-pyrazolo[4,3- |
| | d]pyrimidin-7-yl]-(4-trifluoromethoxy-phenyl)- |
| | amine hydrochloride |
| 54. | (5-Chloro-3-phenyl-1H-pyrazolo[4,3-d]pyrimidin- |
| | 7-yl)-(4-fluoro-phenyl)-amine hydrochloride |
| 55. | (1,3-Dimethyl-5-phenyl-1H-pyrazolo[4,3-d]pyrimidin-7-yl)- |
| | (4-trifluoromethoxy-phenyl)-amine hydrochloride |
| 56. | 4-Benzo[1,3]dioxol-5-yl-6-(4-fluoro-phenyl)-1,3-dimethyl- |
| | 1H-pyrazolo[4,3-c]pyridine |
| 57. | (6-Chloro-pyridin-3-yl)-[5-(4-fluoro-phenyl)-1,3-dimethyl-1H- |
| | pyrazolo[4,3-d]pyrimidin-7-yl]-amine hydrochloride |
| 58. | (4-Chloro-3-trifluoromethyl-phenyl)-[6-(4-fluoro-phenyl)- |
| | 1,3-dimethyl-1H-pyrazolo[4,3-c]pyridin-4-yl]-amine hydrochloride |
| 59. | [6-(4-Fluoro-phenyl)-1,3-dimethyl-1H-pyrazolo[4,3-c] |
| | pyridin-4-yl]-(3-trifluoromethyl-phenyl)-amine hydrochloride |
| 60. | (6-Chloro-pyridin-3-yl)-[6-(4-fluoro-phenyl)-1,3-dimethyl- |
| | 1H-pyrazolo[4,3-c]pyridin-4-yl]-amine hydrochloride |
| 61. | [5-(4-Fluoro-phenyl)-1,3-dimethyl-1H-pyrazolo[4,3-d] |
| | pyrimidin-7-yl]-(4-methanesulfonyl-phenyl)-amine |
| 62. | 7-(3-Methanesulfonyl-phenyl)-1,3-dimethyl-5-phenyl- |
| | 1H-pyrazolo[4,3-d]pyrimidine |
| 63. | (1,3-Dimethyl-5-phenyl-1H-pyrazolo[4,3-d]pyrimidin-7- |
| | yl)-(2-methyl-benzooxazol-5-yl)-amine hydrochloride |
| 64. | 5-(4-Fluoro-phenyl)-7-(3-methanesulfonyl-phenyl)- |
| | 1,3-dimethyl-1H-pyrazolo[4,3-d] |
| | pyrimidine |
| 65. | 6-(4-Fluoro-phenyl)-4-(3-methanesulfonyl-phenyl)-1,3- |
| | dimethyl-1H-pyrazolo[4,3-c]pyridine |
| 66. | (1H-Benzoimidazol-5-yl)-(1,3-dimethyl-5-phenyl-1H-pyrazolo[4,3- |
| | d]pyrimidin-7-yl)-amine hydrochloride |
| 67. | 6-(4-Fluoro-phenyl)-4-(4-methanesulfonyl-phenyl)-1,3-dimethyl- |
| | 1H-pyrazolo[4,3-c]pyridine |
| 68. | 7-Fluoro-1,3-dimethyl-5-phenyl-1H-pyrazolo[4,3-d]pyrimidine |
| 69. | (1,3-Dimethyl-5-phenyl-1H-pyrazolo[4,3-d]pyrimidin-7-yl)-(2- |
| | methyl-1H-benzoimidazol-5-yl)-amine hydrochloride |
| 70. | N-{4-[6-(4-Fluoro-phenyl)-1,3-dimethyl- |
| | 1H-pyrazolo[4,3-c]pyridin- |
| | 4-ylamino]-phenyl}-methanesulfonamide hydrochloride |
| 71. | (3-Fluoro-phenyl)-[6-(4-fluoro-phenyl)-1-phenyl-1H-pyrazolo |
| | [3,4-d]pyrimidin-4-yl]-amine hydrochloride |
| 72. | [6-(4-Fluoro-phenyl)-1-phenyl-1H-pyrazolo[3,4-d]pyrimidin-4- |
| | yl]-(4-trifluoromethoxy-phenyl)-amine hydrochloride |
| 73. | N-[4-(1,3-Dimethyl-5-phenyl-1H-pyrazolo[4,3-d]pyrimidin-7- |
| | ylamino)-phenyl]-methanesulfonamide hydrochloride |
| 74. | 6-(4-fluoro phenyl)-(1,3-dimethyl-6-phenyl-1H-pyrazolo[4,3- |
| | c]pyridin-4-yl)-(4-trifluoromethoxy-phenyl)-amine hydrochloride |
| 75. | [6-(4-Fluoro-phenyl)-1,3-dimethyl-1H-pyrazolo[4,3-c]pyridin-4- |
| | yl]-(4-methanesulfonyl-phenyl)-amine |
| 76. | 4-[5-(4-Fluoro-phenyl)-1,3-dimethyl-1H-pyrazolo[4,3-d]pyrimidin- |
| | 7-ylamino]-N,N-dimethyl-benzenesulfonamide hydrochloride |
| 77. | 4-[5-(4-Fluoro-phenyl)-1,3-dimethyl-1H-pyrazolo[4,3-d]pyrimidin- |
| | 7-ylamino]-benzenesulfonamide hydrochloride |
| 78. | 4-[5-(4-Fluoro-phenyl)-1,3-dimethyl-1H-pyrazolo[4,3-d]pyrimidin- |
| | 7-ylamino]-N-methyl-benzenesulfonamide hydrochloride |
| 79. | 4-[6-(4-Fluoro-phenyl)-1,3-dimethyl-1H-pyrazolo[4,3-c]pyridin-4- |
| | ylamino]-N-methyl-benzenesulfonamide hydrochloride |
| 80. | 4-[5-(4-Fluoro-phenyl)-1,3-dimethyl-1H-pyrazolo[4,3-d]pyrimidin- |
| | 7-ylamino]-benzamide hydrochloride |
| 81. | 3-[5-(4-Fluoro-phenyl)-1,3-dimethyl-1H-pyrazolo[4,3-d]pyrimidin- |
| | 7-ylamino]-benzamide hydrochloride |
| 82. | 3-[5-(4-Fluoro-phenyl)-1,3-dimethyl-1H-pyrazolo[4,3-d]pyrimidin- |
| | 7-ylamino]-N-methyl-benzamide hydrochloride |
| 83. | 6-(4-Fluoro-phenyl)-1,3-dimethyl-4-(4-trifluoromethoxy-phenyl)- |
| | 1H-pyrazolo[4,3-c]pyridine |

Inclusion of a compound in any table disclosed herein is not to be seen as limiting, in that the compound included in a specific table has at least the affect shown for inclusion in the table and may have additional other affects. Nor are the tables to be seen as limiting in that the compounds listed in a particular table are the only compounds disclosed herein that have that affect.

Assays for determining the ability of a compound of the present invention to modulate inflammation, or more specifically, attenuate or inhibit glycated protein- or AGE-induced inflammation are described herein and in U.S. patent application Ser. Nos. 10/026,335 and 09/969,013, which are incorporated herein by reference.

In some assays, for example, the specific expression (i.e., production or activity) of a substance or biological component involved in a known cellular response is measured. The assays provide a measurable response in which the affect of a compound is determined.

One assay, for example, comprises measuring the effect of a compound on a known inflammatory response of cells to a stimulating agent such as, for example, a glycated protein.

In another assay, for example, cytokine expression of stimulated cells can be measured in control cells and cells exposed to a compound described herein. Illustratively, a stimulated cell can be an endothelial cell stimulated with glycated protein. Comparison of the cytokine profile of control cells (i.e., baseline) versus cells exposed to the compound can indicate the affect of the compound on cytokine expression and, hence, inflammation. The cytokine profile can be qualitative and/or quantitative. For example, where the cytokine is a secreted protein, the amount of the cytokine present in the media can be quantitated using antibodies specific to the cytokine. The compound may have an inhibitory effect, stimulatory effect, or no effect at all. Besides cytokines, expression of other factors or parameters can be determined using such assays.

One or more compounds can be added to a screening assay. Combinations or mixtures of compounds can be added. Different amounts and formulations of the compounds can be added to determine the effects on the screening assay.

In one aspect of the present invention, compounds that attenuate or inhibit an inflammatory response of a cell to glycated albumin are used as therapeutic agents. One skilled in the art knows how to measure cytokine expression. The amount and type of cytokine expressed can be determined using immunological methods, such as ELISA assays. The methods of the present invention are not limited by the type of assay used to measure the amount of cytokine expressed, and any methods known to those skilled in the art and later developed can be used to measure the amount of cytokines expressed in response to the stimulating agent and to the compound having an unknown effect.

Correlation of Physiological Parameters and Assays to Diseases and Conditions

Tables 8-11 provide disclosure and references that link or relate the various parameters and assays disclosed herein to general and/or specific conditions or diseases. The references provided in these tables support the specification as fully enabled for treating all the diseases or conditions encompassed herein, based on the inhibiting effect of the compounds provided in the specification, and the predictive nature of the tests provided of the disclosed uses.

Table 8 provides references illustrating the connection between TNF-α and IL-6 in rheumatoid arthritis, vascular inflammation, and atherosclerosis.

Table 9 provides references illustrating the importance of HSPG expression in the prevention of atherosclerosis and diabetic vascular disease.

Table 10 provides references illustrating the role of SMC proliferation in contributing to restenosis and atherosclerosis.

Table 11 provides references illustrating the role of heparanase and TNF-α expression in promoting tumor angiogenesis and metastasis, as well as the use of inhibitors of heparanase and TNF-α expression in treating cancer.

Examples of assays described herein for screening the compounds of the present invention include, but are not limited to, assays that demonstrate: a) inhibition of SMC proliferation, that was used to identify, for example, compounds in Table 4; b) induction of HSPG expression in SMCs; c) induction of heparanase expression in endothelial cells; d) inhibition of AGE-induced inflammatory response in endothelial cells as measured by IL-6 or other inflammatory cytokine expression, that was used to identify, for example, compounds in Table 7; and e) cytotoxicity effects of the disclosed compounds. By using these disclosed assays, the present disclosure is fully enabled for identification of compounds for the treatment or prevention of the diseases disclosed generically or specifically.

TABLE 8
The Role of TNF-α, IL-6, and AGE in Rheumatoid Arthritis,
Vascular Inflammation, and Atherosclerosis.

| | | Reference | Physiological | |
|---|---|---|---|---|
| Author | Title of Reference | Citation | Parameter | Disease |
| Feldmann M | Discovery of TNF-α as a | Joint Bone Spine. | TNF | Arthritis |
| Ref 1 | therapeutic target in rheumatoid | 2002 | inhibition | |
| | arthritis: preclinical and clinical | Jan; 69(1): 12-8 | | |
| | studies | Review | | |
| Choy et al | Therapeutic benefit of blocking | Arthritis Rheum. | IL-6 | Arthritis |
| Ref 2 | interleukin-6 activity with an anti- | 2002 | inhibition | |
| | interleukin-6 receptor monoclonal | Dec; 46(12): 3143-50 | | |
| | antibody in rheumatoid arthritis: a | | | |
| | randomized, double-blind, placebo- | | | |
| | controlled, dose-escalation trial. | | | |
| Wong et al | The role of the interleukin-6 family | Arthritis Rheum. | IL-6 | Arthrits |
| Ref 3 | of cytokines in inflammatory | 2003 | inhibition | |
| | arthritis and bone turnover | May; 48(5): 1177-89. | | |
| | | Review | | |
| Basta et al | Advanced glycation end products | Cardiovasc Res. | AGE-IL6 | Diabetic vascular |
| Ref 4 | and vascular inflammation: | 2004 Sep | inhibition | diseases |
| | implications for accelerated | 1; 63(4): 582-92 | | |
| | atherosclerosis in diabetes | | | |

TABLE 9
The Role of HSPG Induction in the Prevention of Atherosclerosis
and Diabetic Vascular Disease.

| | | Reference | Physiological | |
|---|---|---|---|---|
| Author | Title of Reference | Citation | Parameter | Disease |
| Engelberg H. | Endogenous heparin | Atherosclerosis. | HSPG | Atherosclerosis |
| Ref 5 | activity deficiency: the | 2001 | induction | |
| | ‘missing link’ in | Dec; 159(2): 253-60. | | |
| | atherogenesis? | Review | | |
| Jensen T | Pathogenesis of | Diabetes. 1997 | HSPG | Diabetic |
| Ref 6 | diabetic vascular | Sep; 46 Suppl | induction | vascular |
| | disease: evidence for | 2: S98-100 | | disease |
| | the role of reduced | | | |
| | heparan sulfate | | | |
| | proteoglycan | | | |
| Hollmann J et al, | Relationship of | Artherosclerosis. | HSPG | Atherosclerosis |
| Ref 7 | sulfated | 1989; 9: 154-8 | | |
| | glycosaminoglycans | | | |
| | and cholesterol | | | |
| | content in normal and | | | |
| | atherosclerotic human | | | |
| | aorta | | | |
| Kruse R et al | Cholesterol-dependent | Basic Res | HSPG | Atherosclerosis |
| Ref 8 | changes of | Cardiol. 1996 | | |
| | glycosaminoglycan | Sep-Oct; | | |
| | pattern in human aorta | 91(5): 344-52 | | |

TABLE 10
The Role of SMC Proliferation in Restenosis and Atherosclerosis.

| | | Reference | Physiological | |
|---|---|---|---|---|
| Author | Title of Reference | Citation | Parameter | Disease |
| Chen et al | Electron microscopic | Circulation. 1997 | Smooth | Restenosis |
| Ref 9 | studies of phenotypic | Mar 4; 95(5): 1169-75 | muscle cell | |
| | modulation of smooth | | (SMC) | |
| | muscle cells in | | proliferation | |
| | coronary arteries of | | | |
| | patients with unstable | | | |
| | angina pectoris and | | | |
| | postangioplasty | | | |
| | restenosis | | | |
| Braun-Dullaeus et | Cell cycle progression: | Circulation. 1998 | Smooth | Restenosis |
| al | new therapeutic target | Jul 7; 98(1): 82-9 | muscle cell | |
| Ref 10 | for vascular | | (SMC) | |
| | proliferative disease | | proliferation | |
| Boucher et al | LRP: role in vascular | Science. 2003 | Smooth | Atherosclerosis |
| Ref 11 | wall integrity and | Apr | muscle cell | |
| | protection from | 11; 300(5617): 329-32 | (SMC) | |
| | atherosclerosis | | proliferation | |
| Marx et al | Bench to bedside: the | Circulation. 2001 | Smooth | Restenosis |
| Ref 12 | development of | Aug | muscle cell | |
| | rapamycin and its | 21; 104(8): 852-5 | (SMC) | |
| | application to stent | | proliferation | |
| | restenosis | | | |

TABLE 11
The Role of Heparanase and TNF-α in Promoting Tumor Angiogenesis and
Metastasis and the Use of Heparanase and TNF-α Inhibitors in Treating Cancer.

| | | | Physiological |
|---|---|---|---|
| Author | Title of Reference | Reference Citation | Parameter |
| Vlodavsky I et | Molecular properties and | J Clin Invest. 2001 | Heparanase |
| al | involvement of | Aug; 108(3): 341-7. | inhibition |
| Ref 13 | heparanase in cancer | Review | |
| | metastasis and | | |
| | angiogenesis | | |
| Goldshmidt et | Cell surface expression | Proc Natl Acad Sci USA. | Heparanase |
| al | and secretion of | 2002 Jul | inhibition |
| Ref 14 | heparanase markedly | 23; 99(15): 10031-6 | |
| | promote tumor | | |
| | angiogenesis and | | |
| | metastasis | | |
| Simizu et al | Heparanase as a | Cancer Sci. 2004 | Heparanase |
| Ref 15 | molecular target of | Jul; 95(7): 553-8 | inhibition |
| | cancer chemotherapy | | |
| Szlosarek et al | Tumour necrosis factor α: | The Lancet Oncology | TNFα inhibition |
| Ref 16 | a potential target for | 2003 Sept; 4: 565-73 | |
| | the therapy of solid | | |
| | tumours | | |

Compound/Composition-Coated Medical Devices

The compounds of the present invention can be used alone, in various combinations with one another, and/or in combination with other agents along with delivery devices to effectively prevent and treat the diseases described herein, though particular applications are found in vascular disease, and in particular, vascular disease caused by injury and/or by transplantation. Though this example focuses on vascular disease, provision of the compounds of the present invention with medical devices for treatment of the diseases and conditions capable of being treated with the compounds is contemplated by the present invention.

Various medical treatment devices utilized in the treatment of vascular disease may ultimately induce further complications. For example, balloon angioplasty is a procedure utilized to increase blood flow through an artery and is the predominant treatment for coronary vessel stenosis. However, the procedure typically causes a certain degree of damage to the vessel wall, thereby creating new problems or exacerbating the original problem at a point later in time. Although other procedures and diseases may cause similar injury, exemplary aspects of the present invention will be described with respect to the treatment of restenosis and related complications following percutaneous transluminal coronary angioplasty and other similar arterial/venous procedures, including the joining of arteries, veins, and other fluid carrying conduits in other organs or sites of the body, such as the liver, lung, bladder, kidney, brain, prostate, neck, and legs.

The local delivery of a compound of the present invention and, in some aspects, along with other therapeutic agents, from a stent prevents vessel recoil and remodeling through the scaffolding action of the stent. The effect of a compound provided, with or without other therapeutic agents, helps determine the particular application for which the coated medical device is being administered. For example, compound-coated stents can prevent multiple components of neointimal hyperplasia or restenosis as well as reduce inflammation and thrombosis. Local administration of a compound of the present invention and other therapeutic agents to stented coronary arteries may also have additional therapeutic benefit. For example, higher tissue concentrations of the compounds of the present invention and other therapeutic agents can be achieved utilizing local delivery rather than systemic administration. In addition, reduced systemic toxicity can be achieved utilizing local delivery rather than systemic administration while maintaining higher tissue concentrations. In utilizing local delivery from a stent rather than systemic administration, a single procedure may suffice with better patient compliance. An additional benefit of combination therapeutic agent and/or compound therapy can be to reduce the dose of each of the therapeutic agents, thereby limiting toxicity, while still achieving a reduction in restenosis, inflammation, and thrombosis. Local stent-based therapy is therefore a means of improving the therapeutic ratio (efficacy/toxicity) of anti-restenosis, anti-inflammatory, and anti-thrombotic therapeutic agents.

Although exemplary aspects of the invention will be described with respect to the treatment of restenosis and other related complications, it is important to note that the local delivery of a compound of the present invention, alone or as part of a therapeutic agent combination, can be utilized to treat a wide variety of conditions utilizing any number of medical devices, or to enhance the function and/or life of the device. For example, intraocular lenses, placed to restore vision after cataract surgery, are often compromised by the formation of a secondary cataract. The latter is often a result of cellular overgrowth on the lens surface and can be potentially minimized by combining one or more compounds of the present invention having an effect in preventing unwanted cellular growth with the device. Other medical devices that often fail due to tissue in-growth or accumulation of proteinaceous material in, on and around the device, such as shunts for hydrocephalus, dialysis grafts, colostomy bag attachment devices, ear drainage tubes, leads for pace makers, and implantable defibrillators can also benefit from the combinations of the compounds of the present invention, possibly other pharmaceutical agents, and the devices. Other surgical devices, sutures, staples, anastornosis devices, vertebral disks, bone pins, suture anchors, hemostatic barriers, clamps, screws, plates, clips, vascular implants, tissue adhesives and sealants, tissue scaffolds, various types of dressings, bone substitutes, intraluminal devices, and vascular supports could also provide enhanced patient benefit using this compound-device combination approach. Essentially, any type of medical device can be coated in some fashion with at least one compound of the present invention, alone or as part of a therapeutic agent combination, which enhances treatment over the use of the device or therapeutic agent without combination with the compound.

As disclosed supra, the compounds of the present invention can be administered in combinational therapies with other therapeutic agents, and are not limited to only the other therapeutic agents disclosed herein. Thus, the present invention also contemplates, in addition to various medical devices, the coatings on these devices can be used to deliver a compound of the present invention in combination with other therapeutic agents. This illustrative list of therapeutic agents can be administered through pharmeutical means or in association with medical devices and such therapeutic agents include, but are not limited to, antiproliferative/antimitotic agents including natural products such as vinca alkaloids (e.g., vinblastine, vincristine, and vinorelbine), paclitaxel, epidipodophyllotoxins (e.g., etoposide, teniposide), antibiotics [e.g., dactinomycin (actinomycin D) daunorubicin, doxorubicin, and idarubicin], anthracyclines, mitoxantrone, bleomycins, plicamycin (mithramycin), and mitomycin, enzymes (L-asparaginase which systemically metabolizes L-asparagine and deprives cells which do not have the capacity to synthesize their own asparagine); antiplatelet agents such as G(GP) IIb/IIIa inhibitors and vitronectin receptor antagonists; antiproliferative/antimitotic alkylating agents such as nitrogen mustards (e.g., mechlorethamine, cyclophosphamide and analogs, melphalan, chlorambucil), ethylenimines and methylmelamines (hexamethylmelamine and thiotepa), alkyl sulfonates-busulfan, nirtosoureas [carmustine (BCNU) and analogs, streptozocin], trazenes-dacarbazinine (DTIC); antiproliferative/antimitotic antimetabolites such as folic acid analogs (methotrexate), pyrimidine analogs (e.g., fluorouracil, floxuridine, and cytarabine), purine analogs and related inhibitors [mercaptopurine, thioguanine, pentostatin, and 2-chlorodeoxyadenosine (cladribine)]; platinum coordination complexes (cisplatin, carboplatin), procarbazine, hydroxyurea, mitotane, aminoglutethimide; hormones (e.g., estrogen); anticoagulants (e.g., heparin, synthetic heparin salts and other inhibitors of thrombin); fibrinolytic agents (such as tissue plasminogen activator, streptokinase, and urokinase), aspirin, dipyridamole, ticlopidine, clopidogrel, abciximab; antimigratory; antisecretory (breveldin); anti-inflammatory agents such as adrenocortical steroids (e.g., cortisol, cortisone, fludrocortisone, prednisone, prednisolone, 6α-methylprednisolone, triamcinolone, betamethasone, and dexamethasone), non-steroidal agents (salicylic acid derivatives, i.e., aspirin; para-aminophenol derivatives, i.e., acetominophen; indole and indene acetic acids (indomethacin, sulindac, and etodalac), heteroaryl acetic acids (tolmetin, diclofenac, and ketorolac), arylpropionic acids (ibuprofen and derivatives), anthranilic acids (mefenamic acid, and meclofenamic acid), enolic acids (piroxicam, tenoxicam, phenylbutazone, and oxyphenthatrazone), nabumetone, gold compounds (auranofin, aurothioglucose, gold sodium thiomalate); immunosuppressives, (Cyclosporine, tacrolimus (FK-506), sirolimus (rapamycin), azathioprine, mycophenolate mofetil); angiogenic agents: vascular endothelial growth factor (VEGF), fibroblast growth factor (FGF); angiotensin receptor blockers; nitric oxide donors; anti-sense oligionucleotides and combinations thereof; cell cycle inhibitors, mTOR inhibitors, and growth factor signal transduction kinase inhibitors.

Although any number of stents can be utilized in accordance with the present invention, for simplicity, a limited number of stents will be described in exemplary aspects of the present invention. The skilled artisan will recognize that any number of stents can be utilized in connection with the present invention. In addition, as stated above, other medical devices can be utilized. For example, though stents are described, sleeves outside the vessels are also contemplated, as are other medical devices that can provide a substrate for administration for at least one of the compounds of the present invention.

A stent is commonly used as a tubular structure left inside the lumen of a duct to relieve an obstruction. Typically, stents are inserted into the lumen in a non-expanded form and are then expanded autonomously, or with the aid of a second device in situ. A common method of expansion occurs through the use of a catheter-mounted, angioplasty balloon that is inflated within the stenosed vessel or body passageway in order to shear and disrupt the obstructions associated with the wall components of the vessel and to obtain an enlarged lumen.

A stent may resemble an expandable cylinder and may comprise a fenestrated structure for placement in a blood vessel, duct or lumen to hold the vessel, duct or lumen open, more particularly for protecting a segment of artery from restenosis after angioplasty. The stent can be expanded circumferentially and maintained in an expanded configuration that is circumferentially or radially rigid. The stent can be axially flexible and when flexed at a band, for example, the stent avoids any externally protruding component parts.

The stent can be fabricated utilizing any number of methods. For example, the stent can be fabricated from a hollow or formed stainless steel tube that can be machined using lasers, electric discharge milling, chemical etching or other means. The stent is inserted into the body and placed at the desired site in an unexpanded form. In one aspect, expansion can be effected in a blood vessel by a balloon catheter, where the final diameter of the stent is a function of the diameter of the balloon catheter used. It should be appreciated that a stent in accordance with the present invention can be embodied in a shape-memory material including, for example, an appropriate alloy of nickel and titanium or stainless steel.

Structures formed from stainless steel can be made self-expanding by configuring the stainless steel in a predetermined manner, for example, by twisting it into a braided configuration. In this aspect, after the stent has been formed it can be compressed so as to occupy a space sufficiently small as to permit its insertion in a blood vessel or other tissue by insertion means, wherein the insertion means include a suitable catheter, or flexible rod. Upon emerging from the catheter, the stent can be configured to expand into the desired configuration where the expansion is automatic or triggered by a change in pressure, temperature, or electrical stimulation.

Furthermore, a stent can be modified to comprise one or more reservoirs. Each of the reservoirs can be opened or closed as desired. These reservoirs can be specifically designed to hold the the compound or compound/therapeutic agent combination to be delivered. Regardless of the design of the stent, it is preferable to have the compound or compound/therapeutic agent combination dosage applied with enough specificity and a sufficient concentration to provide an effective dosage in the affected area. In this regard, the reservoir size in the bands is preferably sized to adequately apply the compound or compound/therapeutic agent combination dosage at the desired location and in the desired amount.

In an alternative aspect, the entire inner and outer surface of the stent can be coated with the compound or compound/therapeutic agent combination in therapeutic dosage amounts. The coating techniques may vary depending on the the compound or compound/therapeutic agent combination. Also, the coating techniques may vary depending on the material comprising the stent or other intraluminal medical device.

One or more compounds of the present invention and, in some instances, other therapeutic agents as a combination, can be incorporated onto or affixed to the stent in a number of ways. In one aspect, the compound is directly incorporated into a polymeric matrix and sprayed onto the outer surface of the stent. The compound elutes from the polymeric matrix over time and enters the surrounding tissue. The compound preferably remains on the stent for at least three days up to approximately six months, and more preferably between seven and thirty days.

Any number of non-erodible polymers can be utilized in conjunction with the compound, and such polymeric compositions are well known in the art. In one aspect, the polymeric matrix comprises two layers. The base layer comprises a solution of poly(ethylene-co-vinylacetate) and polybutylmethacrylate. The compound is incorporated into this base layer. The outer layer comprises only polybutylmethacrylate and acts as a diffusion barrier to prevent the compound from eluting too quickly. The thickness of the outer layer or topcoat determines the rate at which the compound elutes from the matrix. Essentially, the compound elutes from the matrix by diffusion through the polymer matrix. Polymers are permeable, thereby allowing solids, liquids and gases to escape therefrom. The total thickness of the polymeric matrix is in the range from about one micron to about twenty microns or greater. It is important to note that primer layers and metal surface treatments can be utilized before the polymeric matrix is affixed to the medical device. For example, acid cleaning, alkaline (base) cleaning, salinization and parylene deposition can be used as part of the overall process described above.

The poly(ethylene-co-vinylacetate), polybutylmethacrylate, and compound solution can be incorporated into or onto the stent in a number of ways. For example, the solution can be sprayed onto the stent or the stent can be dipped into the solution. Other methods include spin coating and plasma polymerization. In one aspect, the solution is sprayed onto the stent and then allowed to dry. In another aspect, the solution can be electrically charged to one polarity and the stent electrically charged to the opposite polarity. In this manner, the solution and stent will be attracted to one another. In using this type of spraying process, waste can be reduced and more precise control over the thickness of the coat can be achieved.

Drug-coated stents are manufactured by a number of companies including Johnson & Johnson, Inc. (New Brunswick, N.J.), Guidant Corp. (Santa Clara, Calif.), Medtronic, Inc. (Minneapolis, Minn.), Cook Group Incorporated (Bloomington, Ind.), Abbott Labs., Inc. (Abbott Park, Ill.), and Boston Scientific Corp. (Natick, Mass.). See e.g., U.S. Pat. No. 6,273, 913; U.S. Patent Application Publication No. 20020051730; WO 02/26271; and WO 02/26139.

Pharmaceutical Compositions

In one aspect, the present invention provides a composition comprising at least one compound as disclosed herein.

In another aspect, this invention provides a pharmaceutical composition, comprising:

at least one compound as disclosed herein; and

optionally comprising a pharmaceutically acceptable additive selected from a carrier, an auxiliary, a diluent, an excipient, a preservative, a solvate, or any combination thereof.

In yet another aspect, this invention provides a pharmaceutical composition, comprising:

at least one compound as disclosed herein; and

optionally comprising a pharmaceutically acceptable additive selected from a carrier, an auxiliary, a diluent, an excipient, a preservative, a solvate, or any combination thereof;

wherein the pharmaceutical composition is in the form of a tablet, a capsule, a syrup, a cachet, a powder, a granule, a solution, a suspension, an emulsion, a bolus, a lozenge, a suppository, a cream, a gel, a paste, a foam, a spray, an aerosol, a microcapsule, a liposome, or a transdermal patch.

In still another aspect, this invention provides a pharmaceutical composition, comprising:

at least one compound as disclosed herein;

optionally comprising a pharmaceutically acceptable additive selected from a carrier, an auxiliary, a diluent, an excipient, a preservative, a solvate, or any combination thereof; and

further comprising an agent selected from a chemotherapeutic agent, an immunosuppressive agent, a cytokine, a cytotoxic agent, an anti-inflammatory agent, an antirheumatic agent, an antidyspilidemic agent, a cardiovascular agent, or any combination thereof.

Accordingly, in addition to the compounds disclosed herein, the pharmaceutical compositions of the present invention can further comprise at least one of any suitable auxiliary such as, but not limited to, diluent, binder, stabilizer, buffers, salts, lipophilic solvents, preservative, adjuvant, or the like. In one aspect of the present invention, pharmaceutically acceptable auxiliaries are employed. Examples and methods of preparing such sterile solutions are well known in the art and can be found in well known texts such as, but not limited to, REMINGTON'S PHARMACEUTICAL SCIENCES (Gennaro, Ed., 18th Edition, Mack Publishing Co. (1990)). Pharmaceutically acceptable carriers can be routinely selected that are suitable for the mode of administration, solubility and/or stability of the compound.

Pharmaceutical Compositions for Oral Administration

For oral administration in the form of a tablet or capsule, a compound can be combined with an oral, non-toxic pharmaceutically acceptable inert carrier such as ethanol, glycerol, water, and the like. Moreover, when desired or necessary, suitable binders, lubricants, disintegrating agents, and coloring agents may also be incorporated into the mixture. Suitable binders include, without limitation, starch; gelatin; natural sugars such as glucose or beta-lactose; corn sweeteners; natural and synthetic gums such as acacia, tragacanth, or sodium alginate, carboxymethylcellulose; polyethylene glycol; waxes; and the like. Lubricants used in these dosage forms include, without limitation, sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

Formulations of the present invention suitable for oral administration can be presented as discrete units such as capsules, cachets, or tablets each containing a predetermined amount of the active ingredient; as a powder or granules; as a solution or a suspension in an aqueous liquid or a non-aqueous liquid; or as an oil-in-water liquid emulsion or a water-in-oil emulsion and as a bolus, and the like.

Routes of Administration

The invention further relates to the administration of at least one compound disclosed herein by the following routes, including, but not limited to oral, parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, iontophoretic means, or transdermal means.

Dosages

A composition comprising at least one compound of the present invention can be administered at a frequency and for a period of time effective to achieve a therapeutic effect, which should be understood in the context of a regimen of repeated administration at such a frequency and over such a period. In some aspects, a composition is administered at a frequency and for a period of time effective to increase a HSPG expression. In some aspects, a composition can be administered in a single daily dose, or a total daily dosage can be administered in divided doses of two, three, or four times daily. Typically and most conveniently, a composition is administered at least once daily, but in certain situations less frequent, e.g., twice weekly or weekly, administration can be effective. For greatest benefit, administration should continue for a prolonged period, for example at least about 3 months, or at least about 6 months, or at least about 1 year, or at least about 2 years, or at least about 3 years. In one aspect, administration continues from a time of initiation for substantially the remainder of the mammal's life.

The selection and/or amounts of individual compounds can, if desired vary over the period of administration. In one aspect, a single composition of this invention is administered to a mammal for the entire period of administration. In other aspects, different compositions comprising at least one compound are administered to the mammal at different times.

The dosages of compounds can be adjusted on a per body weight basis and may thus be suitable for any subject regardless of the subject's size.

In one aspect of this invention, daily oral dose comprises a total compound amount of at least about 0.0001 mg per kg body weight, illustratively about 0.0001 mg to about 1000 mg, about 0.001 mg to about 100 mg, about 0.01 mg to about 10 mg, about 0.1 mg to about 5 mg, or about 1 to about 3 mg per kg body weight.

In another aspect, a daily intravenous injection comprises a total compound amount of at least about 0.0001 mg per kg body weight, illustratively about 0.0001 mg to about 0.5 mg, about 0.001 mg to about 0.25, or about 0.01 to about 0.03 mg per kg body weight.

Illustratively, a tablet for oral administration can be manufactured to comprise a total compound amount of about 0.001 mg, about 0.1 mg, about 0.2 mg, about 0.5 mg, about 1 mg, about 2 mg, about 5 mg, about 10 mg, about 15 mg, about 100 mg, about 150 mg, about 200 mg, about 250 mg, about 300 mg, about 350 mg, about 400 mg, about 450 mg, about 500 mg, about 550 mg, about 600 mg, about 650 mg, about 700 mg, about 800 mg, about 900 mg, or about 1000 mg.

In one aspect, a composition comprises an active ingredient content of at least about 0.01% by weight of the composition, illustratively about 0.01% to about 99%, about 0.05% to about 90%, about 0.1% to about 80%, about 0.5% to about 50% by weight of the composition. The amount of active ingredient that can be combined with other materials to produce a single dosage form varies depending upon the subject treated and the particular mode of administration.

An effective amount of the drug is ordinarily supplied at a dosage level of from about 0.1 mg/kg to about 20 mg/kg of body weight per day. In one aspect, the range is from about 0.2 mg/kg to about 10 mg/kg of body weight per day. In another aspect, the range is from about 0.5 mg/kg to about 10 mg/kg of body weight per day. The compounds can be administered on a regimen of about 1 to about 10 times per day.

Co-administration or sequential administration of the compounds of the present invention and other therapeutic agents can be employed, such as chemotherapeutic agents, immunosuppressive agents, cytokines, cytotoxic agents, nucleolytic compounds, radioactive isotopes, receptors, and pro-drug activating enzymes, which can be naturally occurring or produced by recombinant methods. The combined administration includes co-administration, using separate formulations or a single pharmaceutical formulation, and consecutive administration in either order, wherein preferably there is a time period while both (or all) active therapeutic agents simultaneously exert their biological activities.

It is to be understood that this invention is not limited to the particular methodology, syntheses, formulations, protocols, cell lines, constructs, and reagents described herein and as such can vary. It is also to be understood that the terminology used herein is for the purpose of describing particular aspects only, and is not intended to limit the scope of the present invention.

All publications, patents, and other references mentioned herein are provided for the purpose of describing and disclosing, for example, the constructs and methodologies that are described in these references, which might be used in connection with the presently described invention.

Definitions and Terminology

The groups defined for various symbols used in the formulas of this disclosure, as well as the optional substituents defined on those groups, can be defined as follows. Unless otherwise specified, any recitation of the number of carbon atoms in a particular group is intended to refer to the unsubstituted “base” group, therefore, any substituent recited on a base group is described by its own definition, including its own limitation of the number of carbon atoms. Unless otherwise specified, all structural isomers of a given structure, for example, all enantiomers, diasteriomers, and regioisomers, are included within this definition.

The terms ‘halogen’ or ‘halo’ includes fluorine, chlorine, bromine, or iodine.

The term ‘alkyl’ group is used to refer to both linear and branched alkyl groups. Exemplary alkyl groups include, but are not limited to, methyl, ethyl, propyl, butyl, pentyl, pentyl, hexyl, heptyl, octyl, nonyl, or decyl, and the like. Unless otherwise specified, an alkyl group has from 1 to 10 carbon atoms. Also unless otherwise specified, all structural isomers of a given structure, for example, all enantiomers and all diasteriomers, are included within this definition. For example, unless otherwise specified, the term propyl is meant to include n-propyl and iso-propyl, while the term butyl is meant to include n-butyl, iso-butyl, t-butyl, sec-butyl, and so forth.

‘Haloalkyl’ is a group containing at least one halogen and an alkyl portion as define above. Unless otherwise specified, all structural isomers of a given structure, for example, all enantiomers and all diasteriomers, are included within this definition. Exemplary haloalkyl groups include fluoromethyl, chloromethyl, fluoroethyl, chloroethyl, trilfluoromethyl, and the like. Unless otherwise specified, a haloalkyl group has from 1 to 10 carbon atoms.

A ‘cycloalkyl’ group refers to a cyclic alkyl group which can be mono or polycyclic. Exemplary cycloalkyl groups include cyclopropyl, cyclobutyl, cyclopentyl, cyclohexyl, cycloheptyl, cyclooctyl, cyclononyl, and cyclodecyl. Unless otherwise specified, a cycloalkyl group has from 3 to 10 carbon atoms.

‘Alkoxy’ refers to an —O(alkyl) group, where alkyl is as defined above. Therefore, unless otherwise specified, all isomers of a given structure are included within a definition. Exemplary alkyl groups include methoxy, ethoxy, n-propoxy, iso-propoxy, n-butoxy, iso-butoxy, t-butoxy, and the like. Unless otherwise specified, an alkoxy group has from 1 to 10 carbon atoms.

‘Haloalkoxy’ is an alkoxy group with a halo substituent, where alkoxy and halo groups are as defined above. Exemplary haloalkoxy groups include chloromethoxy, trichloroethoxy, trifloroethoxy, perfluoroethoxy (—OCF₂CF₃), trifluoro-t-butoxy, hexafluoro-t-butoxy, perfluoro-t-butoxy (—OC(CF₃)₃), and the like. Unless otherwise specified, an haloalkoxy group typically has from 1 to 10 carbon atoms.

‘Alkylthio’ refers to an —S(alkyl) goup, where alkyl group is as defined above. Exemplary alkyl groups include methylthio, ethylthio, propylthio, butylthio, iso-propylthio, iso-butylthio, and the like. Unless otherwise specified, an alkylthio group typically has from 1 to 10 carbon atoms.

‘Aryl’ is optionally substituted monocylic or polycyclic aromatic ring system of 6 to 14 carbon atoms. Exemplary groups include phenyl, naphthyl and the like. Unless otherwise specified, an aryl group typically has from 6 to 14 carbon atoms.

‘Heteroaryl’ is an aromatic monocyclic or polycyclic ring system of 4 to 10 carbon atoms, having at least one heteroatom or heterogroup selected from —O—, >N—, —S—, >NH or NR, and the like, wherein R is a substituted or unstubstituted alkyl, aryl, or acyl, as defined herein. In this aspect, >NH or NR are considered to be included when the heteroatom or heterogroup can be >N—. Exemplary heteroaryl groups include as pyrazinyl, isothiazolyl, oxazolyl, pyrazolyl, pyrrolyl, pyridazinyl, thienopyrimidyl, furanyl, indolyl, isoindolyl, benzo[1,3]dioxolyl, 1,3-benzoxathiole, quinazolinyl, pyridyl, thiophenyl and the like. Unless otherwise specified, a heteroaryl group typically has from 4 to 10 carbon atoms. Moreover, the heteroaryl group can be bonded to the heterocyclic core structure at a ring carbon atom, or, if applicable for a N-substituted heteroaryl such as pyrrole, can be bonded to the heterocyclic core structure through the heteroatom that is formally deprotonated to form a direct heteroatom-pyrimdine ring bond.

‘Heterocyclyl’ is a non-aromatic saturated monocyclic or polycyclic ring system of 3 to 10 member having at least one heteroatom or heterogroup selected from —O—, >N—, —S—, >NR, >SO₂, >CO, and the like, wherein R is hydrogen or a substituted or an unstubstituted alkyl, aryl, or acyl, as defined herein. Exemplary heterocyclyl groups include aziridinyl, pyrrolidinyl, piperdinyl, piperazinyl, morpholinyl, thiomorpholinyl, thiazolidinyl, 1,3-dioxolanyl, 1,4-dioxanyl and the like. Unless otherwise specified, a heterocyclyl group typically has from 2 to 10 carbon atoms. A heterocyclyl group can be bonded through a heteroatom that is formally deprotonated or a heterocyclyl group can be bonded through a carbon atom of the heterocyclyl group.

Further, the meaning of certain additional terms and phrases employed in the specification, can be defined as follows.

As used herein, the term “compound” includes both the singular and the plural, and includes any single entity or combined entities that have at least the affect disclosed herein and combinations, fragments, analogs or derivatives of such entities.

As used herein, the term “substance” refers broadly to any material of a particular kind or constitution. Examples of a “substance” can include, without limitation, a chemical element, a molecule, a compound, a mixture, a composition, an emulsion, a chemotherapeutic agent, a pharmacological agent, a hormone, an antibody, a growth factor, a cellular factor, a nucleic acid, a protein, a peptide, a peptidomimetic, a nucleotide, a carbohydrate, and combinations, fragments, analogs or derivatives of such entities.

The term “glycated protein,” as used herein, includes proteins linked to glucose, either enzymatically or non-enzymatically, primarily by condensation of free epsilon-amino groups in the protein with glucose, forming Amadori adducts. Furthermore, glycated protein, as used herein, includes not only proteins containing these initial glycation products, but also glycation products resulting from further reactions such as rearrangements, dehydration, and condensations that form irreversible advanced glycation end products (AGE).

The terms “treatment”, “treating”, “treat”, and the like are used herein to refer generally to any process, application, therapy, etc., wherein a mammal is subject to medical attention with the object of obtaining a desired pharmacological and/or physiological effect for improving the mammal's condition or disease, directly or indirectly. The effect can be therapeutic in terms of a partial or complete stabilization or cure for a disease and/or adverse effect attributable to the disease. The effect also can include, for example, inhibition of disease symptom (i.e., arresting its development) or relieving disease symptom (i.e., causing regression of the disease or symptom).

A used herein, the term “therapeutically-effective amount” refers to that amount of at least one compound as disclosed herein, or their pharmaceutically-acceptable salts thereof, that is sufficient to bring about the biological or medical effect that is being sought in a mammal, system, tissue, or cell.

The term “preventing”, “prevent”, “prevention”, and the like are used herein to refer generally to any process, application, therapy, etc., wherein a mammal is subject to medical attention with the object of obtaining a desired pharmacological and/or physiological effect for preventing onset of clinically evident condition or disease or preventing onset of a preclinically evident stage of a condition or disease. The effect can be prophylactic in terms of completely or partially preventing or reducing the risk of occurance of a condition or disease or symptom thereof.

A used herein, the term “prophylactically-effective amount” refers to that amount of a drug or pharmaceutical agent that will prevent or reduce the risk of occurrence of the biological or medical effect that is sought to be prevented in the cell, tissue, system, or mammal.

As used herein, the term “activation” refers to any alteration of a signaling pathway or biological response including, for example, increases above basal levels, restoration to basal levels from an inhibited state, and stimulation of the pathway above basal levels.

Publications and patents mentioned herein are disclosed for the purpose of describing, for example, the constructs and methodologies that are provided in the publications and patents, which might be used in connection with the present invention. Nothing herein is to be construed as an admission that the inventors are not entitled to antedate such publications, patents, or other disclosure by virtue of prior invention.

To the extent that any definition or usage provided by any document incorporated herein by reference conflicts with the definition or usage provided herein, the definition or usage provided herein controls.

For any particular compound disclosed herein, any general structure presented also encompasses all conformational isomers, regioisomers, stereoisomers and tautomers that can arise from a particular set of substituents. The general structure also emcompasses all enantiomers, diastereomers, and other optical isomers whether in enantiomeric or racemic forms, as well as mixtures of stereoisomers, as the context requires. The general structure also encompasses all salts, including pharmaceutically acceptable and non-pharmaceutically acceptable salts and prodrugs thereof.

When Applicants disclose or claim a range of any type, for example a range of temperatures, a range of numbers of atoms, a molar ratio, or the like, Applicants' intent is to disclose or claim individually each possible number that such a range could reasonably encompass, as well as any sub-ranges and combinations of sub-ranges encompassed therein. For example, when the Applicants disclose or claim a chemical moiety having a certain number of carbon atoms, Applicants' intent is to disclose or claim individually every possible number that such a range could encompass, consistent with the disclosure herein. For example, the disclosure that R is selected independently from an alkyl group having up to 20 carbon atoms, or in alternative language a C₁ to C₂₀ alkyl group, as used herein, refers to an R group that can be selected independently from a hydrocarbyl group having 1, 2, 3, 4, 5, 6, 7, 8, 9, 10, 11, 12, 13, 14, 15, 16, 17, 18, 19, or 20 carbon atoms, as well as any range between these two numbers for example a C₃ to C₈ alkyl group, and also including any combination of ranges between these two numbers for example a C₃ to C₅ and C₇ to C₁₀ hydrocarbyl group. In another example, by the disclosure that the molar ratio typically spans the range from about 0.1 to about 1.1, Applicants intend to recite that the molar ratio can be selected from about 0.1:1, about 0.2:1, about 0.3:1, about 0.4:1, about 0.5:1, about 0.6:1, about 0.7:1, about 0.8:1, about 0.9:1, about 1.0:1, or about 1.1:1.

Applicants reserve the right to proviso out or exclude any individual members of any such group, including any sub-ranges or combinations of sub-ranges within the group, that may be claimed according to a range or in any similar manner, if for any reason Applicants choose to claim less than the full measure of the disclosure, for example, to account for a reference that Applicants may be unaware of at the time of the filing of the application. Further, Applicants reserve the right to proviso out or exclude any individual substituents, compounds, ligands, structures, or groups thereof, or any members of a claimed group, if for any reason Applicants choose to claim less than the full measure of the disclosure, for example, to account for a reference that Applicants may be unaware of at the time of the filing of the application.

The following references disclose certain heterocyclic compounds.

TABLE 12
References disclosing heterocyclic compounds.

| Publication or | |
|---|---|
| Patent No. | Title |
| WO 2005/049617 | Pyrazolopyrimidines |
| WO 2005/049616 | 5,7-Diaminopyrazolo ′4,3-D!Pyrimidines with |
| | PDE-5 Inhibiting Activity |
| WO 2004/094810 | Anti-Detonation Fuel Delivery System |
| EP 1348707 | Pyrazolo[4,3-d]Pyrimidines, Processes for Their |
| | Preparation and Methods for Therapy |
| Journal of Medicinal | Synthesis and potential antipsychotic activity of |
| Chemistry | 1H-imidazole[1,2-c] pyrazole[3,e] pyrimidines |
| 1998, 31(2), 454-61. | |

Applicants reserve the right to proviso out or to restrict from any claim currently presented, or from any claim that may be presented in this or any further application based upon this disclosure, including claims drawn any genus or subgenus disclosed herein, any compound or group of compounds disclosed in any reference provided herein.

The following acronyms, abbreviations, terms and definitions have been used throughout the experimental section. Acronyms or abbreviations: NaH (sodium hydride), EtOAc (ethyl acetate), Na₂SO₄ (sodium sulphate), DSC (differential scanning calorimetry), N (Normal), M (molar), DMF (N,N-dimethylformamide), i-propanol or IPA (isopropyl alcohol or propan-2-ol ), HCl (hydrochloric acid), n-butanol, n-BuOH or BuOH (n-butyl alcohol or butan-1-ol), NaHCO₃ (sodium bicarbonate), POCl₃ (phosphorus oxychloride), NaOH (sodium hydroxide), H₂SO₄ (sulphuric acid), Pd/C (palladium carbon), Et₃N (triethylamine), SOCl₂ (thionyl chloride), DCC (N,N′-dicyclohexylcarbodiimide), DMAP (4-(N,N-dimethylaminopyridine), DMSO (dimethyl sulfoxide), t-BuOH (tert-butyl alcohol), t-BuOK (potassium tert-butoxide), THF (tetrahydrofuran), AlCl₃ (aluminum chloride), K₂CO₃ (potassium carbonate), n-BuLi (n-butyllithium), (PPh₃)₄Pd [tetrakis-(triphenylphosphine)palladium(0)], (PPh₃)₂PdCl₂ [bis-(triphenylphosphine)-palladium(II)chloride], HPLC (high performance liquid chromatography), TLC (thin layer chromatography), g (grams), mmol (millimoles), mL (milliliters), mp or MP (melting point), rt (room temperature), aq (aqueous), min (minutes), h, hr, or hrs (hours), atm (atmosphere), conc. (concentrated), MS, Mass Spec or Mass (mass spectroscopy/spectrometry), NMR (nuclear magnetic resonance), R_{f} (TLC retention factor), Rₜ (HPLC retention time), IR (infrared), and KBr (potassium bromide). NMR abbreviations: br (broad), apt (apparent), s (singlet), d (doublet), t (triplet), q (quartet), dq (doublet of quartets), dd (doublet of doublets), dt (doublet of triplets), m (multiplet), CDCl₃ (deuterated chloroform).

General Synthetic Procedures

Room temperature is defined as an ambient temperature range, typically from about 20° C. to about 35° C. An ice bath (crushed ice and water) temperature is defined as a range, typically from about −5° C. to about 0° C. Temperature at reflux is defined as about ±15° C. of the boiling point of the primary reaction solvent. Overnight is defined as a time range of from about 8 to about 16 hours. Vacuum filtration (water aspirator) is defined as occurring over a range of pressures, typically from about 5 mm Hg to about 15 mm Hg. Dried under vacuum is defined as using a high vacuum pump at a range of pressures, typically from about 0.1 mm Hg to about 5 mm Hg. Neutralization is defined as a typical acid-based neutralization method and measured to a pH range of from about pH 6 to about pH 8, using pH-indicating paper. Brine is defined as a saturated aqueous sodium chloride. Nitrogen atmosphere is defined as positive static pressure of nitrogen gas passed through a Drierite™ column with an oil bubbler system. Concentrated ammonium hydroxide is defined as an approximately 15 M solution. Melting points were measured against a mercury thermometer and are not corrected.

All eluents for column or thin layer chromatography were prepared and reported as volume:volume (v:v) solutions. The solvents, reagents, and the quantities of solvents and/or reagents used for reaction work-up or product isolation can be those that typically would be used by one of ordinary skill in organic chemical synthesis, as would be determined for the specific reaction or product to be isolated. For example: 1) crushed ice quantity typically ranged from about 10 g to about 1000 g depending on reaction scale; 2) silica gel quantity used in column chromatography depended on material quantity, complexity of mixture, and size of chromatography column employed and typically ranged from about 5 g to about 1000 g; 3) extraction solvent volume typically ranged from about 10 mL to about 500 mL, depending upon the reaction size; 4) washes employed in compound isolation ranged from about 10 mL to about 100 mL of solvent or aqueous reagent, depending on scale of reaction; and 5) drying reagents (potassium carbonate, sodium carbonate or magnesium sulfate) ranged from about 5 g to about 100 g depending on the amount of solvent to be dried and its water content.

Spectroscopic and other Instrumental Procedures

NMR. The ¹H spectra described herein were obtained using Varian Gemini 200 MHz spectrometers. Spectrometer field strength and NMR solvent used for a particular sample are indicated in the examples, or on any NMR spectra that are shown as Figures. Typically, ¹H NMR chemical shifts are reported as δ values in parts per million (ppm) downfield from tetramethylsilane (TMS) (δ=0 ppm) as an internal standard. Solid or liquid samples were dissolved in an appropriate NMR solvent (typically CDCl₃ or DMSO-d₆), placed in a NMR sample tube, and data were collected according to the spectrometer instructional manuals. Most samples were analyzed in Variable Temperature mode, typically at about 55° C., though some data for some samples were collected with the probe at ambient probe temperature. NMR data were processed using the software provided by Varian, VNMR 6.1 G version.

The present invention is further illustrated by the following examples, which are not to be construed in any way as imposing limitations upon the scope of this disclosure, but rather are intended to be illustrative only. On the contrary, it is to be clearly understood that resort may be had to various other embodiments, modifications, and equivalents thereof which, after reading the description herein, may suggest themselves to one of ordinary skill in the art without departing from the spirit of the present invention. Thus, the skilled artisan will appreciate how the experiments and Examples may be further implemented as disclosed by variously altering the following examples, substituents, reagents, or conditions. In the following examples, in the disclosure of any measurements, including temperatures, pressures, times, weights, percents, concentrations, ranges, chemical shifts, frequencies, molar ratio, and the like, it is to be understood that such measurements are respectively, “about.”

### EXAMPLES

### Example 1

### Preparation of (3-fluoro-4-methoxy-phenyl)-[5-(4-fluoro-phenyl)-1-methyl-3-propyl-1H-pyrazolo[4,3-d]pyrimidin-7-yl]-amine hydrochloride (E 1)

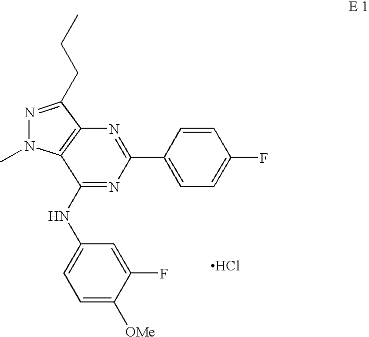

### Step 1: Preparation of 4-(4-fluoro-benzoylamino)-2-methyl-5-propyl-2H-pyrazole-3-carboxylic acid amide (3)

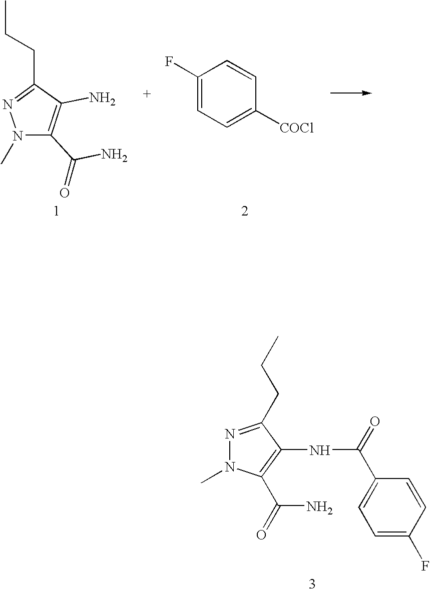

a) Preparation of 4-fluorobenzoyl chloride (2). To a solution of 4-fluorobenzoic acid (10 grams, 71.42 mmol) in dry ethyl acetate (EtOAc) (100 mL) was added thionyl chloride (SOCl₂) (84.9 grams, 714.2 mmol) slowly at 10° C. under nitrogen atmosphere. The mixture was then stirred at 85° C. for 12 hours. After completion of the reaction excess of SOCl₂ was removed by distillation under low vacuum to afford the desired compound 4-fluorobenzoyl chloride (10.8 grams, 95% yield). This was used directly for the next step without further purification.

b) Preparation of 4-(4-fluoro-benzoylamino)-2-methyl-5-propyl-2H-pyrazole-3-carboxylic acid amide (3). To a stirring solution of 4-amino-2-methyl-5-propyl-2H-pyrazole-3-carboxylic acid amide (1) (10 grams, 54.95 mmol) and triethyl amine (Et₃N) (6.94 grams, 68.68 mmol) in dichloromethane (100 mL) was added compound (2) 4-fluorobenzoyl chloride (8.7 grams, 54.94 mmol) slowly at 0° C. under nitrogen atmosphere. The mixture was stirred for 12-15 hours at room temperature. Dichloromethane was removed under vacuum and the mixture was diluted with cold water (about 50 mL) with stirring. White solid separated was filtered, washed with water (2×30 mL) and dried under vacuum to afford the desired product 4-(4-fluoro-benzoylamino)-2-methyl-5-propyl-2H-pyrazole-3-carboxylic acid amide (3) (9.6 grams). Yield: 60%.

¹H NMR (200 MHz, CDCl₃): δ 7.97-7.91 (m, 2H), 7.62(s, D₂O exchangeable, 1H), 7.21-7.17 (m, 2H), 4.01 (s, 3H), 2.53 (t,J=7.8 Hz, 2H), 1.70-1.60 (m, 4H), 0.93 (t,J=7.3 Hz, 3H).

Mass (CI method, I-butane): 305 (M+1, 100).

### Step 2: Preparation of 5-(4-fluoro-phenyl)-1-methyl-3-propyl-1,6-dihydro-pyrazolo[4,3-d]pyrimidin-7-one (4)

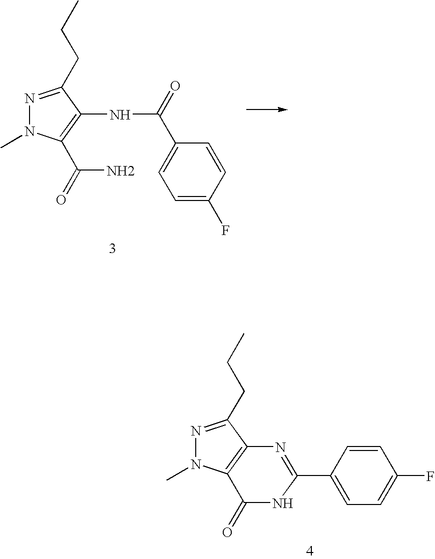

A mixture of 4-(4-fluoro-benzoylamino)-2-methyl-5-propyl-2H-pyrazole-3-carboxylic acid amide (3), obtained in step 1 (9 grams, 29.60 mmol) and potassium-t-butoxide (t-BuOK) (9.94 grams, 88.81 mmol) in t-butanol (t-BuOH) (90 mL) was stirred at 90° C. for 20-24 hours under nitrogen atmosphere. After completion of the reaction solvent was removed completely under vacuum. The residue was diluted with cold water (45 mL) and then acidified with 2N HCl until the pH ˜7. White solid separated was filtered, washed with cold water and dried under vacuum to afford the desired compound 5-(4-fluoro-phenyl)-1-methyl-3-propyl-1,6-dihydro-pyrazolo[4,3-d]pyrimidin-7-one (7 grams). Yield: 83%

¹H NMR (200 MHz, CDCl₃): δ 11.4 (s, D₂O exchangeable, 1H), 8.21-8.14 (m, 2H), 7.25-7.16 (m, 2H), 4.29(s, 3H), 2.93 (t,J=7.3 Hz, 2H), 1.92-1.87 (m, 2H), 1.03 (t,J=7.3 Hz, 3H).

Mass(CI method, i-butane): m/z 287 (M+1, 100).

### Step 3: Preparation of 7-chloro-5-(4-fluoro-phenyl)-1-methyl-3-propyl-1H-pyrazolo[4,3-d]pyrimidine (5)

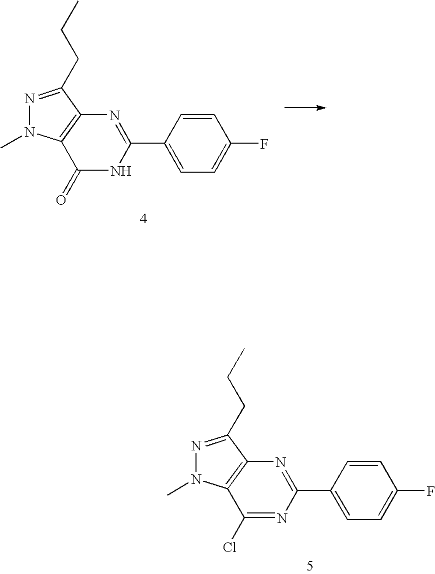

A mixture of compound 5-(4-fluoro-phenyl)-1-methyl-3-propyl-1,6-dihydro-pyrazolo[4,3-d]pyrimidin-7-one (4) obtained in step 2 (4 grams, 13.98 mmol) and phosphorusoxychloride (POCl₃) (40 mL) was stirred at 100° C. under anhydrous condition for 12-14 hours. After completion of the reaction the excess POCl₃ was removed by distillation under low vacuum. The residue was treated with toluene (30 mL) and then concentrated under vacuum. The residue was diluted with aqueous NaHCO₃ solution to reach the pH ˜7-8. The white solid that separated was filtered off, washed with water and dried under vacuum to afford the desired product 7-chloro-5-(4-fluoro-phenyl)-1-methyl-3-propyl-1H-pyrazolo[4,3 -d]pyrimidine (5) (3.52 grams). Yield: 83%.

¹H NMR (200 MHz, CDCl₃): δ 8.52-8.45 (m, 2H), 7.16 (t,J=8.6 Hz, 2H), 4.33 (s, 3H), 3.04 (t,J=7.5 Hz, 2H), 1.9-1.8 (m, 2H), 1.04 (t,J=7.3 Hz, 3H).

Mass(CI method): m/z 305 (M+1, 100).

IR (KBr, cm⁻¹): 3426, 2966, 1522.

### Step 4: Preparation of 3-fluoro-4-methoxyaniline (8)

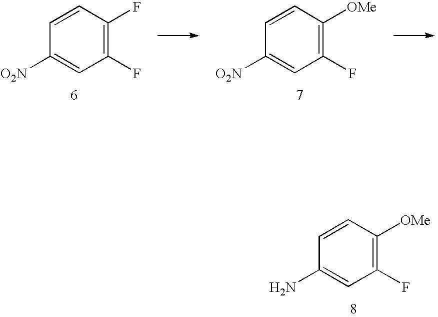

Metallic sodium (1.45 grams, 63 mmol) was added slowly and portion wise to pre cooled (10° C.) methanol (100 mL) with stirring under nitrogen atmosphere. The mixture was stirred at room temperature until all the sodium metal gets dissolved. To this was added 3,4-difluoro nitrobenzene (6) (10 grams, 63 mmol) at room temperature and stirring continued at the same temperature for 2-3 hours. The mixture was then concentrated under vacuum and poured into ice-water (100 mL). The pH of the mixture was adjusted to ˜7 by adding 2N HCl with stirring. The solid separated was filtered off, washed with water and dried under vacuum to afford the product 2-fluoro-4-nitroanisol (7) (9.75 grams). Yield: 91%; Melting point: 102-104° C.

To a mixture of 10% Pd/C (1.5 grams) in ethanol (150 mL) taken in a Parr™ hydrogenation flask was added a solution of 2-fluoro-4-nitroanisole (7) (9.09 grams, 53 mmol) in ethanol (150 mL) slowly. The mixture was then stirred under hydrogen atmosphere (40 psi) for 4 hours at room temperature. After completion of the reaction the mixture was filtered through Celite™ and the residue was washed thoroughly using ethanol (20 mL). The filtrates and washings were collected, combined and evaporated to dryness. The solid obtained was stirred in hexane (50 mL) for 1 hour and filtered to give the desired product 3-fluoro-4-methoxyaniline (8) (6.75 grams).

Yield: 91%; Melting point: 74-76° C.

### Step 5: Preparation of (3-fluoro-4-methoxy-phenyl)-[5-(4-fluoro-phenyl)-1-methyl-3-propyl-1H-pyrazolo[4,3-d]pyrimidin-7-yl]-amine hydrochloride (E 1)

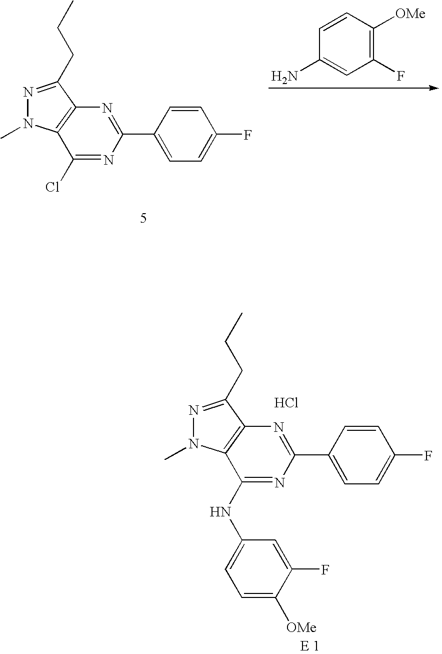

A mixture of compound 7-chloro-5-(4-fluoro-phenyl)-1-methyl-3-propyl-1H-pyrazolo[4,3-d]pyrimidine (5) obtained in step 3 (2.21 grams, 7.25 mmol), 3-fluoro-4-methoxyaniline (1.13 grams, 7.98 mmol) in i-propanol (30 mL) was stirred at 80° C. for 5-6 hours. The yellow solid separated was filtered and washed with i-propanol. The solid thus obtained was stirred in i-propanol at 50-60° C. for 3-4 hours, filtered and dried under vacuum to afford the desired product (3-fluoro-4-methoxy-phenyl)-[5-(4-fluoro-phenyl)-1-methyl-3-propyl-1H-pyrazolo[4,3-d]pyrimidin-7-yl]-amine hydrogen chloride (E 1) (2.78 grams). Yield: 94%.

DSC: 253.67° C.

¹H NMR (200 MHz, DMSO-d₆) δ 9.22 (s, D₂O exchangeable, 1H), 8.32-8.25 (m, 2H), 7.70 (d, J=13.6 Hz, 1H), 7.52 (d, J=8.9 Hz, 1H), 7.36-7.21 (m, 3H), 4.33 (s, 3H), 3.88 (s, 3H), 2.93 (t, J=7.4 Hz, 2H), 1.80 (q, J=7.4 Hz, 2H), 0.97 (t, J=7.3 Hz, 3H).

IR (KBr, cm⁻¹): 3423.9, 2924.9, 1631.1, 1567.9.

Mass (DIP CI method): m/z 410 (M+1, 100).

Alternatively the reaction was also carried out in dry dimethylformamide (DMF) (20 mL) at 80° C. for 5-6 hours. After completion the reaction mixture was poured into cold water (60 mL) and stirred for 10-15 minutes at room temperature. White solid separated was filtered, washed with water (20 mL) and dried under vacuum to afford the desired product E 1 (90% yield).

### Examples 2-52

Unless otherwise indicated, the following compounds presented in Examples 2-52 were prepared by a procedure analogous to that disclosed in Example 1, using analogous starting materials with the appropriate substitution, to afford the corresponding compounds, listed as compounds E 2 through E 52.

### Example 2

### Preparation of (3-chloro-4-methoxy-phenyl)-[5-(4-fluoro-phenyl)-1-methyl-3-propyl-1H-pyrazolo[4,3-d]pyrimidin-7-yl]-amine-hydrochloride (E 2)

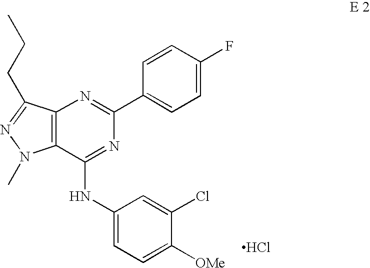

Yield: 88%; Melting point: 253.65° C.; ¹H NMR (200 MHz, DMSO-d₆) δ 9.12 (s, D₂O exchangeable, 1H), 8.33-8.25 (m, 2H), 7.92-7.91 (m, 1H), 7.75-7.70 (m, 1H), 7.35-7.23 (m, 3H), 4.33 (s, 3H), 3.90 (s, 3H), 2.92 (t, J=7.3 Hz, 2H), 1.82 (q, J=7.3 Hz, 2H), 0.97 (t, J=7.5 Hz, 3H); MS: 427 (M⁺−35, 100); IR(cm⁻¹): 3441, 2949, 1626.

### Example 3

### Preparation of (4-fluoro-phenyl)-[5-(4-fluoro-phenyl)-1-methyl-3-propyl-1H-pyrazolo[4,3-d]pyrimidin-7-yl]-amine-hydrochloride (E 3)

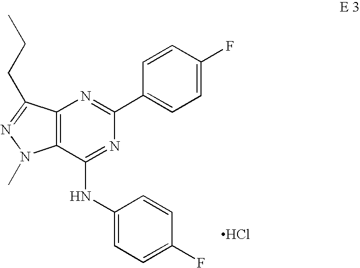

Yield: 81%; Melting point: 161-164° C.; ¹H NMR (400 MHz, DMSO-d₆): δ 8.44-8.36 (m, 2H), 7.70-7.63 (m, 2H), 7.20-7.08 (m, 4H), 6.83 (s, D₂O exchangeable, 1H), 4.33 (s, 3H), 3.01 (t, J=7.3 Hz, 2H), 1.99-1.88 (m, 2H), 1.06 (t, J=7.3 Hz, 3H); MS: 381 (M⁺−35, 100); IR (cm⁻¹): 3445, 2940.

### Example 4

### Preparation of (3,4-dimethoxy-phenyl)-[5-(4-fluoro-phenyl)-1-methyl-3-propyl-1H-pyrazolo[4,3-d]pyrimidin-7-yl]-amine-hydrochloride (E 4)

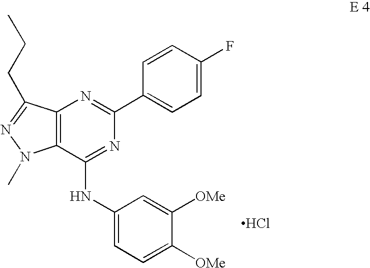

Yield: 82%; Melting point: 178-181° C.; ¹H NMR (400 MHz, DMSO-d₆): δ 8.34-8.23 (m, 2H), 7.97-7.92 (m, 1H), 7.29-7.21 (m, 2H), 6.74-6.64 (m, 2H), 4.30 (s, 3H), 3.83 (s, 6H), 2.88 (t, J=7.3 Hz, 2H), 1.89-1.78 (m, 2H), 0.98 (t, J=7.3 Hz, 3H); MS: 423 (M⁺−35, 100); IR (cm⁻¹): 3440, 1610.

### Example 5

### Preparation of 2-chloro-4-[5-(4-fluoro-phenyl)-1-methyl-3-propyl-1H-pyrazolo[4,3-d]pyrimidin-7-ylamino]-phenol-hydrochloride (E 5)

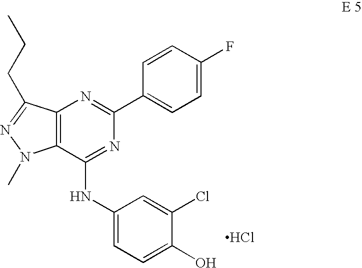

Yield: 66%; Melting point: 200-202° C.; ¹H NMR (200 MHz, DMSO-d₆): δ 10.09 (s, D₂O exchangeable, 1H), 8.79 (s, D₂O exchangeable, 1H), 8.29 (t, J=6.9 Hz, 2H), 7.78 (s, 1H), 7.53 (d, J=8.7 Hz, 1H), 7.27 (t, J=8.5 Hz, 2H), 7.04 (d, J=8.4 Hz, 1H), 4.29 (s, 3H), 2.89 (t, J=7.3 Hz, 2H), 1.85 (q, J=7.2 Hz, 2H), 0.97 (t, J=7.2 Hz, 3H); MS: 412 (M⁺−35, 100%); IR (cm⁻¹): 3451, 3177, 2925.

### Example 6

### Preparation of (4-chloro-3-methoxy-phenyl)-[5-(4-fluoro-phenyl)-1-methyl-3-propyl-1H-pyrazolo[4,3-d]pyrimidin-7-yl]-amine-hydrochloride (E 6)

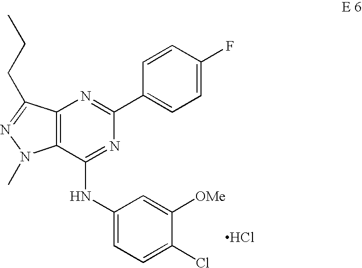

Yield: 67%; Melting point: 230-232° C.; ¹H NMR (200 MHz,DMSO-d₆): δ 9.04 (bs, D₂O exchangeable, 1H), 8.38-8.31 (m, 2H), 7.74 (s, 1H), 7.48-7.26 (m, 4H), 4.32 (s, 3H), 3.88 (s, 3H), 2.91 (t, J=7.3 Hz, 2H), 1.82 (q, J=7.3 Hz, 2H), 0.97 (t, J=7.3 Hz, 3H); MS: 426 (M⁺−35, 100%); IR (cm⁻¹): 3430, 2926.

### Example 7

### Preparation of 2-fluoro-4-[5-(4-fluoro-phenyl)-1-methyl-3-propyl-1H-pyrazolo[4,3-d]pyrimidin-7-ylamino]-phenol-hydrochloride (E 7)

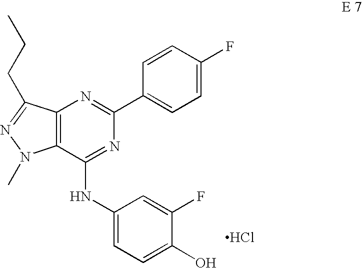

Yield: 43%; Melting point: 161-162° C.; ¹H NMR (200 MHz, DMSO-d₆): δ 9.19 (s, D₂O exchangeable, 1H), 8.30-8.23 (m, 2H), 7.62 (d, J=2.2 Hz, 1H), 7.56-7.28 (m, 3H), 7.04 (t, J=9.3 Hz, 1H), 4.32 (s, 3H), 2.92 (t, J=7.4 Hz, 2H), 1.81 (q, J=7.4 Hz, 2H), 0.97 (q, J=7.3 Hz, 3H); MS: 396 (M⁺−35, 100); IR (cm⁻¹): 3413, 2965.

### Example 8

### Preparation of benzo[1,3]dioxol-5-yl-[5-(4-fluoro-phenyl)-1-methyl-3-propyl-1H-pyrazolo[4,3-d]pyrimidin-7-yl]-amine-hydrochloride (E 8)

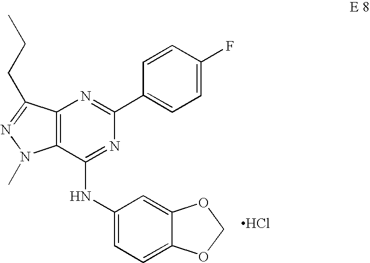

Yield: 53%; Melting point: <260° C.; ¹H NMR (200 MHz, DMSO-d₆): δ 9.20 (s, D₂O exchangeable, 1H), 8.24-8.20 (m, 2H), 7.35-7.32 (m, 3H), 7.17 (d, J=8.2 Hz, 1H), 7.0 (d, J=8.4 Hz, 1H), 6.06 (s, 2H), 4.32 (s, 3H), 2.92 (t, J=7.4 Hz, 2H), 1.81 (q, J=7.4 Hz, 2H), 0.97 (q, J=7.3 Hz, 3H); MS: 406 (M⁺−35, 100); IR (cm⁻¹): 1567, 1243.

### Example 9

### Preparation of (3-chloro-4-methoxy-phenyl)-(1-methyl-3-propyl-5-thiophen-2-yl-1H-pyrazolo[4,3-d]pyrimidin-7-yl)-amine-hydrochloride (E 9)

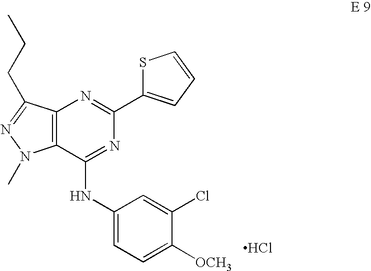

Yield: 79%; Melting point: 258-260° C.; ¹H NMR (200 MHz, DMSO-d₆): δ 9.09 (s, D₂O exchangeable, 1H) 8.03-7.67 (m, 4H), 7.22-7.15 (m, 2H), 4.32 (s, 3H), 3.89 (s, 3H), 2.90 (t, J=7.3 Hz, 2H), 1.80 (q, J=7.3 Hz, 2H), 0.97 (t, J=7.3 Hz, 3H); MS: 414 (M⁺−35, 100%); IR (cm⁻¹): 1652, 1505.

### Example 10

### Preparation of (3-fluoro-4-methoxy-phenyl)-(1-methyl-3-propyl-5-thiophen-2-yl-1H-pyrazolo[4,3-d]pyrimidin-7-yl) -amine hydrochloride (E 10)

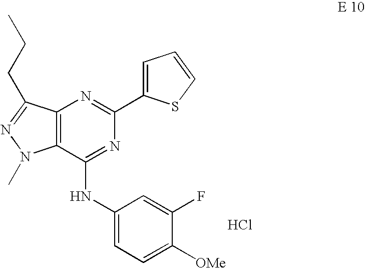

Yield: 52%; Melting point: 238-242° C.; ¹H NMR (200 MHz, DMSO-d₆): δ 9.15 (s, D₂O exchangeable, 1H), 7.92-7.53 (m, 4H), 7.29-7.16 (m, 2H), 4.32 (s, 3H), 3.88 (s, 3H), 2.90 (t, J=7.3 Hz, 2H), 1.80 (q, J=7.3 Hz, 2H), 0.97 (t, J=7.3 Hz, 3H); MS: 398 (M⁺−35, 100); IR (cm⁻¹): 1652, 1505.

### Example 11

### Preparation of (4-chloro-3-methoxy-phenyl)-(1-methyl-3-propyl-5-thiophen-2-yl-1H-pyrazolo[4,3-d]pyrimidin-7-yl)-amine hydrochloride (E 11)

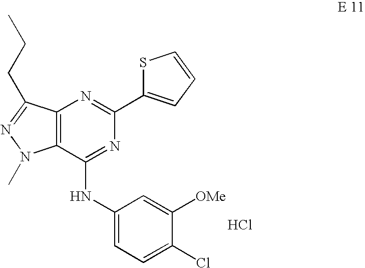

Yield: 60%; Melting point: 200-204° C.; ¹H NMR (200 MHz, CDCl₃): δ 9.19 (s, D₂O exchangeable, 1H) 7.90 (d, J=3.4 Hz, 1H), 7.76-7.64 (m,2H), 7.49-7.39 (m,2H), 7.16 (t, J=3.6 Hz, 1H), 4.33 (s, 3H), 3.92 (s, 3H), 2.90 (t, J=7.6 Hz, 2H), 1.87-1.76 (m, 2H), 0.97 (t, J=7.6 Hz, 3H); MS: 414 (M⁺−35, 100); IR (cm⁻¹): 1627, 1560.

### Example 12

### Preparation of benzo[1,3]dioxol-5-yl-(1-methyl-3-propyl-5-thiophen-2-yl-1H-pyrazolo[4,3-d]pyrimidin-7-yl)-amine hydrochloride (E 12)

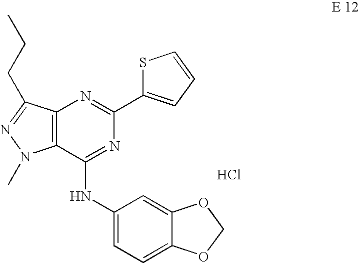

Yield: 48%; Melting point: 246-250° C.; ¹H NMR (200 MHz, CDCl₃): δ 9.12 (s, D₂O exchangeable, 1H) 7.93 (s, 1H), 7.69 (d, J=4.8 Hz, 1H), 7.5 (s, 1H), 7.18 (d, J=5.1 Hz, 2H), 7.0 (d, J=8.5 Hz, 1H), 6.07 (s, 2H), 3.87 (s, 3H), 2.90 (t, J=7.3 Hz, 2H), 1.85-1.74 (m, 2H), 0.97 (t, J=7.3 Hz, 3H); MS: 394 (M⁺−35, 100); IR (cm⁻¹): 1570, 1483.

### Example 13

### Preparation of (3-chloro-4-methoxy-phenyl)-(1,3-dimethyl-5-phenyl-1H-pyrazolo[4,3-d]pyrimidin-7-yl)-amine hydrochloride (E 13)

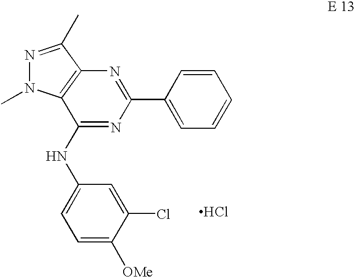

Yield: 69%; Melting point: 234-236° C.; ¹H NMR (200 MHz, DMSO-d₆): δ 9.03 (sb, 1H), 8.28 (d, J=3.9 Hz, 2H), 7.9 (s, 1H), 7.74 (d, J=8.7 Hz, 1H), 7.48-7.46 (m, 3H), 7.25 (d, J=3.6 Hz, 1H), 4.31 (s, 3H), 3.90 (s, 3H), 2.50 (s, 3H).

### Example 14

### Preparation of (1,3-dimethyl-5-phenyl-1H-pyrazolo[4,3-d]pyrimidin-7-yl)-(3-fluoro-4-methoxy-phenyl)-amine hydrochloride (E 14)

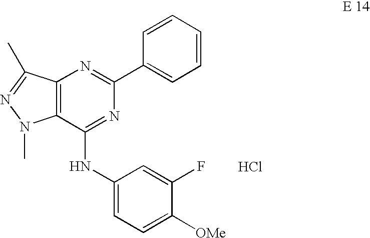

Yield: 72%; ¹H NMR (200 MHz, DMSO-d₆) δ 9.15 (bs, 1H), 8.27-8.25 (m, 2H), 7.78-7.48 (m, 5H), 7.59 (d, J=8.4 Hz, 1H), 4.32 (s, 3H), 3.88 (s, 3H), 2.51 (s, 3H); MS: 364 (M⁺−35, 100); IR (cm⁻¹): 3438.

### Example 15

Preparation of (4-chloro-3-methoxy-phenyl)-(1,3-dimethyl-5-phenyl-1H-pyrazolo[4,3-d]pyrimidin-7-yl)-amine hydrochloride (E 15)

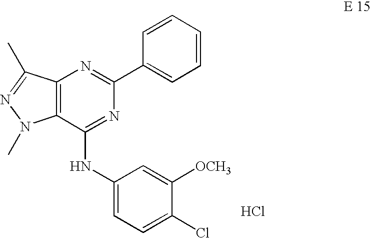

Yield: 63%; Melting point: 222-224° C.; ¹H NMR (200 MHz, DMSO-d₆): δ 9.11 (bs, 1H), 8.33 (d, J=5.0 Hz, 2H), 7.79 (s, 1H), 7.50-7.42 (m, 5H), 4.32 (s, 3H), 3.90 (s, 3H), 2.51 (s, 3H); MS: 380 (M⁺−35, 100); IR (cm⁻¹): 3426.

### Example 16

### Preparation of (3-fluoro-4-methoxy-phenyl)-[5-(4-fluoro-phenyl)-1,3-dimethyl-1H-pyrazolo[4,3-d]pyrimidin-7-yl]-amine hydrochloride (E 16)

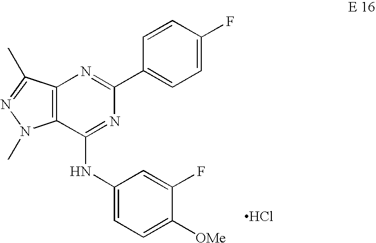

Melting point: 262° C.; ¹H NMR (200 MHz, DMSO-d₆): δ 9.10 (s, b, 1H), 8.30 (t, J=7.2 Hz, 2H), 7.52 (d, J=8.9 Hz, 1H), 7.35 -7.21 (m, 4H), 4.31 (s, 3H), 3.90 (s, 3H), 2.50 (s, 3H); MS: 382 (M⁺−35, 100); IR (cm⁻¹): 3441, 1296.

### Example 17

### Preparation of (3-chloro-4-methoxy-phenyl)-[5-(4-fluoro-phenyl)-1,3-dimethyl-1H-pyrazolo[4,3-d]pyrimidin-7-yl]-amine hydrochloride (E 17)

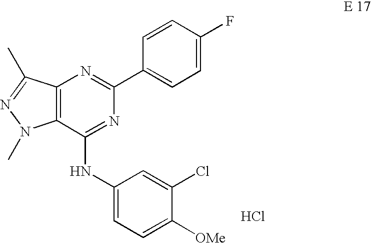

Yield: 70%; Melting point: >240° C.; ¹H NMR (200 MHz, DMSO-d₆): δ 9.06 (bs, 1H), 8.31 (t, J=6.9 Hz, 2H), 7.92 (s, 1H), 7.72 (d, J=6.9 Hz, 1H), 7.34-7.23 (m, 3H), 4.31(s, 3H), 3.90 (s, 3H), 2.50 (s, 3H); MS: 398 (M⁺−35, 100); IR (cm⁻¹): 3438, 1262.

### Example 18

### Preparation of 2-chloro-4-[5-(4-fluoro-phenyl)-1,3-dimethyl-1H-pyrazolo[4,3-d]pyrimidin-7-ylamino]-phenol hydrochloride (E 18)

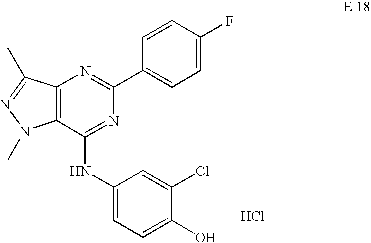

Yield: 45%; Melting point: 238-240° C.; ¹H NMR (200 MHz, DMSO-d₆): δ 9.13 (bs, 1H), 8.32-8.24 (m, 2H), 7.78 (s, 1H), 7.55-7.49 (dd, J₁=2.5, J₂=2.2 Hz, 1H), 7.31 (t, J=8.8 Hz, 2H), 7.06 (d, J=8.7 Hz, 1H), 4.69 (bs, 1H), 4.31 (s, 3H), 2.50 (s, 3H); MS: 384 (M⁺−35, 100); IR (cm⁻¹): 3381, 3194.

### Example 19

### Preparation of benzo[1,3]dioxol-5-yl-[5-(4-fluoro-phenyl)-1,3-dimethyl-1H-pyrazolo[4,3-d]pyrimidin-7-yl]-amine hydrochloride (E 19)

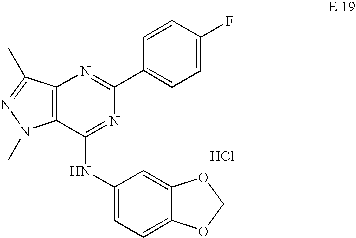

Yield: 62%; Melting point: 230-232° C.; ¹H NMR (200 MHz, DMSO-d₆): δ 9.12 (bs, 1H), 8.30 (d, J=5.9 Hz, 2H), 7.38-7.28 (m, 3H), 7.18 (d, J=8.1 Hz, 1H), 7.00 (d, J=8.5 Hz, 1H), 6.07 (s, 2H), 4.30 (s, 3H), 2.50 (s, 3H); MS: 378 (M⁺−35, 100); IR (cm⁻¹): 3439.

### Example 20

### Preparation of 1-[5-(3,4-dimethoxy-phenyl)-1-methyl-3-propyl-1H-pyrazolo[4,3-d]pyrimidin-7-yl]-piperidin-4-ol (E 20)

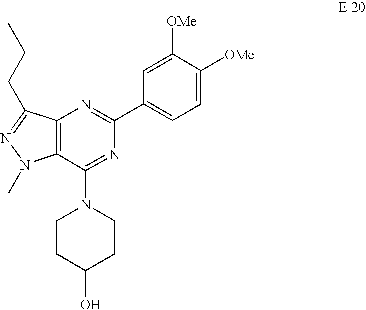

This compound was prepared by using 2-butanol (10 mL) instead of i-propanol at 120° C. for 24 hours, by a procedure analogous to that disclosed in Example 1.

Yield: 24%; Melting point: 142-144° C.; ¹H NMR (400 MHz, CDCl₃) δ 8.10-8.08 (m, 2H), 6.96-6.94 (m, 1H), 4.10 (s, 3H), 4.04 (s, 3H), 4.03 (s, 3H), 4.00-3.9 (m, 1H), 3.48-3.36 (m, 1H), 3.34-3.30 (m, 2H), 3.30 (m, 4H), 3.01 (t, J=7.6 Hz, 2H), 2.14-2.10 (m, 2H), 1.89-1.80 (m, 1H), 1.05 (t, J=7.6 Hz, 3H); MS: 412 (M+1, 100%); IR (cm⁻¹): 3418, 2925, 1547.

### Example 21

### Preparation of (3-chloro-4-methoxy-phenyl)-[5-(3,4-dimethoxy-phenyl)-1-methyl-3-propyl-1H-pyrazolo[4,3-d]pyrimidin-7-yl]-amine hydrochloride (E 21)

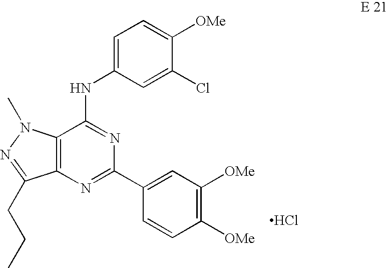

Melting point: 198-201° C.; ¹H NMR (400 MHz, DMSO-d₆) δ 9.29 (bs, D₂O exchangeable, 1H), 7.93-7.82 (m, 3H), 7.72-7.67 (m, 1H), 7.26 (d, J=8.9 Hz, 1H), 7.08 (d, J=8.6 Hz, 1H), 4.34 (s, 3H), 3.89 (s, 3H), 3.83 (s, 3H), 3.81 (s,3H), 2.95 (t, J=7.3 Hz, 2H), 1.87-1.76 (m, 2H), 0.98 (t, J=7.3 Hz, 3H).

### Example 22

### Preparation of 3-[7-(3-chloro-4-methoxy-phenylamino)-1-methyl-3-propyl-1H-pyrazolo[4,3-d]pyrimidin-5-yl]-4-ethoxy-benzenesulfonamide hydrochloride (E 22)

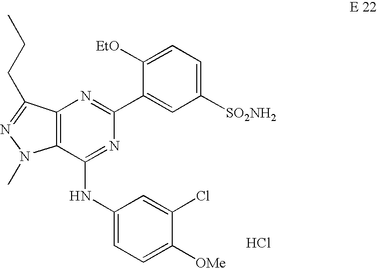

This compound was prepared at 90° C. for 7 hours, by a procedure analogous to that disclosed in Example 1.

Yield: 50%; Melting point: 204-206° C.; ¹H NMR (200 MHz, DMSO-d₆): δ 9.66 (s, D₂O exc. 1H), 7.99 (d, J=7.5 Hz, 2H), 7.72 (d, J=8.7 Hz, 1H), 7.36 (d, J=8.7 Hz, 2H), 7.18 (d, J=8.9 Hz, 1H), 4.39 (s, 3H), 4.22 -4.19 (m, 2H), 3.86 (s, 3H), 2.90 (t, J=7.3 Hz, 2H), 1.78 (q, J=7.3 Hz, 2H), 1.28 (t, J=6.6 Hz, 3H), 0.96 (t, J=7.3 Hz, 3H).

### Example 23

### Preparation of 4-ethoxy-3-[7-(3-fluoro-4-methoxy-phenylamino)-1-methyl-3-propyl-1H-pyrazolo[4,3-d]pyrimidin-5-yl]-benzenesulfonamide hydrochloride (E 23)

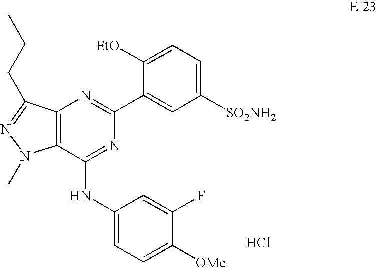

This compound was prepared at 90° C. for 7 hrs, by a procedure analogous to that disclosed in Example 1.

Yield: 79%; Melting point: 218-220° C.; ¹H NMR (200 MHz,DMSO): δ 9.83 (s, D₂O exchangable, 1H), 8.09-7.76 (m,3H), 7.54-7.15 (m, 3H), 4.39 (s, 3H), 4.26-4.19 (m, 2H), 3.84 (s, 3H), 2.91 (t, J=7 Hz, 2H), 1.81-1.74 (m, 2H), 1.29 (t, J=6.7 Hz, 3H), 0.95 (t, J=7 Hz, 3H).

### Example 24

### Preparation of (3-chloro-4-methoxy-phenyl)-[5-(2-ethoxy-phenyl)-1-methyl-3-propyl-1H-pyrazolo[4,3-d]pyrimidin-7-yl]-amine hydrochloride (E 24)

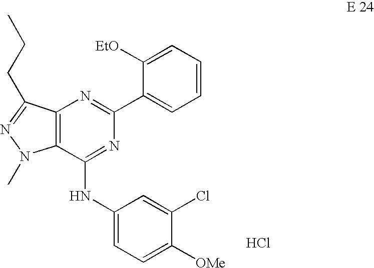

This compound was prepared at 90° C. for 7 hrs, by a procedure analogous to that disclosed in Example 1.

Yield: 62%; Melting point: 202-204° C.; ¹H NMR (200 MHz,CDCL₃): δ 13.6 (s, D₂O exc 1H), 11.8 (s, D₂O exc., 1H), 7.99 (s, 3H), 7.54-7.50 (m, 1H), 7.11-6.87 (m, 3H), 4.64 (s, 3H), 4.38-4.35 (m, 2H), 3.93 (s, 3H), 2.92 (t, J=7.3 Hz, 2H), 1.79-1.59 (m, 5H), 1.01 (t, J=7.3 Hz, 3H).

### Example 25

### Preparation of [5-(2-ethoxy-phenyl)-1-methyl-3-propyl-1H-pyrazolo[4,3-d]pyrimidin-7-yl]-(3-fluoro-4-methoxy-phenyl)-amine hydrochloride (E 25)

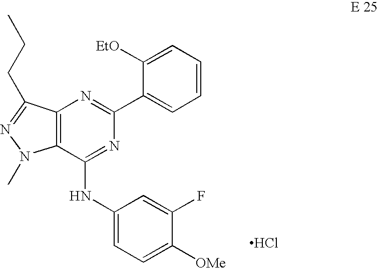

Melting point: 194-196° C.; ¹H NMR (200 MHz, CDCl₃) δ 14.01 (s, D₂O exchangeable, 1H), 11.59 (s, D₂O exchangeable, 1H), 7.85-7.71 (m, 3H), 7.52 (t, J=7.4 Hz, 1H), 7.08 (d, J=8.4 Hz, 1H), 6.98-6.82 (m, 2H), 4.56 (s, 3H), 4.36-4.33 (m, 2H), 3.91 (s, 3H), 2.94 (t, J=7.6 Hz, 2H), 2.17-1.55 (m, 5H), 1.0 (t, J=7.2 Hz, 3H); MS: 436 (M⁺−35, 100%).

IR (cm⁻¹): 3424, 2927, 1591.

### Example 26

### Preparation of 2-chloro-4-(1-methyl-5-phenyl-3-propyl-1H-pyrazolo[4,3-d]pyrimidin-7-ylamino)-phenol hydrochloride (E 26)

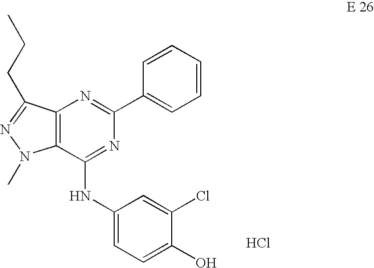

Yield: 51%; Melting point: 180° C.; ¹H NMR (200 MHz, DMSO-d₆): δ 10.07 (bs, D₂O exchangeable, 1H), 8.74 (s, D₂O exchangeable, 1H), 8.30-8.29 (m, 2H), 7.84 (d, J=2.2 Hz, 1H), 7.56-7.42 (m, 4H), 7.03 (d, J=8.8 Hz, 1H) 4.29 (s, 3H), 2.88 (t, J=7.1, 2H), 1.83 (q, J=7.3 Hz, 2H), 0.97 (t, J=7.3 Hz, 3H); MS: 394 (M+1, 100); IR (cm⁻¹):3442, 1609.

### Example 27

### Preparation of (3-chloro-4-methoxy-phenyl)-(1-methyl-5-phenyl-3-propyl-1H-pyrazolo[4,3-d]pyrimidin-7-yl)-amine hydrochloride (E 27)

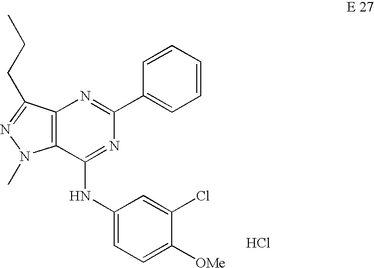

Yield: 96%; Melting point: 238-240° C.; ¹H NMR (400 MHz, DMSO-d₆): δ 9.20 (s, 1H, D₂O exchangeable), 8.23 (dd, J₁=7.2 Hz, J₂=2.4 Hz, 2H), 7.95 (d, J=2.8 Hz, 1H), 7.71 (dd, J₁=8.8 Hz, J₂=2.8 Hz, 1H), 7.47-7.46 (m, 3H), 7.24 (d, J=8.8 Hz, 1H), 4.32 (s, 3H), 3.89 (s, 3H), 2.92 (t, J=7.3 Hz, 2H), 1.80 (m, 2H), 0.96 (t, J=7.3 Hz, 3H); MS: 408 (M⁺−36, 100%); IR (cm⁻¹): 3439, 1626, 1562.

### Example 28

### Preparation of (3-fluoro-4-methoxy-phenyl)-(1-methyl-5-phenyl-3-propyl-1H-pyrazolo[4,3-d]pyrimidin-7-yl)-amine hydrochloride (E 28)

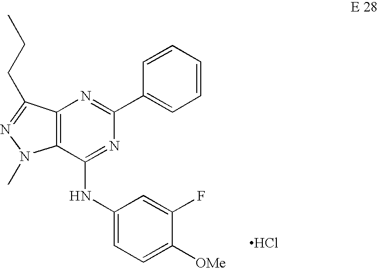

Yield: 74%; Melting point: 248-250° C.; ¹H NMR (200 MHz, DMSO-d₆) δ 9.18 (s, D₂O exchangeable, 1H), 8.24 (d, J=3.9 Hz, 2H), 7.48 (m, 5H), 7.26 (t, J=9.26 Hz, 1H), 3.80 (s, 3H), 1.80 (q, J=7.3 Hz, 2H), 0.98 (t, 7.3 Hz, 3H); MS: 392 (M+1, 100%); IR (cm⁻¹): 3429, 2925, 1629.

### Example 29

### Preparation of (4-chloro-3-methoxy-phenyl)-(1-methyl-5-phenyl-3-propyl-1H-pyrazolo[4,3-d]pyrimidin-7-yl)-amine hydrochloride (E 29)

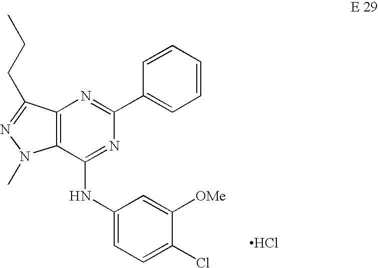

Yield: 73%; Melting point: 228-230° C.; ¹H NMR (200 MHz, DMSO-d₆): δ 9.23 (bs, D₂O exchangeable, 1H), 8.32-8.29 (m, 2H), 7.79 (s, 1H), 7.50-7.37 (m, 5H), 4.34 (s, 3H), 3.89 (s, 3H), 2.94 (t, J=7.3 Hz, 2H), 1.82 (q, J=7.3 Hz, 2H), 0.98 (t, J=7.3 Hz, 3H);

MS: 408 (M⁺, 100%); IR (cm⁻¹): 3423, 2923.

### Example 30

### Preparation of (1,3-dimethyl-5-thiophen-2-yl-1H-pyrazolo[4,3-d]pyrimidin-7-yl)-(3-fluoro-4-methoxy-phenyl)-amine hydrochloride (E 30)

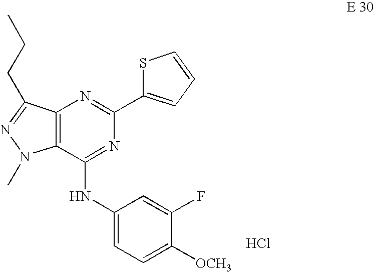

This compound was prepared at 90° C. for 4-5 hrs, by a procedure analogous to that disclosed in Example 1.

Yield: 60%; Melting point: 236-238° C.; ¹H NMR (200 MHz, DMSO-d₆): δ 9.07 (bs, 1H), 7.88-7.68 (m, 2H), 7.59 (d, J=8.4 Hz, 1H), 7.30 (d, J=9.2 Hz, 2H), 7.20 (t, J=4.7 Hz, 1H), 4.33 (s, 3H), 3.91 (s, 3H), 2.53 (s, 3H); Mass (CI method, i-butane): 370 (M⁺, 100).

### Example 31

### Preparation of (4-chloro-3-methoxy-phenyl)-(1,3-dimethyl-5-thiophen-2-yl-1H-pyrazolo[4,3-d]pyrimidin-7-yl)-amine hydrochloride (E 31)

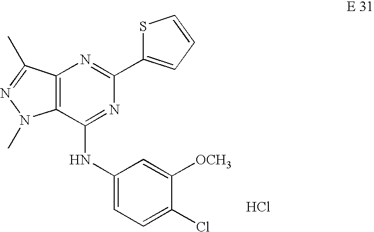

This compound was prepared at 90° C. for 4-5 hrs, by a procedure analogous to that disclosed in Example 1.

Yield: 63%; Melting point: >240° C.; ¹H NMR (200 MHz,DMSO-d₆): δ 9.20 (bs, 1H), 7.90 (d, J=2.6 Hz, 1H), 7.67 (t, J=7.3 Hz, 2H), 7.49-7.39 (m, 2H), 7.17 (t, J=4.8 Hz, 1H), 4.31 (s, 3H), 3.9 (s, 3H), 2.49 (s, 3H); MS: 386 (M⁺−35, 100); IR (cm⁻¹): 3418.

### Example 32

### Preparation of (3-chloro-4-methoxy-phenyl)-(1,3-dimethyl-5-thiophen-2-yl-1H-pyrazolo[4,3-d]pyrimidin-7-yl)-amine hydrochloride (E 32)

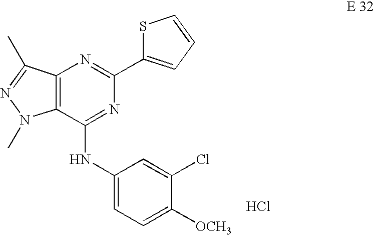

This compound was prepared at 90° C. for 4-5 hrs, by a procedure analogous to that disclosed in Example 1.

Yield: 69%; Melting point: >240° C.; ¹H NMR (200 MHz, DMSO-d₆): δ 9.20 (bs, 1H), 7.90 (d, J=2.6 Hz, 1H), 7.71-7.65 (m, 2H), 7.49-7.39 (m, 2H), 7.17 (t, J=3.9 Hz, 1H), 4.31 (s, 3H), 3.91(s, 3H), 2.49 (s, 3H); MS: 386 (M⁺−35, 100); IR (cm⁻¹): 3426.

### Example 33

Preparation of benzo[1,3]dioxol-5-yl-(1,3-dimethyl-5-thiophen-2-yl-1H-pyrazolo[4,3-d]pyrimidin-7-yl)-amine hydrochloride (E 33)

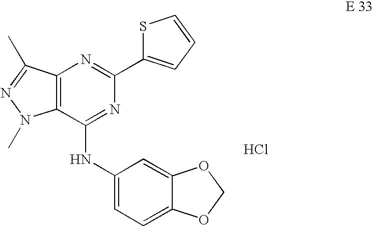

This compound was prepared at 90° C. for 4-5 hrs, by a procedure analogous to that disclosed in Example 1.

Yield: 60%; Melting: >240° C.; ¹H NMR (200 MHz, DMSO-d₆) : δ 8.99 (bs, 1H), 7.83-7.65 (m, 3H), 7.51-7.17 (m, 2H), 6.99 (d, J=8.4,1H), 6.07 (s, 2H), 4.28 (s, 3H), 2.46 (s, 3H); MS: 366 (M⁺−35, 100); IR (cm⁻¹): 3441.

### Example 34

### Preparation of 2-chloro-4-(1,3-dimethyl-5-thiophen-2-yl-]H-pyrazolo[4,3-d]pyrimidin-7-ylamino)-phenol hydrochloride (E 34)

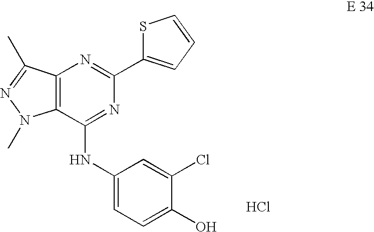

This compound was prepared at 90° C. for 4-5 hrs, by a procedure analogous to that disclosed in Example 1.

Yield: 72%; Melting point: >240° C.; ¹H NMR (200 MHz, DMSO-d₆): δ 9.11 (bs, 1H), 7.91-7.88 (m, 1H), 7.70 (d, J=4.2 Hz, 2H), 7.53 (dd, J₁=2.2, J₂=2.2 Hz, 1H), 7.17 (t, J=4.5 Hz, 1H), 7.05 (d, J=8.7 Hz, 1H), 4.70 (bs, 1H), 4.30 (s, 3H) 2.48 (s, 3H); MS: 372 (M⁺−35, 100); IR (cm⁻¹): 3392, 3077.

### Example 35

### Preparation of (1,3-dimethyl-5-phenyl-1H-pyrazolo[4,3-d]pyrimidin-7-yl)-(3-fluoro-phenyl)-amine hydrochloride (E 35)

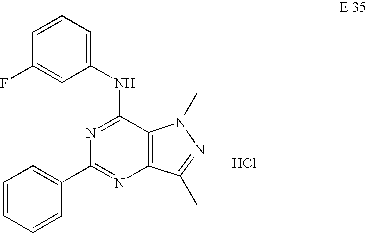

This compound was prepared at 80° C. for 12 hrs, by a procedure analogous to that disclosed in Example 1.

Yield: 58%; MP: 218-220° C.; Purity: 98.50%; ¹H NMR (400 MHz, CDCl₃): δ 8.44 (d, J=7.0, 2H), 7.79 (d, J=11.0, 1H), 7.52-7.32 (m, 6H), 4.29 (s, 3H), 2.61 (s, 3H); MS 334 (M⁺+1, 100%); IR (cm-¹): 3453.7.

### Example 36

### Preparation of [5-(4-fluoro-phenyl)-1-methyl-3-propyl-1H-pyrazolo[4,3-d]pyrimidin-7-yl]-(3-trifluoromethyl-phenyl)-amine hydrochloride (E 36)

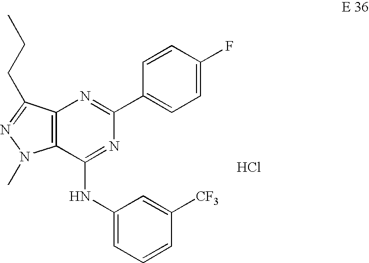

This compound was prepared at 80° C. for 12 hrs, by a procedure analogous to that disclosed in Example 1.

Yield: 74%; MP: 220-222° C.; Purity: 97%; ¹H NMR (400 MHz, DMSO-d₆): δ 9.25 (bs 1H), 8.27-8.24 (m, 2H), 8.04-8.02 (m,2H), 7.67-7.21 (m, 4H), 4.33 (s 3H), 2.82-2.80 (t, J=7.30 Hz 2H), 1.85-1.82 (m, 2H), 0.98-0.95 (t J=7.30 Hz 3H); MS: 430 (M⁺+1, 100%);

IR (cm-¹): 3434, 1587, 1125.

### Example 37

### Preparation of [5-(4-fluoro-phenyl)-1-methyl-3-propyl-1H-pyrazolo[4,3-d]pyrimidin-7-yl]-(4-trifluoromethoxy-phenyl)-amine hydrochloride (E 37)

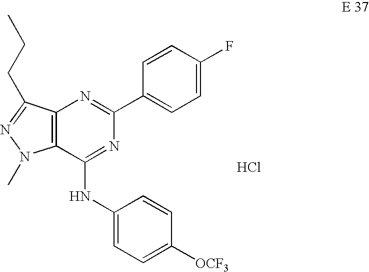

This compound was prepared at 80° C. for 12 hrs, by a procedure analogous to that disclosed in Example 1.

Yield: 76%; MP: 230-232° C.; Purity: 96%; ¹H NMR (400 MHz, DMSO-d₆): δ 9.25 (bs, 1H), 8.32-8.28 (m, 2H), 7.93-7.89 (m, 2H), 7.45 (d, J=8.33, 2H), 7.35-7.27 (m, 2H), 4.33 (s, 3H), 2.94-2.90 (t, J=7.30, 2H), 1.88-1.79 (m, 2H), 1.78-1.74(t. J=7.30, 3H); MS: 446 (M⁺+1, 100%); IR (cm-¹): 3424, 1629, 1506, 1258.

### Example 38

### Preparation of (1,3-dimethyl-5-phenyl-1H-pyrazolo[4,3-d]pyrimidin-7-yl)-(4-trifluoromethoxy-phenyl)-amine hydrochloride (E 38)

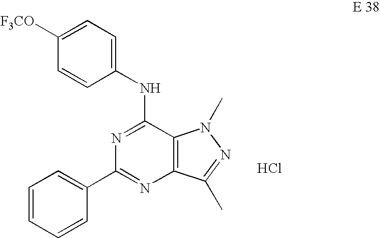

This compound was prepared at 80° C. for 12 hrs, by a procedure analogous to that disclosed in Example 1.

Yield: 39%; MP: 174-176° C.; Purity: 97.64%; ¹H NMR (400 MHz, CDCl₃): δ 8.40 (d, J=5.9, 2H), 7.89 (d, J=8.6, 2H), 7.54-7.41(m, 3H), 7.30-7.28 (m, 2H), 6.93 (bs, —NH), 4.33 (s, 3H), 2.63 (s, 3H); MS: 400 (M⁺+1, 100%); IR (cm-¹): 3459.5.

### Example 39

### Preparation of [5-(4-fluoro-phenyl)-1,3-dimethyl-1H-pyrazolo[4,3-d]pyrimidin-7-yl]-(4-trifluoromethyl-phenyl)-amine hydrochloride (E 39)

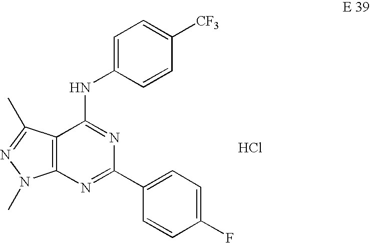

This compound was prepared at 80° C. for 12 hrs, by a procedure analogous to that disclosed in Example 1.

Yield: 55%; MP: 250-252° C.; Purity: 98.52%; ¹H NMR (400 MHz, CDCl₃): δ 10.60 (bs, 1H), 8.12-8.15 (m, 2H), 7.83 (d, J=8.3, 2H), 7.44 (d, J=8.6, 2H), 7.02 (t, J=8.6, 2H), 4.53 (s, 3H), 2.65 (s, 3H); MS: 402 (M⁺+1, 100%); IR (cm-¹): 3441.5.

### Example 40

### Preparation of (6-chloro-pyridin-3-yl)-[5-(4-fluoro-phenyl)-1,3-dimethyl-1H-pyrazolo[4,3-d]pyrimidin-7-yl]-amine hydrochloride (E 40)

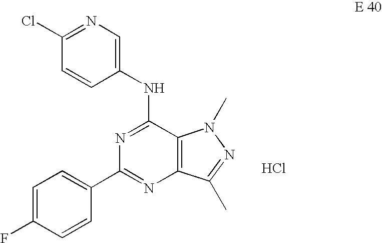

This compound was prepared at 80° C. for 12 hrs, by a procedure analogous to that disclosed in Example 1.

Yield: 29%; MP: 248-250° C.; Purity: 99.65%; ¹H NMR (400 MHz, CDCl₃): δ 8.60 (bs, 1H), 8.85-8.34 (m, 2H), 8.22 (dd, J=2.9, 8.6, 1H), 7.45-7.39 (m, 3H), 7.16 (dd, J=8.6, 15.3, 1H), 4.48 (s, 3H), 2.71 (s, 3H); MS: 369 (M⁺, 100%); IR (cm-¹): 3053.8.

### Example 41

### Preparation of N-{5-[5-(4-fluoro-phenyl)-1,3-dimethyl-1H-pyrazolo[4,3-d]pyrimidin-7-ylamino]-2-hydroxy-phenyl}-acetamide hydrochloride (E 41)

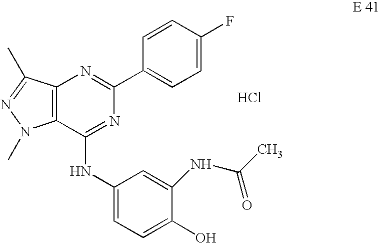

This compound was prepared at 80° C. for 12 hrs, by a procedure analogous to that disclosed in Example 1.

Yield: 54%; MP: 250-252° C.; Purity: 97.54%; ¹H NMR (400 MHz, CDCl₃): δ 9.29 (bs, 1H), 8.33-8.30 (m, 2H), 8.22 (s, 1H), 7.31-7.25 (m, 3H), 6.95 (d, J=8.6, 1H), 4.34 (s, 3H), 2.56 (s, 3H), 2.13 (s, 3H); MS: 407 (M⁺, 100%); IR (cm-¹): 3422.6, 1693.1

### Example 42

### Preparation of (1H-benzoimidazol-5-yl)-(1,3-dimethyl-5-phenyl-1H-pyrazolo[4,3-d]pyrimidin-7-yl)-amine hydrochloride (E 42)

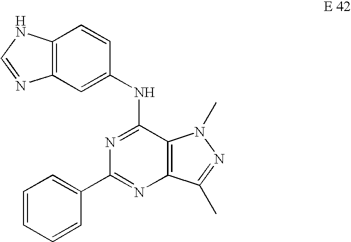

This compound was prepared at 80° C. for 24 hrs, by a procedure analogous to that disclosed in Example 1.

Yield: 44%; Purity: 99.01%; ¹H NMR (400 MHz, DMSO-d₆): δ 9.21 (d, J=12.4, 2H), 8.39 (s, 1H), 8.33(d, J=7.7, 2H), 7.84 (t, J=9.1, 1H), 7.48-7.41 (m, 3H), 4.35 (s, 3H), 2.50 (s,3H); MS: 354 (M⁺, 100%); IR (cm-¹): 3381.4

### Example 43

### Preparation of 4-[5-(4-fluoro-phenyl)-1,3-dimethyl-1H-pyrazolo[4,3-d]pyrimidin-7-ylamino]-N,N-dimethyl-benzenesulfonamide hydrochloride (E 43)

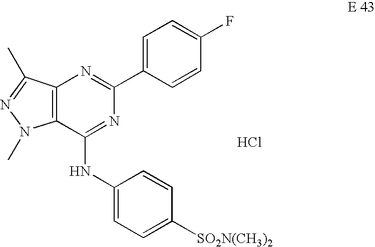

This compound was prepared at 80° C. for 20 hrs, by a procedure analogous to that disclosed in Example 1.

Yield: 32%; MP: 254-256° C.; Purity: 98.85%; ¹H NMR (400 MHz, DMSO-d₆): δ 9.44 (bs, —NH), 8.39-8.36 (m, 2H), 8.09(d, J=8.6, 2H), 7.82 (d, J=8.6, 2H), 7.31 (t, J=8.9, 2H), 4.32 (s, 3H), 2.64 (s, 6H), 2.52 (s, 3H); MS: 441 (M⁺+1, 100%); IR (cm-¹): 3379.9, 1628.4

### Example 44

### Preparation of 4-(1,3-dimethyl-5-phenyl-1H-pyrazolo[4,3-d]pyrimidin-7-ylamino)-benzenesulfonamide hydrochloride (E 44)

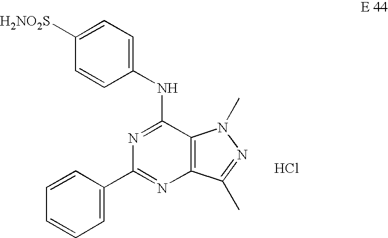

This compound was prepared at 80° C. for 20 hrs, by a procedure analogous to that disclosed in Example 1.

Yield: 53%; Purity: 98.02%; 1H NMR (400 MHz, DMSO-d₆): δ 9.46 (bs, —NH), 8.32 (d, J=6.5, 2H), 8.01 (d, J=8.3, 2H), 7.85 (d, J=8.6, 2H), 7.50-7.48 (m, 3H), 7.30 (bs, —NH₂), 4.33 (s, 3H), 2.53 (s, 3H); MS: 395 (M⁺+1, 100%); IR (cm-¹): 3496.7, 1628.9

### Example 45

### Preparation of 4-[5-(4-fluoro-phenyl)-1,3-dimethyl-1H-pyrazolo[4,3-d]pyrimidin-7-ylamino]-benzenesulfonamide hydrochloride (E 45)

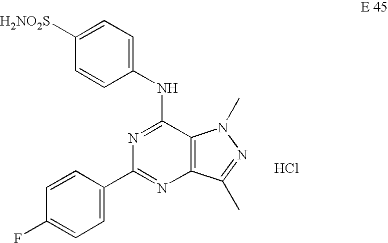

This compound was prepared at 80° C. for 15 hrs, by a procedure analogous to that disclosed in Example 1.

Yield: 67%; Purity: 99.06%; ¹H NMR (400 MHz, DMSO-d₆): δ 9.37 (bs, _NH), 8.38-8.34 (m, 2H), 7.99 (d, J=8.6, 2H), 7.88 (d, J=8.6, 2H), 7.31 (t, J=8.6, 2H), 4.74 (bs, —NH₂), 4.32 (s, 3H), 2.49 (s, 3H); MS: 413 (M⁺+1, 100%); IR (cm-¹): 3198.3, 1627.5

### Example 46

### Preparation of 4-[5-(4-fluoro-phenyl)-1,3-dimethyl-1H-pyrazolo[4,3-d]pyrimidin-7-ylamino]-N-methyl-benzenesulfonamide hydrochloride (E 46)

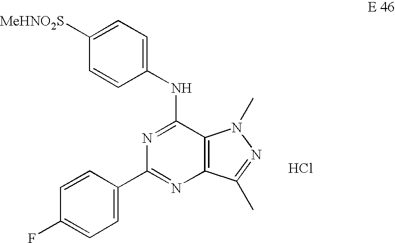

This compound was prepared at 80° C. for 24 hrs, by a procedure analogous to that disclosed in Example 1.

Yield: 65%; Purity: 99.11%; ¹H NMR (400 MHz, DMSO-d₆): δ 9.42 (bs, −1H), 8.38-8.35 (m, 2H), 8.04 (d,J=8.9, 2H), 7.84 (d, J=8.8, 2H), 7.32 (t, J=8.8, 2H), 6.41 (bs, —NH), 4.32 (s, 3H), 2.51 (s, 3H), 2.46 (s, 3H); MS: 427 (M⁺+1, 100%); IR (cm-¹): 3334.2

### Example 47

### Preparation of 4-[5-(4-fluoro-phenyl)-1,3-dimethyl-1H-pyrazolo[4,3-d]pyrimidin-7-ylamino]-benzamide hydrochloride (E 47)

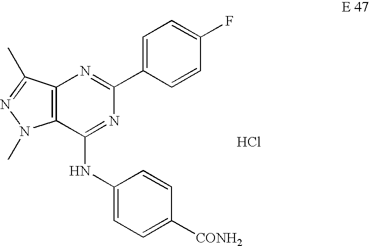

This compound was prepared at 80° C. for 24 hrs, by a procedure analogous to that disclosed in Example 1.

Yield: 55%; MP: 272-274° C.; Purity: 99.19%; ¹H NMR (400 MHz, DMSO-d₆): δ 9.26 (sb, —NH), 8.37-8.33 (m, 2H), 7.97 (d, J=8.8, 2H), 7.87 (d, J=8.6, 2H), 7.33-7.31 (m, 2H), 4.69 (bs, —NH₂), 4.32 (s, 3H), 2.49 (s, 3H); MS: 377 (M⁺+1, 100%); IR (cm-¹):3348.5, 1673.8.

### Example 48

### Preparation of 4-[5-(4-fluoro-phenyl)-1,3-dimethyl-1H-pyrazolo[4,3-d]pyrimidin-7-ylamino]-N-methyl-benzamide hydrochloride (E 48)

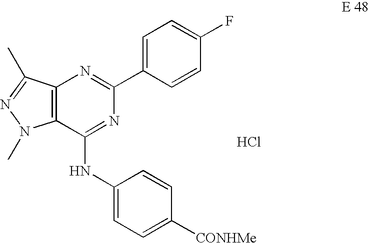

This compound was prepared at 80° C. for 24 hrs, by a procedure analogous to that disclosed in Example 1.

Yield: 53%; Purity: 98.75%; ¹H NMR (400 MHz, DMSO-d₆): δ 9.31 (bs, —NH), 8.37-8.32 (m, 1H), 7.95-7.87 (m, 4H), 7.34-7.28 (m, 3H), 4.33 (s, 3H), 2.80 (s, 3H), 2.49 (s, 3H); MS: 391 (M⁺+1, 100%); IR (cm-¹): 3320.8, 1630.9

### Example 49

### Preparation of 3-[5-(4-fluoro-phenyl)-1,3-dimethyl-1H-pyrazolo[4,3-d]pyrimidin-7-ylamino]-benzamide hydrochloride (E 49)

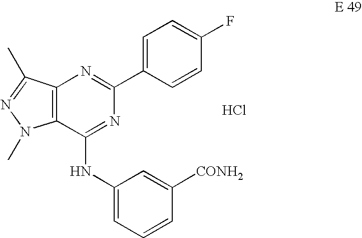

This compound was prepared at 80° C. for 24 hrs, by a procedure analogous to that disclosed in Example 1.

Yield: 44%; MP: 258-260° C.; Purity: 99.33%; ¹H NMR (400 MHz, DMSO-d₆): δ 9.29 (bs, —NH), 8.40-8.35 (m,3H), 7.94 (dd, J=1.8, 8.0, 1H), 7.70 (d, J=7.8, 1H), 7.52 (t, J=7.8,1H), 7.27 (t, J=8.9, 2H), 5.17 (bs, —NH₂), 4.34 (s, 3H), 2.52 (s, 3H); MS: 377 (M⁺+1, 100%); IR (cm-¹): 3387.3, 1658.5.

### Example 50

### Preparation of 3-[5-(4-fluoro-phenyl)-1,3-dimethyl-1H-pyrazolo[4,3-d]pyrimidin-7-ylamino]-N-methyl-benzamide hydrochloride (E 50)

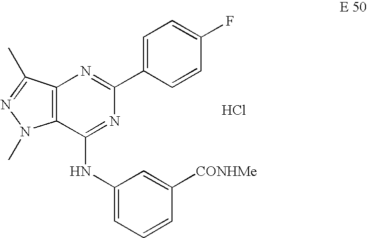

This compound was prepared at 80° C. for 24 hrs, by a procedure analogous to that disclosed in Example 1.

Yield: 50%; MP: 272-274° C.; Purity: 99.39%; ¹H NMR (400 MHz, DMSO-d₆): δ 9.31 (bs, NH), 8.37-8.34 (m, 3H), 7.96-7.93 (m, 1H), 7.65 (d, J=7.8, 1H), 7.52 (t, J=7.8, 1H), 7.30-7.26 (m, 2H), 5.21 (bs, —CONH), 4.34 (s, 3H), 2.82 (d, J=4.3, 3H), 2.52 (s, 3H); MS: 391 (M⁺+1, 100%); IR (cm-¹): 3322.4, 1658.7.

### Example 51

### Preparation of (3-fluoro-4-methoxy-phenyl)-(1-methyl-3-propyl-5-trifluoromethyl-1H-pyrazolo[4,3-d]pyrimidin-7-yl)-amine hydrochloride (E 51)

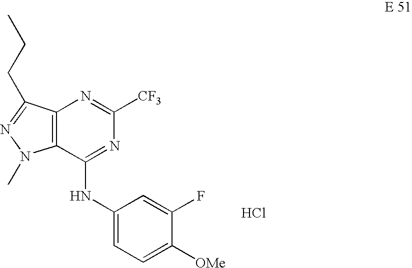

The title compound was prepared at 80° C. for 12 h, by a procedure analogous to that disclosed in Example 1.

Yield: 50%; MP: 136-138° C.; Purity: 98.6%; ¹H NMR (200 MHz, DMSO-d₆): ∂ 9.15 (D₂O exchangeble, NH), 7.65 (d, J=13.7 Hz, 1H), 7.50 (d, J=9.0 Hz, 1H), 7.2 (t, J=9.0 Hz, 1H), 4.34 (s, 3H), 3.86 (s, 3H), 2.86 (t, J=7.3 Hz, 2H), 1.79-1.75 (m, 2H), 0.95 (t, J=7.3 Hz, 3H); MS: 384 (M+1, 100); IR (cm-¹): 3452, 1614.

### Example 52

### Preparation of benzo[1,3]dioxol-5-yl-(1-methyl-3-propyl-5-thiophen-2-yl-1H-pyrazolo[4,3-d]pyrimidin-7-yl)-amine hydrochloride (E 52)

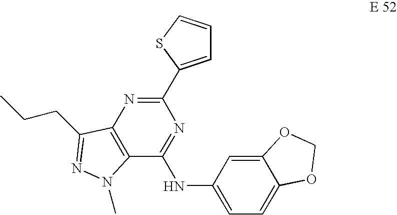

This compound was prepared at 80° C. for 12 h, by a procedure analogous to that disclosed in Example 1.

Yield: 48%; MP: 246-250° C.; Purity: 97.16%; ¹H NMR (200 MHz, CDCl₃) δ 9.12 (s, D₂O exchangeable, 1H) 7.93 (s, 1H), 7.69 (d, J=4.8 Hz, 1H), 7.5 (s, 1H), 7.18 (d, J=5.1 Hz, 2H), 7.0 (d, J=8.5 Hz, 1H), 6.07 (s, 2H), 3.87 (s, 3H), 2.90 (t, J=7.3 Hz, 2H), 1.85-1.74 (m, 2H), 0.97 (t, J=7.3 Hz, 3H); MS: 394 (M⁺35, 100); IR (cm-¹): 1570, 1483.

### Examples 53 and 54

### Preparation of 4-[5-(3,4-dimethoxy-phenyl)-1-methyl-3-propyl-1H-pyrazolo[4,3-d]pyrimidin-7-yl]-2-methyl-phenol (E 53) and 4-[5-(3-hydroxy,4-methoxy-phenyl)-1-methyl-3-propyl-]H-pyrazolo[4,3-d]pyrimidin-7-yl]-2-methyl-phenol (E 54)

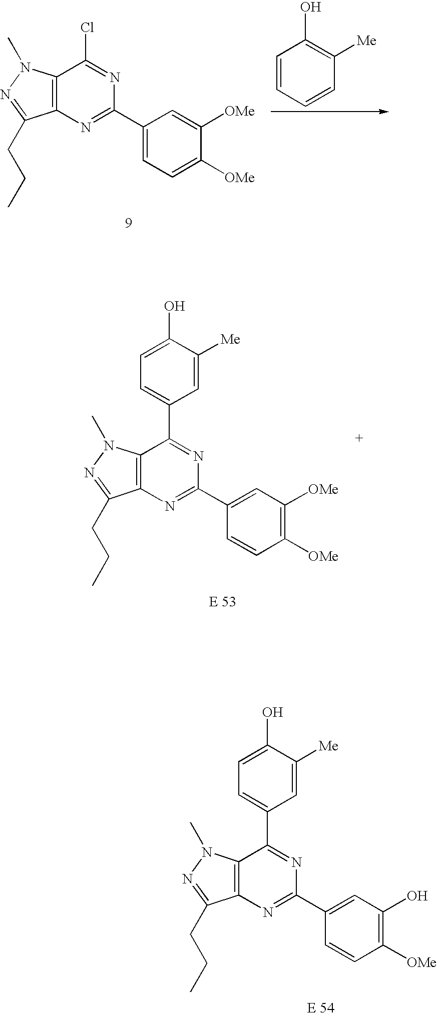

To a stirring mixture of compound 9 (0.5 gram, 1.44 mmol) and aluminum chloride (AlCl₃) (0.67 gram, 5 mmol) in nitrobenzene (10 mL) was added o-cresol (0.16 gram, 1.44 mmol) drop wise under nitrogen atmosphere. The mixture was then stirred at 120° C. for 2.5 hours. The mixture was cooled to room temperature, poured into water (50 mL) and extracted with ethyl acetate (3×30 mL). The organic layers were collected, combined, dried over anhydrous Na₂SO₄ and then concentrated. The residue thus obtained was purified by column chromatography using 20% ethyl acetate/Petroleum ether to yield the desired compounds 4-[5-(3,4-dimethoxy-phenyl)-1-methyl-3-propyl-1H-pyrazolo[4,3-d]pyrimidin-7-yl]-2-methyl-phenol (E 53) (0.20 gram; 33% yield) and 4-[5-(3-hydroxy,4-methoxy-phenyl)-1-methyl-3-propyl-1H-pyrazolo[4,3-d]pyrimidin-7-yl]-2-methyl-phenol (E 54), Yield: 6%. 4-[5-(3,4-dimethoxy-phenyl)-1-methyl-3-propyl-1H-pyrazolo[4,3-d]pyrimidin-7-yl]-2-methyl-phenol (E 53)

Melting point: 198-200° C.; ¹H NMR (400 MHz, DMSO-d₆): δ 9.93 (s, D₂O exchangable, 1H), 8.08-8.04 (m, 2H), 7.61-7.52 (m, 2H) 7.10-7.00 (m, 2H), 3.87-3.81(m, 2H), 3.00 (t, J=7.4 Hz, 2H), 2.26 (s, 3H), 1.0 (t, J=7.3 Hz, 3H); MS: 419 (M+1, 100%); IR (cm⁻¹): 3423, 1606.

### 4-[5-(3-hydroxy,4-methoxy-phenyl)-1-methyl-3-propyl-1H-pyrazolo[4,3-d]pyrimidin-7-yl]-2-methyl-phenol (E 54)

Melting point: 180-182° C.; 1H NMR (400 MHz, DMSO-d₆) δ 9.92 (s, D₂O exchangeable, 1H), 9.35 (s, D₂O exchangeable, 1H), 8.02-7.91 (m, 2H), 7.59-7.52 (m, 2H), 7.04-6.89 (m, 2H), 3.87 (s, 3H), 3.01 (t, J=7.2 Hz, 2H), 2.20 (s, 3H), 1.90 (q, J=7.2 Hz, 2H), 1.01 (t, J=7.2 Hz, 3H).

MS: 405 (M+1, 100%).

IR (cm⁻¹): 3311, 2926.

### Examples 55-57

Unless otherwise indicated, the following compounds presented in Examples 55-57 were prepared by a procedure analogous to that disclosed in Examples 53 and 54, using analogous starting materials with the appropriate substitution, to afford the corresponding compounds, listed as compounds E 55 through E 57.

### Example 55

### Preparation of 2-chloro-4-[5-(4-fluoro-phenyl)-1-methyl-3-propyl-1H-pyrazolo[4,3-d]pyrimidin-7-yl]-phenol (E 55)

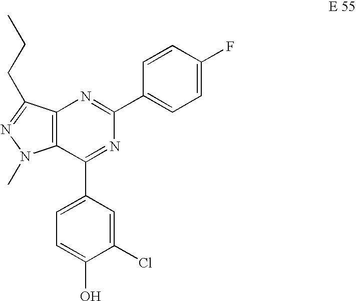

Yield: 20%; Melting point: 182-184° C.; ¹H NMR (200 MHz, DMSO-d₆) δ10.8 (D₂O exchangeable OH), 8.51-8.50 (m, 2H), 7.91 (s, 1H), 7.67 (d, J=6.7 Hz, 1H), 7.35 (t, J=8.7 Hz, 2H), 7.20 (d, J=8.1 Hz, 1H), 3.85 (s, 3H), 3.0 (t, J=7.3 Hz, 2H), 1.91-1.88 (m, 2H), 1.0 (t, J=7.3 Hz, 3H); MS: 397 (M+1, 100); IR (cm⁻¹): 3382, 1602.

### Example 56

### Preparation of 5-(4-fluoro-phenyl)-7-(4-hydroxy-3-methyl-phenyl)-1-methyl-3-propyl-1H-pyrazolo[4,3-d]pyrimidine (E 56)

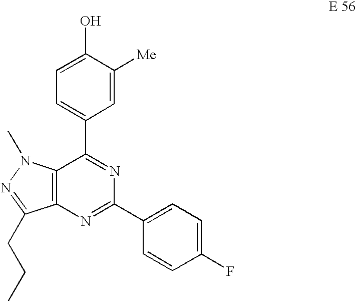

Yield: 40%; ¹H NMR (400 MHz, CDCl₃): δ 8.03-8.00 (m, 1H), 7.58 (s, 1H), 7.51 (d, J=2.3 Hz, 1H), 7.49 (d, J=2.3 Hz, 1H), 7.13 (t, J=6.6 Hz, 2H), 6.96 (d, J=8.2 Hz, 1H), 3.86 (s, 3H), 3.1 (t, J=7.4 Hz, 2H), 2.38 (s, 3H), 1.96 (m, J=7.4 Hz, 2H), 1.08 (t, J=7.4 Hz, 3H); Mass (CI method, i-butane): 377 (M+1, 100); IR: vₘₐₓ (KBr, cm⁻¹): 3175, 1606.

### Example 57

### Preparation of 2-methyl-4-(1-methyl-5-phenyl-3-propyl-1H-pyrazolo[4,3-d]pyrimidin-7-yl)-phenol (E 57)

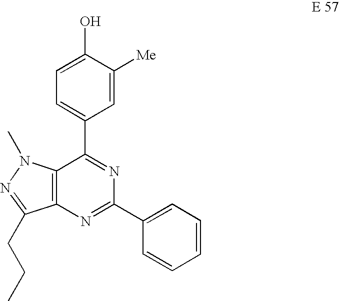

Yield: 56%; Melting point: 204- 206° C.; ¹H NMR (200 MHz,DMSO-d₆): δ 8.5 (d, J=1.7 Hz, 2H), 7.5-7.2 (m, 5H), 6.94 (d, J=8.1 Hz, 1H), 5.3 (bs, 1H), 3.8 (s, 3H), 3.15-3.08 (t, J=7.5 Hz, 2H), 2.3 (s, 3H), 2.04 -1.89 (m, 2H), 1.12-1.04 (t, J=7.4 Hz, 3H); MS: 359 (M+1, 100%; IR (cm⁻¹): 3171, 2956, 1603.

### Example 58

### Preparation of 5-(4-fluoro-phenyl)-7-(4-methoxy-3-methyl-phenyl)-1-methyl-3-propyl-1H-pyrazolo[4,3-d]pyrimidine (E 58)

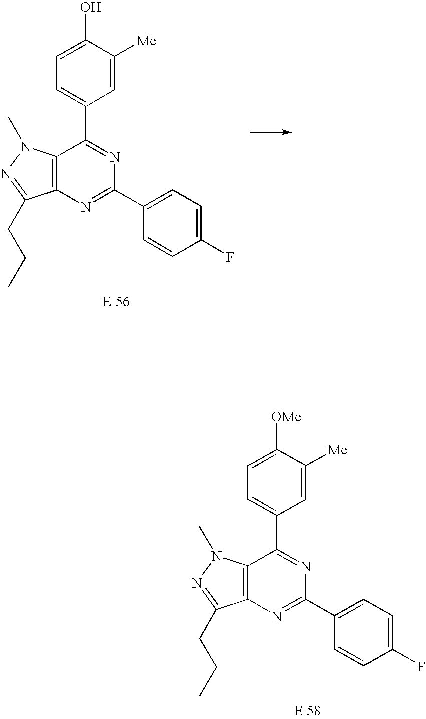

To a stirring mixture of compound E 56 (0.2 gram, 0.53 mmol) and potassium carbonate (K₂CO₃) (0.29 gram, 2.1 mmol) in dry DMF (5 mL) was added methyliodide (89 mg, 0.63 mmol) drop wise under nitrogen atmosphere. The mixture was then stirred at 80° C. for 3 hours. The mixture was cooled to room temperature, poured into water (25 mL) and stirred for 30 min. The solid precipitated was filtered off, washed with petroleum ether and dried under vacuum to afford the desired compound 5-(4-fluoro-phenyl)-7-(4-methoxy-3-methyl-phenyl)-1-methyl-3-propyl-1H-pyrazolo[4,3-d]pyrimidine (E 58) 0.13gram as an off white solid.

Yield: 62%; Melting point 136-138° C.; ¹H NMR (400 MHz, CDCl₃): 7.59 (d, J=3.0 Hz, 2H), 7.15 (t, J=7.0 Hz, 2H), 7.0 (t, J=8.9 Hz, 2H), 3.94 (s, 3H), 3.87 (s, 3H), 3.10 (t, J=7.4 Hz, 2H), 2.34 (s, 3H), 1.97-1.93 (m, 2H), 1.08 (t, J=7.4 Hz, 3H); Mass (CI method, i-butane): 391 (M+1, 100); IR: vₘₐₓ (KBr, cm⁻¹): 3451, 1607.

### Examples 59-61

Unless otherwise indicated, the following compounds presented in Examples 59-61 were prepared by a procedure analogous to that disclosed in Examples 53, 54, and 58, using analogous starting materials with the appropriate substitution, to afford the corresponding compounds, listed as compounds E 59 through E 61.

### Example 59

### Preparation of 7-(3-fluoro-4-methoxy-phenyl)-5-(4-fluoro-phenyl)-1-methyl-3-propyl-1H-pyrazolo[4,3-d]pyrimidine (E 59)

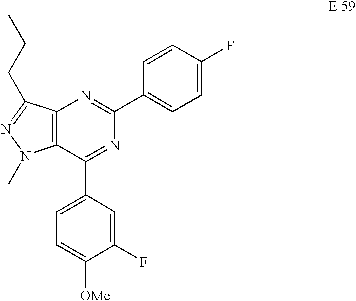

Yield: 54%; Melting point: 174-176° C.; ¹H NMR (400 MHz, CDCl₃) δ 8.57-8.55 (m, 2H), 7.61-7.50 (m, 2H), 7.16 (t, J=7.8 Hz, 2H), 7.14 (d, J=7.4 Hz, 1H), 4.0 (s, 3H), 3.89 (s, 3H), 3.10 (t, J=7.4 Hz, 2H), 1.98-1.95 (m, 2H), 1.08 (t, J=7.4 Hz, 3H); MS: 395 (M+1, 100); IR (cm⁻¹): 2955, 1605.

### Example 60

### Preparation of 7-(4-methoxy-3-methyl-phenyl)-1-methyl-5-phenyl-3-propyl-1H-pyrazolo[4,3-d]pyrimidine (E 60)

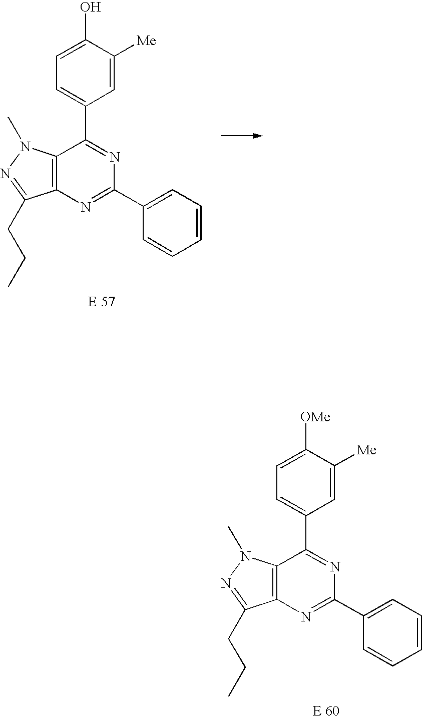

Yield: 43%; Melting point: 158-160° C.; ¹H NMR (400 MHz, CDCl₃): δ 8.60-8.58 (m, 2H), 7.61-7.50 (m, 2H), 7.48-7.40 (m, 3H), 7.02-7.00 (m, 1H), 3.94 (s, 3H), 3.87 (s, 3H), 3.11 (t, J=7.5 Hz, 2H), 2.34 (s, 3H), 2.03-1.94 (m, 2H), 1.08 (t, J=7.5 Hz, 3H); MS: 373 (M+1, 100%); IR (cm⁻¹): 3449, 2294.

### Example 61

### Preparation of 7-(3-fluoro-4-methoxy-phenyl)-1-methyl-5-phenyl-3-propyl-1H-pyrazolo[4,3-d]pyrimidine (E 61)

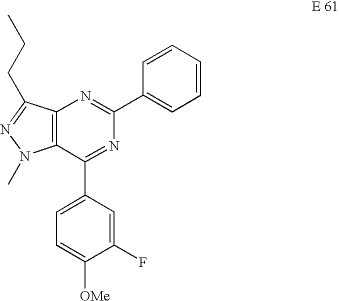

Yield: 25%; Melting point: 158-160° C.; ¹H NMR (400 MHz, CDCl₃): δ 8.59-8.56 (m, 2H), 7.62-7.59 (m, 2H), 7.54-7.42 (m, 3H), 7.16 (t, J=8.3 Hz, 1H), 4.01 (s, 3H), 3.89 (s, 3H), 3.12 (t, J=7.5 Hz, 2H), 2.03-1.94 (m, 2H), 1.08 (t, J=7.5 Hz, 3H); MS: 377 (M+1, 100%); IR (cm⁻¹): 3426, 2957.

### Example 62

### Preparation of 5-(4-fluoro-phenyl)-1-methyl-7-(4-methylsulfanyl-phenyl)-3-propyl-1H-pyrazolo[4,3-d]pyrimidine (E 62)

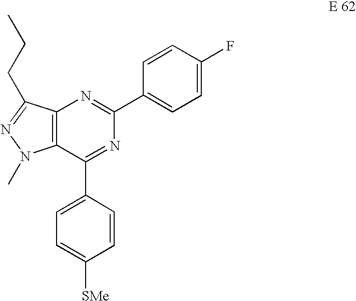

### Step 1: Preparation of 4-methylsulfanyl phenyl boronic acid

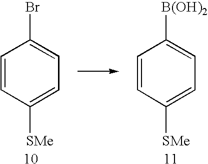

To a cold (−78° C.) and stirring solution of 4-bromothioanisole (10) (3 grams, 14.8 mmol) in THF (15 mL) was added n-BuLi (10 mL) slowly under nitrogen atmosphere. The mixture was the allowed to reach the room temperature and stirring continued for 20 minutes. The mixture was then cooled to −78° C. A solution of triisopropyl borate (10 mL, 17.7 mmol) in THF (10 mL) was added to it slowly. The mixture was stirred for 1 hour at −78° C. and then 2 hours at room temperature. The mixture was acidified with cold 5% HCl, diluted with water (25 mL) and extracted with ethyl acetate. The organic layers were collected, combined, washed with brine solution (20 mL) followed by water (20 mL), dried over anhydrous Na₂SO₄ and concentrated. The residue thus obtained was purified by column chromatography using EtOAc-Hexane to afford the required aryl boronoc acid (11) (170 mg,).

Yield: 10%; ¹H NMR (200 MHz, CDCl₃): δ 8.10 (d, J=7.9 Hz, 2H), 7.33 (d, J=7.9 Hz, 2H), 2.55 (s, 3H, SCH₃), 1.56 (bs, D₂O exchangeable, OH), 1.25 (s, exchangeable, OH).

IR: vₘₐₓ (KBr, cm⁻¹): 3406, 1594.

### Step 2: Preparation of 5-(4-fluoro-phenyl)-1-methyl-7-(4-methylsulfanyl-phenyl)-3-propyl-1H-pyrazolo[4,3-d]pyrimidine (E 62)

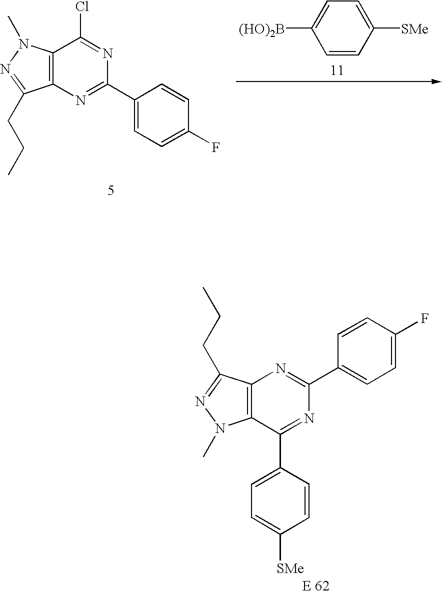

A mixture of compound 5 (0.25 gram, 0.82 mmol), (PPh₃)₄Pd (0.048 gram, 0.04 mmol) and compound 11 (0.14 gram, 0.82 mmol) in dry DMF (3 mL) was stirred under nitrogen atmosphere for 30 min. To this was added a solution of Na₂CO₃ (0.69 gram, 6.56 mmol dissolved in 3.3 mL of water) slowly and the mixture was stirred at 100° C. for 12 hours. The mixture was then cooled to room temperature, diluted with water (15 mL) and extracted with ethyl acetate. Organic layers were collected, combined, washed with brine solution (15 mL) followed by water (2×10 mL), dried over anhydrous Na₂SO₄ and concentrated under vacuum. The residue thus obtained was purified by column chromatography using ethyl acetate-petroleum ether to give the desired compound (0.24 gram)

Yield: 75%; Melting point: 114-116° C.; ¹H NMR (200 MHz, DMSO-d₆): δ 8.61-8.54 (m, 2H), 7.71 (d, J=8.4 Hz, 2H), 7.43 (d, J=8.1 Hz, 2H), 7.15 (t, J=8.4 Hz, 2H), 3.87 (s, 3H), 3.11 (t, J=7.3 Hz, 2H), 2.58 (s, 3H), 2.03-1.92 (m, 2H), 1.08 (t, J=7.3 Hz, 3H).

Mass (CI method, i-butane): 393 (M+1, 100); IR: vₘₐₓ (KBr, cm⁻¹): 1599, 1453.

### Examples 63-68

Unless otherwise indicated, the following compounds presented in Examples 63-68 were prepared by a procedure analogous to that disclosed in Example 62, using analogous starting materials with the appropriate substitution, to afford the corresponding compounds, listed as compounds E 63 through E 68.

### Example 63

### Preparation of 5-(4-fluoro-phenyl)-1-methyl-3-propyl-7-p-tolyl-1H-pyrazolo[4,3-d]pyrimidine (E 63)

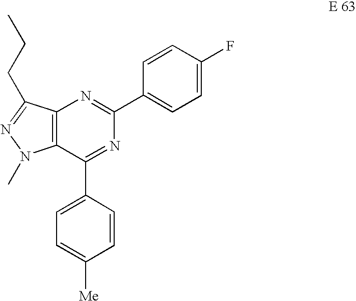

Yield: 42%.; Melting point: 148-150° C.; ¹H NMR (200 MHz, CDCl₃): δ 8.62-8.54 (m, 2H), 7.66 (d, J=8.0 Hz, 2H), 7.39 (d, J=7.9 Hz, 2H), 7.14 (t, J=8.8 Hz, 2H), 3.84 (s, 3H), 3.11 (t, J=7.4 Hz, 2H), 2.49 (s, 3H), 2.04-1.89 (m, 2H), 1.08 (t, J=7.3 Hz, 3H); MS: 361 (M+1, 100); IR (cm⁻¹): 1602, 1549, 1455.

### Example 64

### Preparation of 5-(4-fluoro-phenyl)-1-methyl-7-phenyl-3-propyl-1H-pyrazolo[4,3-d]pyrimidine (E 64)

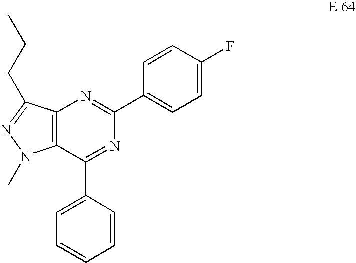

Yield: 51%.; Melting point: 115-118° C.; ¹H NMR (200 MHz, DMSO-d₆): δ 8.65-8.6 (m, 1H), 7.77-7.76 (m, 3H), 7.28-7.25 (m, 5H), 3.83 (s, 3H), 3.11-3.0 (m, 2H), 2.0-1.96 (m, 2H), 1.12-1.05 (t, J=7.6 Hz, 3H); MS: 347 (M+1, 100%); IR (cm⁻¹): 1599, 1453.

### Example 65

### Preparation of 7-benzo[1,3]dioxol-5-yl-1,3-dimethyl-5-phenyl-1H-pyrazolo[4,3-d]pyrimidine (E 65)

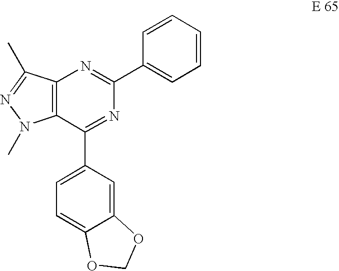

Melting point: 142-144° C.; ¹H NMR (200 MHz, CDCl₃): δ 8.57 (d, J=6.1 Hz, 2H), 7.48 (d, J=7.0 Hz, 2H), 7.33 (s, 1H), 7.26-7.23 (m, 3H), 6.10 (s, 2H), 3.90 (s, 3H), 2.73 (s, 3H); MS: 345 (M+1, 100).

### Example 66

### Preparation of 5-(4-fluoro-phenyl)-1,3-dimethyl-7-phenyl-1H-pyrazolo[4,3-d]pyrimidine (E 66)

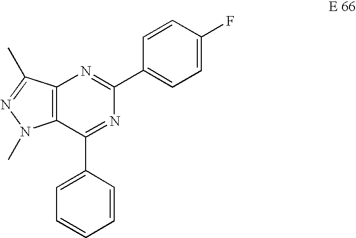

Yield: 76%; Melting point: 150-152° C.; ¹H NMR (200 MHz, CDCl₃): δ 8.62-8.55 (m, 2H), 7.71-7.57 (m, 5H), 7.15 (t, J=8.7 Hz, 2H), 3.85 (s, 3H), 2.74 (s, 3H); MS: 319 (M+1, 100).

### Example 67

### Preparation of 7-(3-methanesulfonyl-phenyl)-1,3-dimethyl-5-phenyl-1H-pyrazolo[4,3-d]pyrimidine (E 67)

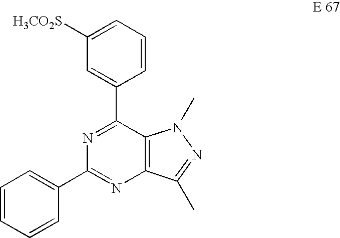

This compound was prepared at 80° C. for 3 hours, by a procedure analogous to that disclosed in Example 62.

Yield: 52%; Melting point: 210-212° C.; Purity: 95.90%; ¹H NMR (400 MHz, CDCl₃): 8.59-8.56 (m, 3H), 8.37-8.12 (m,2H), 7.52 (t, J=1.9, 1H), 7.50-7.44 (m, 3H), 3.85 (s, 3H), 3.14 (s, 3H), 2.74 (s, 3H); MS: 378 (M⁺, 100%); IR (cm−¹): 3020.8, 1680.2

### Example 68

### Preparation of 5-(4-fluoro-phenyl)-7-(3-methanesulfonyl-phenyl)-1,3-dimethyl-1H-pyrazolo[4,3-d]pyrimidine (E 68)

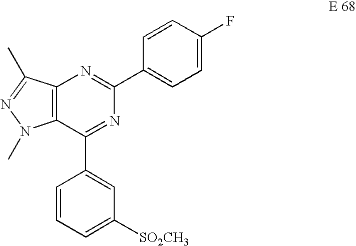

This compound was prepared at 80° C. for 3 h, by a procedure analogous to that disclosed in Example 62.

Melting point: 176-178° C.; Purity: 96.98%; ¹H NMR (400 MHz, CDCl₃): δ 8.60-8.55 (m, 2H), 8.36 (s, 1H), 8.19-8.11 (m, 2H), 7.84 (t, J=7.7, 1H), 7.19-7.14 (m, 2H), 3.85 (s, 3H), 3.14 (s, 3H), 2.62 (s, 3H); MS: 396 (M⁺, 100%); IR (cm−¹): 3446.0, 1601.9

### Example 69

### Preparation of 5-(4-fluoro-phenyl)-7-(4-methanesulfonyl-phenyl)-1-methyl-3-propyl-1H-pyrazolo[4,3-d]pyrimidine (E 69)

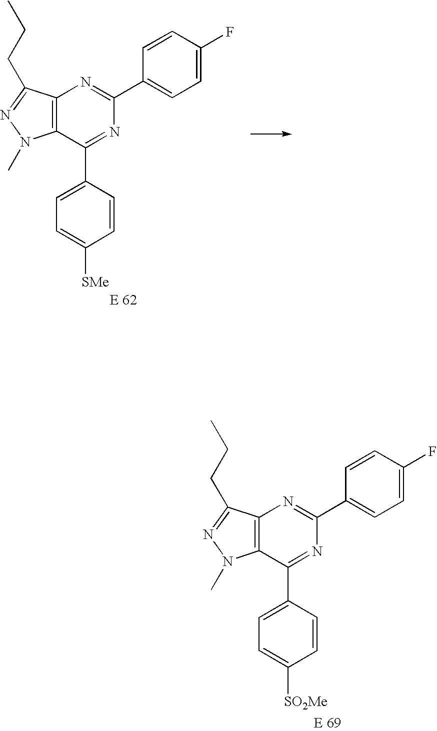

To a mixture of compound E 62 (0.13 gram, 0.32 mmol) and oxone (0.59 gram, 0.96 mmol) in acetone (3 mL) was added water (2 mL) and the mixture was stirred for 6 hours at room temperature under nitrogen atmosphere. After completion of the reaction the mixture was diluted with cold NaHCO₃ solution followed by water (10 mL) and was extracted with ethyl acetate. Organic layers were collected, combined, dried over anhydrous Na₂SO₄ and concentrated under vacuum. The residue thus obtained was purified by column chromatography using ethyl acetate-hexane to give the desired compound (0.10 gram)

Yield : 75%; Melting point: 208-210° C.; ¹H NMR (200 MHz, CDCl₃): δ 8.60-8.53 (m, 2H), 8.19 (d, J=8.14 Hz, 2H), 7.99 (d, J=8.4 Hz, 2H), 7.17 (t, J=8.7 Hz, 2H), 3.84 (s, 3H), 3.17 (s, 3H), 3.13 (t, J=7.6 Hz, 2H), 2.04-1.93 (m, 2H), 1.09 (t, J=7.3 Hz, 3H).

Mass (CI method, i-butane): 425 (M+1, 100); IR: νₘₐₓ (KBr, cm⁻¹): 1151.

### Example 70

### Preparation of 5-(4-fluoro-phenyl)-1-methyl-7-phenylethynyl-3-propyl-1H-pyrazolo[4,3-d]pyrimidine (E 70)

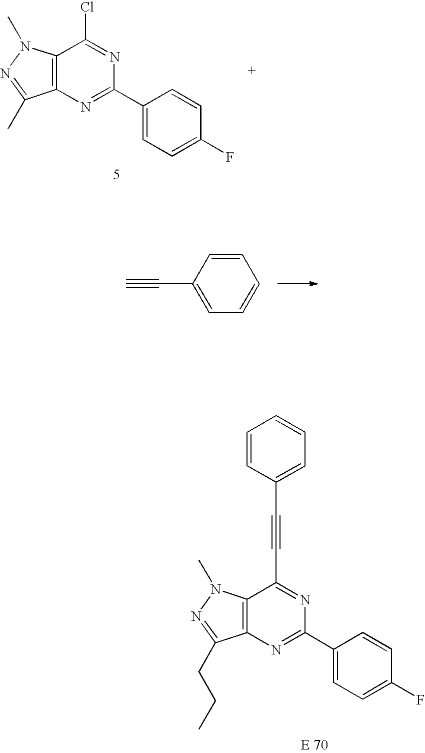

A mixture of compound 5 (0.5 gram, 1.64 mmol), (PPh₃)₂PdCl₂ (46 mg, 0.06 mmol) and triethylamine (1.2 mL, 8.2 mmol) in dry DMF (10 mL) was stirred at room temperature under nitrogen atmosphere for 30 minutes. To this was added phenyl acetylene (0.33 gram, 3.29 mmol) slowly and the mixture was stirred at 100° C. for 30 minutes. The mixture was then cooled to room temperature, diluted with water (25 mL) and extracted with ethyl acetate. Organic layers were collected, combined, washed with brine solution (10 mL) followed by water (2×10 mL), dried over anhydrous Na₂SO₄ and concentrated under vacuum. The residue thus obtained was purified by column chromatography using ethyl acetate-petroleum ether to give the desired compound E 70 (0.30 gram).

Yield: 50%; Melting point: 156-158° C.; ¹H NMR (200 MHz, CDCl₃): δ 8.58-8.50 (m, 2H), 7.74-7.69 (m, 2H) 7.51-7.40 (m, 3H), 7.18 (t, J=8.7 Hz, 2H), 4.43 (s, 3H), 3.06 (t, J=7.3 Hz, 2H), 1.98-1.89 (m, 2H) 1.05 (t, J=7.3 Hz, 3H); Mass (CI method, i-butane): 371 (M+1, 100); IR: νₘₐₓ (KBr, cm⁻¹): 2212, 1542, 1445.

### Example 71

### Preparation of 7-(4-fluoro-phenoxy)-1-methyl-5-phenyl-3-propyl-1H-pyrazolo[4,3-d]pyrimidine (E 71)

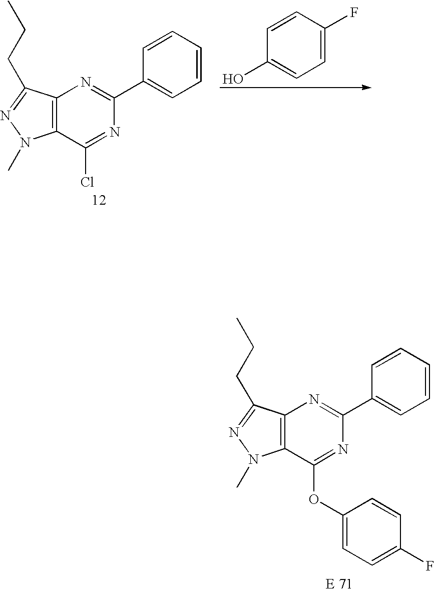

7-(4-Fluoro-phenoxy)-1-methyl-5-phenyl-3-propyl-1H-pyrazolo[4,3-d]pyrimidine (E 71) was prepared by reacting compound 12 (0.25 gram, 0.69 mmol) with 4-fluorophenol (0.08 gram, 0.71 mmol) and K₂CO₃ (0.47 grams, 3.45 mmol) in DMF (5 mL) by heating at 80° C. for 12 hrs.

Yield: 56%; Melting point: 126-128° C.; ¹H NMR (200 MHz, DMSO-d₆) δ 8.24 (t, J=3.7 Hz, 2H), 7.39-7.14 (m, 7H), 4.33 (s, 3H), 3.06 (t, J=7.5 Hz, 2H), 1.98 (q, J=7.5 Hz, 2H), 1.06 (t, J=7.3 Hz, 3H).

### Example 72

### Preparation of (3-chloro-4-methoxy-phenyl)-[6-(4-fluoro-phenyl)-1,3-dimethyl-1H-pyrazolo[4,3-c]pyridin-4-yl]-amine hydrochloride (E 72)

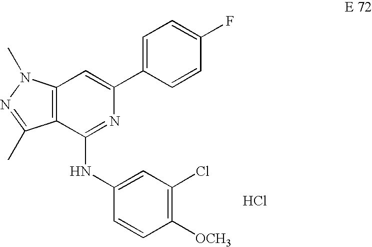

### Step 1: Preparation of (2,5-dimethyl-2H-pyrazolo-3yl)-methanol (14)

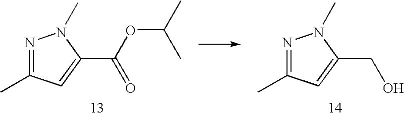

To a solution of compound 13 (4.5 grams, 24.7 mmol) in THF (60 mL) was added lithiumaluminumhydrade (LiAlH₄) (1.22 grams, 321 mmol) in 3-4 portions at 0° C. under nitrogen atmosphere. The resulting mixture was stirred at the same temperature for 2-3 hours and then the excess of LiAlH₄ was quenched by adding a saturated solution of sodium sulfate. The mixture was filtered and the residue was washed with ethyl acetate. The filtrates were collected, combined and concentrated under reduced pressure to afford the desired compound (2,5-dimethyl-2H-pyrazolo-3yl)-methanol (14) 3 grams as a brown solid.

Yield : 86%; ¹H NMR (200 MHz, CDCl₃): δ 6.0 (s, H), 4.57 (d, J=5.9 Hz, 2H), 3.8 (s, 3H), 3.16 (s, OH), 2.24 (s, 3H); Mass (CI method, i-butane): 127 (M+1, 100%); IR: νₘₐₓ (KBr, cm⁻¹): 3281.

### Step 2: Preparation of 2,5-dimethyl-2H-pyrazole-3-carbaldehyde (15)

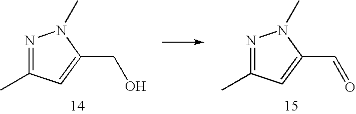

A mixture of compound 14 (3 grams, 23.8 mmol) and pyridinium dichromate (13.4 grams, 35.7 mmol) in dichloromethane (100 mL) was stirred at 25° C. under nitrogen atmosphere for 16 hours. The reaction mixture was then filtered. The filtrate was collected, dried over anhydrous Na₂SO₄ and concentrated under reduced pressure. The crude thus obtained was passed through the silica gel to afford the desired compound 2,5-dimethyl-2H-pyrazole-3-carbaldehyde (15) 1 gram as a brown solid.

Yield: 34%; ¹H NMR (200 MHz, CDCl₃): δ 9.8 (s, H), 6.6(s, H), 4.1 (s, 3H), 2.3 (s, 3H).

Mass (CI method, i-butane): 125 (M+1, 100%); IR: νₘₐₓ (KBr, cm⁻¹): 1688.

### Step 3: Preparation of 3-(2,5-dimethyl-2H-pyrazole-3yl)-2-(4-fluoro phenyl) acrylic acid (16)

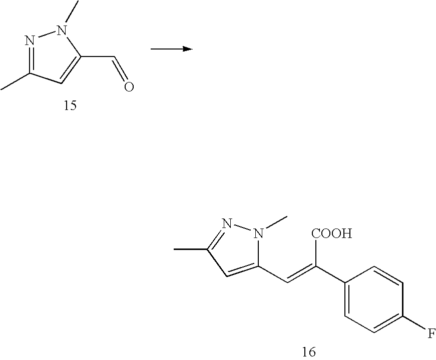

A mixture of compound 15 (1 gram, 8.0 mmol) and 4-fluorophenyl acetic acid (1.24 grams, 8.0 mmol), acetic anhydride (2 mL) and triethylamine (0.84 mL, 6.0 mmol) was refluxed under nitrogen atmosphere for 5-6 h. The excess of acetic anhydride was distilled out at the same temperature. The mixture was then diluted with water (100 mL) and neutralized with 2N hydrochloric acid. The solid precipitated was filtered and dried under vacuum to afford the title compound 3-(2,5-dimethyl-2H-pyrazole-3yl)-2-(4-fluoro phenyl) acrylic acid (16) 1.4 gram as a pale brown solid. Yield: 67%; ¹H NMR (200 MHz, DMSO-d₆): δ 12.75 (s, D₂O exchangeable) 7.66 (s, 1H), 7.26-7.12 (m, 4H), 5.0 (s, H) 3.84 (s, 3H), 2.05 (s, 3H); Mass (CI method, i-butane): 261 (M+1, 80%); IR: νₘₐₓ (KBr, cm⁻¹): 3440, 1695.

### Step 4: Preparation of 3-(2,5-dimethyl-2H-pyrazole-3yl)-2-(4-fluoro phenyl) acrylic azide (17)

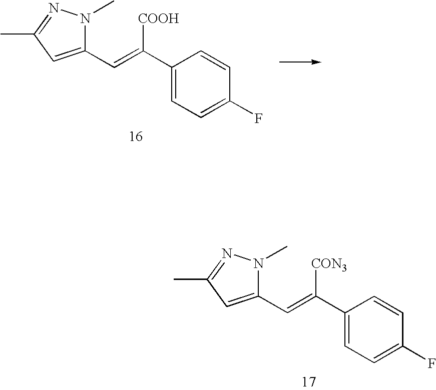

A mixture of compound 16 (1.2 grams, 4.6 mmol) and triethylamine (0.78 gram, 5.0 mmol) in acetone (15 mL) was cooled to 0° C. and a solution of ethyl chloroformate (0.78 gram, 6.4 mmol) in acetone (5 mL) was added dropwise to it followed by the addition of sodium azide solution (0.52 gram, 6.9 mmol, in 5 mL water). The resulting reaction mixture was stirred at room temperature for 1 hour and then poured into ice water (50 mL). The solid precipitated was filtered, washed with excess of water and dried for 15 hours to afford the title compound 3-(2,5-dimethyl-2H-pyrazole-3yl)-2-(4-fluoro phenyl) acrylic azide (17) 0.8 gram as a yellow solid.

Yield : 61%; ¹H NMR (200 MHz, DMSO-d₆): δ 7.79 (s, H), 7.71-7.22 (m, 4H), 7.2 (s, 1H), 3.89 (s, 3H), 2.51 (s, 3H); Mass (CI method, i-butane): 286 (M⁺, 10%); IR: νₘₐₓ (KBr, cm⁻¹): 1666.

### Step 5: Preparation of 6-(4-fluoro Phenyl)-1,3-dimethy-1,5-dihydro pyrazolo[4,3-c]pyridine-4-one (18)

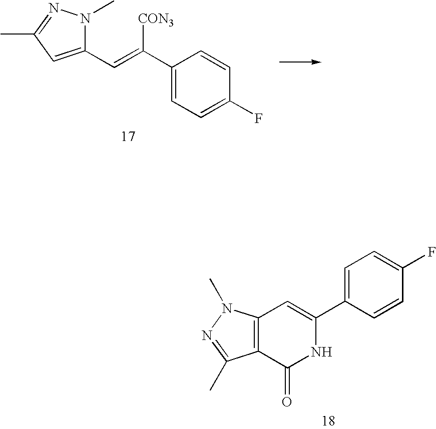

A mixture of compound 17 (0.8 gram, 2.8 mmol) and tributylamine in diphenyl ether (15 mL) was stirred at 250° C. for 30 minutes under nitrogen atmosphere and then diphenyl ether was distilled out at the same temperature. The cooled residue was dissolved in toluene (30 mL) and recrystallized with ethyl acetate to afford the title compound 6-(4-fluoro Phenyl)-1,3-dimethy-1,5-dihydro pyrazolo[4,3-c]pyridine-4-one (18) (0.46 gram) as an off white solid.

Yield : 64%; ¹H NMR (200 MHz, DMSO-d₆): δ 11.09 (s, NH), 7.85-7.78 (m, 2H), 7.37-7.28 (m, 2H), 6.84 (s, 1H), 3.97 (s, 3H), 2.54 (s, 3H); Mass (CI method, i-butane): 258 (M⁺, 100%); IR: νₘₐₓ (KBr, cm⁻¹): 3443, 1672.

### Step 6: Preparation of 4-chloro-6-(4-fluoro phenyl)-1,3-dimethy-1H-pyrazolo[4,3-c]pyridine (19)

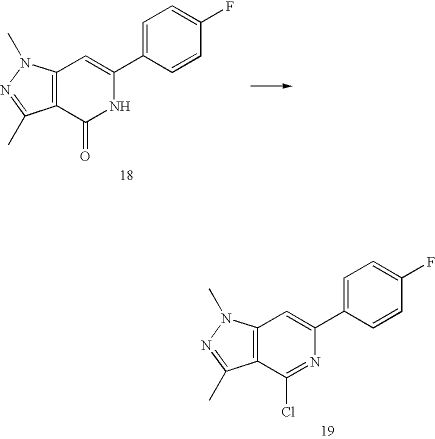

A mixture of compound 18 (0.46 gram, 1.78 mmol) and POCl₃ (10 mL) was stirred at refluxing temperature for 12 hours. The excess of POCl₃ was then distilled out at same temperature. The mixture was diluted with water and neutralized with sodium bicarbonate solution. The solid precipitated was dried under vacuum to afford the title compound 4-chloro-6-(4-fluoro phenyl)-1,3-dimethy-1H-pyrazolo[4,3-c]pyridine; (19) 0.43 gram as an off white solid.

Yield: 100%; ¹H NMR (200 MHz, DMSO-d₆): δ 8.22-8.20 (m, 2H), 7.34 (s, H), 7.39-7.30 (d, J=8.4 Hz, 2H), 4.0 (s, 3H), 2.64 (s, 3H); Mass (CI method, i-butane): 276 (M⁺, 10%); IR: νₘₐₓ (KBr, cm⁻¹): 1610.

### Step 7: Preparation of (3-chloro -4-methoxy phenyl)-[6-(4-fluoro-phenyl)-1,3-dimethy-1H-pyrazolo[4,3-C]pyridine-4yl]amine hydrochloride (E 72)

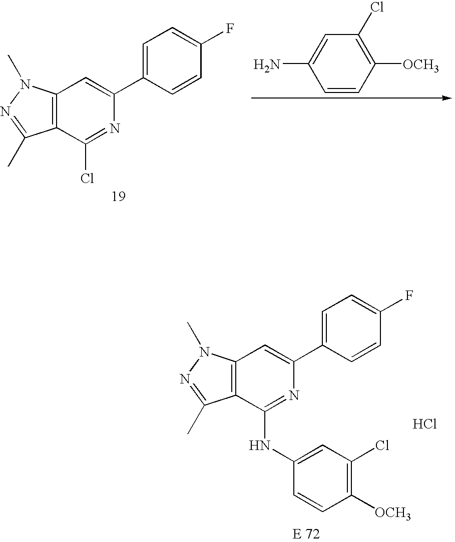

A mixture of compound 19 (0.2 gram, 0.83 mmol) and 3-chloro-4-methoxyaniline (0.18 gram, 1.14 mmol) in n-butanol (10 mL) was stirred at refluxing temperature for 36 hours under nitrogen atmosphere. The reaction mixture was then cooled to room temperature. The solid precipitated was filtered and dried under vacuum to afford the title compound (3-chloro-4-methoxy phenyl)-[6-(4-fluoro-phenyl)-1,3-dimethy-1H-pyrazolo[4,3-C]pyridine-4yl]amine (E 72) 0.2 gram as a off white solid.

Yield: 71%; ¹HNMR: (200 MHz, DMSO-d₆): 8.18-8.15 (m, 2H), 8.11 (s, NH), 8.0 (s, H), 7.72 (d, J=8.7 Hz, H), 7.60 (s, 1H), 7.33-7.20 (m, 2H), 7.15 (d, J=8.7 Hz, 1H), 3.95 (s, 3H), 3.85 (s, 3H), 2.7 (s, 3H); Mass (CI method, i-butane): 397(M⁺, 100%); IR: νₘₐₓ (KBr, cm⁻¹): 3450, 1613.

### Examples 73-78

Unless otherwise indicated, the following compounds presented in Examples 73-78 were prepared by a procedure analogous to that disclosed in Example 70, using analogous starting materials with the appropriate substitution, to afford the corresponding compounds, listed as compounds E 73 through E 78.

### Example 73

### Preparation of (3-fluoro-4-methoxy-phenyl)-[6-(4-fluoro-phenyl)-1,3-dimethyl-1H-pyrazolo[4,3-c]pyridin-4-yl]-amine hydrochloride (E 73)

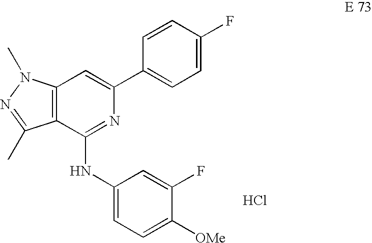

Yield: 44% ; ¹H NMR: (200 MHz, DMSO-d₆): 8.05 (m, 2H), 7.74 (d, J=14.6 Hz, 1H), 7.62 (s, 1H), 7.47-7.14 (m, 4H), 3.97 (s, 3H), 3.85 (s, 3H), 2.64 (s, 3H); Mass (CI method, i-butane): 381(M⁺, 100%); IR: νₘₐₓ (KBr, cm⁻¹): 3433.

### Example 74

### Preparation of 6-(4-fluoro-phenyl)-1,3-dimethyl-1H-pyrazolo[4,3-c]pyridin-4-yl]-(3-trifluoromethyl-phenyl)-amine hydrochloride (E 74)

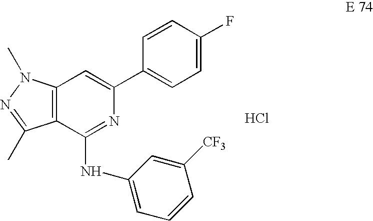

This compound was prepared at 120° C. for 24 h, using a procedure analogous to that disclosed in Example 70.

Yield: 74%; Melting point: 220-222° C.; Purity: 99.49%; ¹H NMR (400 MHz, DMSO-d₆): δ 8.48-8.44 (d, J=14.5, 2H), 8.19-8.15 (m, 2H), 7.99-7.97 (d, J=8.06, 1H), 7.71 (s, 1H), 7.59-7.55 (t, J=8.03, 1H), 7.34-7.25 (m, 2H), 3.97 (s, 3H), 2.74 (s, 3H); MS: 401 (M⁺, 100%); IR (cm⁻¹): 3451.8.

### Example 75

### Preparation of (6-chloro-pyridin-3-yl)-[6-(4-fluoro-phenyl)-1,3-dimethyl-1H-pyrazolo[4,3-c]pyridin-4-yl]-amine hydrochloride (E 75)

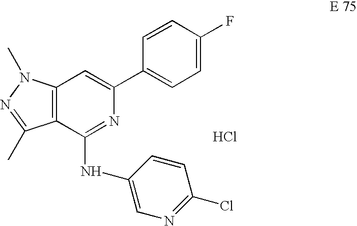

This compound was prepared at 120° C. for 24 hours, using a procedure analogous to that disclosed in Example 70.

Yield: 45%; Melting point: 215-218° C.; Purity: 94.18%; ¹H NMR (400 MHz, DMSO-d₆): δ 8.87 (bs, 1H), 8.43 (s, 1H), 8.27 (d, J=8.9, 1H), 8.14-8.10 (m, 2H), 7.79 (s, 1H), 7.50-7.48 (d, J=8.6, 1H), 7.34-7.28 (m, 2H), 3.97 (s, 3H), 2.72 (s, 3H); MS: 368 (M⁺, 100%); IR (cm⁻¹): 3417.9.

### Example 76

### Preparation of N-(4-[6-(4-fluoro-phenyl)-1,3-dimethyl-1H-pyrazolo[4, 3-c]pyridin-4-ylamino]-phenyl}-methanesulfonamide hydrochloride (E 76)

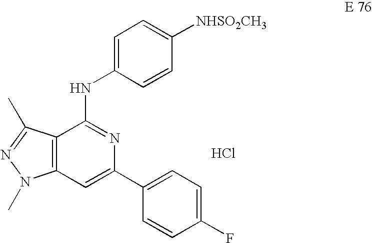

This compound was prepared at 80° C. for 24 h, using a procedure analogous to that disclosed in Example 70.

Yield: 20%; Purity: 99.22%; ¹H NMR (400 MHz, DMSO-d₆): δ 9.56 (bs, —NH), 8.09-8.00 (m, 2H), 7.72 (d, J=8.7, 2H), 7.60 (s, 1H), 7.32 (t, J=8.3, 2H), 7.24 (d, J=8.3, 2H), 3.96 (s, 3H), 2.97 (s, 3H), 2.62 (s, 3H); MS: 425 (M⁺, 100%); IR (cm⁻¹): 3440.6, 1634.6

### Example 77

### Preparation of (1,3-dimethyl-6-(4-fluoro phenyl)-1H-pyrazolo[4,3-c]pyridin-4-yl)-(4-trifluoromethoxy-phenyl)-amine hydrochloride (E 77)

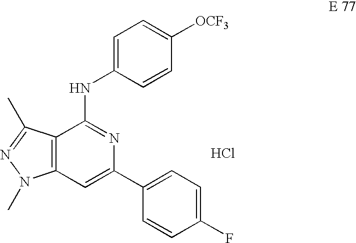

This compound was prepared at 120° C. for 24 hours, using a procedure analogous to that disclosed in Example 70.

Yield: 38%. Melting point: 248-250° C.; Purity: 98.92%; ¹H NMR (400 MHz, DMSO-d₆): δ 8.09 (t, J=6.2, 2H), 7.87 (d, J=8.8, 2H), 7.68 (s, 1H), 7.38-7.30 (m, 4H), 4.27 (bs, —NH), 3.98 (s, 3H), 2.65 (s, 3H); MS: 416 (M⁺, 100%); IR (cm⁻¹): 2939.9.

### Example 78

### Preparation of 4-[6-(4-fluoro-phenyl)-1,3-dimethyl-1H-pyrazolo[4,3-c]pyridin-4-ylamino]-N-methyl-benzenesulfonamide hydrochloride (E 78)

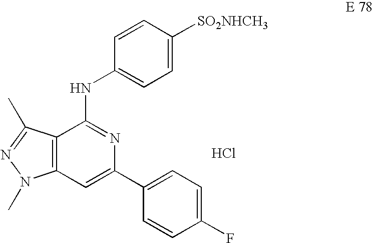

This compound was prepared at 80° C. for 48 h, using a procedure analogous to that disclosed in Example 70.

Yield: 16%; Melting point: 293-295° C.; Purity: 99.42%; ¹H NMR (400 MHz, DMSO-d₆): δ 8.63 (bs, 1H), 8.40-8.12 (m, 2H), 7.98 (d, J=8.6, 2H), 7.75 (d, J=4.3, 2H), 7.32 (t, J=8.4, 2H), 7.23 (s, 1H), 3.98 (s, 3H), 2.72 (s, 3H), 2.43 (s, 3H); MS: 426 M⁺+1, 100%); IR (cm−¹): 3444.0.

### Example 79

### Preparation of (3-chloro-4-methoxy-phenyl)-(1,6-diphenyl-1H-pyrazolo[3,4-d]pyrimidin-4-yl)-amine hydrochloride (E 79)

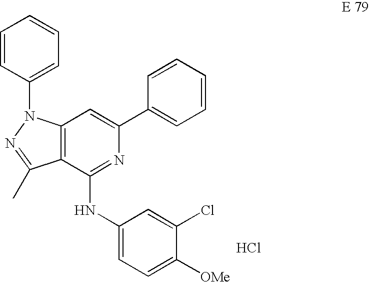

### Step 1: Preparation of 5-amino-3-methyl-1-phenyl-1H-pyrazole-4-carboxylic acid ethyl ester (20)

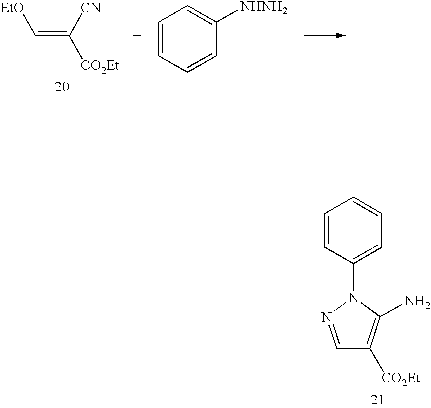

To a solution of compound 20 (7 grams, 41.4 mmol) in ethanol (70 mL) was added phenyl hydrazine (4.4 grams, 41.4 mmol) and the resulting reaction mixture was refluxed for 24 hours under nitrogen atmosphere. Then mixture was cooled to room temperature and concentrated under reduced pressure to afford the title compound 5-(4-fluoro-benzoylamino)-1-phenyl-1H-pyrazole-4-carboxylicacid ethyl ester (21) 8 grams as an off white solid.

Yield: 84%; ¹H NMR (200 MHz, CDCl₃): δ 7.78 (s, H), 7.53-7.50 (m, 5H), 5.3 (s, 2H, D₂O exchangeable), 4.35-4.25 (m, 2H), 1.40-1.32 (t, J=7.3Hz, 3H).

Mass (CI method, i-butane): 232 (M⁺, 100%).

IR: νₘₐₓ (KBr, cm⁻¹): 3396,1683.

### Step 2: Preparation of: 5-(4-fluoro-benzoylamino)-1-phenyl-1H-pyrazole-4-carboxylicacid ethyl ester (22)

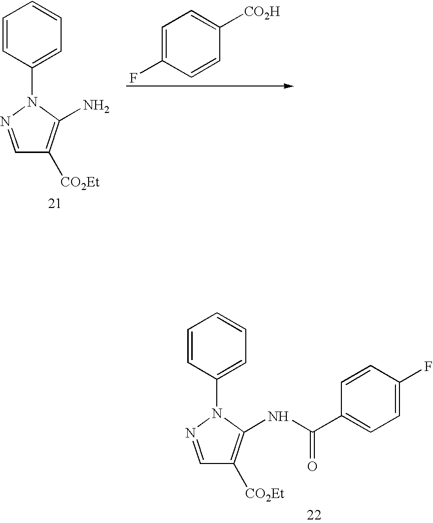

A mixture of compound 21 (4 grams, 17.3 mmol), 4-fluorobenzoic acid (4.8 grams, 34.6 mmol) and dimethylaminopyridine (DMAP) (1.05 grams, 8.6 mmol) in dichloromethane (100 mL) was cooled to 0° C. and dicyclohexyl-carbodiimide (DCC) (7 grams, 34.6 mmol) was added in two to three portions under nitrogen atmosphere. The resulting reaction mixture was stirred at refluxing temperature for 16 hours and then cooled to room temperature. Water was added to the mixture, the separated organic layer was collected, dried over anhydrous Na₂SO₄ and concentrated under reduced pressure. The residue thus obtained was passed through the silica gel to afford the title compound 5-(4-fluoro-benzoylamino)-1-phenyl-1H-pyrazole-4-carboxylicacid ethyl ester (22) 4.5 grams as off white solid.

Yield :74%; ¹H NMR (200 MHz, CDCl₃): δ 9.3 (s, H), 8.02 (s, H), 7.90-7.83 (m, 2H), 7.57-7.09 (m, 7H), 4.38-4.2 (m, 2H), 1.39-1.32 (t, J=7.3Hz, 3H); Mass (CI method, i-butane): 354 (M⁺, 100%); IR: νₘₐₓ (KBr, cm⁻¹): 1716, 1678.

### Step 3: Preparation of 5-(4-fluoro-benzoylamino)-1-phenyl-1H-pyrazole-4-carboxylicacid (23)

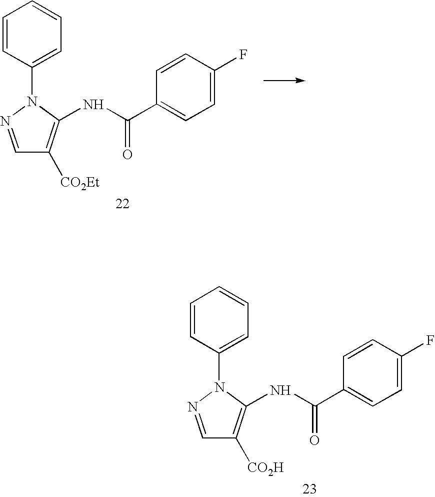

To a solution of compound 22 (4.5 grams, 12.7 mmol) in 1,4-dioxane (100 mL) was added 10% sodium hydroxide solution (2.5 grams, 63.7 mmol in 25 mL) and the resulting reaction mixture was stirred at 60° C. for 5 hours. The reaction mixture was cooled to room temperature and concentrated under reduced pressure. The residue (white solid) obtained was dissolved in water and washed with ethyl acetate. The aqueous layer was neutralized with 2N hydrochloric acid. The solid precipitated was filtered and dried under vacuum to afford the title compound 5-(4-fluoro-benzoylamino)-1-phenyl-1H-pyrazole-4-carboxylicacid (23) 4 grams as an off white solid.

Yield : 97%; ¹H NMR (200 MHz, DMSO-d₆): δ 12.5 (s D₂O exchangeable), 10.5 (s D₂O exchangeable), 8.12 (s, H), 7.98-7.91 (m, 2H), 7.67-7.32 (m, 7H); Mass (CI method, i-butane): 322 (M⁺, 10%); IR: νₘₐₓ (KBr, cm⁻¹): 3220, 1669, 1601.

### Step 4: Preparation of 5-(4-fluoro-benzoylamino)-1-phenyl-1H-pyrazole-4-carbonyl chloride (24)

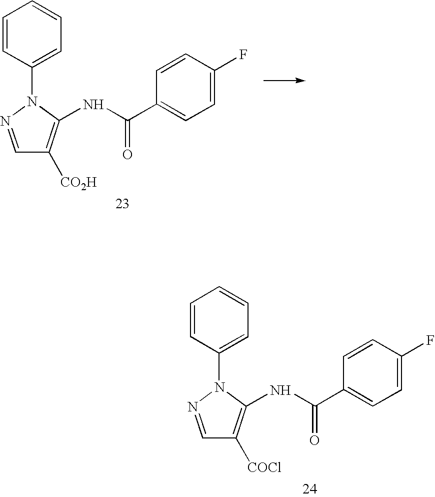

To a suspension of compound 23 (4 grams, 12.3 mmol) in ethyl acetate (50 mL) was added thionyl chloride (7.1 mL, 98.4 mmol) at 0° C. and the resulting reaction mixture was stirred at refluxing temperature for 16 hours. The reaction mixture was cooled to room temperature and solvent removed under reduced pressure to afford the title compound 5-(4-fluoro-benzoylamino)-1-phenyl-1H-pyrazole-4-carbonyl chloride (24) 3.5 grams as an off white solid.

Yield: 83%; ¹H NMR (200 MHz, DMSO-d₆): δ 8.55 (s, H), 8.32-8.25 (m, 2H), 8.06 (d, J=7.9 Hz, 2H), 7.69-7.61 (m, 2H), 7.52-7.43 (m, 3H); Mass (CI method, i-butane): 344 (M⁺, 10%); IR: νₘₐₓ (KBr, cm⁻¹): 1788, 1574.

### Step 5: Preparation of 5-(4-fluoro-benzoylamino)-1-phenyl-1H-pyrazole-4-carboxylic acid amide (25)

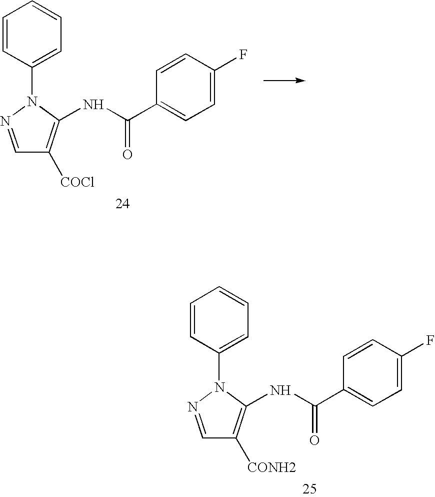

To a solution of compound 24 (3.5 grams, 10.2 mmol) in dioxane (100 mL) was added ammonia solution (100 mL) at 0° C. and the reaction mixture was stirred at the same temperature for 16 hours. The water was added to the mixture, the solid precipitated was filtered off and dried under vacuum to afford the title compound 5-(4-fluoro-benzoylamino)-1-phenyl-1H-pyrazole-4-carboxylic acid amide (25) 1.3 grams as off white solid.

Yield: 40%; ¹H NMR (200 MHz, DMSO-d₆): δ 8.39 (s, H), 8.03-7.88 (m, 3H), 7.66-7.53 (m, H), 7.38-7.15 (m, 5H), 6.8 (s, D₂O exchangeable), 6.28 (s, D₂O exchangeable).

Mass (CI method, i-butane): 326 (M⁺, 10%); IR: νₘₐₓ (KBr, cm⁻¹): 1664, 1597.

### Step 6: Preparation of 6-(4-fluoro-phenyl)-]-phenyl-1,5-dihydro-pyrazolo [3,4-d]-pyrimidin-4-one (26)

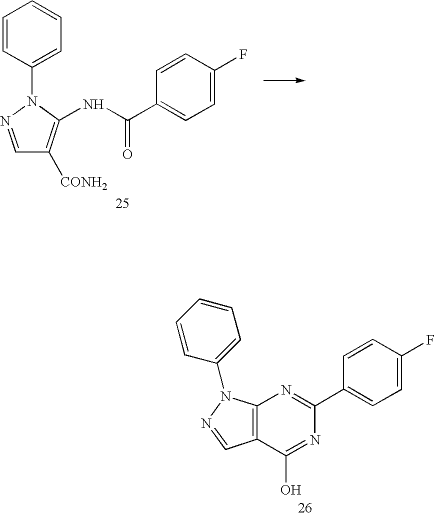

To a suspension of compound 25 (1.3 grams, 4.0 mmol) in t-butanol (20 mL) was added t-BuOK (1.35 grams, 12.0 mmol) and the resulting reaction mixture was stirred at refluxing temperature for 20 hours under nitrogen atmosphere. The reaction mixture was cooled to room temperature and concentrated under vacuum. The white solid obtained was dissolved in water, neutralized with 2N HCl, filtered and dried to afford the title compound 6-(4-fluoro-phenyl)-1-phenyl-1,5-dihydro-pyrazolo [3,4-d]-pyrimidin-4-one (26) 1 gram as a pale brown solid.

Yield: 82%; ¹H NMR (200 MHz, DMSO-d₆): δ 12.66 (s D₂O exchangeable), 8.36 (s, H), 8.29-8.11 (m, 4H), 7.63-7.38 (m, 5H); Mass (CI method, i-butane): 307 (M⁺, 10%).

IR: νₘₐₓ (KBr, cm⁻¹): 1691.

### Step 7: Preparation of 4-chloro-6-(4-fluoro-phenyl)-1-phenyl-1H-pyrazolo[3,4-d]-pyrimidin (27)

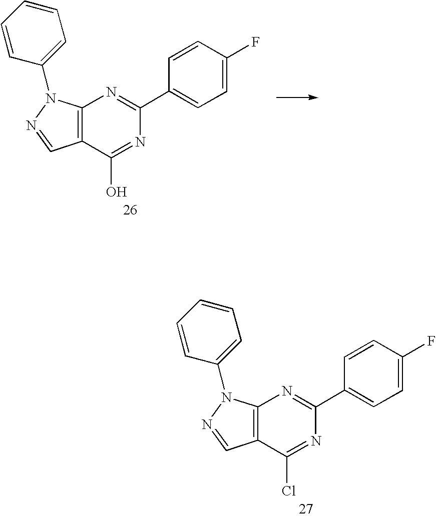

A mixture of compound 26 (1 gram, 3.2 mmol) and POCl₃ (15 mL) was stirred at refluxing temperature for 12 hours. The excess of POCl₃ was then distilled out at the same temperature. The mixture was diluted with water and neutralized with sodium bicarbonate solution. The solid precipitated was dried under vacuum to afford the title compound 4-chloro-6-(4-fluoro-phenyl)-1-phenyl-1H-pyrazolo [3,4-d]-pyrimidin (27) 0.7 gram as an off white solid.

Yield: 66%.

### Step 8: Preparation of (3-chloro-4-methoxy phenyl)-[6-(4-fluoro-phenyl)-1-phenyl-1H-pyrazolo[3,4-d]-pyrimidin-4yl]amine hydrochloride (E 79)

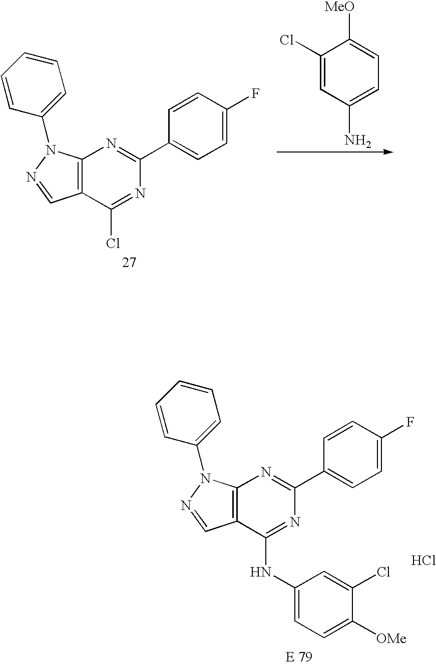

A mixture of compound 27 (0.15 gram, 0.46 mmol) and 3-chloro-4-methoxyaniline (0.109 gram, 0.69 mmol) in n-butanol (10 mL) was stirred at refluxing temperature for 36 hours under nitrogen atmosphere. The reaction mixture was then cooled to room temperature. The solid precipitated was filtered and dried under vacuum to afford the title compound (3-chloro-4-methoxy phenyl)-[6-(4-fluoro-phenyl)-1-phenyl-1H-pyrazolo [3,4-d]-pyrimidin-4yl]amine (E 79) 0.18 gram (as an off-white solid.

Yield: 85%; ¹H NMR: (200 MHz, DMSO-d₆): δ 10.3 (s, NH), 8.5-8.42 (m, 3H), 8.30 (d, J=7.9 Hz, 2H), 8.12 (s, 1H), 7.8 (d, J=8.9 Hz, H), 7.66-7.25 (m, 6H), 3.9 (s, 3H).

Mass (CI method, i-butane): 446 (M⁺, 100%); IR: νₘₐₓ (KBr, cm⁻¹): 3418.

### Examples 80-85

Unless otherwise indicated, the following compounds presented in Examples 80-85 were prepared by a procedure analogous to that disclosed in Example 79, using analogous starting materials with the appropriate substitution, to afford the corresponding compounds, listed as compounds E 80 through E 85.

### Example 80

### Preparation of (3-fluoro-4-methoxy phenyl)-[6-(4-fluoro-phenyl)-1-phenyl-1H-pyrazolo[3,4-d]-pyrimidin-4yl]amine hydrochloride (E 80)

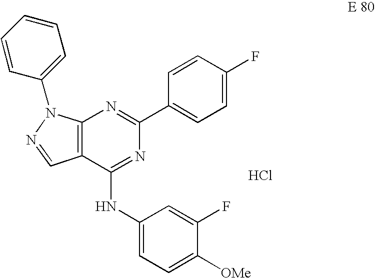

This compound was prepared by refluxing (1-butanol) for 16 h, using a procedure analogous to that disclosed in Example 79.

Yield: 64%; ¹H NMR (200 MHz, DMSO-d₆): δ 10.3 (s, NH), 8.5-8.42 (m, 3H), 8.30 (d, J=7.9 Hz, 2H), 7.88 (d, J=13.3 Hz, H), 7.62-7.2 (m, 7H), 3.88 (s, 3H); Mass (CI method, i-butane): 430 (M⁺, 100%); IR: νₘₐₓ (KBr, cm⁻¹): 3391.

### Example 81

### Preparation of (4-chloro-3-trifluoromethyl-phenyl)-[6-(4-fluoro-phenyl)-1-phenyl-1H-pyrazolo[3,4-d]pyrimidin-4-yl]-amine hydrochloride (E 81)

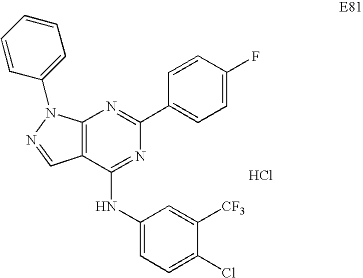

Yield: 61%; Melting point: 203.83° C.; Purity: 99.53%; ¹H NMR (400 MHz, CDCl₃): δ 8.53-8.47 (m, 3H), 8.33-8.30 (m, 2H), 8.0 (s,1H), 7.78-7.75 (m, 1H), 7.59-7.51 (m, 3H), 7.26-7.22 (m, 2H); MS: 484 (M⁺+1, 100%); IR (cm⁻¹): 3430.4.

### Example 82

### Preparation of (1,3-dimethyl-5-phenyl-1H-pyrazolo[4,3-d]pyrimidin-7-yl)-(2-methyl-1H-benzoimidazol-5-yl)-amine hydrochloride (E 82)

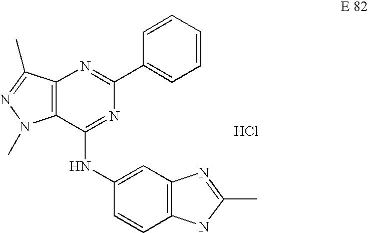

This compound was prepared at 120° C. for 24 h, using a procedure analogous to that disclosed in Example 79.

Yield: 63%; Purity: 99.80%; ¹H NMR (400 MHz, DMSO-d₆): δ 9.31 (bs, —NH), 8.43-7.91 (m, 3H), 7.90 (d, J=7.0, 1H), 7.85 (d, J=7.1, 1H), 7.80-7.44 (m, 3H), 4.36 (s, 3H), 3.44 (s, 3H), 2.83 (s, 3H); MS: 369 (M⁺, 100%); IR (cm⁻¹): 3453.1

### Example 83

### Preparation of (3-fluoro-phenyl)-[6-(4-fluoro-phenyl)-1-phenyl-1H-pyrazolo[3,4-d]pyrimidin-4-yl]-amine hydrochloride (E 83)

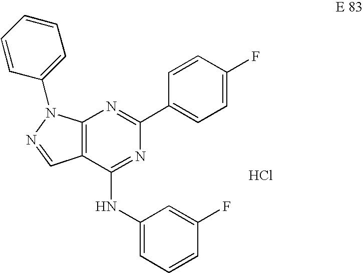

This compound was prepared at 120° C. for 24 h, using a procedure analogous to that disclosed in Example 79.

Yield: 54%; Melting point: 210-213° C.; Purity: 95.83%; ¹H NMR (400 MHz, DMSO-d₆): δ 10.45 (bs, —NH), 8.60 (s, 1H), 8.50-8.47 (m, 2H), 8.32-8.30 (d, J=8.3, 2H), 7.98-7.95 (d, J=11.8, 1H), 7.70-7.38 (m, 7H), 7.02-7.0 (m, 1H); MS: 399 (M⁺, 100%); IR (cm⁻¹): 3422.4

### Example 84

### Preparation of [6-(4-fluoro-phenyl)-1-phenyl-1H-pyrazolo[3,4-d]pyrimidin-4-yl]-(4-trifluoromethoxy-phenyl)-amine hydrochloride (E 84)

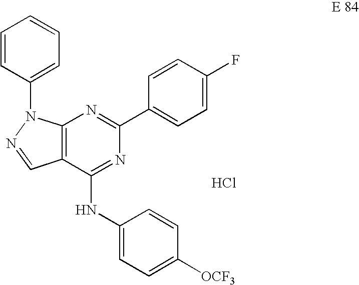

This compound was prepared at 120° C. for 24 h, using a procedure analogous to that disclosed in Example 79.

Yield: 47%; Melting point: 220-222° C.; Purity: 95.04%; ¹H NMR (400 MHz, DMSO-d₆): δ 10.44 (bs, —NH), 8.59 (s, 1H), 8.51-8.47 (m, 2H), 8.32 (m, 2H), 8.09-8.06 (m, 2H), 7.65-7.61 (m, 2H), 7.48-7.42 (m, 2H), 7.40-7.36 (m, 3H); MS: 465 (M⁺, 100%); IR (cm⁻¹): 3377.2

### Example 85

### Preparation of N-[4-(1,3-dimethyl-5-phenyl-1H-pyrazolo[4,3-d]pyrimidin-7-ylamino)-phenyl]-methanesulfonamide hydrochloride (E 85)

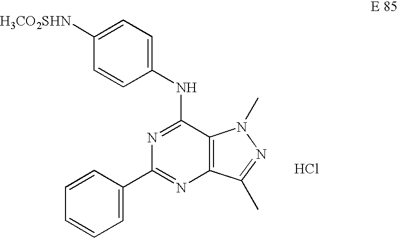

This compound was prepared at 120° C. for 4 h, using a procedure analogous to that disclosed in Example 79.

Yield: 60%; Purity: 99.39%.; ¹H NMR (400 MHz, DMSO-d₆): δ 9.73 (bs, —NH), 7.31 (bs, —NH), 8.27-8.23 (m, 2H), 7.78-7.77 (m, 2H), 7.57-7.47 (m, 3H), 7.33-7.29 (m, 2H), 4.33 (s, 3H), 3.01 (s, 3H), 2.52 (s, 3H); MS: 409 (M⁺+1, 100%); IR (cm⁻¹): 3221.3, 1622.8.

### Example 86

### Preparation of (3-fluoro-4-methoxy-phenyl)-[5-(4-fluoro-phenyl)-1-methyl-3-propyl-1H-pyrazolo[4,3-d]pyrimidin-7-yl]-amine (E 86)

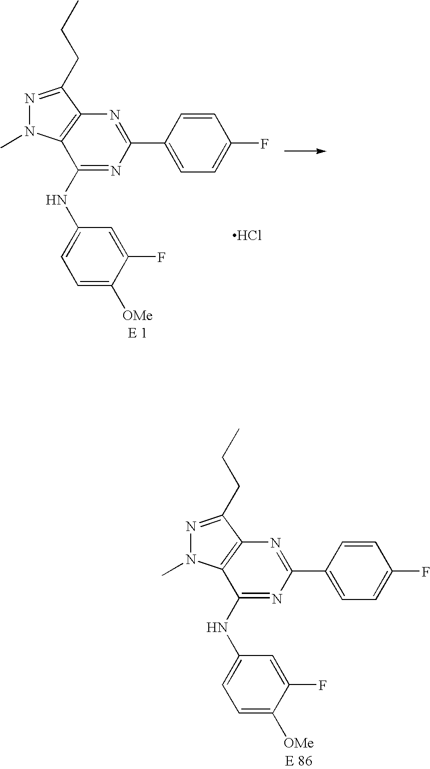

To a cold (10-15° C.) solution of compound E 1 (0.5 gram, 1.22 mmol) in acetic acid (40 mL) was added H₂O₂ (2 mL) dropwise with stirring. Stirring continued at the same temperature for 5 minutes. The mixture was then warmed to room temperature and diluted with cold water (50 mL). Solid precipitated was filtered, washed with water (2×20 mL) and dried under vacuum to afford the desired compound as a white solid (0.43 gram).

Yield: 94%.

### Example 87

### Preparation of (3-chloro-4-methoxy-phenyl)-(1-methyl-5-phenyl-3-propyl-H-pyrazolo[4,3-d]pyrimidin-7-yl)-amine (E 87)

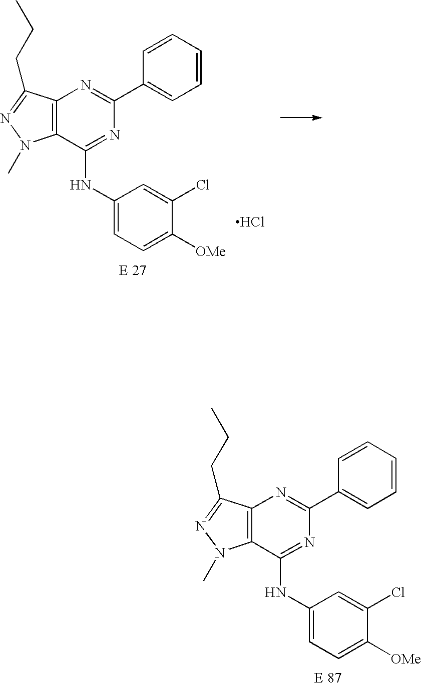

This compound was prepared by a procedure analogous to that disclosed in Example 86, using analogous starting materials with the appropriate substitution, to afford the corresponding compounds, E 87.

### Example 88

### Preparation of 5-(4-fluoro-phenyl)-7-indol-1-yl-1-methyl-3-propyl-1H-pyrazolo[4,3-d]pyrimidine (E 88)

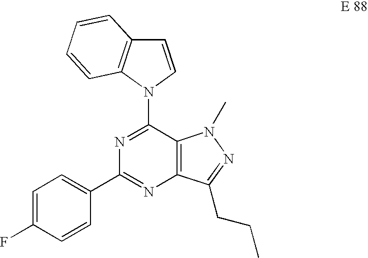

The title compound was prepared by reacting compound 5 (0.98 mmol) with indole (0.98 mmol) in dry DMF (10 mL for 1 gram of compound 5) in presence of NaH (1.48 mmol) at 0-80° C. for 24 h. The mixture was then cooled to room temperature, diluted with water (50 mL) and extracted with EtOAc (2×40 mL). Organic layers were collected, combined, washed with brine solution (35 mL) followed by water (2×30 mL), dried over anhydrous Na₂SO₄ and concentrated under vacuum. The residue thus obtained was purified by column chromatography using EtOAc-petroleum ether to give the desired compound.

Yield: 42%; Purity: 99.32%; Melting point: 114-116° C. ; ¹H NMR (400 MHz, CDCl₃): δ 8.57-8.52 (m, 2H), 7.75-7.71 (m, 2H), 7.62-7.61 (m, 1H), 7.34-7.26 (m, 2H), 7.17-7.13 (m, 2H), 6.86-6.35 (m, 1H), 3.81 (s, 3H), 3.12 (t, J=7.5, 2H), 2.05-1.95 (m, 2H), 1.12 (t, J=7.5, 3H); MS: 386(M+1, 100); IR (cm⁻¹): 3439,2955, 1601.

### Examples 89-90

Unless otherwise indicated, the following compounds presented in Examples 89-90 were prepared by a procedure analogous to that disclosed in Example 88, using analogous starting materials with the appropriate substitution, to afford the corresponding compounds, listed as compounds E 89 and E 90.

### Example 89

### Preparation of 7-(5-chloro-indol-1-yl)-5-(4-fluoro-phenyl)-]-methyl-3-propyl-1H-pyrazolo[4,3-d]pyrimidine (E 89)

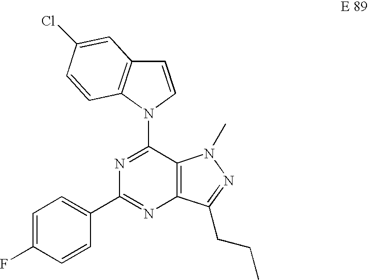

Yield: 15%; Melting point: 146-148° C.; Purity: 98.23%; ¹H NMR (400 MHz, CDCl₃): δ 8.54-8.51 (m, 2H), 7.71-7.70 (m, 1H), 7.66-7.62 (m, 2H), 7.30-7.26 (m, 1H), 7.18-7.14 (m, 2H), 6.80-6.79 (m, 1H), 3.81 (s, 3H), 3.12(t, J=7.5, 2H), 2.0 (q, J=7.5, 2H), 1.10 (t, J=7.2, 3H); MS: 420 (M⁺, 100%); IR (cm⁻¹): 3425, 2954, 1543

### Example 90

### Preparation of 7-indol-1-yl-1,3-dimethyl-5-phenyl-1H-pyrazolo[4,3-d]pyrimidine (E 90)

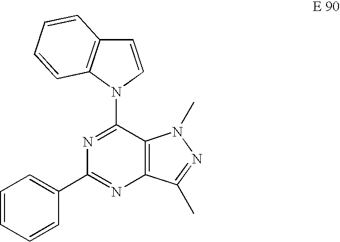

Yield: 61%; Melting point: 141-143° C.; Purity: 98.99%; ¹H NMR (400 MHz, CDCl₃): δ 8.56 (dd, J=1.9, 8.4, 2H), 7.75-7.74 (m, 2H), 7.73 (d, J=1.6, 1H), 7.72-7.30 (m, 5H), 6.85 (d, J=3.5, 1H), 3.81 (s, 3H), 2.75 (s, 3H); MS: 340 (M⁺+1, 100%); IR (cm⁻¹): 3423.4.

### Example 91

### Preparation of 5-chloro-3-phenyl-(-1H-pyrazolo[4,3-d]pyrimidin-7-yl)-(4-fluoro-phenyl)-amine hydrochloride (E 91)

### Step 1: Preparation of 4-Nitroso-5-phenyl-2H-pyrazole-3-carboxylic acid ethyl ester (29)

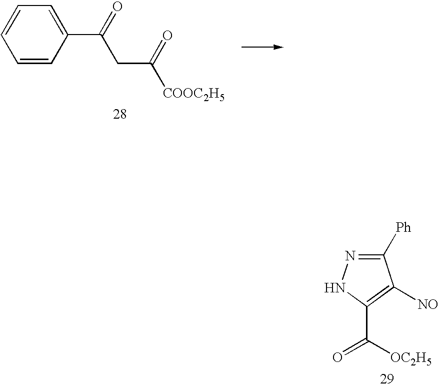

To a solution of HCl (1 mL) and CH₃COOH (5 mL) was added ethyl benzoyl acetate (28) (1 g, 4.5 mmol). To this was added NaNO₂ (0.31 g, 4.5 mmol) dissolved in 3 mL water dropwise at 0° C. This mixture was allowed to stand at room temperature for 20 min. To this mixture was added anhydrous hydrazine (0.22 mL, 4.5 mmol). The mixture was diluted with water (30 mL) and extracted with ethyl acetate (3×20 mL). Organic layers were collected, combined and concentrated to give the desired compound 29 (700 mg, 70%).

### Step 2: Preparation of 4-Amino-5-phenyl-2H-pyrazole-3-carboxylic acid ethyl ester (30)

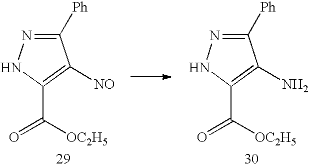

To a solution of 4-Nitroso-5-phenyl-2H-pyrazole-3-carboxylic acid ethyl ester 5 (29) (10 g, 40.8 mmol) in ethanol (300 mL) was added 10% Pd-C (7 gm) and the mixture was stirred at room temperature under hydrogen atmosphere (45 Psi H₂ atm) for 5 hrs. The mixture was filtered through Celite™ and concentrated under vacuum to give the desired compound 30(8 gm, 85% yield).

### Step 3: Preparation of 3-Phenyl-1H-pyrazolo[4,3-d]pyrimidine-5,7-diol (31)

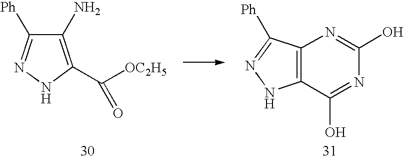

To a mixture of acetic acid (33 mL), water (3.25 mL) and 4-Amino-5-phenyl-2H-pyrazole-3-carboxylic acid ethyl ester (30) (1.5 grams, 6.49 mmol) was added a solution of KOCN (1.5 grams, 19.4 mmol) dissolved in water (5.19 mL) dropwise. The mixture was stirred at room temperature for 16 hrs. The solid seperated was filtered, dissolved in 6% NaOH solution and refluxed for 2 hrs. The mixture was then neutralized with 2N HCl and the solid separated was filtered to give the desired compound 31 (0.6 grams, 42%).

### Step 4: Preparation of 5,7-Dichloro-3-phenyl-1H-pyrazolo[4,3-d]pyrimidine (32)

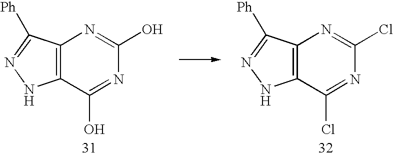

A mixture of 3-Phenyl-1H-pyrazolo[4,3-d]pyrimidine-5,7-diol (31) (0.6 grams, 2.6 mmol) and POCl₃ (10 mL) was refluxed for 60 hours and excess POCl₃ was removed under vacuum. The residue was treated with sodium bicarbonate solution and the solid separated was filtered to give the desired compound 32 (0.3 grams, 44%).

### Step 5: Preparation of 5-chloro-3-phenyl-(-1H-pyrazolo[4,3-d]pyrimidin-7-yl)-(4-fluoro-phenyl)-amine hydrochloride (E 91)

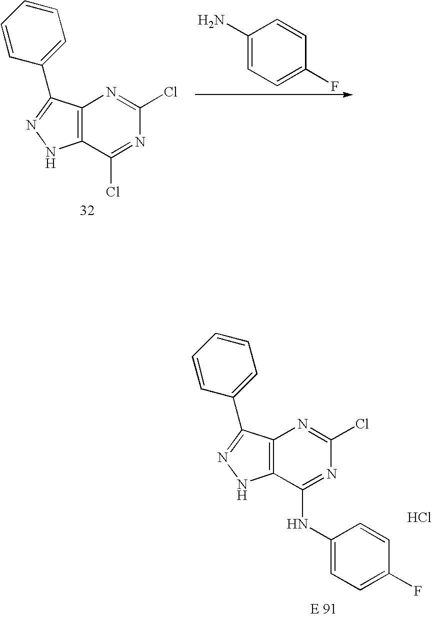

The title compound was prepared by reacting 5,7-dichloro-3-phenyl-1H-pyrazolo[4,3-d]pyrimidine (1.13 mmol) with 4-fluoro aniline (0.56 mmol) in n-butanol in presence of triethylamine (4.54 mmol) at 120° C. for 12 h. The solid separated was filtered and dried under vacuum to afford the desired product.

Yield: 17%; Melting point: 248-250° C.; Purity: 98.79%; ¹H NMR (400 MHz, CDCl₃): δ 8.33-8.31 (m, 1H), 8.23-8.21(m, 1H), 7.97 (s, 1H), 7.33-7.30 (m, 1H), 7.60-7.51 (m, 2H), 7.45-7.41 (m, 1H), 7.34-7.24 (m,2H); MS: 340 (M⁺, 100%); IR (cm⁻¹): 3451, 2929, 1631.

### Example 92

### Preparation of 4-benzo[1,3]dioxol-5-yl-6-(4-fluoro-phenyl)-1,3-dimethyl-1H-pyrazolo[4,3-c]pyridine (E 92)

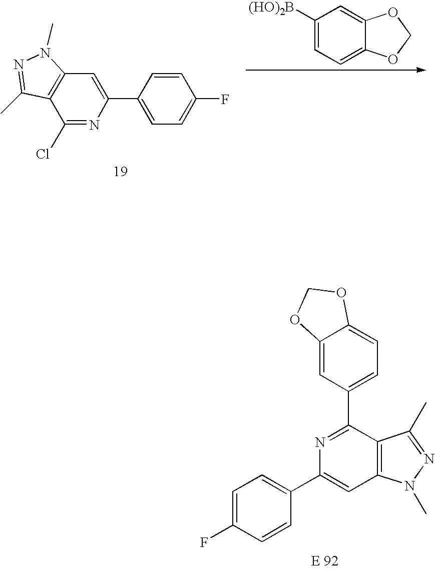

This compound was prepared by reacting compound 19 (0.54 mmol) with 1,3-benzodioxol-5-benzene boronic acid (0.78 mmol) in DMF (10 mL) in the presence of (PPh₃)₄Pd (0.1 mmol), 2N Na₂CO₃ solution (3 mL) at 80° C. for 6 h. The mixture was then cooled to room temperature, diluted with water (50 mL) and extracted with EtOAc (2×30 mL). Organic layers were collected, combined, washed with brine solution (35 mL) followed by water (2×30 mL), dried over anhydrous Na₂SO₄ and concentrated under vacuum. The residue thus obtained was purified by column chromatography using EtOAc-petroleum ether to give the desired compound.

Yield: 72%; Melting point: 166-168° C.; Purity: 93.95%; ¹H NMR (400 MHz, CDCl₃): δ 8.13-8.09 (m, 2H), 7.49 (s, 1H), 7.25-7.24 (m, 2H), 7.18-7.13 (m, 2H), 6.94 (d, J=8.1, 1H), 6.05 (s, 2H), 4.05 (s, 3H), 2.37 (s, 3H); MS: 362 (M⁺+1, 100%); IR (cm−¹): 1596.7, 1443.8

### Examples 93-94

Unless otherwise indicated, the following compounds presented in Examples 93-94 were prepared by a procedure analogous to that disclosed in Example 92, using analogous starting materials with the appropriate substitution, to afford the corresponding compounds, listed as compounds E 93 and E 94.

### Example 93

### Preparation of 6-(4-fluoro-phenyl)-4-(3-methanesulfonyl-phenyl)-1,3-dimethyl-1H-pyrazolo[4,3-c]pyridine (E 93)

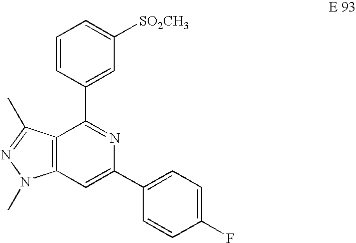

Yield: 56%; Melting point: 186-188; Purity: 99.23%; H NMR (400 MHz, DMSO-d₆): δ 8.31 (s, 1H), 8.12-8.02 (m, 2H), 7.75 (t, J=7.5, 2H), 7.58 (s, 1H), 7.17(t, J=8.3, 3H), 4.08 (s, 3H), 3.11 (s, 3H), 2.33 (s, 3H); MS: 395 (M⁺, 100%); IR (cm⁻¹): 2926.7, 1600.9

### Example 94

### Preparation of 6-(4-fluoro-phenyl)-1,3-dimethyl-4-(4-trifluoromethoxy-phenyl)-1H-pyrazolo[4,3-c]pyridine (E 94)

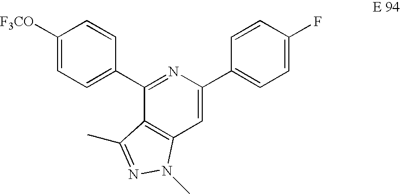

Yield: 52%; Melting point: 92-94° C.; Purity: 99.42%; ¹H NMR (400 MHz, CDCl₃): δ 8.12-8.09 (m, 2H), 7.76-7.73 (m, 2H), 7.54 (s, 1H), 7.37 (d, J=8.3, 2H), 7.16 (t, J=8.5, 2H), 4.07 (s, 3H), 2.32 (s, 3H); MS: 402 (M⁺+1, 100%); IR (cm⁻¹): 2933.7, 1602.2.

### Example 95

### Preparation of (4-chloro-3-trifluoromethyl-phenyl)-[6-(4-fluoro-phenyl)-1,3-dimethyl-1Hpyrazolo[4,3-c]pyridin-4-yl]-amine hydrochloride (E 95)

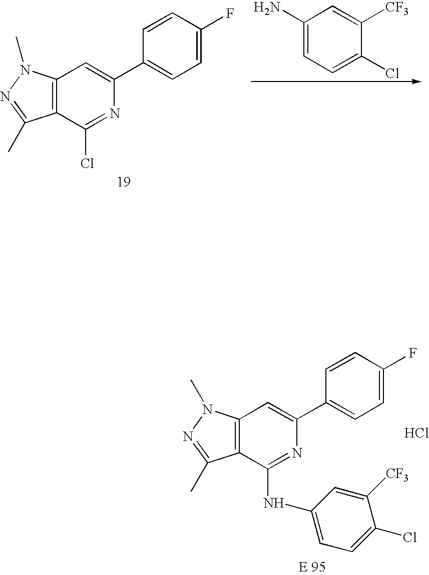

The title compound was prepared by reacting compound 19 (0.90 mmol) with 3-trifluoro methyl, 4-chloro aniline (0.90 mmol) in n-butanol (10 mL for 1 gram of 19) at 120° C. for 24 hours. The solid separated was filtered and dried under vacuum to afford the desired product.

Yield: 66%; Melting point: 223-225° C.; Purity: 99.57%; ¹H NMR (400 MHz, DMSO-d₆): δ 8.57 (s, 1H), 8.16-8.13 (m, 2H), 8.05-8.02 (m, 1H), 7.71 (s,1H), 7.67-7.65 (d, J=8.86, 1H), 7.30-7.26 (m, 2H), 3.97 (s, 3H), 2.73 (s, 3H); MS: 435 (M⁺, 100%); IR (cm⁻¹): 3448.9

### Example 96

### Preparation of [5-(4-fluoro-phenyl)-1,3-dimethyl-1H-pyrazolo[4,3-d]pyrimidin-7-yl]-(4-methanesulfonyl-phenyl)-amine (E 96)

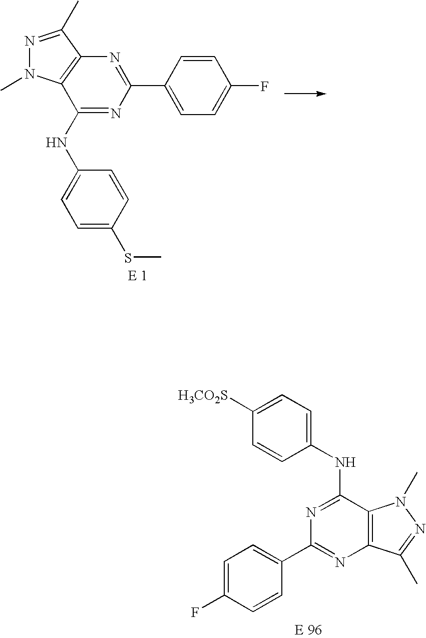

To a mixture of [5-(4-fluoro-phenyl)-1,3-dimethyl-1H-pyrazolo[4,3-d]pyrimidin-7-yl]-(4-methylsulfanyl-phenyl)-amine (0.27 g, 0.71 mmol) and oxone (1.31 grams, 2.13 mmol) in acetone (10 mL), water (4 mL) was added and the mixture was stirred for 20 min at room temperature under nitrogen atmosphere. After completion of the reaction the mixture was diluted with cold NaHCO₃ solution followed by water (10 mL) and was extracted with EtOAc (2×10 mL). Organic layers were collected, combined, dried over anhydrous Na₂SO₄ and concentrated under vacuum. The residue thus obtained was purified by column chromatography using EtOAc-hexane to give the desired compound.

Yield: 86%; Melting point: 216-218° C.; Purity: 93.10%; ¹H NMR (400 MHz, DMSO-d₆): δ 9.38 (bs, —NH), 8.41-8.36 (m, 2H), 8.07 (d, J=8.8, 2H), 7.98 (d, J=8.9, 2H), 7.32 (t, J=8.9, 2H), 4.31 (s, 3H), 3.22 (s, 3H), 2.50 (s, 3H); MS: 412 (M⁺+1, 100%); IR (cm⁻¹): 3423.0, 1600

### Example 97

### Preparation of (1,3-dimethyl-5-phenyl-1H-pyrazolo[4,3-d]pyrimidin-7-yl)-(2-methyl-benzooxazol-5-yl)-amine hydrochloride (E 97)

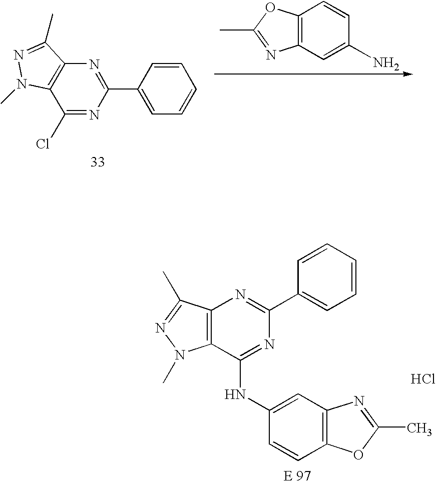

This compound was prepared by reacting compound 33 (0.38 mmol) with 2-methyl-benzooxazol-5-ylamine (0.40 mmol) in i-propanol (10 mL for 1 gram of 33) at 80° C. for 48 hours. The solid separated was filtered and washed with i-propanol. The solid thus obtained was stirred in i-propanol at 50-60° C. for 3-4 hours, filtered and dried under vacuum to afford the desired product.

Yield: 25%; Melting point: 272-274° C.; Purity: 97.35%; 1H NMR (400 MHz, DMSO-d₆): δ 9.53 (bs, —NH), 8.24-8.21 (m, 2H), 8.09 (s, 1H), 7.76-7.69 (m, 2H), 7.49-7.46 (m, 3H), 4.37( s, 3H), 2.65 (s, 3H), 2.54 (s, 3H); MS: 371 (M⁺, 50%); IR (cm−¹)L 3442.7

### Example 98

### Preparation of 6-(4-fluoro-phenyl)-4-(4-methanesulfonyl-phenyl)-1,3-dimethyl-1H-pyrazolo[4,3-c]pyridine (E 98)

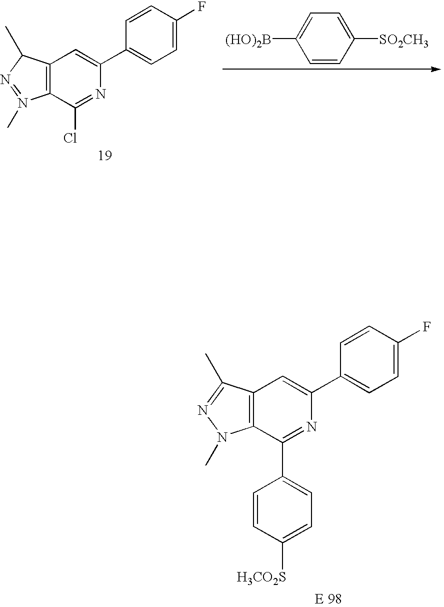

This compound was prepared by reacting compound 19 (0.72 mmol) with 4-methanesulphonyl benzene boronic acid (0.70 mmol) in DMF (10 mL) in the presence of (PPh₃)₄Pd (0.02 mmol), 2N Na₂CO₃ solution (3.5 mL) at 80° C. for 2 h. The mixture was then cooled to room temperature, diluted with water (50 mL) and extracted with EtOAc (2×30 mL). Organic layers were collected, combined, washed with brine solution (35 mL) followed by water (2×30 mL), dried over anhydrous Na₂SO₄ and concentrated under vacuum. The residue thus obtained was purified by column chromatography using EtOAc-petroleum ether to give the desired compound.

Yield: 49%; Melting point: 228-230° C.; Purity: 98.9%; MS: 395 (M⁺, 100); IR (cm−¹):2925.5, 1595

### Example 99

### Preparation of 7-fluoro-1,3-dimethyl-5-phenyl-1H-pyrazolo[4,3-d]pyrimidine (E 99)

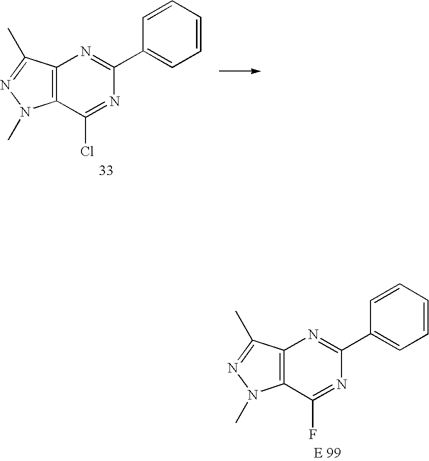

The title compound was prepared by reacting compound 33 (0.77 mmol) with potassium fluoride (4.65 mmol) in the presence of 2 drops of 18-crown-6-ether in acetonitrile (10 mL for 1 gram of 33) at 60° C. for 12 h. The mixture was cooled to room temperature and diluted with water. Solid separated was filtered and dried to give the desired product.

Yield: 21%; Melting point: 110-112° C.; Purity: 96.58%; ¹H NMR (200 MHz, CDCl₃): δ 8.49-8.45 ( m, 2H), 7.49-7.46 (m, 3H), 4.22 (s, 3H), 2.68 (s, 3H); MS: 242 (M⁺, 100%)

### Example 100

### Preparation of [6-(4-fluoro-phenyl)-1,3-dimethyl-1H-pyrazolo[4,3-c]pyridin-4-yl]-(4-methanesulfonyl-phenyl)-amine (E 100)

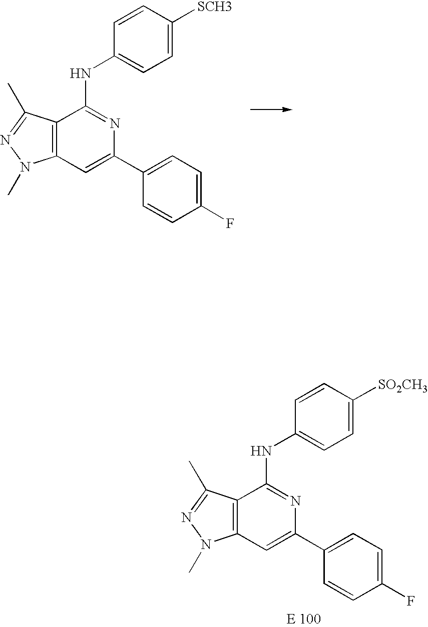

To a mixture of [6-(4-fluoro-phenyl)-1,3-dimethyl-1H-pyrazolo[4,3-c]pyridin-4-yl]-(4-methylsulfanyl-phenyl)-amine (0.20g, 0.53 mmol) and oxone (0.97 grams, 1.58 mmol) in acetone (10 mL), water (5 mL) was added and the mixture was stirred for 20 min at room temperature under nitrogen atmosphere. After completion of the reaction the mixture was diluted with cold NaHCO₃ solution followed by water (10 mL) and was extracted with EtOAc (2×10 mL). Organic layers were collected, combined, dried over anhydrous Na₂SO₄ and concentrated under vacuum. The residue thus obtained was purified by column chromatography using EtOAc-hexane to give the desired compound.

Yield: 23%; Purity: 96.40%; ¹H NMR (400 MHz, DMSO-d₆): δ 8.75 (bs, —NH), 8.21-8.17 (m, 2H), 8.01 (d, J=8.9, 2H), 7.87 (d, J=8.9, 2H), 7.77 (s, 1H), 7.32 (t, J=8.8,2H), 3.98 (s, 3H), 3.17 (s, 3H), 2.71 (s, 3H); MS: 411 (M⁺+1, 100%); IR (cm⁻¹): 3425.5.

### Example 101

### Preparation of 5-(4-fluoro-phenyl)-1,3-dimethyl-7-(4-trifluoromethoxy-phenyl)-1H-pyrazolo[4,3-d]pyrimidine (E 101)

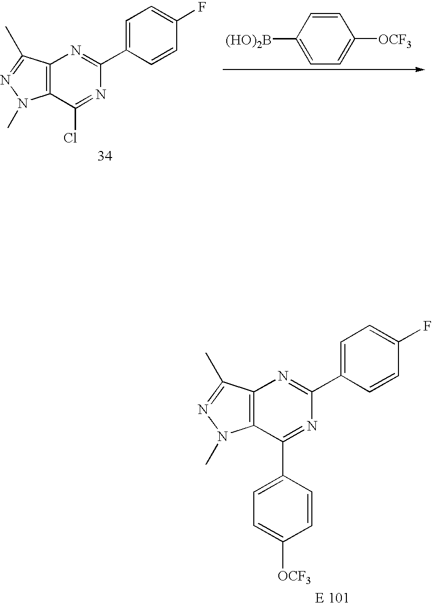

This compound was prepared by reacting compound 34 (0.72 mmol) with 4-trifluoromethoxy benzene boronic acid (0.72 mmol) in DMF (10 mL) in the presence of (PPh₃)₄Pd (0.026 mmol), 2N Na₂CO₃ solution (3 mL) at 80° C. for 2 h. The mixture was then cooled to room temperature, diluted with water (50 mL) and extracted with EtOAc (2×30 mL). Organic layers were collected, combined, washed with brine solution (35 mL) followed by water (2×30 mL), dried over anhydrous Na₂SO₄ and concentrated under vacuum. The residue thus obtained was purified by column chromatography using EtOAc-petroleum ether to give the desired compound.

Yield: 69%; Melting point: 170-172° C.; Purity: 99.45%; ¹H NMR (400 MHz, CDCl₃): δ 8.59-8.56 (m, 2H), 7.82 (dd, J=2.2, 6.8, 2H), 7.45 (dd, J=0.8, 8.6, 2H), 7.18-7.14 (m, 2H), 3.85 (s, 3H), 2.72 (s, 3H); MS: 403 (M⁺+1, 100%); IR (cm⁻¹): 2921.6, 1606.6

### Example 102

### Preparation of (1,3-dimethyl-5-phenyl-1H-pyrazolo[4,3-d]pyrimidin-7-yl)-dimethyl-amine (E 102)

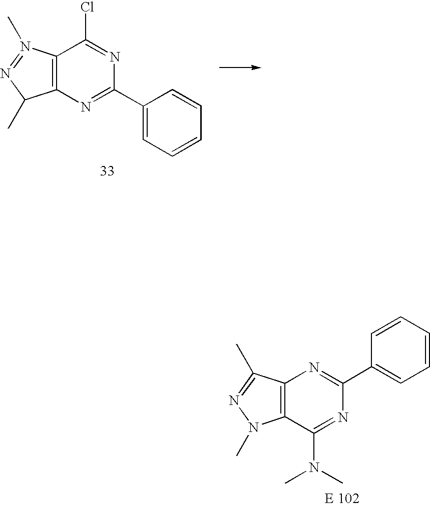

A mixture of 7-chloro-1,3-dimethyl-5-phenyl 1H-pyrazolo[4,3-d]pyrimidine (33) (0.2 gram, 0.83 mmol), 2M Na₂CO₃ solution (1 mL), dimethylformamide (DMF) (10 mL) in the presence of (PPh₃)₄Pd (0.04 gram, 0.4 mmol) was heated at 80° C. for 12 hours under atmosphere. After completion of the reaction the mixture was poured into cold water (50 mL) and extracted with ethyl acetate (3×20 mL), washed with water (2×20 mL), dried over anhydrous Na₂SO₄ and concentrated. The residue thus obtained was purified by column chromatography using ethyl acetate-petroleum ether to afford the title desired compound (1,3-dimethyl-5-phenyl-1H-pyrazolo[4,3-d]pyrimidin-7-yl)-dimethyl-amine (E 102).

Yield: 68%; Melting point: 86-88° C.; ¹HNMR (200 MHz, CDCl₃): δ 8.49 (d, J=7.3 Hz, 2H), 7.46-7.43 (m, 3H), 4.12 (s, 3H), 3.20 (s, 6H), 2.62 (s, 3H).

### Example 103

Determination of Smooth Muscle Cell Proliferation

Primary cultures of human aortic smooth muscle cells were obtained from Clonetics. SMC were initially grown in T-75 flasks prior to seeding in 96 well plates. The 96-well plates were seeded with 4000 cells/well. The following day cells were washed with serum free medium and left in serum free media for 24 hours for serum starvation. The next day cells received growth medium containing serum with or without compound. 24 h post treatment cell proliferation was assayed either assessing the incorporation of radiolabeled thymidine to DNA or using a non-radioactive cell proliferation kit from Promega (CellTiter AQ). Data are provided in the following table.

TABLE 13
Compound activity in smooth muscle cell proliferation assay

| Compound | Activity |
|---|---|
| E 33 | 50% inhibition at 0.54 μM |
| E 19 | 50% inhibition at 0.45 μM |
| E 52 | 50% inhibition at 0.5 μM |
| E 2 | 50% inhibition at 1.62 μM |
| E 1 | 50% inhibition at 0.89 μM |
| E 27 | 50% inhibition at 1.28 μM |
| E 28 | 70% inhibition at 5 μM |
| E 16 | 100% inhibition at 1 μM |
| E 17 | 100% inhibition at 1 μM |
| E 13 | 50% inhibition at 0.38 μM |
| E 14 | 50% inhibition at 0.43 μM |
| E 8 | 50% inhibition at 1 μM |
| E 37 | 50% inhibition at 0.5 μM |
| E 38 | 50% inhibition at 0.23 μM |
| E 39 | 50% inhibition at 0.33 μM |
| E 96 | 50% inhibition at 1.37 μM |
| E 43 | 50% inhibition at 0.93 μM |
| E 44 | 50% inhibition at 2.5 μM |
| E 10 | 96% inhibition at 1 μM |
| E 30 | 50% inhibition at 1.45 μM |
| E 32 | 50% inhibition at 0.94 μM |
| E 72 | 50% inhibition at 1.3 μM |
| E 73 | 50% inhibition at 0.9 μM |
| E 79 | 50% inhibition at 0.48 μM |
| E 80 | 50% inhibition at 0.46 μM |
| E 81 | 50% inhibition at 3.9 μM |

### Example 104

Inflammation Assays

For inflammation assays, human aortic endothelial cells (HAECS) in 96 well plates were washed once with treatment medium (basal medium containing 1% FBS). Cells were treated with an inflammatory agent such as TNFα (0.05 ng/ml) or glycated human serum albumin (US Biologicals) as source of advanced glycation end products (AGEs) (300 μg/ml) for 18-24 h in the presence or absence of specified amount of compound. Cell supernatants were collected and used for the estimation of MCP-1 (monocyte chemoattractant protein 1) or IL-6 (interleukin-6) by ELISA. Cell layers were washed and used for determining the levels of vascular cell adhesion molecule-1 (VCAM-1).

### Example 105

MCP-1 ELISA (Enzyme-Linked Immunosorbent Assay)

MCP-1 ELISA was carried out using Quantikine Human MCP-1 kit as described by the manufacturer (R&D Systems, Inc.). Mouse anti-human MCP-1 was used as the capture antibody and HRP (horse radish peroxidase)-conjugated goat anti-human MCP-1 antibody was used as detection antibody. Culture medium was incubated with the capture antibody (in 96-well plate) for 2 h at room temperature. Wells were washed three times with wash buffer (0.05% Tween-20 in PBS) followed by incubation with detection antibody for 2 h at room temperature. Color development was read at 45 nm in a microplate reader. Data are provided in the following table.

TABLE 14
Compound activity in MCP-1 enzyme-linked immunosorbent assay

| Compound | IC₅₀ in μM |
|---|---|
| E 52 | 8.7 |
| E 2 | 13.4 |
| E 1 | 7.2 |
| E 86 | 9 |
| E 27 | 5.7 |
| E 28 | 7.5 |
| E 13 | 1.5 |
| E 14 | 4 |
| E 8 | 8.7 |
| E 49 | 0.34 |
| E 50 | 0.42 |
| E 10 | 1.6 |
| E 72 | 3.8 |
| E 73 | 4.1 |
| E 100 | 0.48 |
| E 79 | 12.1 |
| E 80 | 5.4 |
| E 81 | 5.2 |

### Example 106

VCAM-1 ELISA

The cells were fixed cells with 100% methanol for 10 min at room temperature. The methanol was removed and the plate was air-dried. 100 ul of 1:1000 diluted primary antibody (polyclonal goat anti-human VCAM-1—R&D Systems #BBA19) was then added and incubated for 2 h at 37 C. The cells were washed with PBS and 100 ul of 1:5000 dilution of secondary antibody (rabbit anti-goat IgG-HRP—Zymed #81-1620) was added and incubated for 1 h at room temperature. Cells were washed and 100 ul of substrate solution (R&D Systems# DY999) was added and incubated for 20 min in the dark at room temp. 50 μl of stop solution (2N sulfuric acid) was added to the wells and absorbency at 450 nm was noted. Data are provided in the following table.

TABLE 15
Compound activity in VCAM-1 enzyme-linked immunosorbent assay

| Compound | IC₅₀ in μM |
|---|---|
| E 52 | 10.9 |
| E 2 | 10.4 |
| E 1 | 11.8 |
| E 86 | 12.7 |
| E 27 | 8.4 |
| E 28 | 8.4 |
| E 8 | 12.4 |
| E 49 | 0.61 |
| E 50 | 1.68 |
| E 10 | 11.8 |
| E 72 | 14 |
| E 73 | 13.7 |
| E 79 | 14.4 |
| E 80 | 11.1 |
| E 81 | 8.9 |

### Example 107

IL-6 ELISA

IL-6 levels in endothelial cell media were determined using DuaSet IL-6 ELISA kit from R&D Systems (Cat No DY206) as described by the manufacturer. Mouse anti-human IL-6 antibody was used as the capture antibody and biotinylated goat anti-human IL-6 was used as detection antibody. Culture medium was incubated with the capture antibody (in 96-well plate) for 2 h at room temperature. Wells were washed three times with wash buffer (0.05% Tween-20 in PBS) followed by incubation with detection antibody for 2 h at room temperature. The wells were then incubated with streptavidin HRP and color development was read at 450 nm in a microplate reader. Data are provided in the following table.

TABLE 16
Compound activity in IL-6 enzyme-linked immunosorbent assay

| Compound | IC₅₀ in μM |
|---|---|
| E 2 | 9 |
| E 1 | 5.4 |
| E 27 | 9.6 |

In another aspect of the present invention, this invention encompasses salts of the compounds disclosed herein, including pharmaceutically acceptable and non-pharmaceutically acceptable salts. It is envisioned that the compounds, compositions, and all the salts disclosed therein, including the non-pharmaceutically acceptable salts, can have uses and applications beyond pharmaceutical applications. For example, the pyrimidine compounds and compositions comprising pryimidine compounds of this invention can be used in a variety of agricultural uses or applications such as herbicides and pesticides, hardness stabilizers in rubber processing, ultraviolet light absorbers, and other uses.

We claim:

1. A compound having the formula: 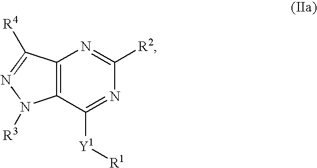
or a salt, a prodrug, a diastereomeric mixture, an enantiomer, a tautomer, a racemic mixture, or any combination thereof, wherein:
Y¹ is >NR⁵, —C≡C—, >O, or a direct a bond between the 6-membered ring and R¹;
wherein when Y¹ is >NR⁵, NR⁵R¹ is a 5-, 6-, or 7-membered heterocyclic ring, which optionally comprises one or two additional heteroatoms selected from >O, >S or >N—, in which NR⁵R¹ is optionally substituted with one, two, or three substituents selected independently from an alkyl, an alkoxy, or a haloalkyl, any of which having up to 10 carbon atoms, or hydroxyl, halogen, or cyano;
R¹ is a substituted or an unsubstituted aryl, heterocyclyl, or heteroaryl, any of which having up to 12 carbon atoms; wherein any heteroaryl or heterocyclyl comprises at least one heteroatom or heterogroup selected from >O, >N—, >S, >NR⁶, >SO₂, or >CO;
R² is a substituted or an unsubstituted alkyl, haloalkyl, aryl, or heteroaryl, any of which having up to 12 carbon atoms; wherein any heteroaryl comprises at least one heteroatom or heterogroup selected from >O, >N—, >S, or >NR⁶;
R³ and R⁴ are selected independently from a substituted or an unsubstituted alkyl or a substituted or an unsubstituted aryl, any of which having up to 12 carbon atoms;
R⁵ is a substituted or an unsubstituted alkyl having up to 12 carbon atoms, or hydrogen;
any of R¹, R², R³, R⁴, and R⁵ is optionally substituted with at least one group selected independently from: 1) an alkyl, an alkoxy, an alkylthio, a haloalkyl, a haloalkoxy, a cycloalkyl, NR⁶R⁷, —CO₂R⁶, —COR⁸, —CONR⁶R⁷, —SO₂R⁸ and —SO₂NR⁶R⁷, NHSO₂R⁸, or NHCOR⁸, any of which having up to 10 carbon atoms; or 2) halogen, hydroxyl, or cyano;
R⁶ and R⁷ are selected independently from an alkyl or an aryl having up to 10 carbon atoms, or hydrogen; and
R⁸ is an alkyl or aryl having up to 10 carbon atoms.

2. A compound according to claim 1, having the formula: 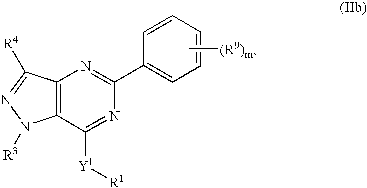
or a salt, a prodrug, a diastereomeric mixture, an enantiomer, a tautomer, a racemic mixture, or any combination thereof, wherein:
Y¹ is >NR⁵, —C≡C—, >O, or a direct a bond between the 6-membered ring and R¹;
R¹ is a substituted or an unsubstituted aryl, heterocyclyl, or heteroaryl, any of which having up to 12 carbon atoms; wherein any heteroaryl or heterocyclyl comprises at least one heteroatom or heterogroup selected from >O, >N—, >S, >NR⁶, >SO₂, or >CO;
R³ and R⁴ are selected independently from a substituted or an unsubstituted alkyl or a substituted or an unsubstituted aryl, any of which having up to 12 carbon atoms;
R⁵ is a substituted or an unsubstituted alkyl having up to 12 carbon atoms, or hydrogen;
R⁹, in each occurrence, is selected independently from: 1) an alkyl, an alkoxy, a haloalkyl, a haloalkoxy, SO₂R⁸, SO₂NR⁶R⁷, CO₂R⁶, COR⁸, or CONR⁶R⁷, any of which having up to 10 carbon atoms; or 2) halogen;
m is an integer from 0 to 3, inclusive;
any of R¹, R³, and R⁴ can be optionally substituted with at least one group selected independently from: 1) an alkyl, an alkoxy, an alkylthio, a haloalkyl, a haloalkoxy, a cycloalkyl, NR⁶R⁷, —CO₂R⁶, —COR⁸, —CONR⁶R⁷, —SO₂R⁸ and —SO₂NR⁶R⁷, NHSO₂R⁸, or NHCOR⁸, any of which having up to 10 carbon atoms; or 2) halogen, hydroxyl, or cyano;
R⁶ and R⁷ are selected independently from an alkyl or an aryl having up to 10 carbon atoms, or hydrogen; and
R⁸ is an alkyl or aryl having up to 10 carbon atoms.

3. A compound according to claim 1, having the formula: 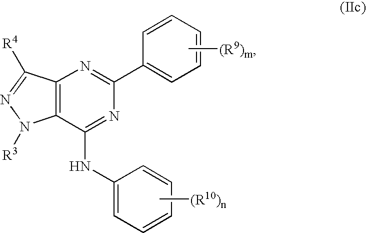
or a salt, a prodrug, a diastereomeric mixture, an enantiomer, a tautomer, a racemic mixture, or any combination thereof, wherein:
R⁹ and R¹⁰, in each occurrence, are selected independently from: 1) an alkyl, an alkoxy, a haloalkyl, a haloalkoxy, NR⁶R⁷, CO₂R⁶, COR⁸, CONR⁶R⁷, SO₂R⁸, SO₂NR⁶R⁷, NHSO₂R⁸, or NHCOR⁸, any of which having up to 10 carbon atoms; or 2) halogen or cyano;
m and n are selected independently from an integer from 0 to 3, inclusive;
R⁶ and R⁷ are selected independently from an alkyl or an aryl having up to 10 carbon atoms, or hydrogen;
R⁸ is an alkyl or aryl having up to 10 carbon atoms;
R³ and R⁴ are selected independently from a substituted or an unsubstituted alkyl or a substituted or an unsubstituted aryl, any of which having up to 12 carbon atoms; and
any of R³ and R⁴ can be optionally substituted with at least one group selected independently from: 1) an alkyl, an alkoxy, a haloalkyl, a haloalkoxy, or a cycloalkyl, any of which having up to 10 carbon atoms; or 2) halogen or hydroxyl.

4. A compound according to Claim 3, wherein the compound is:
(3-Chloro-4-methoxy-phenyl)-[5-(3,4-dimethoxy-phenyl)-1-methyl-3-
propyl-1H-pyrazolo[4,3-d]pyrimidin-7-yl]-amine hydrochloride;
(3-Chloro-4-methoxy-phenyl)-[5-(4-fluoro-phenyl)-1-methyl-3-propyl-1H-
pyrazolo[4,3-d]pyrimidin-7-yl]-amine hydrochloride;
(3-Fluoro-4-methoxy-phenyl)-[5-(4-fluoro-phenyl)-1-methyl-3-propyl-1H-
pyrazolo[4,3-d]pyrimidin-7-yl]-amine hydrochloride;
(3-Fluoro-4-methoxy-phenyl)-[5-(4-fluoro-phenyl)-1-methyl-3-propyl-1H-
pyrazolo
[4,3-d]pyrimidin-7-yl]-amine;
(4-Fluoro-phenyl)-[5-(4-fluoro-phenyl)-1-methyl-3-propyl-1H-
pyrazolo[4,3-d]pyrimidin-7-yl]-amine hydrochloride;
(3,4-Dimethoxy-phenyl)-[5-(4-fluoro-phenyl)-1-methyl-3-propyl-1H-
pyrazolo[4,3-d]pyrimidin-7-yl]-amine hydrochloride;
2-Chloro-4-(1-methyl-5-phenyl-3-propyl-1H-pyrazolo[4,3-d]pyrimidin-7-
ylamino)-phenol hydrochloride;
(3-Chloro-4-methoxy-phenyl)-(1-methyl-5-phenyl-3-propyl-1H-
pyrazolo[4,3-d]pyrimidin-7-yl)-amine hydrochloride;
(3-Chloro-4-methoxy-phenyl)-(1-methyl-5-phenyl-3-propyl-1H-
pyrazolo[4,3-d]pyrimidin-7-yl)-amine;
(3-Fluoro-4-methoxy-phenyl)-(1-methyl-5-phenyl-3-propyl-1H-
pyrazolo[4,3-d]pyrimidin-7-yl)-amine hydrochloride;
2-Chloro-4-[5-(4-fluoro-phenyl)-1-methyl-3-propyl-1H-pyrazolo[4,3-
d]pyrimidin-7-ylamino]-phenol hydrochloride;
3-[7-(3-Chloro-4-methoxy-phenylamino)-1-methyl-3-propyl-1H-
pyrazolo[4,3-d]pyrimidin-5-yl]-4-ethoxy-
benzenesulfonamide hydrochloride;
4-Ethoxy-3-[7-(3-fluoro-4-methoxy-phenylamino)-1-methyl-3-propyl-1H-
pyrazolo[4,3-d]pyrimidin-5-yl]-benzenesulfonamide hydrochloride;
(3-Chloro-4-methoxy-phenyl)-[5-(2-ethoxy-phenyl)-1-methyl-3-propyl-1H-
pyrazolo[4,3-d]pyrimidin-7-yl]-amine hydrochloride;
[5-(2-Ethoxy-phenyl)-1-methyl-3-propyl-1H-pyrazolo[4,3-d]pyrimidin-7-
yl]-(3-fluoro-4-methoxy-phenyl)-amine hydrochloride;
(3-Fluoro-4-methoxy-phenyl)-[5-(4-fluoro-phenyl)-1,3-dimethyl-1H-
pyrazolo[4,3-d]pyrimidin-7-yl]-amine hydrochloride;
(3-Chloro-4-methoxy-phenyl)-[5-(4-fluoro-phenyl)-1,3-dimethyl-1H-
pyrazolo[4,3-d]pyrimidin-7-yl]-amine hydrochloride;
2-Chloro-4-[5-(4-fluoro-phenyl)-1,3-dimethyl-1H-pyrazolo[4,3-
d]pyrimidin-7-ylamino]-phenol hydrochloride;
(4-Chloro-3-methoxy-phenyl)-(1-methyl-5-phenyl-3-propyl-1H-
pyrazolo[4,3-d]pyrimidin-7-yl)-amine hydrochloride;
(4-Chloro-3-methoxy-phenyl)-[5-(4-fluoro-phenyl)-1-methyl-3-propyl-1H-
pyrazolo[4,3-d]pyrimidin-7-yl]-amine hydrochloride;
(3-Chloro-4-methoxy-phenyl)-(1,3-dimethyl-5-phenyl-1H-pyrazolo[4,3-
d]pyrimidin-7-yl)-amine hydrochloride;
(1,3-Dimethyl-5-phenyl-1H-pyrazolo[4,3-d]pyrimidin-7-yl)-(3-fluoro-4-
methoxy-phenyl)-amine hydrochloride;
(4-Chloro-3-methoxy-phenyl)-(1,3-dimethyl-5-phenyl-1H-pyrazolo[4,3-
d]pyrimidin-7-yl)-amine hydrochloride;
2-Fluoro-4-[5-(4-fluoro-phenyl)-1-methyl-3-propyl-1H-pyrazolo[4,3-
d]pyrimidin-7-ylamino]-phenol hydrochloride;
Benzo[1,3]dioxol-5-yl-[5-(4-fluoro-phenyl)-1-methyl-3-propyl-1H-
pyrazolo[4,3-d]pyrimidin-7-yl]-amine hydrochloride;
(1,3-Dimethyl-5-phenyl-1H-pyrazolo[4,3-d]pyrimidin-7-yl)-(3-fluoro-
phenyl)-amine hydrochloride;
[5-(4-Fluoro-phenyl)-1-methyl-3-propyl-1H-pyrazolo[4,3-d]pyrimidin-
7-yl]-(3-trifluoromethyl-phenyl)-amine hydrochloride;
[5-(4-Fluoro-phenyl)-1-methyl-3-propyl-1H-pyrazolo[4,3-d]pyrimidin-
7-yl]-(4-trifluoromethoxy-phenyl)-amine hydrochloride;
(1,3-Dimethyl-5-phenyl-1H-pyrazolo[4,3-d]pyrimidin-7-yl)-(4-
trifluoromethoxy-phenyl)-amine hydrochloride;
[5-(4-Fluoro-phenyl)-1,3-dimethyl-1H-pyrazolo[4,3-d]pyrimidin-7-yl]-(4-
rifluoromethyl-phenyl)-amine hydrochloride;
(6-Chloro-pyridin-3-yl)-[5-(4-fluoro-phenyl)-1,3-dimethyl-1H-
pyrazolo[4,3-d]pyrimidin-7-yl]-amine hydrochloride;
N-{5-[5-(4-Fluoro-phenyl)-1,3-dimethyl-1H-pyrazolo[4,3-d]pyrimidin-7-
ylamino]-2-hydroxy-phenyl}-acetamide hydrochloride;
[5-(4-Fluoro-phenyl)-1,3-dimethyl-1H-pyrazolo[4,3-d]pyrimidin-7-yl]-(4-
methanesulfonyl-phenyl)-amine;
(1,3-Dimethyl-5-phenyl-1H-pyrazolo[4,3-d]pyrimidin-7-yl)-(2-methyl-
benzooxazol-5-yl)-amine hydrochloride;
N-[4-(1,3-Dimethyl-5-phenyl-1H-pyrazolo[4,3-d]pyrimidin-7-ylamino)-
phenyl]-methanesulfonamide hydrochloride;
4-[5-(4-Fluoro-phenyl)-1,3-dimethyl-1H-pyrazolo[4,3-d]pyrimidin-7-
ylamino]-N,N-dimethyl-benzenesulfonamide hydrochloride;
4-(1,3-Dimethyl-5-phenyl-1H-pyrazolo[4,3-d]pyrimidin-7-ylamino)-
benzenesulfonamide hydrochloride;
3-[5-(4-Fluoro-phenyl)-1,3-dimethyl-1H-pyrazolo[4,3-d]pyrimidin-7-
ylamino]-benzamide hydrochloride;
3-[5-(4-Fluoro-phenyl)-1,3-dimethyl-1H-pyrazolo[4,3-d]pyrimidin-7-
ylamino]-N-methyl-benzamide hydrochloride;
4-[5-(4-Fluoro-phenyl)-1,3-dimethyl-1H-pyrazolo[4,3-d]pyrimidin-7-
ylamino]-benzenesulfonamide hydrochloride;
4-[5-(4-Fluoro-phenyl)-1,3-dimethyl-1H-pyrazolo[4,3-d]pyrimidin-7-
ylamino]-N-methyl-benzenesulfonamide hydrochloride;
4-[5-(4-Fluoro-phenyl)-1,3-dimethyl-1H-pyrazolo[4,3-d]pyrimidin-7-
ylamino]-benzamide hydrochloride;
4-[5-(4-Fluoro-phenyl)-1,3-dimethyl-1H-pyrazolo[4,3-d]pyrimidin-7-
ylamino]-N-methyl-benzamide hydrochloride; or
any combination thereof.

5. A compound according to claim 1, having the formula: 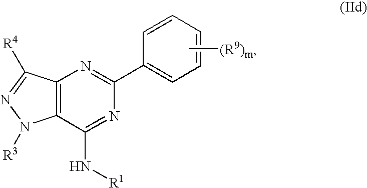
or a salt, a prodrug, a diastereomeric mixture, an enantiomer, a tautomer, a racemic mixture, or any combination thereof, wherein:
R¹ is a substituted or an unsubstituted aryl, or a substituted or an unsubstituted heteroaryl, any of which having up to 12 carbon atoms; wherein any heteroaryl comprises at least one heteroatom or heterogroup selected from >O, >N—, >S, or >NR⁶;
R⁹, in each occurrence, is selected independently from: 1) an alkyl, an alkoxy, a haloalkyl, a haloalkoxy, SO₂R⁸, SO₂NR⁶R⁷, NR⁶R⁷, CO₂R⁶, COR⁸, or CONR⁶R⁷, any of which having up to 10 carbon atoms; or 2) halogen;
m is an integer from 0 to 3, inclusive;
R³ and R⁴ are selected independently from a substituted or an unsubstituted alkyl or a substituted or an unsubstituted aryl, any of which having up to 12 carbon atoms; and
any of R¹, R³, and R⁴ can be optionally substituted with at least one group selected independently from: 1) an alkyl, an alkoxy, a haloalkyl, or a haloalkoxy, any of which having up to 10 carbon atoms; or 2) halogen or hydroxyl.

6. A compound according to claim 5, wherein R¹ is an indole, a benzimidazole, a benzoxazole, a benzo[1,3]dioxole, or a pyridine.

7. A compound according to claim 5, where the compound is:
(1H-Benzoimidazol-5-yl)-(1,3-dimethyl-5-phenyl-1H-pyrazolo[4,3-
d]pyrimidin-7-yl)-amine hydrochloride;
(1,3-Dimethyl-5-phenyl-1H-pyrazolo[4,3-d]pyrimidin-7-yl)-(2-methyl-1H-
benzoimidazol-5-yl)-amine hydrochloride;
Benzo[1,3]dioxol-5-yl-[5-(4-fluoro-phenyl)-1,3-dimethyl-1H-pyrazolo[4,3-
d]pyrimidin-7-yl]-amine hydrochloride; or
any combination thereof.

8. A compound according to claim 1, having the formula: 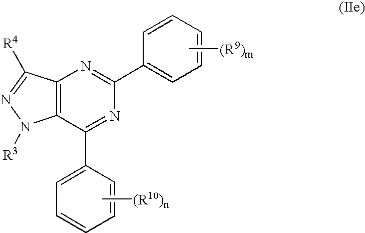
or a salt, a prodrug, a diastereomeric mixture, an enantiomer, a tautomer, a racemic mixture, or any combination thereof, wherein:
R⁹ and R¹⁰, in each occurrence, are selected independently from: 1) an alkyl, an alkoxy, a haloalkyl, a haloalkoxy, NR⁶R⁷, CO₂R⁶, COR⁸, CONR⁶R⁷, SO₂R⁸, SO₂NR⁶R⁷, NHSO₂R⁸, or NHCOR⁸, any of which having up to 10 carbon atoms; or 2) halogen or cyano;
m and n are selected independently from an integer from 0 to 3, inclusive;
R⁶ and R⁷ are selected independently from an alkyl or an aryl having up to 10 carbon atoms, or hydrogen;
R⁸ is an alkyl or aryl having up to 10 carbon atoms;
R³ and R⁴ are selected independently from a substituted or an unsubstituted alkyl or a substituted or an unsubstituted aryl, any of which having up to 12 carbon atoms; and
any of R³ and R⁴ can be optionally substituted with at least one group selected independently from: 1) an alkyl, an alkoxy, a haloalkyl, a haloalkoxy, or a cycloalkyl, any of which having up to 10 carbon atoms; or 2) halogen or hydroxyl.

9. A compound according to Claim 8, where the compound is:
4-[5-(3,4-Dimethoxy-phenyl)-1-methyl-3-propyl-1H-pyrazolo[4,3-
d]pyrimidin-7-yl]-2-methyl-phenol;
2-Methyl-4-(1-methyl-5-phenyl-3-propyl-1H-pyrazolo[4,3-d]pyrimidin-
7-yl)-phenol;
4-[5-(3-hydroxy,4-methoxy-phenyl)-1-methyl-3-propyl-1H-pyrazolo[4,3-
d]pyrimidin-7-yl]-2-methyl-phenol;
2-Chloro-4-[5-(4-fluoro-phenyl)-1-methyl-3-propyl-1H-pyrazolo[4,3-
d]pyrimidin-7-yl]-phenol;
7-(4-Methoxy-3-methyl-phenyl)-1-methyl-5-phenyl-3-propyl-1H-
pyrazolo[4,3-d]pyrimidine;
5-(4-fluoro-phenyl)-1,3-dimethyl-7-phenyl-1H-pyrazolo[4,3-d]pyrimidine;
5-(4-Fluoro-phenyl)-1-methyl-3-propyl-7-p-tolyl-1H-pyrazolo[4,3-
d]pyrimidine;
7-(3-Fluoro-4-methoxy-phenyl)-1-methyl-5-phenyl-3-propyl-1H-
pyrazolo[4,3-d]pyrimidine;
5-(4-Fluoro-phenyl)-1-methyl-7-phenyl-3-propyl-1H-pyrazolo[4,3-
d]pyrimidine;
5-(4-Fluoro-phenyl)-1-methyl-7-(4-methylsulfanyl-phenyl)-3-propyl-1H-
pyrazolo[4,3-d]pyrimidine;
7-(3-Fluoro-4-methoxy-phenyl)-5-(4-fluoro-phenyl)-1-methyl-3-propyl-
1H-pyrazolo[4,3-d]pyrimidine;
5-(4-Fluoro-phenyl)-7-(4-methoxy-3-methyl-phenyl)-1-methyl-3-propyl-
1H-pyrazolo[4,3-d]pyrimidine;
5-(4-Fluoro-phenyl)-7-(4-methanesulfonyl-phenyl)-1-methyl-3-propyl-
1H-pyrazolo[4,3-d]pyrimidine;
7-(3-Methanesulfonyl-phenyl)-1,3-dimethyl-5-phenyl-1H-
pyrazolo[4,3-d]pyrimidine;
5-(4-Fluoro-phenyl)-7-(3-methanesulfonyl-phenyl)-1,3-dimethyl-1H-
pyrazolo[4,3-d]Pyrimidine;
5-(4-Fluoro-phenyl)-1,3-dimethyl-7-(4-trifluoromethoxy-phenyl)-1H-
pyrazolo[4,3-d]pyrimidine; or
any combination thereof.

10. A compound according to claim 1, having the formula: 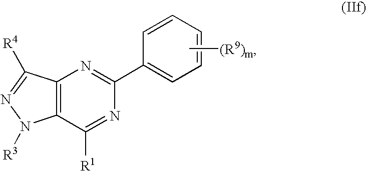
or a salt, a prodrug, a diastereomeric mixture, an enantiomer, a tautomer, a racemic mixture, or any combination thereof, wherein:
R¹ is a substituted or an unsubstituted heteroaryl, or a substituted or an unsubstituted heterocyclyl, comprising at least one heteroatom or heterogroup selected from —O—, >N—, —S—, >NR⁶, >CO, or >SO₂, any of which having up to 10 carbon atoms;
R⁹, in each occurrence, is selected independently from: 1) an alkyl, an alkoxy, a haloalkyl, a haloalkoxy, SO₂R⁸, SO₂NR⁶R⁷, NR⁶R⁷, CO₂R⁶, COR⁸, or CONR⁶R⁷, any of which having up to 10 carbon atoms; or 2) halogen;
m is an integer from 0 to 3, inclusive;
R³ and R⁴ are selected independently from a substituted or an unsubstituted alkyl or a substituted or an unsubstituted aryl, any of which having up to 12 carbon atoms;
any of R¹, R³, and R⁴ can be optionally substituted with at least one group selected independently from: 1) an alkyl, an alkoxy, an alkylthio, a haloalkyl, a haloalkoxy, a cycloalkyl, NR⁶R⁷, CO₂R⁶, COR⁸, CONR⁶R⁷, SO₂R⁸, SO₂NR⁶R⁷, NHSO₂R⁸, or NHCOR⁸, any of which having up to 10 carbon atoms; or 2) halogen or hydroxyl;
R⁶ and R⁷ are selected independently from an alkyl or an aryl having up to 10 carbon atoms, or hydrogen; and
R⁸ is an alkyl or aryl having up to 10 carbon atoms.

11. A compound according to claim 10, wherein R¹ is an indole, a benzo[1,3]dioxole, or a piperidine.

12. A compound according to Claim 10, wherein the compound is:
1-[5-(3,4-Dimethoxy-phenyl)-1-methyl-3-propyl-1H-pyrazolo[4,3-
d]pyrimidin-7-yl]-piperidin-4-ol;
5-(4-Fluoro-phenyl)-7-indol-1-yl-1-methyl-3-propyl-1H-pyrazolo[4,3-
d]pyrimidine;
7-(5-Chloro-indol-1-yl)-5-(4-fluoro-phenyl)-1-methyl-3-propyl-1H-
pyrazolo[4,3-d]pyrimidine;
7-Indol-1-yl-1,3-dimethyl-5-phenyl-1H-pyrazolo[4,3-d]pyrimidine;
7-Benzo[1,3]dioxol-5-yl-1,3-dimethyl-5-phenyl-1H-pyrazolo[4,3-
d]pyrimidine; or
any combination thereof.

13. A compound according to claim 1, having the formula: 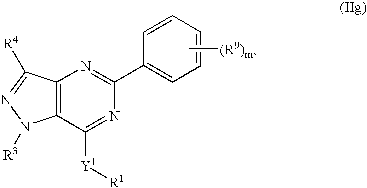
or a salt, a prodrug, a diastereomeric mixture, an enantiomer, a tautomer, a racemic mixture, or any combination thereof, wherein:
Y¹ is —C≡C—, >O, or a direct a bond between the 6-membered ring and R¹;
R¹ is a substituted or an unsubstituted aryl or heteroaryl, any of which having up to 12 carbon atoms; wherein any heteroaryl comprises at least one heteroatom or heterogroup selected from >O, >N—, >S, or >NR⁶;
R⁹, in each occurrence, is selected independently from: 1) an alkyl, an alkoxy, a haloalkyl, a haloalkoxy, SO₂R⁸, SO₂NR⁶R⁷, NR⁶R⁷, CO₂R⁶, COR⁸, or CONR⁶R⁷, any of which having up to 10 carbon atoms; or 2) halogen;
m is an integer from 0 to 3, inclusive;
R³ and R⁴ are selected independently from a substituted or an unsubstituted allyl or a substituted or an unsubstituted aryl, any of which having up to 12 carbon atoms;
any of R¹, R³, and R⁴ can be optionally substituted with at least one group selected independently from: 1) an alkyl, an alkoxy, an alkylthio, a haloalkyl, a haloalkoxy, a cycloalkyl, NR⁶R⁷, CO₂R⁸, COR⁸, CONR⁶R⁷, SO₂R⁸, SO₂NR⁶R⁷, NHSO₂R⁸, or NHCOR⁸, any of which having up to 10 carbon atoms; or 2) halogen or hydroxyl;
R⁶ and R⁷ are selected independently from an alkyl or an aryl having up to 10 carbon atoms, or hydrogen; and
R⁸ is an alkyl or aryl having up to 10 carbon atoms.

14. A compound according to claim 13, wherein the compound is:
7-(4-Fluoro-phenoxy)-1-methyl-5-phenyl-3-propyl-1H-pyrazolo[4,3-
d]pyrimidine;
5-(4-Fluoro-phenyl)-1-methyl-7-phenylethynyl-3-propyl-1H-pyrazolo[4,3-
d]pyrimidine; or
any combination thereof.

15. A compound according to claim 1, having the formula: 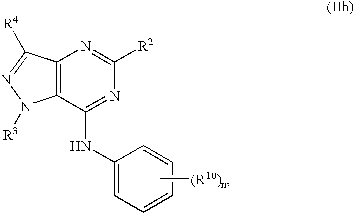
or a salt, a prodrug, a diastereomeric mixture, an enantiomer, a tautomer, a racemic mixture, or any combination thereof, wherein:
R² is a substituted or an unsubstituted haloalkyl or heteroaryl, any of which having up to 12 carbon atoms; wherein any heteroaryl comprises at least one heteroatom or heterogroup selected from >O, >N—, >S, or >NR⁶;
R¹⁰, in each occurrence, is selected independently from: 1) an alkyl, an alkoxy, a haloalkyl, a haloalkoxy, NR⁶R⁷, OCH₂O, CO₂R⁶, COR⁶, CONR⁶R⁷, SO₂R⁶, SO₂NR⁶R⁷, NHSO₂R⁶, or NHCOR⁶, any of which having up to 10 carbon atoms; or 2) halogen or cyano;
n is an integer from 0 to 3, inclusive;
R³ and R⁴ are selected independently from a substituted or an unsubstituted alkyl or a substituted or an unsubstituted aryl, any of which having up to 12 carbon atoms; and
any of R², R³, and R⁴ can be optionally substituted with at least one group selected independently from: 1) an alkyl, an alkoxy, an alkylthio, a haloalkyl, a haloalkoxy, or a cycloalkyl, any of which having up to 10 carbon atoms; or 2) halogen or hydroxyl.

16. A compound according to claim 15, wherein R² is a thiophene or CF₃.

17. A compound according to claim 15, wherein the compound is:
(3-Chloro-4-methoxy-phenyl)-(1-methyl-3-propyl-5-thiophen-2-yl-1H-
pyrazolo[4,3-d]pyrimidin-7-yl)-amine hydrochloride;
(3-Fluoro-4-methoxy-phenyl)-(1-methyl-3-propyl-5-thiophen-2-yl-1H-
pyrazolo[4,3-d]pyrimidin-7-yl)-amine hydrochloride;
(1,3-Dimethyl-5-thiophen-2-yl-1H-pyrazolo[4,3-d]pyrimidin-7-yl)-(3-
fluoro-4-methoxy-phenyl)-amine hydrochloride;
(4-Chloro-3-methoxy-phenyl)-(1,3-dimethyl-5-thiophen-2-yl-1H-
pyrazolo[4,3-d]pyrimidin-7-yl)-amine hydrochloride;
(3-Chloro-4-methoxy-phenyl)-(1,3-dimethyl-5-thiophen-2-yl-1H-
pyrazolo[4,3-d]pyrimidin-7-yl)-amine hydrochloride;
2-Chloro-4-(1,3-dimethyl-5-thiophen-2-yl-1H-pyrazolo[4,3-d]pyrimidin-7-
ylamino)-phenol hydrochloride;
(3-Fluoro-4-methoxy-phenyl)-(1-methyl-3-propyl-5-trifluoromethyl-1H-
pyrazolo[4,3-d]pyrimidin-7-yl)-amine hydrochloride;
(4-Chloro-3-methoxy-phenyl)-(1-methyl-3-propyl-5-thiophen-2-yl-1H-
pyrazolo[4,3-d]pyrimidin-7-yl)-amine hydrochloride; or
any combination thereof.

18. A compound according to claim 1, having the formula: 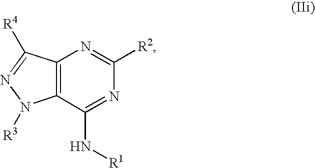
or a salt, a prodrug, a diastereomeric mixture, an enantiomer, a tautomer, a racemic mixture, or any combination thereof, wherein:
R¹ and R² are independently a substituted or an unsubstituted heteroaryl, any of which having up to 12 carbon atoms; wherein any heteroaryl comprises at least one heteroatom or heterogroup selected from >O, >N—, >S, or >NR⁶;
R³ and R⁴ are selected independently from a substituted or an unsubstituted alkyl or a substituted or an unsubstituted aryl, any of which having up to 12 carbon atoms; and
any of R¹, R², R³, and R⁴ can be optionally substituted with at least one group selected independently from: 1) an alkyl, an alkoxy, an alkylthio, a haloalkyl, a haloalkoxy, a cycloalkyl, NR⁶R⁷, CO₂R⁶, COR⁸, CONR⁶R⁷, SO₂R⁸, SO₂NR⁶R⁷, NHSO₂R⁸, or NHCOR⁸, any of which having up to 10 carbon atoms; or 2) halogen or hydroxyl;
R⁶ and R⁷, in each occurrence, are selected independently from an alkyl or an aryl having up to 10 carbon atoms, or hydrogen; and
R⁸ is an alkyl or aryl having up to 10 carbon atoms.

19. A compound according to claim 18, wherein the compound is:
Benzo[1,3]dioxol-5-yl-(1-methyl-3-propyl-5-thiophen-2-yl-1H-
pyrazolo[4,3-d]pyrimidin-7-yl)-amine hydrochloride;
Benzo[1,3]dioxol-5-yl-(1,3-dimethyl-5-thiophen-2-yl-1H-pyrazolo[4,3-d]
pyrimidin-7-yl)-amine hydrochloride;
Benzo[1,3]dioxol-5-yl-(1-methyl-3-propyl-5-thiophen-2-yl-1H-
pyrazolo[4,3-d]pyrimidin-7-yl)-amine hydrochloride; or
any combination thereof.

20. A compound according to claim 1, having the formula: 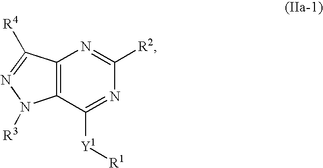
or a salt, a prodrug, a diastereomeric mixture, an enantiomer, a tautomer, a racemic mixture, or any combination thereof, wherein: 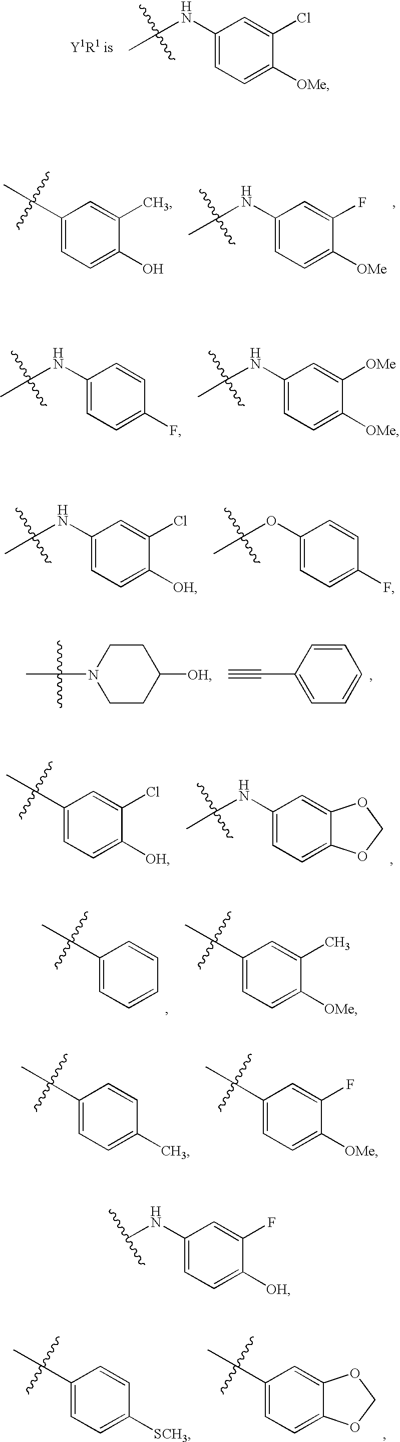 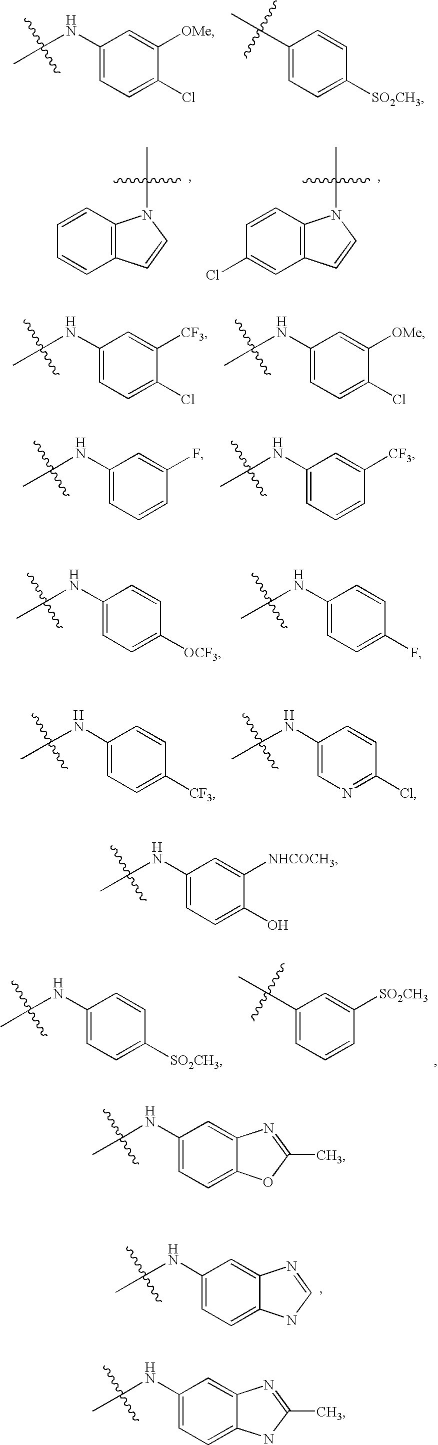 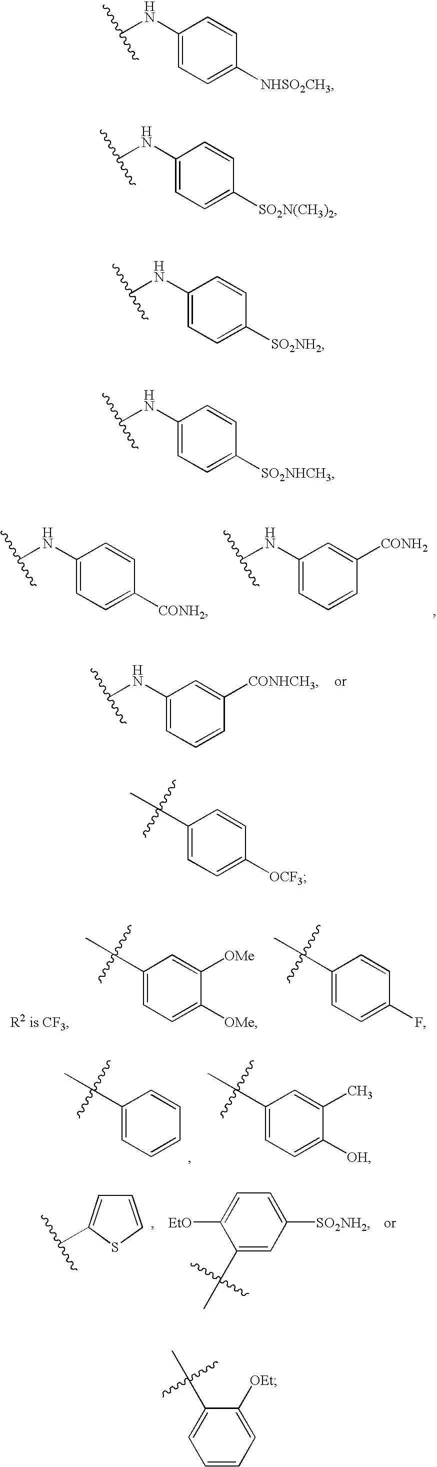
R³ is CH₃; and
R⁴ is CH₂CH₂CH₃, CH₂CH₃, or CH₃.

21. A method of treating a condition or disease state mediated by a high expression of TNF-alpha in a human or an animal, comprising administering an effective amount of at least one compound according to claim 1 to the human or the animal, sufficient to reduce TNF-alpha levels.

22. A method of treating a condition or disease state mediated by an increased proliferation of smooth muscle cells in a human or an animal, comprising administering an effective amount of at least one compound according to claim 1 to the human or the animal, sufficient to reduce smooth muscle cell proliferation.

23. A method of treating atherosclerosis, arthritis, restenosis, diabetic nephropathy, or dyslipidemia in a human or an animal, comprising administering an effective amount of at least one compound according to claim 1.

24. A composition comprising a pharmaceutically acceptable carrier and at least one compound having the formula: 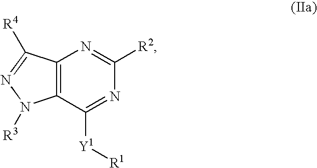
or a salt, a prodrug, a diastereomeric mixture, an enantiomer, a tautomer, a racemic mixture, or any combination thereof, wherein:
Y¹ is >NR⁵, —C≡C—, >O, or a direct a bond between the 6-membered ring and R¹;
wherein when Y¹ is >NR⁵, NR⁵R¹ is a 5-, 6-, or 7-membered heterocyclic ring, which optionally comprises one or two additional heteroatoms selected from >O, >S or >N—, in which NR⁵R¹ is optionally substituted with one, two, or three substituents selected independently from an alkyl, an alkoxy, or a haloalkyl, any of which having up to 10 carbon atoms, or hydroxyl, halogen, or cyano;
R¹ is a substituted or an unsubstituted aryl, heterocyclyl, or heteroaryl, any of which having up to 12 carbon atoms; wherein any heteroaryl or heterocyclyl comprises at least one heteroatom or heterogroup selected from >O, >N—, >S, >NR⁶, >SO₂, or >CO;
R² is a substituted or an unsubstituted alkyl, haloalkyl, aryl, or heteroaryl, any of which having up to 12 carbon atoms; wherein any heteroaryl comprises at least one heteroatom or heterogroup selected from >O, >N—, >S, or >NR⁶;
R³ and R⁴ are selected independently from a substituted or an unsubstituted alkyl or a substituted or an unsubstituted aryl, any of which having up to 12 carbon atoms;
R⁵ is a substituted or an unsubstituted alkyl having up to 12 carbon atoms, or hydrogen;
any of R¹, R², R³, R⁴, and R⁵ is optionally substituted with at least one group selected independently from: 1) an alkyl, an alkoxy, an alkylthio, a haloalkyl, a haloalkoxy, a cycloalkyl, NR⁶R⁷, —CO₂R⁶, —COR⁸, —CONR⁶R⁷, —SO₂R⁸ and —SO₂NR⁶R⁷, NHSO₂R⁸, or NHCOR⁸, any of which having up to 10 carbon atoms; or 2) halogen, hydroxyl, or cyano;
R⁶ and R⁷ are selected independently from an alkyl or an aryl having up to 10 carbon atoms, or hydrogen; and
R⁸ is an alkyl or aryl having up to 10 carbon atoms;
or a pharmaceutically acceptable salt, a prodrug, a diastereomeric mixture, an enantiomer, a tautomer, a racemic mixture, or any combination thereof.

25. The composition as claimed in claim 24, further comprising:
optionally, a pharmaceutically acceptable auxiliary;
optionally, a pharmaceutically acceptable preservative;
optionally, a pharmaceutically acceptable excipient;
optionally, a pharmaceutically acceptable diluent; and
optionally, a pharmaceutically acceptable solvate.

26. The composition as claimed in claim 24, further comprising an agent selected from an immunosuppressive agent, an anti-inflammatory agent, an antirheumatic agent, an antidyspilidemic agent, or any combination thereof.

27. The composition as claimed in claim 24, wherein the composition is in the form of a tablet, a capsule, a cachet, a powder, a granule, a solution, a suspension, an emulsion, a bolus, a lozenge, a suppository, a pessary, a tampon, a cream, a gel, a paste, a foam, a spray, an aerosol, a microcapsule, a liposome, a transdermal patch, a pastille, a paste, or a mouthwash.

28. A compound having the formula: 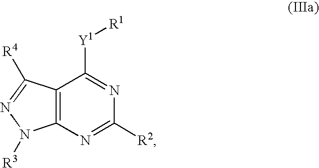
or a salt, a prodrug, a diastereomeric mixture, an enantiomer, a tautomer, a racemic mixture, or any combination thereof, wherein:
Y¹ is >NR⁵, —C≡C—, >O, or a direct a bond between the 6-membered ring and R¹;
R¹ is a substituted or an unsubstituted aryl, heterocyclyl, or heteroaryl, any of which having up to 12 carbon atoms; wherein any heteroaryl or heterocyclyl comprises at least one heteroatom or heterogroup selected from >O, >N—, >S, >NR⁶, >SO₂, or >CO;
R² is a substituted or an unsubstituted alkyl, haloalkyl, aryl, or heteroaryl, any of which having up to 12 carbon atoms; wherein any heteroaryl comprises at least one heteroatom or heterogroup selected from >O, >N—, >S, or >NR⁶;
R³ is a substituted or an unsubstituted alkyl or a substituted or an unsubstituted aryl, any of which having up to 12 carbon atoms;
R⁴ is a substituted or an unsubstituted alkyl or a substituted or an unsubstituted aryl, any of which having up to 12 carbon atoms, or hydrogen;
R⁵ is a substituted or an unsubstituted alkyl having up to 12 carbon atoms, or hydrogen;
any of R¹, R², R³, and R⁴ can be optionally substituted with at least one group selected independently from: 1) an alkyl, an alkoxy, an alkylthio, a haloalkyl, a haloalkoxy, a cycloalkyl, NR⁶R⁷, CO₂R⁶, COR⁸, CONR⁶R⁷, SO₂R⁸, SO₂NR⁶R⁷, NHSO₂R⁸, or NHCOR⁸, any of which having up to 10 carbon atoms; or 2) halogen, hydroxyl, or cyano;
R⁶ and R⁷ are selected independently from an alkyl or an aryl having up to 10 carbon atoms, or hydrogen; and
R⁸ is an alkyl or aryl having up to 10 carbon atoms.

29. A compound according to claim 28, wherein
R² is a substituted or an unsubstituted haloalkyl, aryl, or thiophenyl, any of which having up to 12 carbon atoms;
R³ is an alkyl having up to 6 carbon atoms or a phenyl;
R⁴ is an alkyl having up to 6 carbon atoms, phenyl, or hydrogen;
any of R¹ or R² is optionally substituted with at least one group selected independently from an alkyl, an alkoxy, an alkylthio, a haloalkyl, a haloalkoxy, CONR⁶R⁷, SO₂R⁸, SO₂NR⁶R⁷, NHSO₂R⁸, or NHCOR⁸, any of which having up to 10 carbon atoms;
R⁶ and R⁷ are selected independently from an alkyl or an aryl having up to 10 carbon atoms, or hydrogen; and
R⁸ is an alkyl or aryl having up to 10 carbon atoms.

30. A compound according to claim 28, having the formula: 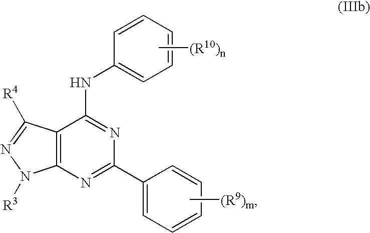
or a salt, a prodrug, a diastereomeric mixture, an enantiomer, a tautomer, a racemic mixture, or any combination thereof, wherein:
R³ and R⁴ are selected independently from hydrogen, methyl, ethyl, propyl, or phenyl;
R⁹ and R¹⁰, in each occurrence, are selected independently from: 1) an alkyl, an alkoxy, a haloalkyl, a haloalkoxy, NR⁶R⁷, CO₂R⁶, COR⁸, CONR⁶R⁷, SO₂R⁸, or SO₂NR⁶R⁷, any of which having up to 10 carbon atoms; or 2) halogen or cyano;
m and n are selected independently from an integer from 0 to 3, inclusive;
R⁶ and R⁷ are selected independently from H or methyl; and
R⁸ is methyl.

31. A compound according to claim 30, wherein the compound is:
(3-Chloro-4-methoxy-phenyl)-(1,6-diphenyl-1H-pyrazolo[3,4-d]pyrimidin-
4-yl)-amine hydrochloride;
(3-Fluoro-4-methoxy phenyl)-[6-(4-fluoro-phenyl)-1-phenyl-1H-pyrazolo
[3,4-d]-pyrimidin-4yl]amine hydrochloride;
(4-Chloro-3-trifluoromethyl-phenyl)-[6-(4-fluoro-phenyl)-1-phenyl-1H-
pyrazolo[3,4-d]pyrimidin-4-yl]-amine hydrochloride;
(3-Fluoro-phenyl)-[6-(4-fluoro-phenyl)-1-phenyl-1H-pyrazolo[3,4-
d]pyrimidin-4-yl]-amine hydrochloride;
[6-(4-Fluoro-phenyl)-1-phenyl-1H-pyrazolo[3,4-d]pyrimidin-4-yl]-(4-
trifluoromethoxy-phenyl)-amine hydrochloride; or
any combination thereof.

32. A compound according to claim 28, having the formula: 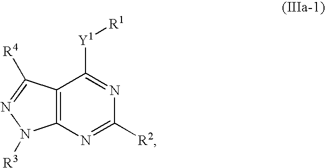
or a salt, a prodrug, a diastereomeric mixture, an enantiomer, a tautomer, a racemic mixture, or any combination thereof, wherein:

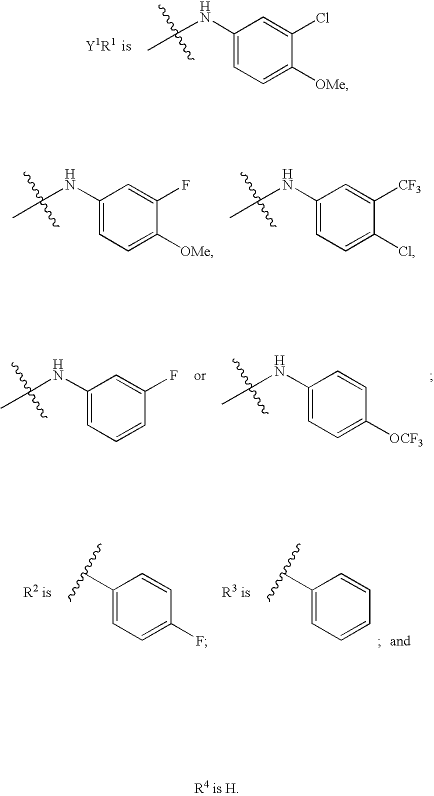

33. A method of treating a condition or disease state mediated by a high expression of TNF-alpha in a human or an animal, comprising administering an effective amount of at least one compound according to claim 28 to the human or the animal, sufficient to reduce TNF-alpha levels.

34. A method of treating a condition or disease state mediated by an increased proliferation of smooth muscle cells in a human or an animal, comprising administering an effective amount of at least one compound according to claim 28 to the human or the animal, sufficient to reduce smooth muscle cell proliferation.

35. A method of treating atherosclerosis, arthritis, restenosis, diabetic nephropathy, or dyslipidemia in a human or an animal, comprising administering an effective amount of at least one compound according to claim 28.

36. A composition comprising a pharmaceutically acceptable carrier and at least one compound having the formula: 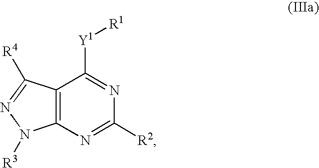
or a salt, a prodrug, a diastereomeric mixture, an enantiomer, a tautomer, a racemic mixture, or any combination thereof, wherein:
Y¹ is >NR⁵, —C≡C—, >O, or a direct a bond between the 6-membered ring and R¹;
R¹ is a substituted or an unsubstituted aryl, heterocyclyl, or heteroaryl, any of which having up to 12 carbon atoms; wherein any heteroaryl or heterocyclyl comprises at least one heteroatom or heterogroup selected from >O, >N—, >S, >NR⁶, >SO₂, or >CO;
R² is a substituted or an unsubstituted alkyl, haloalkyl, aryl, or heteroaryl, any of which having up to 12 carbon atoms; wherein any heteroaryl comprises at least one heteroatom or heterogroup selected from >O, >N—, >S, or >NR⁶;
R³ is a substituted or an unsubstituted alkyl or a substituted or an unsubstituted aryl, any of which having up to 12 carbon atoms;
R⁴ is a substituted or an unsubstituted alkyl or a substituted or an unsubstituted aryl, any of which having up to 12 carbon atoms, or hydrogen;
R⁵ is a substituted or an unsubstituted alkyl having up to 12 carbon atoms, or hydrogen;
any of R¹, R², R³, and R⁴ can be optionally substituted with at least one group selected independently from: 1) an alkyl, an alkoxy, an alkylthio, a haloalkyl, a haloalkoxy, a cycloalkyl, NR⁶R⁷, CO₂R⁶, COR⁸, CONR⁶R⁷, SO₂R⁸, SO₂NR⁶R⁷, NHSO₂R⁸, or NHCOR⁸, any of which having up to 10 carbon atoms; or 2) halogen, hydroxyl, or cyano;
R⁶ and R⁷ are selected independently from an alkyl or an aryl having up to 10 carbon atoms, or hydrogen; and
R⁸ is an alkyl or aryl having up to 10 carbon atoms;
or a pharmaceutically acceptable salt, a prodrug, a diastereomeric mixture, an enantiomer, a tautomer, a racemic mixture, or any combination thereof.

37. The composition as claimed in claim 36, further comprising:
optionally, a pharmaceutically acceptable auxiliary;
optionally, a pharmaceutically acceptable preservative;
optionally, a pharmaceutically acceptable excipient;
optionally, a pharmaceutically acceptable diluent; and
optionally, a pharmaceutically acceptable solvate.

38. The composition as claimed in claim 36, further comprising an agent selected from an immunosuppressive agent, an anti-inflammatory agent, an antirheumatic agent, an antidyspilidemic agent, or any combination thereof.

39. The composition as claimed in claim 36, wherein the composition is in the form of a tablet, a capsule, a cachet, a powder, a granule, a solution, a suspension, an emulsion, a bolus, a lozenge, a suppository, a pessary, a tampon, a cream, a gel, a paste, a foam, a spray, an aerosol, a microcapsule, a liposome, a transdermal patch, a pastille, a paste, or a mouthwash.

40. A compound having the formula: 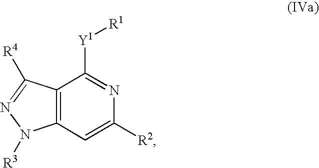
or a salt, a prodrug, a diastereomeric mixture, an enantiomer, a tautomer, a racemic mixture, or any combination thereof, wherein:
Y¹ is >NR⁵, —C≡C—, >O, or a direct a bond between the 6-membered ring and R¹;
R¹ is a substituted or an unsubstituted aryl, heterocyclyl, or heteroaryl, any of which having up to 12 carbon atoms; wherein any heteroaryl or heterocyclyl comprises at least one heteroatom or heterogroup selected from >O, >N—, >S, >NR⁶, >SO₂, or >CO;
R² is a substituted or an unsubstituted alkyl, haloalkyl, aryl, or heteroaryl, any of which having up to 12 carbon atoms; wherein any heteroaryl comprises at least one heteroatom or heterogroup selected from >O, >N—, >S, or >NR⁶;
R³ and R⁴ are selected independently from a substituted or an unsubstituted alkyl or a substituted or an unsubstituted aryl, any of which having up to 12 carbon atoms;
R⁵ is an alkyl having up to 12 carbon atoms or hydrogen;
any of R¹, R², R³, and R⁴ is optionally substituted with at least one group selected independently from: 1) an alkyl, an alkoxy, an alkylthio, a haloalkyl, a haloalkoxy, a cycloalkyl, NR⁶R⁷, CO₂R⁶, COR⁸, CONR⁶R⁷, SO₂R⁸, SO₂NR⁶R⁷, NHSO₂R⁸, or NHCOR⁸, any of which having up to 10 carbon atoms; or 2) halogen, hydroxyl, or cyano;
R⁶ and R⁷ are selected independently from an alkyl or an aryl having up to 10 carbon atoms, or hydrogen; and
R⁸ is an alkyl or aryl having up to 10 carbon atoms.

41. A compound according to claim 40, having the formula:
4-Benzo[1,3]dioxol-5-yl-6-(4-fluoro-phenyl)-1,3-dimethyl-1H-
pyrazolo[4,3-c]pyridine;
(6-Chloro-pyridin-3-yl)-[6-(4-fluoro-phenyl)-1,3-dimethyl-1H-
pyrazolo[4,3-c]pyridin-4-yl]-amine hydrochloride;
6-(4-Fluoro-phenyl)-4-(3-methanesulfonyl-phenyl)-1,3-dimethyl-1H-
pyrazolo[4,3-c]pyridine;
6-(4-Fluoro-phenyl)-4-(4-methanesulfonyl-phenyl)-1,3-dimethyl-1H-
pyrazolo[4,3-c]pyridine;
6-(4-Fluoro-phenyl)-1,3-dimethyl-4-(4-trifluoromethoxy-phenyl)-1H-
pyrazolo[4,3-c]pyridine;
any combination thereof.

42. A compound according to claim 40, having the formula: 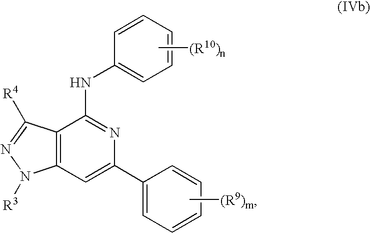
or a salt, a prodrug, a diastereomeric mixture, an enantiomer, a tautomer, a racemic mixture, or any combination thereof, wherein:
R³ and R⁴ are selected independently from methyl, ethyl, propyl, or phenyl;
R⁹ and R¹⁰, in each occurrence, are selected independently from: 1) an alkyl, an alkoxy, a haloalkyl, a haloalkoxy, NR⁶R⁷, CO₂R⁸, COR⁸, CONR⁶R⁷, SO₂R⁸, or SO₂NR⁶R⁷, any of which having up to 10 carbon atoms; or 2) halogen or cyano;
m and n are selected independently from an integer from 0 to 3, inclusive;
R⁶ and R⁷ are selected independently from H or methyl; and
R⁸ is methyl.

43. A compound according to claim 42, wherein the compound is:
(3-Chloro-4-methoxy-phenyl)-[6-(4-fluoro-phenyl)-1,3-dimethyl-1H-
pyrazolo[4,3-c] pyridin-4-yl]-amine hydrochloride;
(3-Fluoro-4-methoxy-phenyl)-[6-(4-fluoro-phenyl)-1,3-dimethyl-1H-
pyrazolo[4,3-c]pyridin-4-yl]-amine hydrochloride;
(4-Chloro-3-trifluoromethyl-phenyl)-[6-(4-fluoro-phenyl)-1,3-dimethyl-
1H-pyrazolo[4,3-c]pyridin-4-yl]-amine hydrochloride;
[6-(4-Fluoro-phenyl)-1,3-dimethyl-1H-pyrazolo[4,3-c]pyridin-4-yl]-(3-
trifluoromethyl-phenyl)-amine hydrochloride;
[6-(4-Fluoro-phenyl)-1,3-dimethyl-1H-pyrazolo[4,3-c]pyridin-4-yl]-(4-
methanesulfonyl-phenyl)-amine;
(1,3-dimethyl-6-(4-fluoro phenyl)-1H-pyrazolo[4,3-c]pyridin-4-yl)-(4-
trifluoromethoxy-phenyl)-amine hydrochloride;
4-[6-(4-Fluoro-phenyl)-1,3-dimethyl-1H-pyrazolo[4,3-c]pyridin-4-
ylamino]-N-methyl-benzenesulfonamide hydrochloride;
N-{4-[6-(4-Fluoro-phenyl)-1,3-dimethyl-1H-pyrazolo[4,3-c]pyridin-4-
ylamino]-phenyl}-methanesulfonamide hydrochloride; or
any combination thereof.

44. A compound according to claim 40, having the formula: 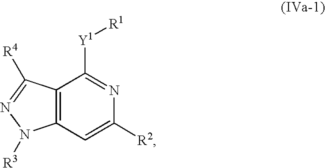
or a salt, a prodrug, a diastereomeric mixture, an enantiomer, a tautomer, a racemic mixture, or any combination thereof, wherein: 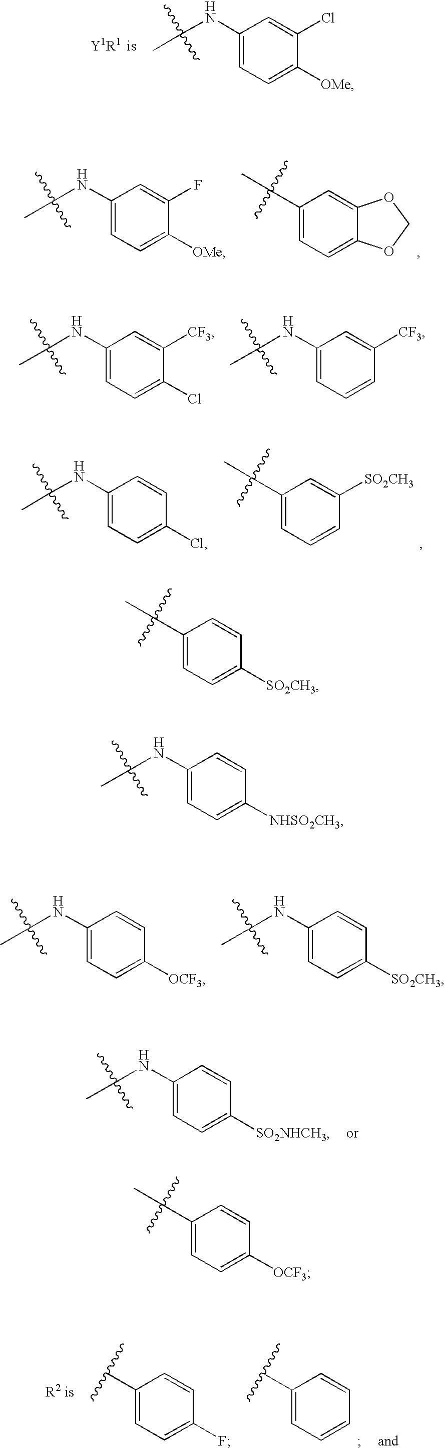
R³ and R⁴ are CH₂CH₂CH₃, CH₂CH₃, or CH₃.

45. A method of treating a condition or disease state mediated by a high expression of TNF-alpha in a human or an animal, comprising administering an effective amount of at least one compound according to claim 40 to the human or the animal, sufficient to reduce TNF-alpha levels.

46. A method of treating a condition or disease state mediated by an increased proliferation of smooth muscle cells in a human or an animal, comprising administering an effective amount of at least one compound according to claim 40 to the human or the animal, sufficient to reduce smooth muscle cell proliferation.

47. A method of treating atherosclerosis, arthritis, restenosis, diabetic nephropathy, or dyslipidemia in a human or an animal, comprising administering an effective amount of at least one compound according to claim 40.

48. A composition comprising a pharmaceutically acceptable carrier and at least one compound having the formula: 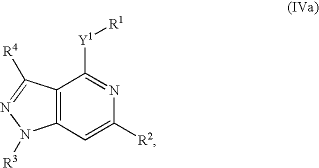
or a salt, a prodrug, a diastereomeric mixture, an enantiomer, a tautomer, a racemic mixture, or any combination thereof, wherein:
Y¹ is >NR⁵, —C≡C—, >O, or a direct a bond between the 6-membered ring and R¹;
R¹ is a substituted or an unsubstituted aryl, heterocyclyl, or heteroaryl, any of which having up to 12 carbon atoms; wherein any heteroaryl or heterocyclyl comprises at least one heteroatom or heterogroup selected from >O, >N—, >S, >NR⁶, >SO₂, or >CO;
R¹ is a substituted or an unsubstituted alkyl, haloalkyl, aryl, or heteroaryl, any of which having up to 12 carbon atoms; wherein any heteroaryl comprises at least one heteroatom or heterogroup selected from >O, >N—, >S, or >NR⁶;
R³ and R⁴ are selected independently from a substituted or an unsubstituted alkyl or a substituted or an unsubstituted aryl, any of which having up to 12 carbon atoms;
R⁵ is an alkyl having up to 12 carbon atoms or hydrogen;
any of R¹, R², R³, and R⁴ is optionally substituted with at least one group selected independently from: 1) an alkyl, an alkoxy, an alkylthio, a haloalkyl, a haloalkoxy, a cycloalkyl, NR⁶R⁷, CO₂R⁶, COR⁸, CONR⁶R⁷, SO₂R⁸, SO₂NR⁶R⁷, NHSO₂R⁸, or NHCOR⁸, any of which having up to 10 carbon atoms; or 2) halogen, hydroxyl, or cyano;
R⁶ and R⁷ are selected independently from an alkyl or an aryl having up to 10 carbon atoms, or hydrogen; and
R⁸ is an alkyl or aryl having up to 10 carbon atoms;
or a pharmaceutically acceptable salt, a prodrug, a diastereomeric mixture, an enantiomer, a tautomer, a racemic mixture, or any combination thereof.

49. The composition as claimed in claim 48, further comprising:
optionally, a pharmaceutically acceptable auxiliary;
optionally, a pharmaceutically acceptable preservative;
optionally, a pharmaceutically acceptable excipient;
optionally, a pharmaceutically acceptable diluent; and
optionally, a pharmaceutically acceptable solvate.

50. The composition as claimed in claim 48, further comprising an agent selected from an immunosuppressive agent, an anti-inflammatory agent, an antirheumatic agent, an antidyspilidemic agent, or any combination thereof.

51. The composition as claimed in claim 48, wherein the composition is in the form of a tablet, a capsule, a cachet, a powder, a granule, a solution, a suspension, an emulsion, a bolus, a lozenge, a suppository, a pessary, a tampon, a cream, a gel, a paste, a foam, a spray, an aerosol, a microcapsule, a liposome, a transdermal patch, a pastille, a paste, or a mouthwash.
